(19) United States
(12) Patent Application Publication
(10) Pub. No.: US 2008/0132484 A1
(43) Pub. Date: Jun. 5, 2008

(54) Method for Identifying Inhibitors Using a Homology Model of Polo-Like Kinase 1

(75) Inventors: Campbell McInnes, Dundee (GB); Janice McLachlan, Dundee (GB); Mokdad Mezna, Dundee (GB); Peter Martin Fischer, Angus (GB)

Correspondence Address: LAHIVE & COCKFIELD, LLP, ONE POST OFFICE SQUARE, BOSTON, MA, 02109-2127, US

(73) Assignee: CYCLACEL LIMITED, Dundee (GB)

(21) Appl. No.: 10/579,006

(22) PCT Filed: Nov. 12, 2004
(86) PCT No.: PCT/GB2004/004762

§ 371 Date: Nov. 15, 2007

(30) Foreign Application Priority Data
Nov. 12, 2003 (GB) 0326396.9

(51) Int. Cl. A61K 31/553; C12Q 1/48; A61K 31/366; A61K 31/505; C12N 5/00; G06T 15/00; G06F 17/00; C12N 9/12; A61P 35/00; A61K 31/352; G06F 19/00

(52) U.S. Cl. 514/211.08; 435/7.1; 435/4; 702/19; 514/453; 514/456; 514/256; 435/375; 435/194; 345/419; 707/100; 707/E17.001

(57) ABSTRACT

The present invention relates to a homology model for PLK, and the use thereof in assays for the identification of small molecule PLK modulators. The invention further relates to PLK modulators identified by said assays, and their use in the treatment of PLK-related disorders such as proliferative disorders.

The present invention relates to polo-like kinases (PLKs) and small molecule inhibitors thereof. More specifically, the invention relates to a method for designing and identifying small molecule inhibitors using a homology model for PLK.

### BACKGROUND TO THE INVENTION

The Polo-like kinase family consists of key cell cycle regulatory enzymes with integral roles in controlling entry into and progression through mitosis. Many tumour cells express high levels of PLK1 and are responsive to antisense oligonucleotides targeting this protein.

Initiation of mitosis requires activation of M-phase promoting factor (MPF), i.e. the complex between CDK1 and B-type cyclins [1]. The latter accumulate during the S and G2 phases of the cell cycle and promote the inhibitory phosphorylation of the MPF complex by WEE1, MIK1, and MYT1 kinases. At the end of the G2 phase, corresponding dephosphorylation by the dual-specificity phosphatase CDC25C triggers the activation of MPF [2]. In interphase, cyclin B localizes to the cytoplasm and becomes phosphorylated during prophase, followed by nuclear translocation. The nuclear accumulation of active MPF during prophase is thought to be important for initiating M-phase events [3]. However, nuclear MPF is kept inactive by WEE1 unless counteracted by CDC25C. The phosphatase CDC25C itself, localized to the cytoplasm during interphase, accumulates in the nucleus in prophase. The nuclear entry of both cyclin B and CDC25C are promoted through phosphorylation by PLK1 [4]. This kinase is thus an important regulator of M-phase initiation.

In humans, there exist three closely related polo-like kinases (PLKs) [5]. They contain a highly homologous N-terminal catalytic kinase domain and their C-termini contain two or three conserved regions, the polo boxes. The function of the polo boxes remains incompletely understood but polo box-dependent PLK1 activity is required for proper metaphase/anaphase transition and cytokinesis [6]. Of the three PLKs, PLK1 is the best characterized; it regulates a number of cell division cycle effects, including the onset of mitosis, DNA-damage checkpoint activation, regulation of the anaphase promoting complex, phosphorylation of the proteasome, and centrosome duplication and maturation. Mammalian PLK2 (also known as SNK) and PLK3 (also known as PRK and FNK) were originally shown to be immediate early gene products. PLK kinase activity appears to peak during late S and G2 phase. It is also activated during DNA damage checkpoint activation and severe oxidative stress. PLK3 also plays an important role in the regulation of microtubule dynamics and centrosome function in the cell and deregulated PLK3 expression results in cell cycle arrest and apoptosis [7]. PLK2 is the least-well understood homologue of the three PLKs. Both PLK2 and PLK3 may have additional important post-mitotic functions [8].

The fact that human PLKs regulate some fundamental aspects of mitosis was shown by anti-PLK1 antibody microinjection of human tumour cells [9]. This treatment had no effect on DNA replication but impaired cell division. Cells were arrested in mitosis and showed abnormal distribution of condensed chromatin and monoastral microtubules nucleated from duplicated but unseparated centrosomes. By contrast, non-immortalized human cells arrested as single, mononucleated cells in G2. Moreover, when PLK1 function was blocked through adenovirus-mediated delivery of a dominant-negative gene, tumour-selective apoptosis in many tumour cell lines was observed, whereas again normal epithelial cells, although arrested in mitosis, escaped the mitotic catastrophe seen in tumour cells [10]. PLK1 activity is thus necessary for the functional maturation of centrosomes in late G2/early prophase and subsequent establishment of a bipolar spindle. Furthermore, these results suggest the presence in normal cells of a centrosome-maturation checkpoint that is sensitive to PLK1 impairment. Depletion of cellular PLK1 through the small interfering RNA (siRNA) technique also confirmed that this protein is required for multiple mitotic processes and completion of cytokinesis [11]. A potential therapeutic rationale for PLK inhibition is also suggested by work with PLK1-specific antisense oligonucleotides, which were shown to induce growth inhibition in cancer cells both in vitro and in vivo [12]. Constitutive expression of PLK1 in mammalian cells was shown to lead to malignant transformation [13]. Furthermore, overexpression of PLK1 is frequently observed in human tumours and PLK1 expression is of prognostic value for patients suffering from various types of tumours [14-16].

Although the therapeutic potential of pharmacological PLK inhibition has been appreciated [17], very little has been reported to date concerning small-molecule PLK inhibitors that may be useful as drugs. The only characterized biochemical PLK1 inhibitor is scytonemin, a symmetric indolic marine natural product [18,19]. Scytonemin inhibits phosphorylation of CDC25C by recombinant PLK1 with an IC₅₀ value of about 2 μM (at an ATP concentration of 10 μM). Inhibition is apparently reversible and the mechanism with respect to ATP of mixed-competitive mode. Similar potency against other protein serine/threonine- and dual specificity cell-cycle kinases, including MYT1, CHK1, CDK1/cyclin B, and PKC, was observed. Scytonemin showed pronounced anti-proliferative effects on various human cell lines in vitro.

The present invention seeks to elucidate small molecule PLK inhibitors, and in particular, provides a method for designing and identifying such inhibitors. The invention also seeks to elucidate further information on the 3-dimensional structure of the PLK binding domain and the nature of the binding interactions between PLK and such small molecule inhibitors.

### STATEMENT OF INVENTION

The present invention relates to a homology model for PLK, and the use thereof in the identification of small molecule PLK inhibitors.

As used herein, the term “model” refers to a structural model such as a three dimensional (3D) structural model (or representation thereof) comprising PLK. Preferably, the model comprising PLK is built from all or a portion of the structure co-ordinates presented in Table 2. The homology model of the invention enables candidate compounds to be identified that bind spatially and preferentially to PLK, particularly to the active site of PLK.

Aspects of the invention are presented in the accompanying claims and are further described in the following paragraphs.

### DETAILED DESCRIPTION

### Assays Based on the PLK1 Homology Model

A first aspect of the invention relates to a method of screening for a modulator of PLK, wherein the method comprises using the structure co-ordinates of Table 2.

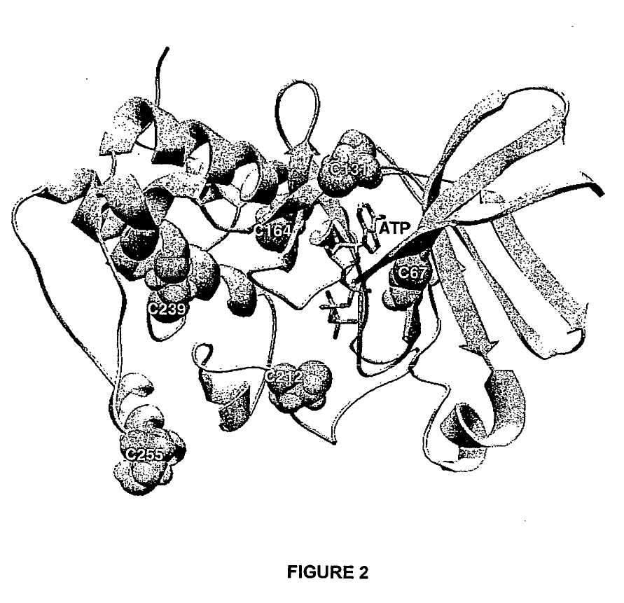

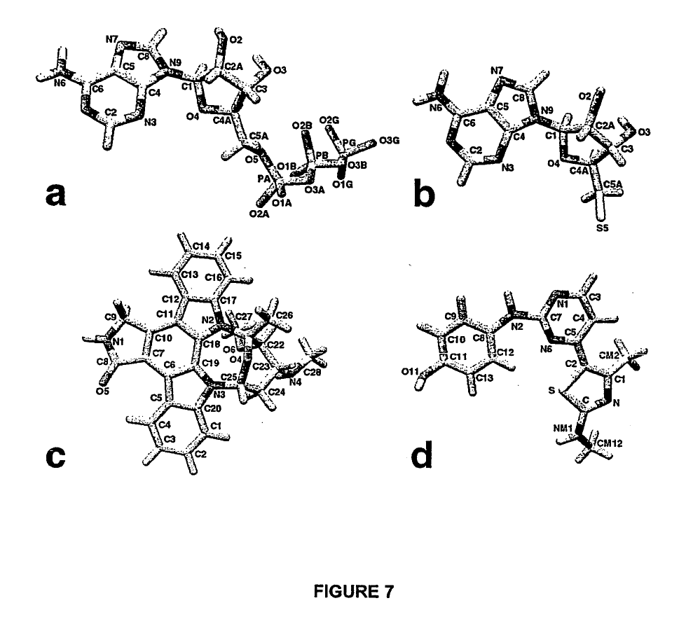

Since no experimental three-dimensional structures of PLK kinase domains are known, a PLK1 kinase domain homology model was constructed (Example 1). This model provides a plausible complex with the natural ligand ATP in the active site (FIG. 2), as well as with two non-selective ATP-competitive kinase inhibitors, which were also found to inhibit PLK1, namely staurosporine [32] (IC₅₀ w.r.t. PLK1=0.4 μM) and 4-[4-(4-methyl-2-methylamino-thiazol-5-yl)-pyrimidin-2-ylamino]-phenol [33] (IC₅₀ w.r.t. PLK1=4 μM) (FIG. 7).

Of particular interest in the PLK1 kinase domain structure are Cys⁶⁷ and Cys¹³³, both of which line the ATP binding site. Cys¹³³ is located in the so-called hinge region, which is present in many kinases, and connects the N- and C-terminal lobes of the kinase domain. Its side chain projects away from the ATP-binding pocket, although its backbone NH and CO functions are probably involved in H-bonding with the purine system of ATP. The side chain of Cys⁶⁷ on the PLK1 N-terminal lobe, on the other hand, points into the ATP-binding pocket and probably contributes directly to ATP binding via contacts with the ribose and/or triphosphate moieties. The position occupied by Cys⁶⁷ in PLK1 is usually occupied by valine in other kinases and there contributes van der Waals contacts to ATP binding. A second unusual residue, Phe¹⁸³, which is commonly leucine in other kinases, also makes significant contributions to ATP binding through interactions with the purine system. These two key differences strongly suggest that they can be exploited in the generation of ATP-competitive inhibitors selective for PLK1. The presence of Cys⁶⁷ in the pocket opens up the possibility that covalent or irreversible inhibitors could be developed.

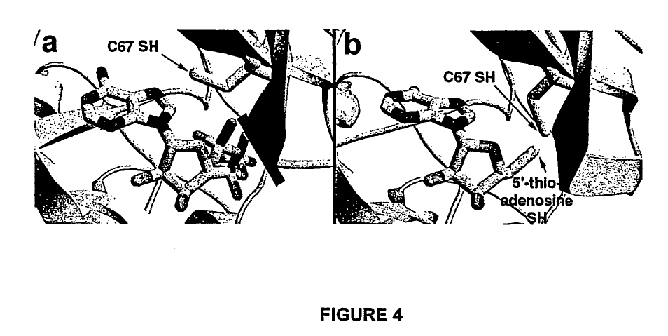

As discussed above, Cys⁶⁷ of PLK1 is of particular interest, since in the modelled PLK1-ATP complex structure it is positioned closely to the ribose ring of ATP (FIG. 4a). More specifically, a close contact between the Cys⁶⁷ thiol group and the 5′-O of the ribose portion of ATP is observed. A suitable adenosine-derived covalent inhibitor would thus be 5′-thioadenosine. Modelling (FIG. 4b) of this compound into the active site of PLK1 suggests that a simple rotation of the C^{α}—C^{β} bond of Cys⁶⁷ should accommodate this inhibitor in such a way as to bring the sulfur atoms of Cys⁶⁷ and 5′-thioadenosine into disulfide-bonding distance without large perturbations of the bound adenine portion.

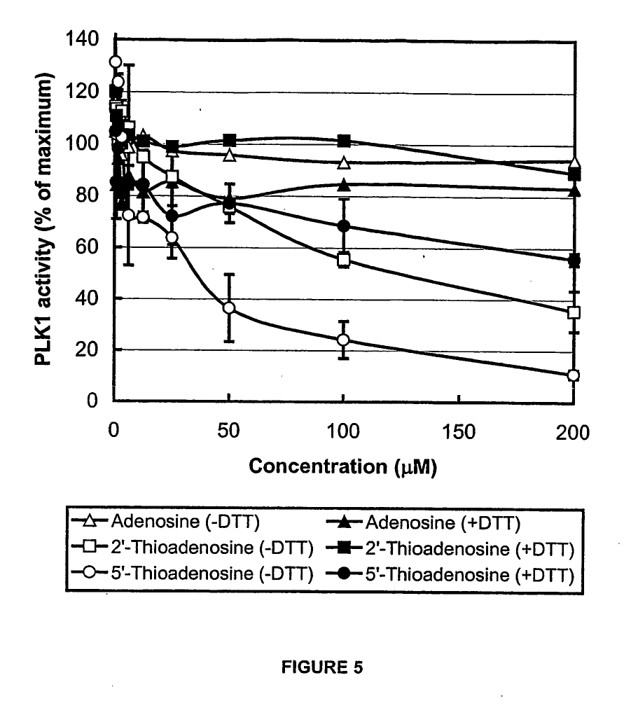

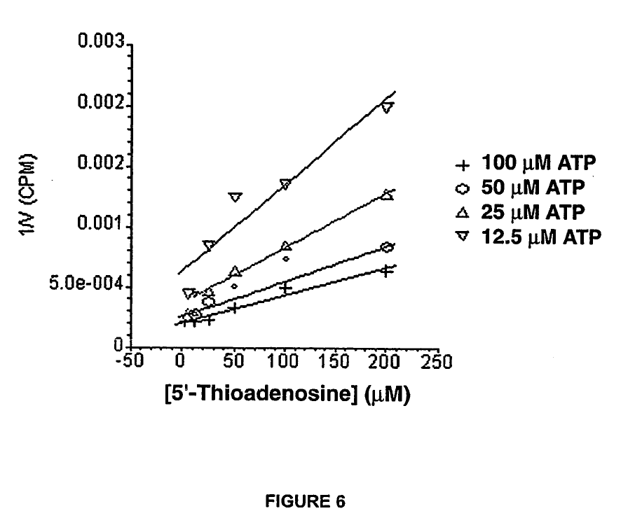

In order to test the hypothesis that Cys⁶⁷ may indeed be involved in ATP binding by PLK1, the effect of non-specific thiol modifying agents such as thimerosal [34], N-ethylmaleimide, and iodoacetamide on PLK1 enzymatic activity was studied. All these reagents were found to inhibit CDC25C phosphorylation by PLK1 to some extent, indicating the involvement of Cys residues in enzymatic activity. The fact that such inhibition could be abolished in the presence of an excess of the reducing agent dithiothreitol, which specifically reduces disulfide bonds and competes with Cys thiol groups for thiol modifying agents [35], is consistent with this notion (Example 8). Adenosine derivatives were studied next (FIG. 5). Unmodified adenosine did not inhibit PLK1 function at concentrations up to 0.2 mM, whereas 2′- and 5′-thioadenosines did. 5-Thioadenosine was about 3-fold more potent than its analogue 2′-thioadenosine, supporting the hypothesis that the 5′-OH of the ribose ring is better oriented to react with Cys⁶⁷. Again a lack of inhibition was observed in the presence of DTT. Kinetic analysis of PLK1 inhibition (Example 14) showed that with e.g. 5′-thioadenosine (FIG. 6) this was dependent on ATP concentration but not competitive with ATP as would be the case for a reversible competitive ATP antagonist. The effects of the above thiol modifying reagents on a closely related serine/threonine kinase were also studied. Casein kinase II (CKII) was selected based on its sensitivity to certain inhibitors [36], e.g. wortmannin and LY294002 [37], which were also found to be capable of inhibiting PLK1 (IC₅₀ with respect to PLK1 of <0.1 μM and <5 μM, respectively). No significant inhibition of CKII enzymatic activity was observed at concentrations up to 0.2 mM with thimerosal, N-ethylmaleimide, iodoacetamide, adenosine, 2′-thioadenosine, or 5′-thioadenosine using the assay described in Example 4.

In summary, these results suggest that PLK-specific ATP antagonists can be developed that derive their potency and PLK selectivity from a combination of non-covalent binding to the unique ATP-binding pocket of PLK1 and covalent binding to the Cys⁶⁷ thiol group.

Observations from Modelled Structures of PLK1 Inhibitors

Studies were also carried out on purvalanol A and various flavonoid molecules. Further details of these studies are outlined in the accompanying examples section

The interactions of the potent Cdk2 inhibitors, staurosporine and purvalanol A with the PLK1 ATP cavity reveal why both of these inhibitors are non-selective for the two kinases. Staurosporine makes similar H-bond and van der Waals contacts in both structures, however is rotated by about 30° in the PLK1 structure with regards to Cdk2. The non-bonded energies for this inhibitor indicate a rough correlation with the observed IC50's as shown by the ludi energetic scores of 456 (H-bond 131, lipophilic 307) with PLK1 and 726 (H-bond 230, lipophilic 478) for Cdk2 (higher value indicates more favourable binding). Analysis of these scores indicates that the less favourable H-bond interactions in the PLK1 context contribute significantly to the lower inhibition. Unfavourable hydrophobic contacts result in rotation of the inhibitor and less optimal geometry of the hinge H-bonds.

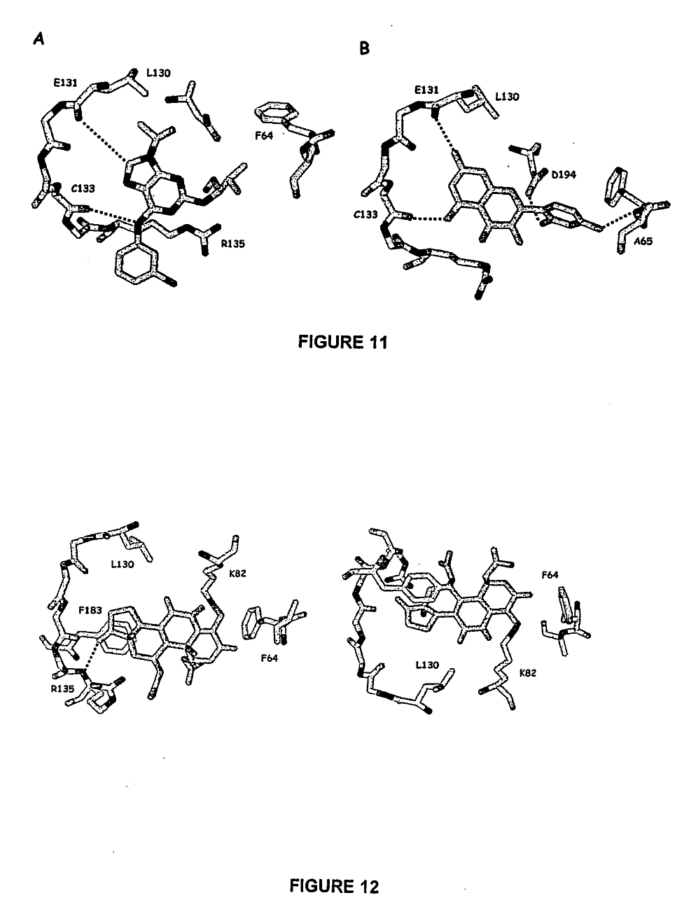

Purvalanol A also makes similar contacts with both enzymes with H-bonds from the aniline N, a H-bond like interaction from the purine C, and favourable contacts with the L130 “gatekeeper” residue (FIG. 11A) and thus demonstrates the structural basis for binding to both kinases. Again less optimal van der Waals contacts in the PLK1 case result in less optimal H-bond interactions with the interdomain connecting hinge.

Molecular docking of morin hydrate, the most potent in the flavonoid series, with the PLK1 homology model gives significant insight into the interactions of this compound with ATP binding site. A binding inode that is consistent with known kinase inhibitor interactions was observed and the inhibitor makes numerous van der Waals and H-bond contacts (FIG. 11B). These include the two hydroxyls on the aromatic section of the flavonoid ring acting as H-bond donors to the carbonyls of C133 and E131. The flavonoid ring makes van der Waals interactions with L130, the gatekeeper residue and the 1,3 substituted catechol ring, makes H-bond contacts to the sidechain of D194 and the backbone amide of A65. Analysis of the activities of the other structural homologues in this series (Table 13) indicates that this observed pose of morin bound to PLK1 is consistent with the structure-activity relationship. Datescetin, which is identical to morin except lacks the ortho-hydroxyl is inactive suggesting a significant role for the 3′-hyrdoxyl. Quercetin however has partial activity and contains the 3-hydroxyl but has no 1 hydroxyl. None of the other analogues in the series contains both the 1 and 3 position hydroxyls and therefore explains their loss of activity. The importance of both hydroxyl suggested by the SAR data is confirmed by the energetic contributions of H-bond interactions of these groups to the binding to the ATP cleft as shown in the docked structure. Placement of the hydroxyls on other positions in the ring would not allow optimal H-bond formation and thus indicates a structural rationale for their lack of potency in inhibiting PLK1 kinase activity.

Overall the postulated binding modes of the identified PLK1 inhibitors are energetically reasonable, consistent with observed structure-activity relationships and with the interactions of known kinase inhibitors. These results are therefore useful in design and synthesis of analogues of these structures which are optimized for PLK1 inhibition and selectivity.

### Implications of the Discovery of Potent PLK1 Kinase Inhibitors

While the role of Cdks in the regulation of the cell cycle is very well established and comprehensively studied, PLKs clearly orchestrate events of the whole cell cycle [5]. However, very little is known about the physiological substrates for this class of enzymes. During mitosis and cytokinesis, PLKs are reported to associate with various structures involved in spindle formation and assembly including the centrosomes and kinetochores. Recent reports demonstrated the link between PLK1 in particular with microtubule and microtubule-associated functions. Thus it is of a paramount importance to identify all the physiological substrates as well as all the posttranslational modifying enzymes for PLKs in order to understand their exact role in the cell cycle.

Over the last five years considerable efforts have been made in order to investigate the significance of PLK1 deregulation in the human health. A plethora of information is available strongly suggesting the oncogenicity of aberrantly expressed PLK1. As of yet, there is no direct evidence to prove the tumourogenic effects of the deregulated PLK1 activity and the challenge is therefore to determine the exact functions of PLK1 and subsequently determine the best routes for modulating this activity.

In the present study we sought to identify inhibitors of PLK1 in vitro and which could potentially applied to determine the cellular phenotype and consequences of reducing PLK1 kinase activity. The only inhibitor reported prior to this study is Scytonemin, a symmetric indolic marine natural product that is a micromolar non-specific ATP competitor [48]. Here we show for the first time that wortmannin is a very potent inhibitor of PLK1 while staurosporine and purvalanol A showed moderate inhibition.

Detailed examination indicated that while staurosporine inhibited PLK1 activity in an ATP dependent fashion, wortmannin inhibition was totally independent of ATP suggesting a different mode of binding. These results suggest a similar mode of inhibition to that reported previously for Phosphatidylinositol 3′OH kinase where wortmannin forms a covalent interaction with a Lysine residue (K833) positioned in the ATP binding pocket of the enzyme. Secondary structure analysis and homology modelling of the catalytic domain of PLK1 revealed the existence of a lysine residue (K82) projecting into the ATP binding cleft. It was therefore hypothesised that wortmannin covalently modifies this Lys residue and prevents ATP binding. It should be noted that previous reports clearly demonstrated that a single point mutation of K82 completely abolished the kinase activity of PLK1 since it required in the phosphotransfer step [49]. The observation from molecular modelling that the inhibitor docks in an orientation compatible with covalent interaction with K82, tolerates formation of the bond and energy minimisation without structural distortion and interacts similarly to the PI3 kinase binding mode additionally confirms the validity of the homology structure. The high plausibility of this model therefore strongly supports the experimental data indicating irreversible binding of Wortmannin and is consistent with the hypothesis for reactivity with K82.

In addition to the identification of wortmannin, staurosporine, and purvalanol A as inhibitors of PLK1 kinase, the described flavonoid compounds are potential tool compounds for in vitro cellular screening in order to determine a phenotype of PLK1 inhibition. They also represent starting points for designing potent and selective small molecule inhibitors of this enzyme.

Preferred embodiments of the invention will now be described.

In one preferred embodiment of the invention, the method comprises the steps of:
(a) providing at least a portion of the structure co-ordinates of Table 2;
(b) employing at least a portion of the structure co-ordinates of Table 2 to design or select or synthesise a putative modulator of PLK;
(c) contacting the putative modulator of PLK with PLK or a mutant, variant, homologue, derivative or fragment thereof, in the presence of a substrate of PLK; and
(d) determining whether said putative modulator of PLK modulates PLK.

In a preferred embodiment, at least a portion of the structure co-ordinates of Table 2 and/or the putative modulator of PLK and/or the substrate are provided on a machine-readable data storage medium comprising a data storage material encoded with machine readable data.

In a preferred embodiment, the putative modulator of PLK is selected from a library of compounds. Preferably, the library is an in silico library. Suitable in silico libraries will be familiar to those skilled in the art, and include the Available Chemical Directory (MDL Inc), the Derwent World Drug Index (WDI), BioByteMasterFile, the National Cancer Institute database (NCI), and the Maybridge catalogue.

In another preferred embodiment, the putative modulator of PLK is selected from a database.

In another preferred embodiment, the putative modulator of PLK is designed de novo.

In yet another preferred embodiment, the putative modulator of PLK is designed from a known PLK modulator.

Preferably, the design or selection of the putative modulator of PLK is performed in conjunction with computer modelling.

In one particularly preferred embodiment, the putative modulator of PLK inhibits PLK activity.

More preferably, the PLK is PLK1.

In a further preferred embodiment, the putative modulator of PLK is useful in the prevention and/or treatment of a PLK related disorder.

Even more preferably, the PLK related disorder is a proliferative disorder.

More preferably still, the proliferative disorder is selected from cancer, leukemia, glomerulonephritis, rheumatoid arthritis, psoriasis and chronic obstructive pulmonary disorder.

A second aspect of the invention relates to an assay for a candidate compound capable of modulating PLK, said assay comprising the steps of:
(a) contacting said candidate compound with PLK;
(b) detecting whether said candidate compound forms associations with one or more amino acid residues corresponding to PLK amino acid residues L59, G60, A65, C67, A80, K82, L130, E131, C133, R135, F183 and D194.

In one preferred embodiment, said candidate compound is selected by performing rational drug design with a 3-dimensional model of PLK in conjunction with computer modelling.

In an even more preferred embodiment, the assay comprises detecting whether said candidate compound forms an association with the amino acid residue corresponding to PLK amino acid residue C67.

A third aspect of the invention relates to the use of a compound selected from the following:
(i) 5′-thioadenosine, or a derivative thereof;
(ii) staurosporine, wortmannin, purvalanol A, LY294002, quercetin, morin hydrate or derivatives thereof; and
(iii) 4-[4-(4-methyl-2-methylaminothiazol-5-yl)-pyrimidin-2-ylamino]-phenol; 4-[4-(2,4-dimethyl-thiazol-5-yl)-pyrimidin-2-ylamino]-phenol or 4-[4-(2-amino-4-methyl-thiazol-5-yl)-pyrimidin-2-ylamino]-phenol;
   or a pharmaceutically acceptable salt thereof, in an assay for identifying candidate compounds capable of modulating PLK.

Preferably, the compound of (ii) is staurosporine, wortmannin, purvalanol A, LY294002, or morin hydrate. More preferably, the compound of (ii) is staurosporine, wortmannin, purvalanol A, even more preferably staurosporine or wortmannin.

Preferably, the assay is a competitive binding assay.

More preferably, the assay comprises contacting a candidate compound with PLK in the presence of a compound selected from:
(i) 5′-thioadenosine, or a derivative thereof;
(ii) staurosporine, wortmannin, purvalanol A, LY294002, quercetin, morin hydrate, or derivatives thereof; and
(iii) 4-[4-(4-methyl-2-methylaminothiazol-5-yl)-pyrimidin-2-ylamino]-phenol, 4-[4-(2,4-dimethyl-thiazol-5-yl)-pyrimidin-2-ylamino]-phenol or 4-[4-(2-amino-4-methyl-thiazol-5-yl)-pyrimidin-2-ylamino]-phenol;
   or a pharmaceutically acceptable salt thereof, and detecting any change in the interaction between (i), (ii) or (iii) and PLK.

Another aspect of the invention relates to a computer for producing a three-dimensional representation of PLK wherein said computer comprises:
(a) a computer-readable data storage medium comprising a data storage material encoded with computer-readable data, wherein said data comprises the structure co-ordinates of Table 2;
(b) a working memory for storing instructions for processing said computer-readable data;
(c) a central-processing unit coupled to said working memory and to said computer-readable data storage medium for processing said computer-machine readable data into said three-dimensional representation; and
(d) a display coupled to said central-processing unit for displaying said three-dimensional representation.

Another aspect of the invention relates to a machine-readable data storage medium comprising a data storage material encoded with machine readable data, wherein the data is defined by at least a portion of the structure co-ordinates of Table 2.

A further aspect of the invention relates to the use of the above-described computer or machine readable data storage medium to predict the structure and/or function of potential modulators of PLK.

Another aspect relates to the use of at least a portion of the structure co-ordinates of Table 2 to screen for modulators of PLK.

A further aspect relates to the use of at least a portion of the structure co-ordinates of Table 2 to solve the structure of the crystalline form of any other protein with significant amino acid sequence homology to any functional domain of PLK.

Preferably, the structure of the crystalline form of any other protein with significant amino acid sequence homology to any functional domain of PLK is solved using molecular replacement.

Yet another aspect of the invention relates to the use of at least a portion of the structure co-ordinates of Table 2 in molecular design techniques to design, select and synthesise modulators of PLK.

A further aspect of the invention relates to the use of at least a portion of the structure co-ordinates of Table 2 in the development of compounds that can isomerise to reaction intermediates in the chemical reaction of a substrate or other compound that binds to PLK.

Another aspect of the invention relates to the use of at least a portion of the structure co-ordinates of Table 2 to screen small molecule databases for chemical entities or compounds that modulate PLK.

### PLK Modulators

A further aspect of the invention relates to a PLK modulator identified by the above-described method, or a candidate compound identified by the above-described assay.

Preferably, the PLK modulator or candidate compound of the invention inhibits PLK activity.

More preferably, the PLK modulator or candidate compound of the invention is capable of forming a covalent bond with the amino acid residue corresponding to PLK amino acid residue C67.

More preferably still, the PLK modulator or candidate compound of the invention is capable of forming a disulfide bond with the thiol group of the amino acid residue corresponding to PLK amino acid residue C67.

In one preferred embodiment, the PLK modulator or candidate compound of the invention is an irreversible antagonist.

The present invention permits the use of molecular design techniques to design, select and synthesise chemical entities and compounds, including PLK modulating compounds, capable of binding to PLK, in whole or in part.

By way of example, the structure co-ordinates of Table 2 may be used to design compounds that bind to PLK and may alter the physical properties of the compounds (eg. solubility) or PLK itself. This invention may be used to design compounds that act as modulators, such as competitive inhibitors—of PLK by binding to all or a portion of the active site of PLK Compounds may also be designed that act as non-competitive inhibitors of PLK. These non-competitive inhibitors may bind to all or a portion of PLK already bound to its substrate and may be more potent and specific than known PLK inhibitors that compete only for the PLK active site. Similarly, non-competitive inhibitors that bind to and inhibit PLK whether or not it is bound to another chemical entity may be designed using the structure co-ordinates of PLK described herein.

The present invention may also allow the development of compounds that can isomerise to reaction intermediates in the chemical reaction of a substrate or other compound that binds to PLK. Thus, the time-dependent analysis of structural changes in PLK during its interaction with other molecules may be performed. The reaction intermediates of PLK may also be deduced from the reaction product in co-complex with PLK. Such information is especially useful to design improved analogues of known PLK modulators or to design new PLK modulators based on the reaction intermediates of the PLK enzyme and PLK-modulator complex. This may provide a new route for designing PLK modulators with high specificity and stability. Preferably, this provides a new route for designing PLK modulators with high specificity, high stability and low toxicity.

Small molecule databases or candidate compounds may be screened for chemical entities or compounds that can bind in whole, or in part, to PLK. Thus, in a preferred embodiment, the putative PLK modulator is from a library of compounds or a database. In this screening, the quality of fit of such entities or compounds to the binding site may be judged by various methods—such as shape complementarity or estimated interaction energy (Meng, E. C. et al., J. Comp. Chem., 13, pp. 505-524 (1992)).

The structure co-ordinates of Table 2, or portions thereof may also be useful in solving the structure of crystal forms of PLK. They may also be used to solve the structure of PLK mutants, PLK variants, PLK homologues, PLK derivatives, PLK fragments and PLK complexes.

Preferably, the structure co-ordinates of Table 2 may be used to solve the structure of the crystalline form of proteins having significant amino acid sequence homology to any functional domain of PLK. By way of example, molecular replacement may be used. In this method, the unknown crystal structure, whether it is a crystal form of PLK, a PLK mutant, a PLK variant, a PLK homologue (eg. another protein with significant amino acid sequence homology to any functional domain of PLK), a PLK derivative, a PLK fragment or a PLK co-complex may be determined using the PLK structure co-ordinates of the present invention. This method will provide a more accurate structural form for the unknown crystal more quickly and efficiently than attempting to determine such information ab initio.

In a preferred embodiment of the present invention, the PLK crystal of unknown structure further comprises an entity bound to the PLK protein or a portion thereof, for example, an entity that is an inhibitor of PLK.

The crystal structures of such complexes may be solved by molecular replacement or in combination with MAD (Multiwavelength Anomalous Dispersion) and/or MIRAS (Multiple Isomorphous Replacement with Anomalous Scattering) procedures—and compared with that of wild-type PLK. Potential sites for modification within the binding sites of the enzyme may thus be identified. This information provides an additional tool for determining the most efficient binding interactions, for example, increased hydrophobic interactions, between PLK. and a chemical entity or compound. The structures and complexes of PLK may be refined using computer software—such as X-PLOR (Meth. Enzymol., vol. 114 & 115, H. W. Wyckoff et al., eds., Academic Press (1985)), MLPHARE (Collaborative computational project Number 4. The CCP4 Suite: Programs for Protein Crystallography (1994) Acta Crystallogr. D 50, 760-763) and SHARP [De La Fortelle, E. & Bricogne, G. Maximum-likelihood heavy-atom parameters refinement in the MIR and MAD methods (1997) Methods Enzymol. 276, 472-494). Preferably, the complexes are refined using the program CNS (Brünger et al. (1998) Acta Crystallogr. D 54, 905-921). During the final stages of refinement water molecules, ions and inhibitor molecules may be inserted in the structure. This information may thus be used to optimise known classes of PLK modulators, eg. PLK inhibitors, and more importantly, to design and synthesise novel classes of PLK modulators.

The overall figure of merit may be improved by iterative solvent flattening, phase combination and phase extension with the program SOLOMON [Abrahams, J. P. & Leslie, A. G. W. Methods used in structure determination of bovine mitochondrial F1 ATPase. (1996) Acta Crystallogr. D 52, 110-119].

The structure co-ordinates of the homology model of the present invention may also facilitate the identification of related proteins or enzymes analogous to PLK in function, structure or both, thereby further leading to novel therapeutic modes for treating or preventing PLK related diseases.

The design of compounds that bind to or modulate PLK according to the present invention generally involves consideration of two factors. Firstly, the compound must be capable of physically and structurally associating with PLK. Non-covalent molecular interactions important in the association of PLK with its substrate may include hydrogen bonding, van der Waals and hydrophobic interactions. Secondly, the compound must be able to assume a conformation that allows it to associate with PLK. Although certain portions of the compound may not directly participate in the association with PLK, those portions may still influence the overall conformation of the molecule. This may have a significant impact on potency. Such conformational requirements include the overall three-dimensional structure and orientation of the chemical entity or compound in relation to all or a portion of a binding site of PLK, or the spacing between functional groups of a compound comprising several chemical entities that directly interact with PLK.

The potential modulating or binding effect of a chemical compound on PLK may be analysed prior to its actual synthesis and testing by the use of computer modelling techniques. If the theoretical structure of the given compound suggests insufficient interaction and association with PLK, then synthesis and testing of the compound may be obviated. However, if computer modelling indicates a strong interaction, the molecule may be synthesised and tested for its ability to bind to PLK and modulate (eg. inhibit) using the fluorescent substrate assay of Thornberry et al. (2000) Methods Enzymol. 322, pp 100-110. In this manner, synthesis of inactive compounds may be avoided.

A modulating or other binding compound of PLK may be computationally evaluated and designed by means of a series of steps in which chemical entities or candidate compounds are screened and selected for their ability to associate with PLK.

A person skilled in the art may use one of several methods to screen chemical entities or candidate compounds for their ability to associate with PLK and more particularly with the individual binding sites of PLK. This process may begin by visual inspection of, for example, the active site on the computer screen based on the PLK co-ordinates of the present invention. Selected chemical entities or candidate compounds may then be positioned in a variety of orientations, or docked, with PLK. Docking may be accomplished using software such as Quanta and Sybyl, followed by energy minimisation and molecular dynamics with standard molecular mechanics force fields—such as CHARMM and AMBER.

Specialised computer programs may also assist in the process of selecting chemical entities or candidate compounds. These include but are not limited to MCSS (Miranker and Karplus (1991) Proteins: Structure, Function and Genetics, 11, pp. 29-34); GRID (Goodford (1985) J. Med. Chem., 28, pp. 849-857) and AUTODOCK (Goodsell and Olsen (1990), Proteins: Structure. Function, and Genetics, 8, pp. 195-202.

Once suitable chemical entities or candidate compounds have been selected, they may be assembled into a single compound, such as a PLK modulator. Assembly may proceed by visual inspection of the relationship of the chemical entities or candidate compounds in relation to the structure co-ordinates of PLK. This may be followed by manual model building using software—such as Quanta, Sybyl, 0, HOOK or CAVEAT [Jones, T. A., Zou, J. Y., Cowan, S. W. & Kjeldgaard, M. Improved methods for building protein models in electron density maps and the location of errors in these models (1991) Acta Crystallogr. A 47, 110-119].

Refinement of the model may be carried out using the program CNS [Brünger, A. T. et al. Crystallography & NMR System: A new software suite for macromolecular structure determination. (1998) Acta Crystallogr. D 54, 905-921].

Various programs may be used by a skilled person to connect the individual chemical entities or candidate compounds, such as 3D Database systems (Martin (1992) J. Med. Chem., 35, pp. 2145-2154) and CAVEAT (Bartlett et al. (1989) Royal Chem. Soc. 78, pp. 182-196).

Rather than build a PLK inhibitor one chemical entity at a time, modulating or other PLK binding compounds may be designed as a whole or de novo using either an empty binding site or optionally including some portion(s) of a known inhibitor(s). Such compounds may be designed using programs that may include but are not limited to LEGEND (Nishibata and Itai (1991) Tetrahedron, 47, p. 8985) and LUDI (Bohm (1992) J. Comp. Aid. Molec. Design, 6, pp. 61-78).

Other molecular modelling techniques may also be employed in accordance with this invention—such as those described by Cohen et al., J. Med. Chem., 33, pp. 883-894 (1990); Navia and Murcko (1992) Current Opinions in Structural Biology, 2, pp. 202-210 (1992).

Once a compound has been designed or selected by the above methods, the efficiency with which that compound may bind to PLK may be computationally evaluated. Specific computer software may be used to evaluate the efficiency of binding (eg. to evaluate compound deformation energy and electrostatic interaction), such as QUANTA/CHARMM (Accelrys Inc., USA) and Insight II/Discover (Biosym Technologies Inc., San Diego, Calif., USA). These programs may be implemented, for instance, using a suitable workstation. Other hardware systems and software packages will be known to those persons skilled in the art.

Once a PLK-modulating compound has been selected or designed, as described above, substitutions may be made (eg. in atoms or side groups) to improve or modify the binding properties. The substitutions may be conservative ie. the replacement group may have approximately the same size, shape, hydrophobicity and charge as the original group. Such substituted chemical compounds may then be analysed for efficiency of binding to PLK by the same computer methods described above.

Candidate compounds and modulators of PLK etc. which are identified using the methods of the present invention may be screened in assays. Screening can be, for example in vitro, in cell culture, and/or in vivo. Biological screening assays preferably centre on activity-based response models, binding assays (which measure how well a compound binds), and bacterial, yeast and animal cell lines (which measure the biological effect of a compound in a cell). The assays can be automated for high capacity-high throughput screening (HTS) in which large numbers of compounds can be tested to identify compounds with the desired activity.

Current screening technologies are described in Handbook of Drug Screening, edited by Ramakrishna Seethala, Prabhavathi B. Fernandes. New York, N.Y., Marcel Dekker, (2001).

### Modulating PLK

As herein, the term “modulating” or “modulates” refers to preventing, suppressing, inhibiting, alleviating, restorating, elevating, increasing or otherwise affecting PLK.

The term “PLK modulator” may refer to a single entity or a combination of entities.

The PLK modulator may be an antagonist or an agonist of PLK.

As used herein, the term “agonist” means any entity, which is capable of interacting (eg. binding) with PLK and which is capable of increasing a proportion of the PLK that is in an active form, resulting in an increased biological response.

As used herein, the term “antagonist” means any entity, which is capable of interacting (eg. binding) with PLK and which is capable of decreasing (eg. inhibiting) a proportion of the PLK that is in an active form, resulting in a decreased biological response.

Preferably, the PLK modulators of the present invention are antagonists of PLK.

The modulator of PLK may be an organic compound or other chemical. The modulator of PLK may be a compound, which is obtainable from or produced by any suitable source, whether natural or artificial. The modulator of PLK may be an amino acid molecule, a polypeptide, or a chemical derivative thereof, or a combination thereof. The modulator of PLK may even be a polynucleotide molecule, which may be a sense or an anti-sense molecule. The modulator of PLK may even be an antibody.

The modulator of PLK may be designed or obtained from a library of compounds, which may comprise peptides, as well as other compounds, such as small organic molecules.

By way of example, the modulator of PLK may be a natural substance, a biological macromolecule, or an extract made from biological materials such as bacteria, fungi, or animal (particularly mammalian) cells or tissues, an organic or an inorganic molecule, a synthetic agent, a semi-synthetic agent, a structural or functional mimetic, a peptide, a peptidomimetic, a derivatised agent, a peptide cleaved from a whole protein, or a peptide synthesised synthetically (such as, by way of example, either using a peptide synthesiser or by recombinant techniques or combinations thereof, a recombinant agent, an antibody, a natural or a non-natural agent, a fusion protein or equivalent thereof and mutants, derivatives or combinations thereof).

Typically, the modulator of PLK will be an organic compound. Typically, the organic compounds will comprise two or more hydrocarbyl groups. Here, the term “hydrocarbyl group” means a group comprising at least C and H and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. For some applications, preferably the modulator of PLK comprises at least one cyclic group. The cyclic group may be a polycyclic group, such as a non-fused polycyclic group. For some applications, the modulator of PLK comprises at least the one of said cyclic groups linked to another hydrocarbyl group.

The modulator of PLK may contain halo groups, for example, fluoro, chloro, bromo or iodo groups, or one or more of alkyl, alkoxy, alkenyl, alkylene and alkenylene groups, each of which may be branched or unbranched.

The modulator of PLK may be a structurally novel modulator of PLK, or may be an analogue of a known modulator of PLK.

Preferably, the PLK modulators have improved properties over those previously available, for example, fewer side effects.

The modulator of PLK may be a mimetic, or may be chemically modified.

The modulator of PLK may be capable of displaying other therapeutic properties.

The modulator of PLK may be used in combination with one or more other pharmaceutically active agents. If combinations of active agents are administered, then they may be administered simultaneously, separately or sequentially.

### Candidate Compounds

As used herein, the term “candidate compound” includes, but is not limited to, a compound which may be obtainable from or produced by any suitable source, whether natural or not.

The candidate compound may be designed or obtained from a library of compounds, which may comprise peptides, as well as other compounds, such as small organic molecules and particularly new lead compounds. By way of example, the candidate compound may be a natural substance, a biological macromolecule, or an extract made from biological materials—such as bacteria, fingi, or animal (particularly mammalian) cells or tissues, an organic or an inorganic molecule, a synthetic candidate compound, a semi-synthetic candidate compound, a structural or functional mimetic, a peptide, a peptidomimetic, a derivatised candidate compound, a peptide cleaved from a whole protein, or a peptide synthesised synthetically, for example, either using a peptide synthesiser or by recombinant techniques or combinations thereof, a recombinant candidate compound, a natural or a non-natural candidate compound, a fusion protein or equivalent thereof and mutants, derivatives or combinations thereof. The candidate compound may even be a compound that is a modulator of PLK, such as a known inhibitor of PLK, that has been modified in some way eg. by recombinant DNA techniques or chemical synthesis techniques.

Typically, the candidate compound will be prepared by recombinant DNA techniques and/or chemical synthesis techniques.

Once a candidate compound capable of interacting PLK has been identified, further steps may be carried out to select and/or to modify the candidate compounds and/or to modify existing compounds, such that they are able to modulate PLK.

In one aspect, the modulator of PLK may act as a model (for example, a template) for the development of other compounds.

A further aspect relates to the use of candidate compounds or PLK modulators identified by the assays and methods of the invention in one or more model systems, for example, in a biological model, a disease model, or a model for PLK inhibition. Such models may be used for research purposes and for elucidating further details of the biological, physicochemical, pharmacological and/or pharmacokinetic activity of a particular candidate compound. By way of example, the candidate compounds or PLK modulators of the present invention may be used in biological models or systems in which the cell cycle is known to be of particular significance, e.g. in models relating to cell fertilization, especially in animals.

### Mimetic

As used herein, the term “mimetic” relates to any chemical which includes, but is not limited to, a peptide, polypeptide, antibody or other organic chemical which has the same qualitative activity or effect as a known compound. That is, the mimetic is a functional equivalent of a known compound.

### Chemical Synthesis Methods

Preferably, the modulator of PLK of the present invention may be prepared by chemical synthesis techniques.

It will be apparent to those skilled in the art that sensitive functional groups may need to be protected and deprotected during synthesis of a compound of the invention. This may be achieved by conventional techniques, for example as described in “Protective Groups in Organic Synthesis” by T W Greene and P G M Wuts, John Wiley and Sons Inc. (1991), and by P. J. Kocienski, in “Protecting Groups”, Georg Thieme Verlag (1994).

It is possible during some of the reactions that any stereocentres present could, under certain conditions, be racemised, for example if a base is used in a reaction with a substrate having an having an optical centre comprising a base-sensitive group. This is possible during e.g. a guanylation step. It should be possible to circumvent potential problems such as this by choice of reaction sequence, conditions, reagents, protection/deprotection regimes, etc. as is well-known in the art.

The compounds and salts may be separated and purified by conventional methods.

Separation of diastereomers may be achieved by conventional techniques, e.g. by fractional crystallisation, chromatography or H.P.L.C. of a stereoisomeric mixture of a compounds or suitable salts or derivatives thereof. An individual enantiomer of a compound may also be prepared from a corresponding optically pure intermediate or by resolution, such as by H.P.L.C. of the corresponding racemate using a suitable chiral support or by fractional crystallisation of the diastereomeric salts formed by reaction of the corresponding racemate with a suitably optically active acid or base.

PLK, modulators of PLK or variants, homologues, derivatives, fragments or mimetics thereof may be produced using chemical methods to synthesise the PLK or the modulator of PLK in whole or in part. For example, a PLK peptide or a modulator of PLK that is a peptide can be synthesised by solid phase techniques, cleaved from the resin, and purified by preparative high performance liquid chromatography (e.g., Creighton (1983) Proteins Structures And Molecular Principles, WH Freeman and Co, New York N.Y.). The composition of the synthetic peptides may be confirmed by amino acid analysis or sequencing (e.g., the Edman degradation procedure; Creighton, supra).

Synthesis of peptides (or variants, homologues, derivatives, fragments or mimetics thereof) may be performed using various solid-phase techniques (Roberge J Y et al (1995) Science 269: 202-204) and automated synthesis may be achieved, for example, using the ABI 43 1 A Peptide Synthesizer (Perkin Elmer) in accordance with the instructions provided by the manufacturer. Additionally, the amino acid sequences comprising the modulator of PLK, may be altered during direct synthesis and/or combined using chemical methods with a sequence from other subunits, or any part thereof, to produce a variant modulator of PLK.

### Chemical Modification

In one embodiment, the modulator of PLK may be a chemically modified modulator of PLK. The chemical modification of a modulator of PLK may either enhance or reduce interactions between the modulator of PLK and the target, such as hydrogen bonding interactions, charge interactions, hydrophobic interactions, van der Waals interactions or dipole interactions.

### Process

Another aspect of the invention relates to a process comprising the steps of:
(a) performing the method according to the invention, or an assay according to the invention;
(b) identifying one or more modulators of PLK; and
(c) preparing a quantity of said one or more PLK modulators.

A further aspect of the invention relates to a process comprising the steps of:
(a) performing the method according to the invention, or an assay according to the invention;
(b) identifying one or more PLK modulators; and
(c) preparing a pharmaceutical composition comprising said one or more identified PLK modulators.

A further aspect relates to a process comprising the steps of:
(a) performing the method according to the invention, or an assay according to the invention;
(b) identifying one or more PLK modulators;
(c) modifying said one or more PLK modulators; and
(d) optionally preparing a pharmaceutical composition comprising said one or more PLK modulators.

### Pharmaceutical Compositions

Another aspect of the invention relates to a pharmaceutical composition comprising a PLK modulator or candidate compound of the invention and a pharmaceutically acceptable carrier, diluent, excipient or adjuvant or any combination thereof. Even though the PLK modulators or candidate compounds (including their pharmaceutically acceptable salts, esters and pharmaceutically acceptable solvates) can be administered alone, they will generally be administered in admixture with a pharmaceutical carrier, excipient or diluent, particularly for human therapy. The pharmaceutical compositions may be for human or animal usage in human and veterinary medicine.

Examples of such suitable excipients for the various different forms of pharmaceutical compositions described herein may be found in the “Handbook of Pharmaceutical Excipients, 2^{nd} Edition, (1994), Edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s).

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

### Salts/Esters

The PLK modulators or candidate compounds of the present invention can be present as salts or esters, in particular pharmaceutically acceptable salts or esters.

Pharmaceutically acceptable salts of the PLK modulators or candidate compounds of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. sulphuric acid, phosphoric acid or hydrohalic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

### Enantiomers/Tautomers

In all aspects of the present invention previously discussed, the invention includes, where appropriate all enantiomers and tautomers of the PLK modulators or candidate compounds of the invention. The man skilled in the art will recognise compounds that possess an optical properties (one or more chiral carbon atoms) or tautomeric characteristics. The corresponding enantiomers and/or tautomers may be isolated/prepared by methods known in the art.

### Stereo and Geometric Isomers

Some of the PLK modulators or candidate compounds of the invention may exist as stereoisomers and/or geometric isomers, e.g. they may possess one or more asymmetric and/or geometric centres and so may exist in two or more stereoisomeric and/or geometric forms. The present invention contemplates the use of all the individual stereoisomers and geometric isomers of those agents, and mixtures thereof. The terms used in the claims encompass these forms, provided said forms retain the appropriate functional activity (though not necessarily to the same degree).

The present invention also includes all suitable isotopic variations of the PLK modulators or candidate compounds, or pharmaceutically acceptable salts thereof. An isotopic variation of a PLK modulator or candidate compound of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the PLK modulators or candidate compounds of the present invention can generally be prepared by conventional procedures using appropriate isotopic variations of suitable reagents.

### Solvates

The present invention also includes solvate forms of the PLK modulators or candidate compounds, for example, hydrates. The terms used in the claims encompass these forms.

### Polymorphs

The invention furthermore relates to PLK modulators or candidate compounds of the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

### Prodrugs

The invention further includes PLK modulators or candidate compounds of the present invention in prodrug form. Such prodrugs are generally compounds of the invention wherein one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. Examples of such modifications include ester (for example, any of those described above), wherein the reversion may be carried out be an esterase etc. Other such systems will be well known to those skilled in the art.

### Therapeutic Use

The PLK modulators or candidate compounds of the present invention have been found to possess anti-proliferative activity and are therefore believed to be of use in the treatment of proliferative disorders, such as cancers, leukaemias or other disorders associated with uncontrolled cellular proliferation such as psoriasis and restenosis.

A further aspect of the invention therefore relates to a method of treating a proliferative disorder, said method comprising administering to a subject in need thereof a compound selected from the following:
(i) 5′-thioadenosine, or a derivative thereof;
(ii) staurosporine, wortmannin, purvalanol A, LY294002, quercetin, morin hydrate or derivatives thereof; and
(iii) 4-[4-(4-methyl-2-methylaminothiazol-5-yl)-pyrimidin-2-ylamino]-phenol, 4-[4-(2,4-dimethylthiazol-5-yl)-pyrimidin-2-ylamino]-phenol or 4-[4-(2-amino-4-methyl thiazol-5-yl)-pyrimidin-2-ylamino]-phenol;
   or a pharmaceutically acceptable salt thereof, in an amount sufficient to inhibit PLK such that said proliferative disorder is treated.

Another aspect relates to a method of treating a proliferative disorder comprising inhibiting PLK by administering to a subject in need thereof, a therapeutically effective amount of a compound selected from the following:
(i) 5′-thioadenosine, or a derivative thereof;
(ii) staurosporine, wortmannin, purvalanol A, LY294002, quercetin, morin hydrate or derivatives thereof; and
(iii) 4-[4-(4-methyl-2-methylaminothiazol-5-yl)-pyrimidin-2-ylamino]-phenol, 4-[4-(2,4-dimethyl-thiazol-5-yl)-pyrimidin-2-ylamino]-phenol or 4-[4-(2-amino-4-methyl-thiazol-5-yl)-pyrimidin-2-ylamino]-phenol;
   or a pharmaceutically acceptable salt thereof, such that treatment of the proliferative disorder occurs.

Another aspect of the invention relates to a method of preventing and/or treating a PLK related disorder comprising administering a PLK modulator or candidate compound of the invention and/or a pharmaceutical composition according to the invention, wherein said PLK modulator, said candidate compound or said pharmaceutical, is capable of causing a beneficial preventative and/or therapeutic effect.

Preferably, for this aspect, the PLK modulator or candidate compound is selected from the following:
(i) 5′-thioadenosine, or a derivative thereof:
(ii) staurosporine, wortmannin, purvalanol A, LY294002, quercetin, morin hydrate, or derivatives thereof; and
(iii) 4-[4-(4-methyl-2-methylaminothiazol-5-yl)-pyrimidin-2-ylamino]-phenol, 4-[4-(2,4-dimethyl-thiazol-5-yl)-pyrimidin-2-ylamino]-phenol or 4-[4-2-amino-4-methyl-thiazol-5-yl)-pyrimidin-2-ylamino]-phenol;
   or a pharmaceutically acceptable salt thereof

A further aspect of the invention relates to the use of a PLK modulator or candidate compound according to the invention in the preparation of a medicament for treating a PLK related disorder. Preferably, the PLK related disorder is a proliferative disorder, more preferably cancer.

As used herein the phrase “preparation of a medicament” includes the use of the compound directly as the medicament in addition to its use in a screening programme for further therapeutic agents or in any stage of the manufacture of such a medicament.

Another aspect relates to a method of treating a PLK dependent disorder in a subject in need thereof, said method comprising administering to said subject a compound selected from the following:
(i) 5′-thioadenosine, or a derivative thereof;
(ii) staurosporine, wortmannin, purvalanol A, LY294002, quercetin, morin hydrate, or derivatives thereof; and
(iii) 4-[4-(4-methyl-2-methylaminothiazol-5-yl)-pyrimidin-2-ylamino)-phenol, 4-[4-(2,4-dimethyl-thiazol-5-yl)-pyrimidin-2-ylamino]-phenol or 4-[4-(2-amino-4-methyl-thiazol-5-yl)-pyrimidin-2-ylamino]-phenol;
   or a pharmaceutically acceptable salt thereof, in an amount sufficient to inhibit PLK.

Preferably, the PLK dependent disorder is a disorder associated with increased PLK activity. Even more preferably, the disorder is cancer.

The term “proliferative disorder” is used herein in a broad sense to include any disorder that requires control of the cell cycle, for example cardiovascular disorders such as restenosis and cardiomyopathy, auto-immune disorders such as glomerulonephritis and rheumatoid arthritis, dermatological disorders such as psoriasis, anti-inflammatory, anti-fungal, antiparasitic disorders such as malaria, emphysema and alopecia. In these disorders, the compounds of the present invention may induce apoptosis or maintain stasis within the desired cells as required.

Preferably, the proliferative disorder is a cancer or leukaemia.

In another preferred embodiment, the proliferative disorder is psoriasis.

The compounds of the invention may inhibit any of the steps or stages in the cell cycle, for example, formation of the nuclear envelope, exit from the quiescent phase of the cell cycle (G0), G1 progression, chromosome decondensation, nuclear envelope breakdown, START, initiation of DNA replication, progression of DNA replication, termination of DNA replication, centrosome duplication, G2 progression, activation of mitotic or meiotic functions, chromosome condensation, centrosome separation, microtubule nucleation, spindle formation and function, interactions with microtubule motor proteins, chromatid separation and segregation, inactivation of mitotic functions, formation of contractile ring, and cytokinesis functions. In particular, the compounds of the invention may influence certain gene functions such as chromatin binding, formation of replication complexes, replication licensing, phosphorylation or other secondary modification activity, proteolytic degradation, microtubule binding, actin binding, septin binding, microtubule organising centre nucleation activity and binding to components of cell cycle signalling pathways.

As defined herein, an anti-proliferative effect within the scope of the present invention may be demonstrated by the ability to inhibit cell proliferation in an in vitro whole cell assay, for example using any of the cell lines A549, HeLa, HT-29, MCF7, Saos-2, CCRF-CEM, HL-60 and K-562, or by showing kinase inhibition in an appropriate assay. These assays, including methods for their performance, are described in more detail in the accompanying Examples. Using such assays it may be determined whether a compound is anti-proliferative in the context of the present invention.

In one preferred embodiment, the compound of the invention is administered orally.

In one embodiment of the invention, the compound of the invention is administered in an amount sufficient to inhibit at least one PLK enzyme.

In a more preferred embodiment of the invention, the compound of the invention is administered in an amount sufficient to inhibit PLK1.

In one particularly preferred embodiment, the compounds of the invention are ATP-antagonistic inhibitors of PLK1.

In the present context ATP antagonism refers to the ability of an inhibitor compound to diminish or prevent PLK catalytic activity, i.e. phosphotransfer from ATP to a macromolecular PLK substrate, by virtue of reversibly or irreversibly binding at the enzyme's active site in such a manner as to impair or abolish ATP binding.

In another preferred embodiment, the compound of the invention is administered in an amount sufficient to inhibit PLK2 and/or PLK3.

Yet another aspect relates to a method of inhibiting PLK in a cell comprising contacting said cell with an amount of a compound selected from the following:
(i) 5′-thioadenosine, or a derivative thereof;
(ii) staurosporine, wortmannin, purvalanol A, LY294002, quercetin, morin hydrate, or derivatives thereof; and
(iii) 4-[4-(4-methyl-2-methylaminothiazol-5-yl)-pyrimidin-2-ylamino]-phenol, 4-[4-(2,4-dimethyl-thiazol-5-yl)-pyrimidin-2-ylamino]-phenol or 4-[4-(2-amino-4-methyl thiazol-5-yl)pyrimidin-2-ylamino]-phenol;
   or a pharmaceutically acceptable salt thereof, such that PLK is inhibited in said cell.

Preferably, the cell is a cancer cell.

### Administration

The pharmaceutical compositions of the present invention may be adapted for oral, rectal, vaginal, parenteral, intramuscular, intraperitoneal, intraarterial, intrathecal, intrabronchial, subcutaneous, intradermal, intravenous, nasal, buccal or sublingual routes of administration.

For oral administration, particular use is made of compressed tablets, pills, tablets, gellules, drops, and capsules. Preferably, these compositions contain from 1 to 250 mg and more preferably from 10-100 mg, of active ingredient per dose.

Other forms of administration comprise solutions or emulsions which may be injected intravenously, intraarterially, intrathecally, subcutaneously, intradermally, intraperitoneally or intramuscularly, and which are prepared from sterile or sterilisable solutions. The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

Injectable forms may contain between 10-1000 mg, preferably between 10-250 mg, of active ingredient per dose.

Compositions may be formulated in unit dosage form, i.e., in the form of discrete portions containing a unit dose, or a multiple or sub-unit of a unit dose.

### Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

Depending upon the need, the agent may be administered at a dose of from 0.01 to 30 mg/kg body weight, such as from 0.1 to 10 mg/kg, more preferably from 0.1 to 1 mg/kg body weight.

In an exemplary embodiment, one or more doses of 10 to 150 mg/day will be administered to the patient for the treatment of malignancy.

### PLK Fragment

Another aspect of the invention relates to a fragment of PLK, or a homologue, mutant, or derivative thereof, comprising a ligand binding domain, said ligand binding domain being defined by the amino acid residue structural coordinates selected from one or more of the following: L59, G60, A65, C67, A80, K82, L130, E131, C133, R135, F183 and D194.

As used herein, the term “ligand binding domain (LBD)” means the ligand binding region of PLK which is responsible for ligand binding. The term “ligand binding domain” also includes a homologue of the ligand binding domain, or a portion thereof.

As used herein, the term “portion thereof” means the structural co-ordinates corresponding to a sufficient number of amino acid residues of the PLK sequence (or homologue thereof) that are capable of interacting with a candidate compound capable of binding to the LBD. This term includes ligand binding domain amino acid residues having amino acid residues from about 4 Å to about 5 Å of a bound compound or fragment thereof. Thus, for example, the structural co-ordinates provided in the homology model may contain a subset of the amino acid residues in the LBD which may be useful in the modelling and design of compounds that bind to the LBD.

In one preferred embodiment, the fragment of PLK, or a homologue, mutant or derivative thereof, corresponds to a portion of the structure co-ordinates of Table 2.

Another aspect of the invention relates to the use of the above-described fragment of PLK, or a homologue, mutant, or derivative thereof, in an assay for identifying candidate compounds capable of modulating PLK.

The PLK proteins produced by a host recombinant cell may be secreted or may be contained intracellularly depending on the nucleotide sequence and/or the vector used.

As will be understood by those skilled in the art, expression vectors containing a PLK encoding nucleotide sequence or a mutant, variant, homologue, derivative or fragment thereof, may be designed with signal sequences which direct secretion of the PLK coding sequences through a particular prokaryotic or eukaryotic cell membrane.

The PLK encoding sequence may be fused (eg. ligated) to nucleotide sequences encoding a polypeptide domain which will facilitate purification of soluble proteins (Kroll D J et al (1993) DNA Cell Biol 12:441-53). Preferably, the polypeptide domain which facilitates purification of soluble proteins is fused in frame with the PLK encoding sequence. Such purification facilitating domains include, but are not limited to, metal chelating peptides—such as histidine-tryptophan modules that allow purification on inmobilised metals (Porath J (1992) Protein Expr Purif 3, 263-281), protein A domains that allow purification on immobilised immunoglobulin, and the domain utilised in the FLAGS extension/affinity purification system (Immunex Corp, Seattle, Wash.). The inclusion of a cleavable linker sequence such as Factor XA or enterokinase (Invitrogen, San Diego, Calif.) between the purification domain and PLK is useful to facilitate purification.

### Nucleotide Sequences

As used herein, the term “nucleotide sequence” refers to nucleotide sequences, oligonucleotide sequences, polynucleotide sequences and variants, homologues, fragments and derivatives thereof (such as portions thereof) which comprise the nucleotide sequences encoding PLK.

The nucleotide sequence may be DNA or RNA of genomic or synthetic or recombinant origin, which may be double-stranded or single-stranded whether representing the sense or antisense strand or combinations thereof.

Preferably, the term nucleotide sequence is prepared by use of recombinant DNA techniques (e.g. recombinant DNA). The nucleotide sequences may include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present invention, it is to be understood that the nucleotide sequences described herein may be modified by any method available in the art.

It will be understood by a skilled person that numerous different nucleotide sequences can encode the same protein as a result of the degeneracy of the genetic code. In addition, it is to be understood that skilled persons may, using routine techniques, make nucleotide substitutions that do not substantially affect the activity encoded by the nucleotide sequence of the present invention to reflect the codon usage of any particular host organism in which the target is to be expressed. Thus, the terms “variant”, “homologue” or “derivative” in relation to nucleotide sequences include any substitution of, variation of, modification of, replacement of, deletion of or addition of one (or more) nucleic acids from or to the sequence providing the resultant nucleotide sequence encodes a functional protein according to the present invention (or even a modulator of PLK according to the present invention if said modulator comprises a nucleotide sequence or an amino acid sequence).

### Amino Acid Sequences

As used herein, the term “amino acid sequence” is synonymous with the term “polypeptide” and/or the term “protein”. In some instances, the term “amino acid sequence” is synonymous with the term “peptide”.

The amino acid sequence may be isolated from a suitable source, or it may be made synthetically or it may be prepared by use of recombinant DNA techniques.

### Variants/Homologues/Derivatives/Fragments

The PLK described herein is intended to include any polypeptide, which has the activity of the naturally occurring PLK and includes all vertebrate and mammalian forms. Such terms also include polypeptides that differ from naturally occurring forms of PLK by having amino acid deletions, substitutions, and additions, but which retain the activity of PLK.

The term “variant” is used to mean a naturally occurring polypeptide or nucleotide sequences which differs from a wild-type or a native sequence.

The term “fragment” indicates that a polypeptide or nucleotide sequence comprises a fraction of a wild-type or a native sequence. It may comprise one or more large contiguous sections of sequence or a plurality of small sections. The sequence may also comprise other elements of sequence, for example, it may be a fusion protein with another protein. Preferably the sequence comprises at least 50%, more preferably at least 65%, more preferably at least 80%, most preferably at least 90% of the wild-type sequence.

The present invention also encompasses the use of variants, homologues and derivatives of nucleotide and amino acid sequences. Here, the term “homologue” means an entity having a certain homology with amino acid sequences or nucleotide sequences. Here, the term “homology” can be equated with “identity”.

In the present context, an homologous sequence is taken to include an amino acid sequence which may be at least 75, 85 or 90% identical, preferably at least 95 or 98% identical to the subject sequence. Although homology can also be considered in terms of similarity (i.e. amino acid residues having similar chemical properties/functions), it is preferred to express homology in terms of sequence identity.

An homologous sequence is taken to include a nucleotide sequence which may be at least 75, 85 or 90% identical, preferably at least 95 or 98% identical to the subject sequence.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However, for some applications, it is preferred to use the GCG Bestfit program. A new tool, called BLAST 2 Sequences is also available for comparing protein and nucleotide sequence (see FEMS Microbiol Lett 1999 174(2): 247-50; FEMS Microbiol Lett 1999 177(1): 187-8)

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix, such as BLOSUM62. Once the software has produced an optimal alignment, it is possible to calculate % homology, preferably % sequence identity. The software typically does this as part of the sequence comparison and generates a numerical result.

The sequences may also have deletions, insertions or substitutions of amino acid residues, which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Homologous substitution (substitution and replacement are both used herein to mean the interchange of an existing amino acid residue, with an alternative residue) may occur i.e. like-for-like substitution such as basic for basic, acidic for acidic, polar for polar etc. Non-homologous substitution may also occur i.e. from one class of residue to another or alternatively involving the inclusion of unnatural amino acids such as ornithine hereinafter referred to as Z), diaminobutyric acid ornithine (hereinafter referred to as B), norleucine ornithine (hereinafter referred to as 0), pyriylalanine, thienylalanine, naphthylalanine and phenylglycine.

Replacements may also be made by unnatural amino acids include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, β-alanine*, L-α-amino butyric acid*, L-γ-amino butyric acid*, L-α-amino isobutyric acid*, L-ε-amino caproic acid*, 7-amino heptanoic acid*, L-methionine sulfone^{#}*, L-norleucine*, L-norvaline*, p-nitro-L-phenylalanine*, L-hydroxyproline^{#}, L-thioproline*, methyl derivatives of phenylalanine (Phe) such as 4 methyl-Phe*, pentamethyl-Phe*, L-Phe (4-amino)^{#}, L-Tyr (methyl)*, L-Phe (4-isopropyl)*, L-Tic (1,2,3,4-tetrahydroisoquinoline-3-carboxyl acid)*, L-diaminopropionic acid ^{#} and L-Phe (4-benzyl)*. The notation * has been utilised for the purpose of the discussion above (relating to homologous or non-homologous substitution), to indicate the hydrophobic nature of the derivative whereas # has been utilised to indicate the hydrophilic nature of the derivative, #* indicates amphipathic characteristics.

The term “derivative” or “derivatised” as used herein includes chemical modification of an entity, such as candidate compound or a PLK modulator. Illustrative of such chemical modifications would be replacement of hydrogen by a halo group, an alkyl group, an acyl group or an amino group.

Variant amino acid sequences may include suitable spacer groups that may be inserted between any two amino acid residues of the sequence including alkyl groups such as methyl ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D C, Trends Biotechnol. (1995) 13(4), 132-134.

### Mutant

As used herein, the term “mutant” refers to PLK comprising one or more changes in the wild-type PLK sequence.

The term “mutant” is not limited to amino acid substitutions of the amino acid residues in PLK, but also includes deletions or insertions of nucleotides which may result in changes in the amino acid residues in the amino acid sequence of PLK.

The present invention also enables the solving of the crystal structure of PLK mutants. More particularly, by virtue of the present invention, the location of the active site of PLK based on the structural coordinates of Table 2 permits the identification of desirable sites for mutation. For example, one or more mutations may be directed to a particular site—such as the active site—or combination of sites of PLK. Similarly, only a location on, at or near the enzyme surface may be replaced, resulting in an altered surface charge of one or more charge units, as compared to the wild-type enzyme. Alternatively, an amino acid residue in PLK may be chosen for replacement based on its hydrophilic or hydrophobic characteristics.

Such mutants may be characterised by any one of several different properties as compared with wild-type PLK. For example, such mutants may have altered surface charge of one or more charge units, or have an increased stability to subunit dissociation, or an altered substrate specificity in comparison with, or a higher specific activity than, wild-type PLK.

The mutants may be prepared in a number of ways that are known by a person skilled in the art. For example, mutations may be introduced by means of oligonucleotide-directed mutagenesis or other conventional methods. Alternatively, mutants of PLK may be generated by site specific replacement of a particular amino acid with an unnaturally occurring amino acid. This may be achieved by growing a host organism capable of expressing either the wild-type or mutant polypeptide on a growth medium depleted of one or more natural amino acids but enriched in one or more corresponding unnaturally occurring amino acids.

### Host Cells

As used herein, the term “host cell” refers to any cell that comprises nucleotide sequences that are of use in the present invention, for example, nucleotide sequences encoding PLK.

Host cells may be transformed or transfected with a nucleotide sequence contained in a vector e.g. a cloning vector. Preferably, said nucleotide sequence is carried in a vector for the replication and/or expression of the nucleotide sequence. The cells will be chosen to be compatible with the said vector and may for example be prokaryotic (for example bacterial), fungal, yeast or plant cells.

The gram-negative bacterium E. coli is widely used as a host for cloning nucleotide sequences. This organism is also widely used for heterologous nucleotide sequence expression. However, large amounts of heterologous protein tend to accumulate inside the cell. Subsequent purification of the desired protein from the bulk of E. coli intracellular proteins can sometimes be difficult.

In contrast to E. coli, bacteria from the genus Bacillus are very suitable as heterologous hosts because of their capability to secrete proteins into the culture medium. Other bacteria suitable as hosts are those from the genera Streptomyces and Pseudomonas.

Depending on the nature of the polynucleotide and/or the desirability for further processing of the expressed protein, eukaryotic hosts including yeasts or other fungi may be preferred. In general, yeast cells are preferred over fungal cells because yeast cells are easier to manipulate. However, some proteins are either poorly secreted from the yeast cell, or in some cases are not processed properly (e.g. hyperglycosylation in yeast). In these instances, a different fungal host organism should be selected.

Examples of expression hosts are fungi—such as Aspergillus species (such as those described in EP-A-0184438 and EP-A-0284603) and Trichoderma species; bacteria—such as Bacillus species (such as those described in EP-A-0134048 and EP-A-0253455), Streptomyces species and Pseudomonas species; yeasts—such as Kluyveromyces species (such as those described in EP-A-0096430 and EP-A-0301670) and Saccharomyces species; and mammalian cells—such as CHO-K1 cells.

The use of host cells may provide for post-translational modifications as may be needed to confer optimal biological activity on recombinant expression products of the present invention.

Aspects of the present invention also relate to host cells comprising the PLK constructs of the present invention. The PLK constructs may comprise a nucleotide sequence for replication and expression of the sequence. The cells will be chosen to be compatible with the vector and may for example be prokaryotic (for example bacterial), fungal, yeast or plant cells.

In a preferred embodiment, the host cells are mammalian cells, such as CHO-K1 cells.

### Vector

Aspects of the present invention relate to a vector comprising a nucleotide sequence, such as a nucleotide sequence encoding PLK or a modulator of PLK, administered to a subject.

Preferably, PLK or the modulator of PLK is prepared and/or delivered using a genetic vector.

As it is well known in the art, a vector is a tool that allows or facilitates the transfer of an entity from one environment to another. In accordance with the present invention, and by way of example, some vectors used in recombinant DNA techniques allow entities, such as a segment of DNA (such as a heterologous DNA segment, such as a heterologous cDNA segment), to be transferred into a host and/or a target cell for the purpose of replicating the vectors comprising nucleotide sequences and/or expressing the proteins encoded by the nucleotide sequences. Examples of vectors used in recombinant DNA techniques include, but are not limited to, plasmids, chromosomes, artificial chromosomes or viruses.

The term “vector” includes expression vectors and/or transformation vectors.

The term “expression vector” means a construct capable of in vivo or in vitro/ex vivo expression.

The term “transformation vector” means a construct capable of being transferred from one species to another.

### Regulatory Sequences

In some applications, nucleotide sequences are operably linked to a regulatory sequence which is capable of providing for the expression of the nucleotide sequence, such as by a chosen host cell. By way of example, a vector comprising the PLK nucleotide sequence is operably linked to such a regulatory sequence i.e. the vector is an expression vector.

The term “operably linked” refers to a juxtaposition wherein the components described are in a relationship permitting them to function in their intended manner. A regulatory sequence “operably linked” to a coding sequence is ligated in such a way that expression of the coding sequence is achieved under conditions compatible with the control sequences.

The term “regulatory sequences” includes promoters and enhancers and other expression regulation signals.

The term “promoter” is used in the normal sense of the art, e.g. an RNA polymerase binding site.

Enhanced expression of a nucleotide sequence, for example, a nucleotide sequence encoding PLK, may also be achieved by the selection of heterologous regulatory regions, e.g. promoter, secretion leader and terminator regions, which serve to increase expression and, if desired, secretion levels of the protein of interest from the chosen expression host and/or to provide for the inducible control of the expression of PLK. In eukaryotes, polyadenylation sequences may be operably connected to the PLK nucleotide sequence.

Preferably, the PLK nucleotide sequence is operably linked to at least a promoter.

Aside from the promoter native to the gene encoding the PLK nucleotide sequence, other promoters may be used to direct expression of the PLK polypeptide. The promoter may be selected for its efficiency in directing the expression of the PLK nucleotide sequence in the desired expression host.

In another embodiment, a constitutive promoter may be selected to direct the expression of the PLK nucleotide sequence. Such an expression construct may provide additional advantages since it circumvents the need to culture the expression hosts on a medium containing an inducing substrate.

Hybrid promoters may also be used to improve inducible regulation of the expression construct.

The promoter can additionally include features to ensure or to increase expression in a suitable host. For example, the features can be conserved regions such as a Pribnow Box or a TATA box. The promoter may even contain other sequences to affect (such as to maintain, enhance, decrease) the levels of expression of the PLK nucleotide sequence. For example, suitable other sequences include the Sh1-intron or an ADH intron. Other sequences include inducible elements—such as temperature, chemical, light or stress inducible elements. Also, suitable elements to enhance transcription or translation may be present.

### Expression Vector

Preferably, nucleotide sequences, such as nucleotide sequences encoding PLK or modulators of PLK, are inserted into a vector that is operably linked to a control sequence that is capable of providing for the expression of the coding sequence by the host cell.

Nucleotide sequences produced by a host recombinant cell may be secreted or may be contained intracellularly depending on the sequence and/or the vector used. As will be understood by those of skill in the art, expression vectors can be designed with signal sequences, which direct secretion of the nucleotide sequence through a particular prokaryotic or eukaryotic cell membrane.

Preferably, the expression vectors are stably expressed in CHO cells as described previously (Ehlers et al. (1996) Biochemistry 35, 9549-9559). More preferably, the expression vectors are pLEN-tACEΔ36g (1, 2, 3, 4) and pLEN-tACEΔ36g (1,3).

### Fusion Proteins

PLK or a modulator of PLK may be expressed as a fusion protein to aid extraction and purification and/or delivery of the modulator of PLK or the PLK protein to an individual and/or to facilitate the development of a screen for modulators of PLK.

Examples of fusion protein partners include glutathione-S-transferase (GST), 6×His, GAL4 (DNA binding and/or transcriptional activation domains) and β-galactosidase.

It may also be convenient to include a proteolytic cleavage site between the fusion protein partner and the protein sequence of interest to allow removal of fusion protein sequences. Preferably, the fusion protein will not hinder the activity of the protein of interest.

The fusion protein may comprise an antigen or an antigenic determinant fused to the substance of the present invention. In this embodiment, the fusion protein may be a non-naturally occurring fusion protein comprising a substance, which may act as an adjuvant in the sense of providing a generalised stimulation of the immune system. The antigen or antigenic determinant may be attached to either the amino or carboxy terminus of the substance.

### Organism

The term “organism” in relation to the present invention includes any organism that could comprise PLK and/or modulators of PLK. Examples of organisms may include mammals, fungi, yeast or plants.

Preferably, the organism is a mammal. More preferably, the organism is a human.

### Transformation

As indicated earlier, the host organism can be a prokaryotic or a eukaryotic organism. Examples of suitable prokaryotic hosts include E. coli and Bacillus subtilis. Teachings on the transformation of prokaryotic hosts are well documented in the art, for example see Sambrook et al (Molecular Cloning: A Laboratory Manual, 2nd edition, 1989, Cold Spring Harbor Laboratory Press) and Ausubel et al., Current Protocols in Molecular Biology (1995), John Wiley & Sons, Inc. Examples of suitable eukaryotic hosts include mammalian cells.

If a prokaryotic host is used then the nucleotide sequence, such as the PLK nucleotide sequence, may need to be suitably modified before transformation—such as by removal of introns.

Thus, the present invention also relates to the transformation of a host cell with a nucleotide sequence, such as PLK or a modulator of PLK. Host cells transformed with the nucleotide sequence may be cultured under conditions suitable for the expression and recovery of the encoded protein from cell culture. The protein produced by a recombinant cell may be secreted or may be contained intracellularly depending on the sequence and/or the vector used. As will be understood by those of skill in the art, expression vectors containing coding sequences can be designed with signal sequences which direct secretion of the coding sequences through a particular prokaryotic or eukaryotic cell membrane. Other recombinant constructions may join the coding sequence to nucleotide sequence encoding a polypeptide domain, which will facilitate purification of soluble proteins (Kroll D J et al (1993) DNA Cell Biol 12:441-53) e.g. 6-His or Glutathione-S-transferase.

### Transfection

Vectors comprising for example, the PLK nucleotide sequence, may be introduced into host cells, for example, mammalian cells, using a variety of methods.

Typical transfection methods include electroporation, DNA biolistics, lipid-mediated transfection, compacted DNA-mediated transfection, liposomes, immunoliposomes, lipofectin, cationic agent-mediated, cationic facial amphiphiles (CFAs) (Nature Biotech. (1996) 14, 556), multivalent cations such as spermine, cationic lipids or polylysine, 1, 2,-bis(oleoyloxy)-3-(trimethylammonio) propane (DOTAP)-cholesterol complexes (Wolff and Trubetskoy 1998 Nature Biotechnology 16: 421) and combinations thereof.

Uptake of nucleic acid constructs by mammalian cells is enhanced by several known transfection techniques for example those including the use of transfection agents. Example of these agents include cationic agents (for example calcium phosphate and DEAE-dextran) and lipofectants (for example Lipofectam™ and Transfectam™). Typically, nucleic acid constructs are mixed with the transfection agent to produce a composition.

Such methods are described in many standard laboratory manuals—such as Sambrook et al., Molecular Cloning: A Laboratory Manual, 2d ed. (1989) Cold Spring Harbor Laboratory Press, Cold Spring Harbor, N.Y.

The present invention is further described by way of example, and with reference to the following figures wherein:

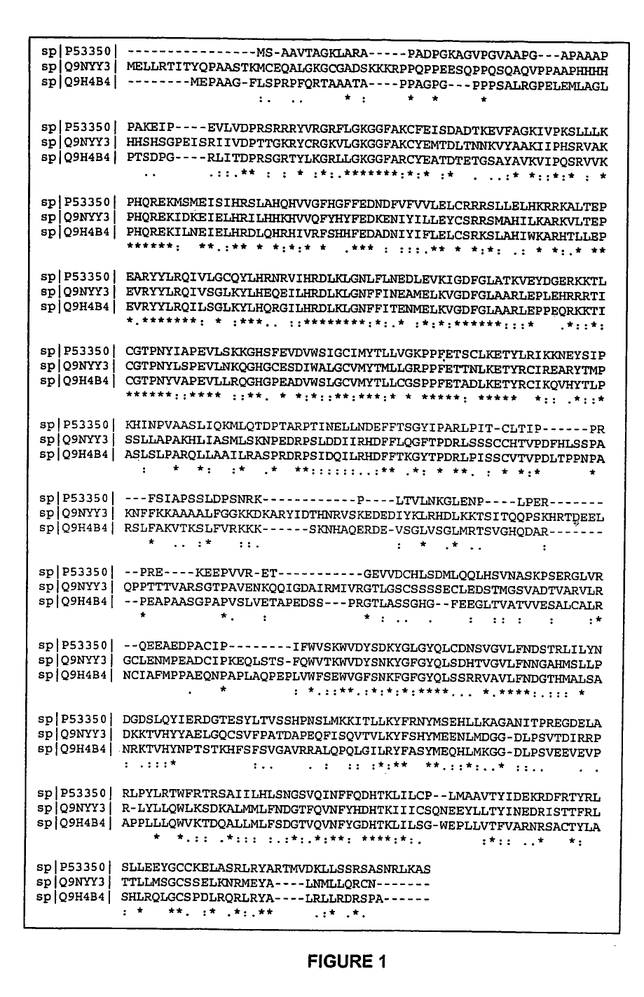

FIG. 1 shows multiple sequence alignment (Clustal W) of human PLK1 (P53350), PLK2 (Q9NYY3), and PLK3 (Q9H4B4).

FIG. 2 shows a schematic view of PLK1 homology model in complex with ATP (stick model, labelled). The protein structure is indicated with a ribbon (loops, thin; helices, thick; sheets, arrows). The Cys residues are shown with space-filled atoms and are labelled.

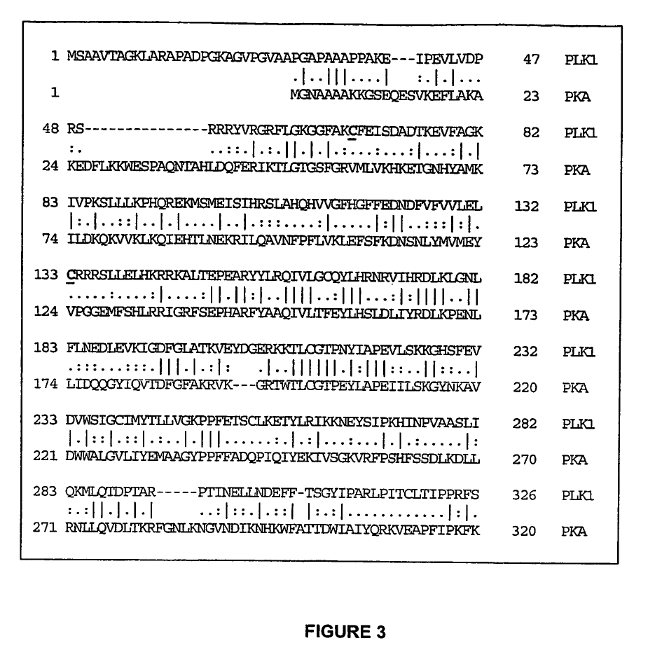

FIG. 3 shows sequence alignment of PLK1 and PKA kinase domains.

FIG. 4 shows modelled complex between PLK1 and ATP (a) and 5′-thioadenosine (b). The positions of the thiol groups (SH) of Cys⁶⁷ and 5′thioadenosine are indicated.

FIG. 5 shows dose response curves of PLK1 activity inhibition by various adenosine derivatives in the absence or presence of the reducing agent dithiothreitol (+DTT or −DTT).

FIG. 6 shows kinetic analysis of PLK1 inhibition by 5′-thioadenosine.

FIG. 7 shows modelled PLK1-bound conformations of ATP (a); 5′-thioadenosine (b); staurosporine (c); and 4-[4-4-methyl-2-methylamino-thiazol-5-yl)-pyrimidin-2-ylamino]-phenol (d). Non-H atoms are labelled.

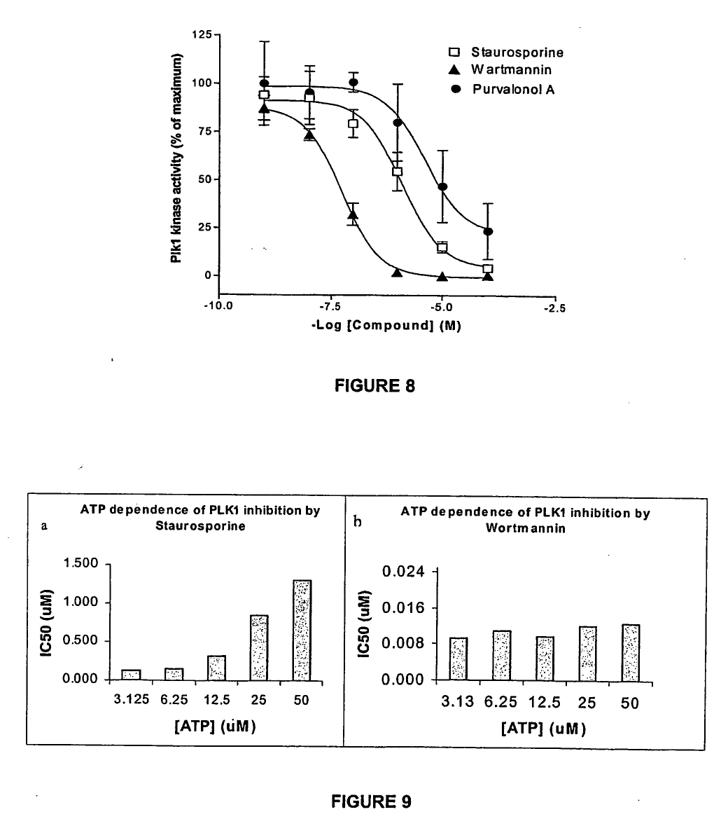

FIG. 8 shows dose response curves for Purvalanol A, staurosporine and wortmannin.

FIG. 9 shows the ATP dependence of PLK1 inhibition by staurosporine (a) and wortmannin (b).

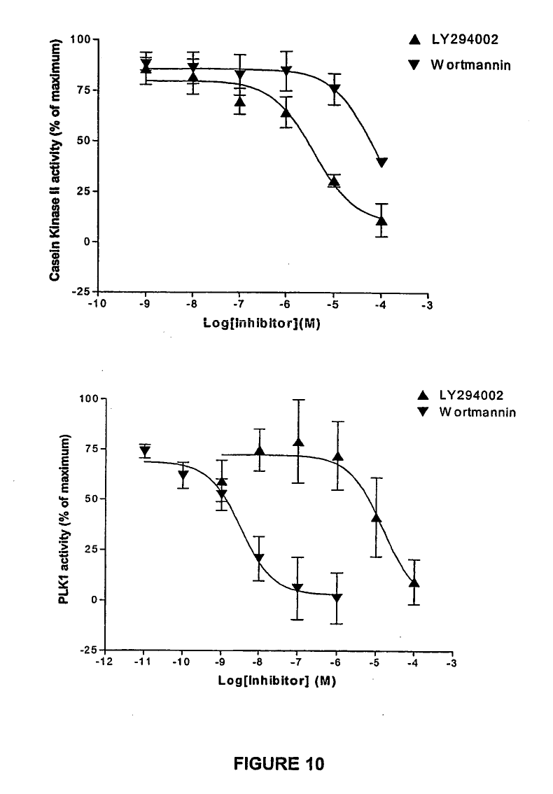

FIG. 10 shows the Inhibition of PLK1 and Casein Kinase II by Wortmannin and LY294002.

FIG. 11 shows docked structures of A) purvalanol A and B) morin hydrate with the ATP binding site of the PLK1 model structure.

FIG. 12 shows modelled structure of wortmannin covalently bound to K82 in the ATP cleft of PLK1. The right panel view is rotated by 180° along the y axis relative to the left view.

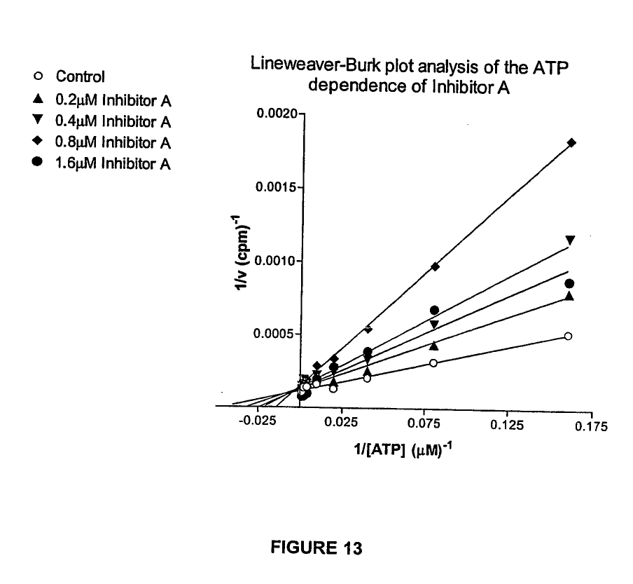

FIG. 13 shows a Lineweaver-Burk plot analysis of the ATP dependence of Inhibitor A.

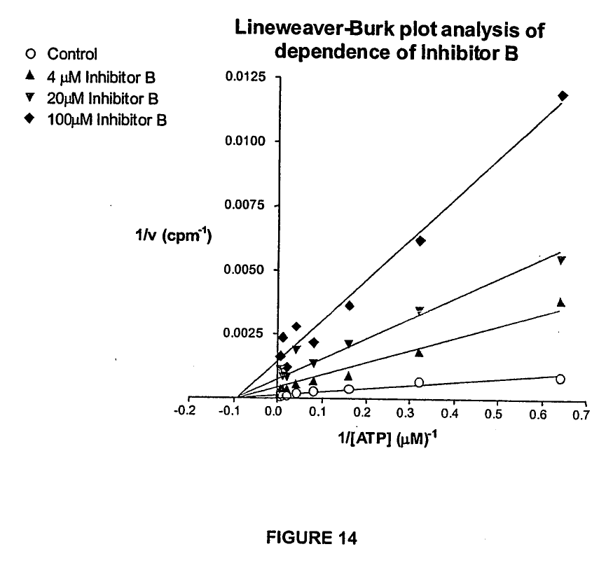

FIG. 14 shows a Lineweaver-Burk plot analysis of the ATP dependence of Inhibitor B.

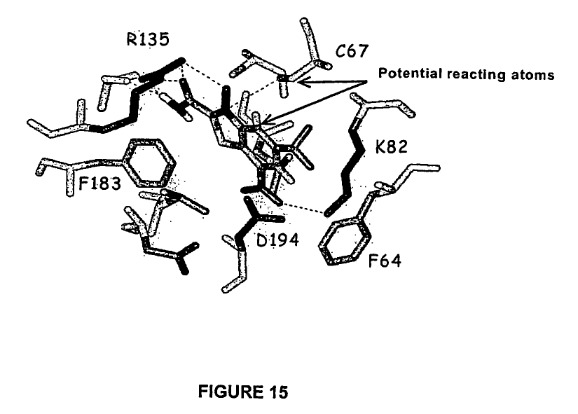

FIG. 15 shows the modelled structure of Inhibitor B in the binding pocket of PLK1, showing the close proximity of the potential reactive atoms of Inhibitor B to the cysteine (C67) residue of PLK1.

### EXAMPLES

### General Methods

The methods described here may employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Using Antibodies: A Laboratory Manual: Portable Protocol NO. I by Edward Harlow, David Lane, Ed Harlow (1999, Cold Spring Harbor Laboratory Press, ISBN 0-87969-544-7); Antibodies: A Laboratory Manual by Ed Harlow (Editor), David Lane (Editor) (1988, Cold Spring Harbor Laboratory Press, ISBN 0-87969-3,4-2), 1855. Handbook of Drug Screening, edited by Ramakrishna Seethala, Prabhavathi B. Fernandes (2001, New York, N.Y., Marcel Dekker, ISBN 0-8247-0562-9); and Lab Ref: A Handbook of Recipes, Reagents, and Other Reference Tools for Use at the Bench, Edited Jane Roskams and Linda Rodgers, 2002, Cold Spring Harbor Laboratory, ISBN 0-87969-630-3. Each of these general texts is herein incorporated by reference.

### Example 1

### Construction of PLK1 Homology Model

The homology model for PLK1 kinase domain was generated using the program module Homology within the molecular modelling package Insight II (Accelrys, San Diego, Calif.) [38]. The sequence containing the kinase domain of PLK1 (residues 1-356) was employed in a FASTA sequence and structural search [39] in order to find the closest sequence-related kinase for which experimental structural information was available. For this search, the BLOSUM 50 scoring matrix [40] and a specific residue string value of 2 was employed. The closest match of known structure proved to be that of cAMP-dependent protein kinase (protein kinase A, PKA) with a sequence identity of 30% and similarity of close to 50% (FIG. 3). Although these values are typically low for homology model building, the structural conservation of protein kinases was thought to allow a valid structure to be generated. Sequence alignment of PLK1 kinase domain with PKA in addition to CDK2 and ERK2 (which also were among the most homologous structures) indicated that the minimal kinase domain included residues 52-308. For the sequence alignment, the PAM 120 multiple scoring matrix [41] was used with a dimension block of 0.6, a high significance p value of 0.0001, a not significant p value of 0.1, and a pair-wise threshold of 60. Using a combination of the three structures to generate coordinates for the regions that had the highest identity in each kinase (Table 1), a model structure for the kinase domain was constructed. The strategy generally involved using PKA to define the structurally conserved regions (SCRs) from which the coordinates were subsequently transferred. This was then followed by loop construction where the non-SCRs were generated by de-novo building and subsequent evaluation of the most realistic coordinates (in terms of energetics of the loop itself and the exclusion of loops leading to overlapping atoms). After loop building was completed for missing coordinates, the raw coordinates were then refined using successive rounds of end repair splice repairing using an omega force constant of 50, energy minimization (100 steps of steepest descent to a derivative of 5). The model was then completed through using a further minimisation and 1 ps of molecular dynamics to more fully explore the conformational space of the loop regions. The final model structure was then checked against databases of protein structures for bond length and dihedral angle violations. The results indicated that these as a whole were within acceptable limits with >80% of residues having phi-psi plots with the allowed region in Ramachandran space [42]. The coordinate file for the final PLK1 homology model—ATP complex in Brookhaven Protein Databank (PDB) format [43] is shown in Table 2.

### Example 2

### Production of Recombinant Human PLK1

The human PLK1 (SwissProt accession number P53350, [44]) open reading frame (ORF) was amplified by PCR from a human foetal lung cDNA library (Clontech). An Nhe I restriction endonuclease site was introduced upstream of the ORF, by inclusion in the sense PCR primer. An Eco RI restriction endonuclease site was introduced downstream of the ORF, by inclusion in the antisense PCR primer. The PCR product generated was cloned into pCR4-Topo (Invitrogen), and sequenced. The ORF was then sub-cloned as an Nhe I/Eco RI fragment into pSSP1, a derivative of bacmid transfer vector pFastBac HTa (Invitrogen). The PLK1 ORF was cloned into pSSP1 such that the resulting PLK1 translation product would have a 19 amino acid N-terminal tag (MSYYHHHHHHGMASDDDDK) containing a hexahistidine tag and an enterokinase cleavage site. The pSSP1-Plk1 expression cassette was transferred into bacmid DNA by transposition in E. coli DH10Bac (Invitrogen). Purified recombinant bacmid DNA was transfected into Sf9 cells, to produce an infective stock of recombinant baculovirus. Following subsequent amplification and titering of the baculoviral stock, this was used to infect Sf9 cells at a multiplicity of infection of approximately 3. His-tagged PLK1 was expressed by incubating the infected cells at 27° C., with shaking. Two days after infection, the cells were collected by centrifugation. Prior to purification, PLK1 expression was confirmed by Western blotting. To the cell pellet from 150 ml Sf9 insect cell culture 10 mL lysis buffer [10 mM Tris-HCl pH 8.0, 150 ml NaCl, 20 mM β-mercaptoethanol, 1 mM PMSF, 1 mM benzamidine, protease inhibitor cocktail (Sigma; 1:1,000 diluted), 20 mM imidazole], supplemented with 2 mM NaF and 1 mM Na₃VO₄, was added; the mixture was sonicated (6×20 s) on ice and centrifuged for 15 min at 15,000 r.p.m. The supernatant was filtered (0.45 μm filter) and the filtrate was applied to a pre-equilibrated (with 20 mL lysis buffer) 1.2-mL Ni-NTA agarose column (Qiagen). After incubation for 2 h at 4° C., the non-bound fraction was eluted with was buffer (as lysis buffer but 300 mM NaCl and without imidazole). Protein was eluted with elution buffer (as lysis buffer but 100 mM NaCl, 250 mM imidazole, 0.02% Nonidet P-40). Pooled fractions containing target protein were applied to an equilibrated (with dialysis buffer) 5-mL HiTrap™ desalting column (Amersham Biosciences) and eluted with dialysis buffer (25 mM Tris/MES pH 7.6, 1 mM β-mercaptoethanol, 0.01% Tween-20, 10 mM MgCl₂, 50 μM ATP, 100 mM NaCl, 1 mM PMSF, 1 mM benzamidine, 10% glycerol). Pooled fractions containing pure target protein were centrifuged 15,000 r.p.m. for 15 min. The supernatant PLK1 stock solution was stored at −70° C.

### Example 3

### Construction, Expression and Purification of a Cdc25C Fragment

Using standard techniques, a full-length Cdc25C clone was isolated by PCR from HeLa mRNA and inserted on a BamHI-HindIII fragment into pRsetA. The amino terminal Cdc25C fragment (encoding residues 1-300) was excised from this vector and inserted into pET28a (between the NcoI and BamHI sites). Expression was under the control of the T7 promoter, and the encoded protein contains a HiS₆ tag at the carboxy terminus. The vector was transformed into E. coli strain BRL(DE3) pLysS for expression experiments. The protein was expressed in BL21(DE3) RIL bacteria cells, grown in LB media at 37° C. until optical density at 600 nm of 0.6 was reached. The expression was induced with 1 mM IPTG and the bacterial culture was grown further for 3 h. The bacteria were harvested by centrifugation and the cell pellet was re-suspended in 50 mM Tris pH 7.5 and 10% sucrose, snap-frozen, and stored at −70° C. until used.

Purification of the protein was then carried out by lysing the bacterial pellet in 10 mL of lysis buffer (10 mM Tris-HCl, pH 8.0, 150 mM NaCl, S mM P-mercaptoethanol, and 20 mM imidazole) supplemented with a cocktail of protease inhibitors, sonicated 6 times at 20-s bursts. The lysate was then centrifuged for 15 min at 15,000 r.p.m. and filtered through a 0.45-μm filter. The sample was then loaded onto a Ni-NTA agarose column, washed several times then the Cdc25C protein fragment was eluted with a buffer containing 10 mM Tris-HCl, pH 8.0, 100 mM NaCl, 5 mM β-mercaptoethanol, 0.02% Nonidet P-40, and 250 mM imidazol. The eluate was then dialysed, concentrated, snap-frozen in liquid nitrogen, and stored at −70° C. until used.

### Example 4

### PLK1 Assay

PLK1 kinase activity was assayed using human CDC25C phosphatase as a substrate [4]. The assays were carried out using 96-well microtitre plates by incubating CDC25C (2 μg/well) with 1 μg/well of purified human recombinant PLK1 and varying concentrations of the candidate compound in a total volume of 25 mL of 20 mM Tris/HCl buffer pH 7.5, supplemented with 25 mM β-glycerophosphate, 5 mM EGTA, 1 mM DTT, and 1 mM NaVO₃. Reaction was initiated by the addition of 100 μM ATP and 0.5 μCi of [γ-³²P]-ATP. The reaction mixture was incubated at 30° C. for 1 h, then stopped with 75 mM aq orthophosphoric acid, transferred onto a 96-well P81 filter plate (Whatman), dried, and the extent of CDC25C phosphorylation was assessed by scintillation counting using a Packard TopCount plate reader.

### Example 5

### Casein Kinase II (CKII) Assay

Human recombinant CKII activity was assayed using the peptide H-Arg-Arg-Arg-Glu-Glu-Glu-Thr-Glu-Glu-Glu-OH as a substrate. The assays were carried out using 96-well microtitre plates by incubating the peptide substrate (10 μM) with 20 Units/well of CKII (New England Biolabs) and varying concentrations of the candidate compound in a total volume of 25 μL of 25 mM MOPS buffer pH 7.0, supplemented with 25 mM β-glycerophosphate, 5 mM EGTA, 1 mM DTT, and 1 mM NaVO₃. Reaction was initiated by the addition of 100 μM ATP and 0.25 μCi of [γ-³²P]-ATP. The reaction mixture was incubated at 30° C. for 15 minutes, then stopped with 75 mM aq orthophosphoric acid, transferred onto a 96-well P81 filter plate (Whatman), dried, and the extent of peptide phosphorylation was assessed by scintillation counting using a Packard TopCount plate reader.

### Example 6

### Chemical Kinase Inhibitors

Wortmannin and LY294002 were acquired from CN Biosciences Ltd., UK. Staurosporine, quercetin, and myricetin were from Sigma Chemicals, UK. All other flavonoid compounds were purchased from Indofine Chemical Company, Inc., Somerville, N.J., USA.

### Example 7

### Synthesis of Compounds

4-[4-(4-methyl-2-methylaminothiazol-5-yl)-pyrimidin-2-ylamino]-phenol, 4-[4-(2,4-dimethyl-thiazol-5-yl)-pyrimidin-2-ylamino]-phenol and 4-[4-(2-amino-4-methyl-thiazol-5-yl)-pyrimidin-2-ylamino]-phenol were synthesised in accordance with the methodology described in WO 01/72745. Staurosporine and derivatives thereof (such as CGP 41251 and UCN-01) are described in the literature [see for example, Gescher A., Gen Pharmacol. 1998, 31, p 721-8].

### Synthesis of 5′-deoxy-5-thio-adenosine (4)

5′-Deoxy-5-thio-adenosine (4) is a known compound [45] and it can be prepared readily from commercially available 2′,3′-isopropylideneadenosine 1 as shown in Scheme 1 [46].

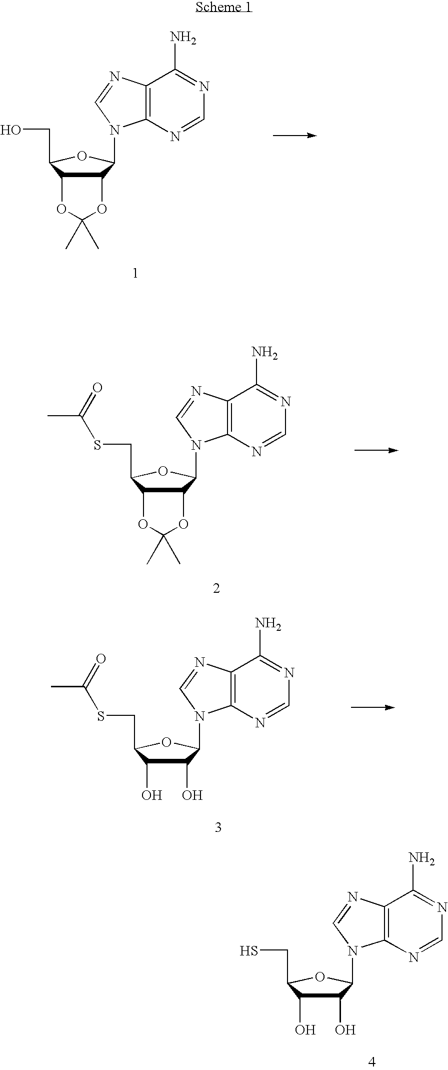

### 5′-Deoxy-5′-acetylthio-2′,3′-O-isopropylideneadenosine (2)

Diethyl azodicarboxyl-ate (3.4 mL, 21.73 mmol) was added drop-wise over 5 min to an ice-cold solution of triphenylphosphine (5.7 g, 21.73 mmol). The solution was stirred for 30 min at 0° C. prior to the addition of 2′,3′-O-isopropylideneadenosine (1; 3.0 g, 9.76 mmol) and stirring was then continued for a further 10 min to produce a yellow suspension. To the suspension a solution of thioacetic acid (1.6 mL, 21.73 mmol) in absol tetrahydrofuran (5 mL) was added drop-wise and stirring was then continued for a further 1 h at 0° C. During this time the yellow suspension became a darker yellow solution. After stirring for 1 h the solvent was removed under reduced pressure and the resulting yellowish residue was purified by flash chromatography on silica gel [350 g, CHCl₃/THF (4:1 v/v) and then CHCl₃/CH₃OH (9:1 v/v)]. The fractions containing the product were combined and the solvent removed under reduced pressure. The residue was dried in vacuo (0.5 mbar) to furnish pure protected thionucleoside 2 (3.2 g, 90%) as a white foam; TLC R_{f} (CH₂Cl₂/CH₃OH, 9:1 v/v)=0.6, mp=56-57° C.; ¹H-NMR (CDCl₃): δ 1.39 (s, 6H, CH₃), 2.34 (s, 3H, COCH₃), 3.18 and 3.29 (AB part of ABX spectrum, J_{5′a-H,4′-H}=J_{5′b-H, 4′-H}=6.5 Hz, J_{gem}=13.5 Hz, 2H, 5′a-H, 5′b-H), 4.34 (dt, J_{4′-H, 3′-H}=3 Hz, J_{4′a-H, 5a′-H}=J_{4′-H, 5′b-H}=7 Hz, 1H, 4′-H), 4.97 (dd, J_{3′-H, 4′-H}=3 Hz, J_{3′-H, 2′-H}=6.5 Hz, 1H, 3′-H), 5.51 (dd, J_{2′-H, 1′-H}=2 Hz, J_{2′-H, 3′-H}=6.5 Hz, 1H, 2′-H), 6.07 (d, J_{1′-H, 2′-H}=Hz, 1H, 1′-H), 5.9 (s, br., 2H, NH₂), 7.90 (s, 1H, 8-H) and 8.36 (s, 1H, 2-H); ¹³C-NMR (CDCl₃): δ 25.56 (q, CH₃), 27.33 (q, CH₃), 30.79 (q, COCH₃), 31.60 (t, C-5′), 84.24 (d, C-3′), 84.43 (d, C-2′), 86.47 (d, C-4′), 91.07 (d, C-1′), 114.75 (s, C(CH₃)₂), 120.53 (s, C-5), 140.09 (d, C-8), 149.42 (s, C-4), 153.45 (d, C-2), 155.92 (s, C-6) and 194.79 (s, CO); ESMS; m/z: 366.0 [M+H⁺]; [α]^{D} (CDCl₃)=−13.2.

### 5′-Deoxy-5′acetyl-thioadenosine (3)

A solution of compound 2 (200 mg, 0.54 mmol) was stirred in a mixture of formic acid and water (10 ml, 1:1) at room temperature. The progress of the reaction was monitored by reversed-phase HPLC. After 50 h reaction time the solvent was evaporated under reduced pressure. Traces of formic acid were removed by co-evaporating 5 times with absolute ethanol to produce an off-white powder, which was purified by silica gel flash chromatography [30 g, CH₂Cl₂/CH₃OH (4:1 v/v)]. The fractions containing the product were combined, the solvent removed under reduced pressure and the product further dried in vacuo (0.5 mbar) to title compound 3 (150 mg, 86%); TLC R_{f} (CH₂Cl₂:CH₃ OH, 9:1 v/v)=0.24; ¹H-NMR (CDCl₃): δ 2.32 (s, 3H, COCH₃), 3.15 and 3.34 (AB part of ABX spectrum, J_{5′-H, 4′-H}=5.5 Hz, J_{5′b-H, 4′-H}=7 Hz, J_{gem}=14 Hz, 2H, 5′a-H, 5′b-H), 3.9 (ddd, J_{4′-H, 3′-H}=3.5 Hz, J_{5′a-H, 4′-H}=6 Hz, J_{5′b-H, 4′-H}=7.5 Hz, 1H, 4′-H), 4.08 (m, 1H, 3′-H), 4.76 (t, J_{2′-H, 1′-H}=J_{2′-H, 3′-H}=J_{2′-H, 2′-OH}=6 Hz, 1H, 2′-H), 5.37 (s, 1H, D₂0 exchangeable, 3′-OH), 5.51 (s, 1H, D₂0 exchangeable, 2′-OH), 5.85 (d, J_{1′-H, 2′-H}=6 Hz, 1H, 1′-H), 7.28 (s, br., 2H, D₂0 exchangeable, 6-NH₂), 8.14 (s, 1H, 2-H) and 8.53 (s, 1H, 8-H); ESMS; m/z: 326.5 [M+H⁺].

### 5′-Deoxy-5′-thioadenosine (4)

To eliminate traces of oxygen, a mixture of CH₃OH/H₂O (5:2) was degassed by first passing nitrogen gas (for 15 min) and secondly ammonia gas (for 15 min) through the mixture. Nucleoside 3 (50 mg, 0.16 mmol) was solubilised in the ammonia-saturated CH₃OH/H₂0 mixture (7 mL) under N₂, and the mixture stirred at 0° C. After 1.5 h the reaction mixture was frozen using liquid nitrogen and the solvent removed by drying in vacuo to afford title compound 4 (25 mg, 55%); TLC R_{f} (CH₂Cl₂/CH₃OH, 7:1 v/v)=0.85; mp=109-110° C., ¹H-NMR [(D₆ DMSO)]: δ 2.57 (s, br., 1H, 5′-SH), 2.75-2.80 (m, 2H, 5′a-H, 5′b-H), 3.98 (dt, J_{4′-H, 3′-H}=3 Hz, J_{4′-H, 5′a-H}=J_{4′-H, 5′-bH}=6 Hz, 1H, 4′-H), 4.18 (q, J_{3′-H, 2′-H}=J_{3′-H, 4′-H}=J_{3′-H, 3′-OH}=4 Hz, 1H, 3′-H), 4.78 (q, J_{2′-H, 1′-H}=J_{2′-H, 3′-H}=J_{2′-H, 2′-OH}=5 Hz, 1H, 2′-H), 5.28 (d, J_{3′-OH, 3′-OH}=5 Hz, 1H, 3′-OH), 5.48 (d, J_{2′-OH, 2′-H}=6 Hz, 1H, 2′-OH), 5.88 (d, J_{1′-H, 2′-H}=6 Hz, 1H, 1′-H), 7.28 (s, br., 2H, 6-NH₂), 8.14 (s, 1H, 2-H) and 8.35 (s, 1H, 8-H); ESMS; m/z: 283.92 [M+H⁺]; [α]_{D} (DMSO)=−29.3.

### Example 8

### Inhibition of PLK1 Enzymatic Activity by Adenosine, Thioadenosines, and Thiol-Reactive Compounds

Adenosine, N-ethyhnaleimide, iodoacetamide, and thimerosal were obtained from Sigma Chemical Co. 2′-Thioadenosine was obtained from Calbiochem. 5′-Thioadenosine was prepared as described in Example 7. All compounds were made up as 10 mM stocks in neat dimethylsulfoxide and fresh dilutions to the desired concentrations were made in assay buffer prior to the assay. The candidate compounds were incubated with the enzyme in the kinase assay buffer for the duration of the assay, usually 1 hour at 30° C. (refer Example 4). For each compound duplicate samples, one of which contained dithiothreitol (DTT) at 1 mM final concentration, were assayed. The results are summarized in Table 3 and FIG. 5.

### Example 9

### Inhibition of PLK1 Enzymatic Activity by Other Small Molecules

The effects of staurosporine, a promiscuous kinase inhibitor, and wortmannin, a specific PI-3 kinase inhibitor, were also tested for the inhibition of PLK1 activity. The results showed that while staurosporine caused moderate inhibition of PLK1, wortmannin was considerably more potent, with a very similar activity to that reported for its PI-3 kinase inhibition. The PLK1 IC₅₀ values for staurosporine and wortmannin in the biochemical assay were 0.8±0.2 and 0.18±0.1 μM, respectively (FIG. 8).

In order further to investigate the possibility of other protein kinase inhibitors affecting PLK1 enzymatic activity, a library of trisubstituted purine CDK2 inhibitors was tested in the in vitro assay. It was found that purvalanol A, a potent ATP antagonist of several CDKs also inhibited PLK1 with an activity (IC₅₀) of 5 μl.

### Example 10

### Kinetic Analysis of PLK1 Inhibition by Staurosporine and Wortmannin

In order to determine the nature of inhibition of PLK1 activity by staurosporine and wortmannin, a full investigation of the dependence on ATP concentration of the inhibition by these two compounds was carried out (FIG. 9). The results obtained showed that staurosporine inhibition was a fully ATP-competitive, whereas that of wortmannin was completely ATP-independent. This situation mirrors the previously reported mechanism of inhibition of PI-3 kinase by wortmannin through irreversible covalent modification of Lys833 in the ATP-binding site [37]. Staurosporine, on the other hand, was also reported to be less potent against PI-3 kinase (IC₅₀ of 10 μM) [37].

### Example 11

### Flavonoids Inhibit PLK1 Activity In Vitro

Based on the results clearly demonstrating that wortmannin is very potent against PLK1, we sought to test whether any other known PI3 kinase inhibitors have an effect on PLK1 activity. A number of flavonoid compounds including LY294002, Quercetin and Myricetin which were previously reported to cause a moderate inhibition of PI3 kinase activity (IC₅₀ values of 1.4, 3.8 and 1.8 μM respectively, [37]) were screened against PLK1 (Table 12). Interestingly, the results showed that indeed LY294002 was equally potent against PLK1 giving an IC50 value of 5-10 μM. Quercetin on the other hand was less potent (64 μM) whilst Myricetin was inactive against PLK1 (>100 μM IC₅₀).

Table 13 shows a summary of screening of 8 additional flavonoid compounds against PLK1. Of these morin hydrate was the most potent with an IC₅₀ of 12 μM.

As dose-response inhibition for a number of closely related flavonoid inhibitors was obtained, it was possible to determine a structure-activity relationship for this compound class. Each of the other 10 compounds screened contains an identical core structure to morin and only vary on the extent of hydroxyl substitutions on the flavonoid. Comparing the inactive inhibitor, datescetin with morin, reveals that the R3′ hydroxyl is important for binding (since it is absent in Datescetin). The lower potency of quercetin on PLK1 (64 μM) and its lack of a R1′hydroxyl also suggests that it makes intermolecular contacts in the ATP cleft. The lack of inhibition of myricetin and kaepmpferol which also lack this group is consistent this observation although it is likely that the additional OH group at R2′ in myricetin interferes with binding. Comparison of luteolin with the weak inhibitor, quercetin suggests that the R3 hydroxyl makes a contribution due to the absence of this group in the former compounds. The inactivity of gangolin, which has no substituents on the 2^{nd} ring is expected, however the weak inhibition of robinetin is unusual. This compound is similar to the inactive myricetin however does not have an R1 hydroxyl suggesting that this group makes unfavourable interactions and removing it results in tighter interaction. The weak inhibition observed for robinetin is probably at the threshold of sensitivity of the kinase assay and therefore may not be reliable. The inactivity of daidzein, fisetin and kaempferide is in line with the impotency of other similar compounds in this series.

In addition, based on literature reports [36] we found that out of 25 kinases tested, Casein Kinase II was the second most sensitive to inhibition by LY294002. The effects of wortmannin and LY294002 against Casein kinase II were tested and compared that to PLK1 inhibition (FIG. 10).

### Example 12

### Sequence and Structural Comparison of PLK1 with Other Protein Kinases

In order to obtain more information on the kinase domain of PLK1 and further characterise the residues that comprise the ATP binding pocket, a sequence similarity and homology analysis was performed (FIG. 3). A FASTA search of protein kinases with available 3-D structural information revealed that the closest structural matches for the kinase domain included Cdk2 and ERK2, however the AGC kinase, PKA had the highest homology (over 40% similarity and 30% identity) As a consequence of the similarities of PLK1 and PKA, several commonly used PKA inhibitors were tested to determine if any correlation exists between the structural similarities and mode of inhibition of these two enzymes.

To this end, commercially available PKA inhibitors H89, A3 hydrochloride, KT5720 and 4-cyano 3-methylisoquinoline were screened against PLK1 and the results were compared to the published values against PKA. Surprisingly, none of these compounds caused any inhibition of PLK1, even at concentrations as high as 1 mM. Moreover, Balanol a very potent inhibitor of the ACG family of protein kinases [47] was tested here to show no detectable inhibition of PLK1. Put together, these result clearly demonstrate that despite the fact the PLK1 has the greatest homology with PKA, their mode and mechanism of inhibition by small molecule ATP competitors appear to be vastly different (Table 14).

### Example 13

### Molecular Modelling of the Interactions of Inhibitors with PLK1 Kinase Domain

As mentioned above, the closest structural homologue to the kinase domain of PLK1 is protein kinase A. Despite the relatively low sequence identity between these two enzymes, the structural conservation of the protein kinase fold allowed the construction of a homology model structure of PLK1. This hypothetical structure was then used in flexible docking calculations with the identified PLK1 ATP competitive ligands to determine if representative kinase binding modes could be identified and thus enable validation of model. Positioning of the trisubstituted purine derivative, purvalanol A was undertaken using the automated docking routine, Affinity (I2000, Accelrys) that allows for flexibility in both the receptor binding site and in the ligand itself. The use of this ligand is expedient as it is a potent Cdk2 inhibitor and its complex crystal structure has been previously determined. While it is possible that purvalanol A binds to PLK1 in a different way, its Cdk2 pose is nonetheless suggestive of how the purines interact with the mitotic kinase. Investigation of numerous predicted structures of purvalanol A with PLK1 indeed revealed an energetically favourable pose that formed similar contacts to those observed in the Cdk2 bound structure (FIG. 11A).

The hinge region H-bonds observed in the Cdk2 complex (E81, L83) were formed with C133 of PLK1 and in addition the isopropyl group interacts with the deep cleft of the ATP pocket (L130 corresponding to F80 in Cdk2). As a cross-validation, purvalanol A was also docked into the structure of PKA that was used as the template for the PLK1 model. This result confirmed that no binding mode forming kinase inhibitory contacts was observed with PKA and therefore was consistent with the lack of inhibition of this inhibitor. In order to probe the structural basis for the lower potency of staurosporine against PLK1, this compounds was modelled into the homology structure. A similar binding mode to that observed in Cdk2 was observed. Wortmannin also was modelled in the ATP cleft of the PLK1 homology structure to determine if the structural basis for its irreversible inhibition could be predicted. Docking of this inhibitor revealed an energetically favourable binding mode that placed the reactive functionality in close proximity to K82 of PLK1. Formation of the covalent bond between Wortmannin and K82, followed by energy minimisation to convergence resulted in a plausible low energy complex structure that was consistent with its interactions in the PI3 kinase experimental structure (FIG. 12).

In order to further examine, the interactions of the newly characterised PLK1 inhibitors, the flavonoid compound LY294002 was additionally docked into the PLK1 kinase domain. As this compound has been developed as a PI3 kinase inhibitor and since its co-crystal structure has been solved, a useful benchmark is available to probe the model structure. This time however, comparison of the structural ensemble of docked poses showed that no energetically realistic binding mode closely representing that observed with PI3K. Comparison of the primary structure of PI3K and PLK1 shows that these two enzymes have a low sequence identity (15%) and diverge considerably in the residues lining the ATP cleft. It is thus very possible that LY294002 forms different non-bonded interactions in the PLK1 context. Evaluation of the most energetically favourable structure for this inhibitor indicates a plausible binding mode with the PLK1 catalytic domain however is substantially different from the binding mode observed in the PI3K structure.

Due to the observed activity of morin hydrate on PLK1 and since activity data was available for a number of close structural analogues, this compound was additionally docked into the PLK1 kinase domain. Examination of the structural ensemble generated by molecular dynamics docking indicates that energetically plausible poses representative of “kinase inhibitors” from crystal structures are observed and are consistent with the activities of other molecules in this series (FIG. 11B).

### Example 14

### ATP-Dependence of PLK1 Inhibition by 5′-thioadenosine

The kinase assay described in Example 4 was used. ATP dependence of the effects of adenosine, 2′-thioadenosine, 5-′thioadenosine, and thimerosal was investigated at 12.5, 25, 50, and 100 μM ATP. The results showed that none of these compounds were classical competitive inhibitors with respect to ATP, as would be expected from a covalent inhibitor. Results of the kinetic analysis with 5′-thioadenosine are shown in FIG. 6.

### Example 15

### Contact Models of PLK1 Kinase Domain with Bound Ligands

The homology model described in Example 1 was used as the basis for the docking of ATP, 5′-thioadenosine, and two additional ATP-competitive kinase inhibitors we have found to inhibit PLK1. The conformations of these ligands in the PLK1 ATP-binding pocket are depicted in FIG. 7. Descriptions of the PLK1-ligand complex structures in the form of interatomic distances between the residues lining the ATP-binding pocket of PLK1 and the ligands were obtained using the molecular modelling programs Quanta2000 (Accelrys, Calif., USA) and Maestro (Schrodinger Inc., Oregon, USA). The output from the former lists all contacts between PLK1 and ligands that are less than 3.5 Å. In the latter case a listing of all PLK1-ligand contacts not involving H atoms is given, together with the interatomic distances. Also given is a measure of the quality of the contacts. Only favourable contacts are listed and the closer the value of the contact cut-off ratio to 1.3, the better the contact. Results are summarized in Table 4 (Maestro) & Table 5 (Quanta) for ATP, in Table 6 (Maestro) & Table 7 (Quanta) for 5′-thioadenosine, in Table 8 (Maestro) & Table 9 (Quanta) for staurosporine, and in Table 10 (Maestro) & Table 11 (Quanta) for 4-[4-(4-methyl-2-methylamino-thiazol-5-yl)-pyrimidin-2-ylamino]-phenol. The ligand atom numbering is shown in FIG. 7.

### Example 16

### Covalent Inhibition of PLK1 by Benzthiazole N-Oxide Derivative

The homology model of the invention was further validated by studies using two known inhibitors of PLK, Inhibitors A and B, the structures of which are shown below.

As is shown in FIG. 13, the selective PLK1 inhibitor A (IC₅₀ for PLK1 activity is 0.5 μM at 10 μM ATP) competes with ATP for binding to the active site of the enzyme. Furthermore, upon varying the concentration of inhibitor as well as of ATP, the kinetic analysis shows that the binding of the inhibitor is fully reversible, as the K_{M, ATP} (intercepts on the abscissa in the Lineweaver-Burk plot) vary, with no change in the reaction velocity V, X of the enzyme (common intersect on the ordinate).

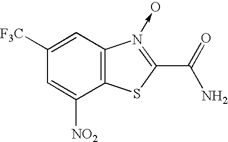

### Inhibitor A

### 7-Nitro-3-oxy-5-trifluoromethyl-benzothiazole-2-carboxylic acid amide

The closely related analogue Inhibitor B, which only differs from A by the presence of a SCF₃ group rather than a CF₃ group, shows different behaviour. The kinetic analysis for this compound suggests that the inhibitor affects the Vₘₐₓ of the enzyme, without altering the apparent affinity for ATP (K_{M, ATP}) (FIG. 14). This shows that the inhibitor is non-competitive with respect to ATP and hence strongly suggests that it is binding covalently to the PLK1 ATP binding site.

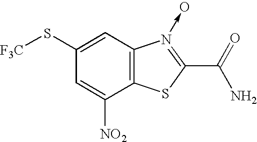

### Inhibitor B

### 7-Nitro-3-oxy-5-trifluoromethylsulfanyl-benzothiazole-2-carboxylic acid amide

This covalent binding would most likely be with the cysteine residue (C67) in the binding pocket of PLK1 and is supported through the close proximity of the potential reactive atoms of Inhibitor B to the cysteine in the modelled structure of inhibitor A shown in FIG. 15.

Various modifications and variations of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention.

### REFERENCES

[1] Nurse, P. (1990) Nature, 344, 503-508.
[2] Nigg, E. A. (2001) Nat. Rev. Mol. Cell. Biol., 2, 21-32.
[3] Takizawa, C. G. and Morgan, D. O. (2000) Curr. Opin. Cell Biol., 12, 658-665.
[4] Roshak, A. K., Capper, E. A., Imburgia, C., Fornwald, J., Scott, G. and Marshall, L. A. (2000) Cell. Signalling, 12, 405-411.
[5] Glover, D. M., Hagan, I. M. and Tavares, A. A. (1998) Genes Dev., 12, 3777-3787.
[6] Seong, Y.-S., Kamijo, K., Lee, J.-S., Fernandez, E., Kuriyama, R., Miki, T. and Lee, K. S. (2002) J. Biol. Chem., 277, 32282-32293.
[7] Wang, Q., Xie, S., Chen, J., Fukusawa, K., Naik, U., Traganos, F., Darzynkiewicz, Z., Jhanwar-Uniyal, M. and Dai, W. (2002) Mol. Cell. Biol., 22, 3450-3459.
[8] Kauselmann, G., Weiler, M., Wulff, P., Jessberger, S., Konietzko, U., Scafidi, J., Staubli, U., Bereiter-Hahn, J., Strebhardt, K. and Kuhl, D. (1999) EMBO J., 18, 5528-5539.
[9] Lane, H. A. and Nigg, E. A. (1996) J. Cell. Biol., 135, 1701-1713.
[10] Cogswell, J. P., Brown, C. E., Bisi, J. E. and Neill, S. D. (2000) Cell Growth Differ., 11, 615-623.
[11] Liu, X. and Erikson, R. L. (2003) Proc. Natl. Acad. Sci. USA, 100, 5789-5794.
[12] Spankuch-Schmitt, B., Wolf, G., Solbach, C., Loibl, S., Knecht, R., Stegmuller, M., von Minckwitz, G., Kaufmann, M. and Strebhardt, K. (2002) Oncogene, 21, 3162-3171.
[13] Smith, M. R., Wilson, M. L., Hamanaka, R., Chase, D., Kung, H., Longo, D. L. Ferris, D. K. (1997) Biochem. Biophys. Res. Commun., 234, 397-405.
[14] Takahashi, T., Sano, B., Nagata, T., Kato, H., Sugiyama, Y., Kunieda, K., Kimura, M., Okano, Y. and Saji, S. (2003) Cancer Science, 94, 148-152.
[15] Tokumitsu, Y., Mori, M., Tanaka, S., Akazawa, K., Nakano, S. and Niho, Y. (1999) Int. J. Oncol., 15, 687-692.
[16] Wolf, G., Elez, R., Doermer, A., Holtrich, U., Ackermann, H., Stutte, H. J., Altmannsberger, H.-M., Rübsamen-Waigmann, H. and Strebhardt, K. (1997) Oncogene, 14, 543-549.
[17] Kraker, A. J. and Booher, R. N. (1999) In Annual Reports in Medicinal Chemistry (Vol. 34) (Doherty, A. M., ed.), pp. 247-256, Academic Press.
[18] Stevenson, C. S., Capper, E. A., Roshak, A. K., Marquez, B., Eichman, C., Jackson, J. R., Mattern, M., Gerwick, W. H., Jacobs, R. S. and Marshall, L. A. (2002) J. Pharmacol. Exp. Ther., 303, 858-866.
[19] Stevenson, C. S., Capper, E. A., Roshak, A. K., Marquez, B., Grace, K., Gerwick, W. H., Jacobs, R. S. and Marshall, L. A. (2002) Inflammation Research, 51, 112-114.
[20] Singh, J. (1994) Protein Engineering, 7, 849-858.
[21] Singh, J., Dobrusin, E. M., Fry, D. W., Haske, T., Whitty, A. and McNamara, D. J. (1997) J. Med. Chem., 40, 1130-1135.
[22] Fry, D. W., Bridges, A. J., Denny, W. A., Doherty, A., Greis, K. D., Hicks, J. L., Hook, K. E., Keller, P. R., Leopold, W. R., Loo, J. A., McNamara, D. J., Nelson, J. M., Sherwood, V., Smaill, J. B., Trumpp-Kallmeyer, S. and Dobrusin, E. M. (1998) Proc. Natl. Acad. Sci. USA, 95, 12022-12027.
[23] Smaill, J. B., Showalter, H. D. H., Zhou, H., Bridges, A. J., McNamara, D. J., Fry, D. W., Nelson, J. M., Sherwood, V., Vincent, P. W., Roberts, B. J., Elliott, W. L.
and Denny, W. A. (2001) J. Med. Chem., 44, 429-440.
[24] Tsou, H.-R., Mamuya, N., Johnson, B. D., Reich, M. F., Gruber, B. C., Ye, F., Nilakantan, R., Shen, R., Discafani, C., DeBlanc, R, Davis, R., Koehn, F. E., Greenberger, L. M., Wang, Y.-F. and Wissner, A. (2001) J. Med. Chem., 44, 2719-2734.
[25] Denny, W. A. (2002) Pharmacology & Therapeutics, 93, 253-261.
[26] Smaill, J. B., Rewcastle, G. W., Loo, J. A., Greis, K. D., Chan, O. H., Reyner, E. L., Lipka, E., Showalter, H. D. H., Vincent, P. W., Elliott, W. L. and Denny, W. A. (2000) J. Med. Chem., 43, 1380-1397.
[27] Allen, L. F., Lenehan, P. F., Eiseman, I. A., Elliott, W. L. and Fry, D. W. (2002) Seminars in Oncology, 29 (3, Suppl. 11), 11-21.
[28] Torrance, C. J., Jackson, P. E., Montgomery, E., Kinzler, K. W., Vogelstein, B., Wissner, A., Nunes, M., Frost, P. and Discafani, C. M. (2000) Nat. Med., 6(9), 1024-1028.
[29] Wissner, A., Overbeek, E., Reich, M. F., Floyd, M. B., Johnson, B. D., Mamuya, N., Rosfjord, E. C., Discafani, C., Davis, R., Shi, X., Rabindran, S. K., Gruber, B. C., Ye, F., Hallett, W. A., Nilakantan, R., Shen, R., Wang, Y.-F., Greenberger, L. M. and Tsou, H.-R. (2003) J. Med. Chem., 46, 49-63.
[30] Stamos, J., Sliwkowski, M. X. and Eigenbrot, C. (2002) J. Biol. Chem., 277, 46265-46272.
[31] Brenner, S. E. (2001) Nat. Rev. Genet., 2, 801-809.
[32] Gescher, A. (1998) General Pharmacology, 31, 721-728.
[33] Fischer, P. M. and Wang, S. (2001) In PCT Intl. Patent Appl. WO 2001072745, Cyclacel Limited, UK.
[34] Elferink, J. G. R. (1999) Gen. Pharmacol., 33, 1-6.
[35] Cleland, W. W. (1964) Biochemistry, 4, 480-485.
[36] Davies, S. P., Reddy, H., Caivano, M. and Cohen, P. (2000) Biochem. J., 351, 95-105.
[37] Walker, E. H., Pacold, M. E., Perisic, O., Stephens, L., Hawkins, P. T., Wymann, M. P. and Williams, R. L. (2000) Mol. Cell, 6, 909-919.
[38] Greer, J. (1990) Proteins, 7, 317-334.
[39] Pearson, W. R. (2000) Methods Mol. Biol., 132, 185-219.
[40] Henikoff, S. and Henikoff, J. G. (1992) Proc. Natl. Acad. Sci. USA, 89, 10915-10919.
[41] Altschul, S. F. (1991) J. Mol. Biol., 219, 555-565.
[42] Venkatachalam, C. M. and Ramachandran, G. N. (1973) Ann. Rev. Biochem., 38, 45-77.
[43] Westbrook, J. D. and Fitzgerald, P. M. D. (2003) Methods Biochem. Anal., 44, 161-179.
[44] Hamanaka, R., Maloid, S., Smith, M. R., O'Connell, C. D., Longo, D. L. and Ferris, D. K. (1994) Cell Growth Differ., 5, 249-257.
[45] Marriott, J. H., Mottahedeh, M. and Reese, C. B. (1990) Tetrahedron Lett., 31, 7485-7488.
[46] Pignot, M., Pljevaljcic, G. and Weinhold, E. (2000) Eur. J. Org. Chem., 549-555.
[47] Setyawan J., Koide K., Diller T. C., Bunnage M. E., Taylor S. S., Nicolaou K. C., Brunton L. L. (1999) Mol. Pharmacol. 56. 370-376.
[48] Jacobs, R. S., Stevenson, C. S., Gerwick, W. H., Marshall, L. A. (2001) Therapeutic applications of Scytonemin, a polo-like kinase (PLK) inhibitor. WO 2001062900; University of California, USA: PCT Int. Appl., 64 pp.
[49] Mundt, K. E., Golsteyn, R. M., Lane, H. A., Nigg E. A. (1997) Biochem. Biophys. Res. Comm. 239, 377-385.

TABLE 1
Sequence comparison between PLK1 and CDK2, ERK-2, or PKA kinase
domains, respectively.

| | Sequence identity(%) | | |
|---|---|---|---|
| PLK1 sequence segment | CDK2 | ERK-2 | PKA |
| 1-50 | 0 | 8 | 12 |
| 51-100 | 14 | 20 | 28 |
| 101-150 | 18 | 8 | 20 |
| 151-200 | 44 | 48 | 44 |
| 201-250 | 30 | 30 | 42 |
| 251-306 | 18 | 20 | 22 |

TABLE 2
PDB coordinate file of PLK1-ATP homology model

| | | | | | | | | | | |
|---|---|---|---|---|---|---|---|---|---|---|
| ATOM | 1 | N | ARG | 52 | 108.414 | 117.322 | 91.897 | 1.00 | 0.00 | N |
| ATOM | 2 | CA | ARG | 52 | 109.182 | 116.827 | 90.698 | 1.00 | 0.00 | C |
| ATOM | 3 | C | ARG | 52 | 108.390 | 116.045 | 89.578 | 1.00 | 0.00 | C |
| ATOM | 4 | O | ARG | 52 | 108.985 | 115.798 | 88.530 | 1.00 | 0.00 | O |
| ATOM | 5 | CB | ARG | 52 | 110.589 | 116.233 | 91.053 | 1.00 | 0.00 | C |
| ATOM | 6 | CG | ARG | 52 | 110.801 | 114.702 | 91.020 | 1.00 | 0.00 | C |
| ATOM | 7 | CD | ARG | 52 | 112.287 | 114.328 | 91.157 | 1.00 | 0.00 | C |
| ATOM | 8 | NE | ARG | 52 | 112.450 | 112.916 | 90.739 | 1.00 | 0.00 | N |
| ATOM | 9 | CZ | ARG | 52 | 113.551 | 112.190 | 90.870 | 1.00 | 0.00 | C |
| ATOM | 10 | NH1 | ARG | 52 | 114.666 | 112.630 | 91.370 | 1.00 | 0.00 | N |
| ATOM | 11 | NH2 | ARG | 52 | 113.501 | 110.971 | 90.474 | 1.00 | 0.00 | N |
| ATOM | 12 | 1H | ARG | 52 | 107.626 | 116.687 | 92.087 | 1.00 | 0.00 | H |
| ATOM | 13 | 2H | ARG | 52 | 109.037 | 117.350 | 92.717 | 1.00 | 0.00 | H |
| ATOM | 14 | HE | ARG | 52 | 111.635 | 112.458 | 90.308 | 1.00 | 0.00 | H |
| ATOM | 15 | HA | ARG | 52 | 109.432 | 117.749 | 90.134 | 1.00 | 0.00 | H |
| ATOM | 16 | 1HB | ARG | 52 | 111.303 | 116.678 | 90.331 | 1.00 | 0.00 | H |
| ATOM | 17 | 2HB | ARG | 52 | 110.945 | 116.616 | 92.029 | 1.00 | 0.00 | H |
| ATOM | 18 | 1HG | ARG | 52 | 110.209 | 114.203 | 91.813 | 1.00 | 0.00 | H |
| ATOM | 19 | 2HG | ARG | 52 | 110.408 | 114.292 | 90.070 | 1.00 | 0.00 | H |
| ATOM | 20 | 1HD | ARG | 52 | 112.925 | 114.977 | 90.524 | 1.00 | 0.00 | H |
| ATOM | 21 | 2HD | ARG | 52 | 112.620 | 114.481 | 92.204 | 1.00 | 0.00 | H |
| ATOM | 22 | 2HH1 | ARG | 52 | 114.619 | 113.601 | 91.675 | 1.00 | 0.00 | H |
| ATOM | 23 | 1HH1 | ARG | 52 | 115.438 | 111.966 | 91.428 | 1.00 | 0.00 | H |
| ATOM | 24 | 1HH2 | ARG | 52 | 112.572 | 110.717 | 90.120 | 1.00 | 0.00 | H |
| ATOM | 25 | 2HH2 | ARG | 52 | 114.330 | 110.391 | 90.596 | 1.00 | 0.00 | H |
| ATOM | 26 | N | TYR | 53 | 107.105 | 115.659 | 89.725 | 1.00 | 0.00 | N |
| ATOM | 27 | CA | TYR | 53 | 106.360 | 114.857 | 88.698 | 1.00 | 0.00 | C |
| ATOM | 28 | C | TYR | 53 | 104.944 | 115.448 | 88.356 | 1.00 | 0.00 | C |
| ATOM | 29 | O | TYR | 53 | 104.213 | 115.917 | 89.234 | 1.00 | 0.00 | O |
| ATOM | 30 | CB | TYR | 53 | 106.221 | 113.387 | 89.193 | 1.00 | 0.00 | C |
| ATOM | 31 | CG | TYR | 53 | 107.481 | 112.506 | 89.105 | 1.00 | 0.00 | C |
| ATOM | 32 | CD1 | TYR | 53 | 108.238 | 112.270 | 90.254 | 1.00 | 0.00 | C |
| ATOM | 33 | CD2 | TYR | 53 | 107.859 | 111.902 | 87.899 | 1.00 | 0.00 | C |
| ATOM | 34 | CE1 | TYR | 53 | 109.362 | 111.450 | 90.197 | 1.00 | 0.00 | C |
| ATOM | 35 | CE2 | TYR | 53 | 108.977 | 111.069 | 87.849 | 1.00 | 0.00 | C |
| ATOM | 36 | CZ | TYR | 53 | 109.729 | 110.848 | 89.000 | 1.00 | 0.00 | C |
| ATOM | 37 | OH | TYR | 53 | 110.838 | 110.047 | 88.972 | 1.00 | 0.00 | O |
| ATOM | 38 | H | TYR | 53 | 106.610 | 115.929 | 90.587 | 1.00 | 0.00 | H |
| ATOM | 39 | HA | TYR | 53 | 106.932 | 114.835 | 87.749 | 1.00 | 0.00 | H |
| ATOM | 40 | 1HB | TYR | 53 | 105.807 | 113.374 | 90.220 | 1.00 | 0.00 | H |
| ATOM | 41 | 2HB | TYR | 53 | 105.431 | 112.881 | 88.609 | 1.00 | 0.00 | H |
| ATOM | 42 | HD1 | TYR | 53 | 107.971 | 112.729 | 91.194 | 1.00 | 0.00 | H |
| ATOM | 43 | HD2 | TYR | 53 | 107.294 | 112.078 | 86.995 | 1.00 | 0.00 | H |
| ATOM | 44 | HE1 | TYR | 53 | 109.966 | 111.296 | 91.080 | 1.00 | 0.00 | H |
| ATOM | 45 | HE2 | TYR | 53 | 109.268 | 110.610 | 86.916 | 1.00 | 0.00 | H |
| ATOM | 46 | HH | TYR | 53 | 111.034 | 109.782 | 88.067 | 1.00 | 0.00 | H |
| ATOM | 47 | N | VAL | 54 | 104.539 | 115.358 | 87.076 | 1.00 | 0.00 | N |
| ATOM | 48 | CA | VAL | 54 | 103.182 | 115.765 | 86.588 | 1.00 | 0.00 | C |
| ATOM | 49 | C | VAL | 54 | 102.488 | 114.515 | 85.933 | 1.00 | 0.00 | C |
| ATOM | 50 | O | VAL | 54 | 102.989 | 113.950 | 84.954 | 1.00 | 0.00 | O |
| ATOM | 51 | CB | VAL | 54 | 103.294 | 116.991 | 85.608 | 1.00 | 0.00 | C |
| ATOM | 52 | CG1 | VAL | 54 | 101.959 | 117.391 | 84.930 | 1.00 | 0.00 | C |
| ATOM | 53 | CG2 | VAL | 54 | 103.822 | 118.277 | 86.288 | 1.00 | 0.00 | C |
| ATOM | 54 | H | VAL | 54 | 105.247 | 114.977 | 86.430 | 1.00 | 0.00 | H |
| ATOM | 55 | HA | VAL | 54 | 102.552 | 116.097 | 87.438 | 1.00 | 0.00 | H |
| ATOM | 56 | HB | VAL | 54 | 104.004 | 116.714 | 84.802 | 1.00 | 0.00 | H |
| ATOM | 57 | 1HG1 | VAL | 54 | 101.183 | 117.679 | 85.665 | 1.00 | 0.00 | H |
| ATOM | 58 | 2HG1 | VAL | 54 | 102.083 | 118.243 | 84.234 | 1.00 | 0.00 | H |
| ATOM | 59 | 3HG1 | VAL | 54 | 101.536 | 116.567 | 84.325 | 1.00 | 0.00 | H |
| ATOM | 60 | 2HG2 | VAL | 54 | 104.802 | 118.112 | 86.769 | 1.00 | 0.00 | H |
| ATOM | 61 | 3HG2 | VAL | 54 | 103.964 | 119.104 | 85.567 | 1.00 | 0.00 | H |
| ATOM | 62 | 1HG2 | VAL | 54 | 103.140 | 118.642 | 87.079 | 1.00 | 0.00 | H |
| ATOM | 63 | N | ARG | 55 | 101.311 | 114.102 | 86.439 | 0.00 | 0.00 | N |
| ATOM | 64 | CA | ARG | 55 | 100.503 | 113.002 | 85.830 | 0.00 | 0.00 | C |
| ATOM | 65 | C | ARG | 55 | 99.683 | 113.480 | 84.579 | 0.00 | 0.00 | C |
| ATOM | 66 | O | ARG | 55 | 98.913 | 114.441 | 84.665 | 0.00 | 0.00 | O |
| ATOM | 67 | CB | ARG | 55 | 99.533 | 112.395 | 86.882 | 0.00 | 0.00 | C |
| ATOM | 68 | CG | ARG | 55 | 100.151 | 111.773 | 88.159 | 0.00 | 0.00 | C |
| ATOM | 69 | CD | ARG | 55 | 99.092 | 111.038 | 88.997 | 0.00 | 0.00 | C |
| ATOM | 70 | NE | ARG | 55 | 99.641 | 110.761 | 90.348 | 0.00 | 0.00 | N |
| ATOM | 71 | CZ | ARG | 55 | 98.978 | 110.180 | 91.339 | 0.00 | 0.00 | C |
| ATOM | 72 | NH1 | ARG | 55 | 97.772 | 109.704 | 91.239 | 0.00 | 0.00 | N |
| ATOM | 73 | NH2 | ARG | 55 | 99.572 | 110.091 | 92.474 | 0.00 | 0.00 | N |
| ATOM | 74 | HE | ARG | 55 | 100.615 | 111.041 | 90.530 | 1.00 | 0.00 | H |
| ATOM | 75 | H | ARG | 55 | 100.954 | 114.668 | 87.214 | 0.00 | 0.00 | H |
| ATOM | 76 | HA | ARG | 55 | 101.187 | 112.185 | 85.519 | 0.00 | 0.00 | H |
| ATOM | 77 | 1HB | ARG | 55 | 98.926 | 111.617 | 86.379 | 0.00 | 0.00 | H |
| ATOM | 78 | 2HB | ARG | 55 | 98.793 | 113.166 | 87.182 | 0.00 | 0.00 | H |
| ATOM | 79 | 1HG | ARG | 55 | 100.628 | 112.570 | 88.763 | 0.00 | 0.00 | H |
| ATOM | 80 | 2HG | ARG | 55 | 100.970 | 111.072 | 87.909 | 0.00 | 0.00 | H |
| ATOM | 81 | 1HD | ARG | 55 | 98.785 | 110.098 | 88.493 | 0.00 | 0.00 | H |
| ATOM | 82 | 2HD | ARG | 55 | 98.176 | 111.658 | 89.090 | 0.00 | 0.00 | H |
| ATOM | 83 | 1HH1 | ARG | 55 | 97.371 | 109.276 | 92.070 | 0.00 | 0.00 | H |
| ATOM | 84 | 2HH1 | ARG | 55 | 97.378 | 109.802 | 90.301 | 0.00 | 0.00 | H |
| ATOM | 85 | 1HH2 | ARG | 55 | 99.060 | 109.677 | 93.250 | 0.00 | 0.00 | H |
| ATOM | 86 | 2HH2 | ARG | 55 | 100.498 | 110.524 | 92.448 | 0.00 | 0.00 | H |
| ATOM | 87 | N | GLY | 56 | 99.823 | 112.791 | 83.436 | 1.00 | 0.00 | N |
| ATOM | 88 | CA | GLY | 56 | 99.062 | 113.119 | 82.194 | 1.00 | 0.00 | C |
| ATOM | 89 | C | GLY | 56 | 97.780 | 112.295 | 81.942 | 1.00 | 0.00 | C |
| ATOM | 90 | O | GLY | 56 | 96.678 | 112.843 | 81.956 | 1.00 | 0.00 | O |
| ATOM | 91 | H | GLY | 56 | 100.528 | 112.039 | 83.459 | 1.00 | 0.00 | H |
| ATOM | 92 | 1HA | GLY | 56 | 98.786 | 114.192 | 82.166 | 1.00 | 0.00 | H |
| ATOM | 93 | 2HA | GLY | 56 | 99.729 | 112.995 | 81.322 | 1.00 | 0.00 | H |
| ATOM | 94 | N | ARG | 57 | 97.923 | 110.991 | 81.655 | 1.00 | 0.00 | N |
| ATOM | 95 | CA | ARG | 57 | 96.765 | 110.087 | 81.374 | 1.00 | 0.00 | C |
| ATOM | 96 | C | ARG | 57 | 97.000 | 108.655 | 81.967 | 1.00 | 0.00 | C |
| ATOM | 97 | O | ARG | 57 | 98.134 | 108.174 | 82.064 | 1.00 | 0.00 | O |
| ATOM | 98 | CB | ARG | 57 | 96.526 | 110.079 | 79.834 | 1.00 | 0.00 | C |
| ATOM | 99 | CG | ARG | 57 | 95.213 | 109.398 | 79.373 | 1.00 | 0.00 | C |
| ATOM | 100 | CD | ARG | 57 | 94.996 | 109.479 | 77.856 | 1.00 | 0.00 | C |
| ATOM | 101 | NE | ARG | 57 | 93.701 | 108.821 | 77.548 | 1.00 | 0.00 | N |
| ATOM | 102 | CZ | ARG | 57 | 93.241 | 108.542 | 76.337 | 1.00 | 0.00 | C |
| ATOM | 103 | NH1 | ARG | 57 | 93.863 | 108.827 | 75.232 | 1.00 | 0.00 | N |
| ATOM | 104 | NH2 | ARG | 57 | 92.101 | 107.952 | 76.262 | 1.00 | 0.00 | N |
| ATOM | 105 | HE | ARG | 57 | 93.108 | 108.556 | 78.347 | 1.00 | 0.00 | H |
| ATOM | 106 | H | ARG | 57 | 98.881 | 110.643 | 81.765 | 1.00 | 0.00 | H |
| ATOM | 107 | HA | ARG | 57 | 95.857 | 110.497 | 81.863 | 1.00 | 0.00 | H |
| ATOM | 108 | 1HB | ARG | 57 | 96.511 | 111.124 | 79.467 | 1.00 | 0.00 | H |
| ATOM | 109 | 2HB | ARG | 57 | 97.390 | 109.607 | 79.326 | 1.00 | 0.00 | H |
| ATOM | 110 | 1HG | ARG | 57 | 95.204 | 108.329 | 79.670 | 1.00 | 0.00 | H |
| ATOM | 111 | 2HG | ARG | 57 | 94.345 | 109.854 | 79.890 | 1.00 | 0.00 | H |
| ATOM | 112 | 1HD | ARG | 57 | 94.981 | 110.534 | 77.515 | 1.00 | 0.00 | H |
| ATOM | 113 | 2HD | ARG | 57 | 95.830 | 108.976 | 77.325 | 1.00 | 0.00 | H |
| ATOM | 114 | 2HH1 | ARG | 57 | 94.757 | 109.292 | 75.385 | 1.00 | 0.00 | H |
| ATOM | 115 | 1HH1 | ARG | 57 | 93.404 | 108.559 | 74.363 | 1.00 | 0.00 | H |
| ATOM | 116 | 1HH2 | ARG | 57 | 91.716 | 107.762 | 77.189 | 1.00 | 0.00 | H |
| ATOM | 117 | 2HH2 | ARG | 57 | 91.741 | 107.720 | 75.338 | 1.00 | 0.00 | H |
| ATOM | 118 | N | PHE | 58 | 95.918 | 107.949 | 82.336 | 1.00 | 0.00 | N |
| ATOM | 119 | CA | PHE | 58 | 95.987 | 106.512 | 82.726 | 1.00 | 0.00 | C |
| ATOM | 120 | C | PHE | 58 | 96.279 | 105.555 | 81.519 | 1.00 | 0.00 | C |
| ATOM | 121 | O | PHE | 58 | 95.611 | 105.617 | 80.481 | 1.00 | 0.00 | O |
| ATOM | 122 | CB | PHE | 58 | 94.718 | 106.129 | 83.545 | 1.00 | 0.00 | C |
| ATOM | 123 | CG | PHE | 58 | 93.362 | 106.079 | 82.812 | 1.00 | 0.00 | C |
| ATOM | 124 | CD1 | PHE | 58 | 92.925 | 104.888 | 82.221 | 1.00 | 0.00 | C |
| ATOM | 125 | CE1 | PHE | 58 | 91.712 | 104.847 | 81.539 | 1.00 | 0.00 | C |
| ATOM | 126 | CZ | PHE | 58 | 90.924 | 105.992 | 81.449 | 1.00 | 0.00 | C |
| ATOM | 127 | CE2 | PHE | 58 | 91.347 | 107.179 | 82.043 | 1.00 | 0.00 | C |
| ATOM | 128 | CD2 | PHE | 58 | 92.561 | 107.221 | 82.725 | 1.00 | 0.00 | C |
| ATOM | 129 | H | PHE | 58 | 95.026 | 108.372 | 82.064 | 1.00 | 0.00 | H |
| ATOM | 130 | HA | PHE | 58 | 96.818 | 106.419 | 83.454 | 1.00 | 0.00 | H |
| ATOM | 131 | 1HB | PHE | 58 | 94.904 | 105.145 | 84.019 | 1.00 | 0.00 | H |
| ATOM | 132 | 2HB | PHE | 58 | 94.636 | 106.807 | 84.415 | 1.00 | 0.00 | H |
| ATOM | 133 | HD1 | PHE | 58 | 93.540 | 103.999 | 82.262 | 1.00 | 0.00 | H |
| ATOM | 134 | HE1 | PHE | 58 | 91.388 | 103.929 | 81.068 | 1.00 | 0.00 | H |
| ATOM | 135 | HZ | PHE | 58 | 89.986 | 105.961 | 80.913 | 1.00 | 0.00 | H |
| ATOM | 136 | HE2 | PHE | 58 | 90.738 | 108.067 | 81.966 | 1.00 | 0.00 | H |
| ATOM | 137 | HD2 | PHE | 58 | 92.882 | 108.149 | 83.179 | 1.00 | 0.00 | H |
| ATOM | 138 | N | LEU | 59 | 97.255 | 104.662 | 81.698 | 0.00 | 0.00 | N |
| ATOM | 139 | CA | LEU | 59 | 97.536 | 103.546 | 80.752 | 0.00 | 0.00 | C |
| ATOM | 140 | C | LEU | 59 | 96.581 | 102.327 | 81.038 | 0.00 | 0.00 | C |
| ATOM | 141 | O | LEU | 59 | 95.715 | 102.031 | 80.211 | 0.00 | 0.00 | O |
| ATOM | 142 | CB | LEU | 59 | 99.079 | 103.309 | 80.853 | 0.00 | 0.00 | C |
| ATOM | 143 | CG | LEU | 59 | 99.808 | 102.416 | 79.817 | 0.00 | 0.00 | C |
| ATOM | 144 | CD1 | LEU | 59 | 99.468 | 100.925 | 79.926 | 0.00 | 0.00 | C |
| ATOM | 145 | CD2 | LEU | 59 | 99.633 | 102.897 | 78.369 | 0.00 | 0.00 | C |
| ATOM | 146 | H | LEU | 59 | 97.696 | 104.671 | 82.627 | 0.00 | 0.00 | H |
| ATOM | 147 | HA | LEU | 59 | 97.324 | 103.877 | 79.716 | 0.00 | 0.00 | H |
| ATOM | 148 | 1HB | LEU | 59 | 99.334 | 102.939 | 81.865 | 0.00 | 0.00 | H |
| ATOM | 149 | 2HB | LEU | 59 | 99.594 | 104.291 | 80.798 | 0.00 | 0.00 | H |
| ATOM | 150 | HG | LEU | 59 | 100.892 | 102.501 | 80.047 | 0.00 | 0.00 | H |
| ATOM | 151 | 1HD1 | LEU | 59 | 99.582 | 100.542 | 80.956 | 0.00 | 0.00 | H |
| ATOM | 152 | 2HD1 | LEU | 59 | 98.436 | 100.688 | 79.606 | 0.00 | 0.00 | H |
| ATOM | 153 | 3HD1 | LEU | 59 | 100.153 | 100.321 | 79.304 | 0.00 | 0.00 | H |
| ATOM | 154 | 1HD2 | LEU | 59 | 100.279 | 102.327 | 77.675 | 0.00 | 0.00 | H |
| ATOM | 155 | 2HD2 | LEU | 59 | 98.592 | 102.786 | 78.012 | 0.00 | 0.00 | H |
| ATOM | 156 | 3HD2 | LEU | 59 | 99.915 | 103.961 | 78.256 | 0.00 | 0.00 | H |
| ATOM | 157 | N | GLY | 60 | 96.676 | 101.691 | 82.223 | 0.00 | 0.00 | N |
| ATOM | 158 | CA | GLY | 60 | 95.847 | 100.505 | 82.595 | 0.00 | 0.00 | C |
| ATOM | 159 | C | GLY | 60 | 95.373 | 100.470 | 84.075 | 0.00 | 0.00 | C |
| ATOM | 160 | O | GLY | 60 | 95.769 | 101.287 | 84.920 | 0.00 | 0.00 | O |
| ATOM | 161 | H | GLY | 60 | 97.447 | 102.031 | 82.805 | 0.00 | 0.00 | H |
| ATOM | 162 | 1HA | GLY | 60 | 96.432 | 99.582 | 82.395 | 0.00 | 0.00 | H |
| ATOM | 163 | 2HA | GLY | 60 | 94.967 | 100.400 | 81.931 | 0.00 | 0.00 | H |
| ATOM | 164 | N | LYS | 61 | 94.466 | 99.529 | 84.393 | 1.00 | 0.00 | N |
| ATOM | 165 | CA | LYS | 61 | 93.868 | 99.405 | 85.758 | 1.00 | 0.00 | C |
| ATOM | 166 | C | LYS | 61 | 93.299 | 97.972 | 86.042 | 1.00 | 0.00 | C |
| ATOM | 167 | O | LYS | 61 | 92.266 | 97.584 | 85.486 | 1.00 | 0.00 | O |
| ATOM | 168 | CB | LYS | 61 | 92.744 | 100.472 | 85.958 | 1.00 | 0.00 | C |
| ATOM | 169 | CG | LYS | 61 | 92.217 | 100.597 | 87.406 | 1.00 | 0.00 | C |
| ATOM | 170 | CD | LYS | 61 | 91.152 | 101.703 | 87.529 | 1.00 | 0.00 | C |
| ATOM | 171 | CE | LYS | 61 | 90.610 | 101.830 | 88.958 | 1.00 | 0.00 | C |
| ATOM | 172 | NZ | LYS | 61 | 89.603 | 102.910 | 89.003 | 1.00 | 0.00 | N |
| ATOM | 173 | 1HZ | LYS | 61 | 89.236 | 102.999 | 89.961 | 1.00 | 0.00 | H |
| ATOM | 174 | 2HZ | LYS | 61 | 90.040 | 103.799 | 88.719 | 1.00 | 0.00 | H |
| ATOM | 175 | 3HZ | LYS | 61 | 88.831 | 102.687 | 88.359 | 1.00 | 0.00 | H |
| ATOM | 176 | H | LYS | 61 | 94.523 | 98.723 | 83.745 | 1.00 | 0.00 | H |
| ATOM | 177 | HA | LYS | 61 | 94.668 | 99.606 | 86.500 | 1.00 | 0.00 | H |
| ATOM | 178 | 1HB | LYS | 61 | 93.112 | 101.471 | 85.646 | 1.00 | 0.00 | H |
| ATOM | 179 | 2HB | LYS | 61 | 91.900 | 100.252 | 85.275 | 1.00 | 0.00 | H |
| ATOM | 180 | 1HG | LYS | 61 | 91.791 | 99.629 | 87.739 | 1.00 | 0.00 | H |
| ATOM | 181 | 2HG | LYS | 61 | 93.058 | 100.804 | 88.098 | 1.00 | 0.00 | H |
| ATOM | 182 | 1HD | LYS | 61 | 91.583 | 102.672 | 87.205 | 1.00 | 0.00 | H |
| ATOM | 183 | 2HD | LYS | 61 | 90.322 | 101.497 | 86.825 | 1.00 | 0.00 | H |
| ATOM | 184 | 1HE | LYS | 61 | 90.160 | 100.873 | 89.292 | 1.00 | 0.00 | H |
| ATOM | 185 | 2HE | LYS | 61 | 91.431 | 102.052 | 89.669 | 1.00 | 0.00 | H |
| ATOM | 186 | N | GLY | 62 | 93.868 | 97.257 | 87.026 | 0.00 | 0.00 | N |
| ATOM | 187 | CA | GLY | 62 | 93.338 | 95.929 | 87.443 | 0.00 | 0.00 | C |
| ATOM | 188 | C | GLY | 62 | 93.782 | 95.448 | 88.844 | 0.00 | 0.00 | C |
| ATOM | 189 | O | GLY | 62 | 93.881 | 96.219 | 89.801 | 0.00 | 0.00 | O |
| ATOM | 190 | H | GLY | 62 | 94.799 | 97.595 | 87.296 | 0.00 | 0.00 | H |
| ATOM | 191 | 1HA | GLY | 62 | 93.630 | 95.190 | 86.668 | 0.00 | 0.00 | H |
| ATOM | 192 | 2HA | GLY | 62 | 92.228 | 95.918 | 87.437 | 0.00 | 0.00 | H |
| ATOM | 193 | N | GLY | 63 | 94.055 | 94.138 | 88.968 | 1.00 | 0.00 | N |
| ATOM | 194 | CA | GLY | 63 | 94.411 | 93.510 | 90.288 | 1.00 | 0.00 | C |
| ATOM | 195 | C | GLY | 63 | 95.817 | 93.707 | 90.924 | 1.00 | 0.00 | C |
| ATOM | 196 | O | GLY | 63 | 96.231 | 92.889 | 91.746 | 1.00 | 0.00 | O |
| ATOM | 197 | H | GLY | 63 | 93.953 | 93.598 | 88.103 | 1.00 | 0.00 | H |
| ATOM | 198 | 1HA | GLY | 63 | 93.677 | 93.819 | 91.056 | 1.00 | 0.00 | H |
| ATOM | 199 | 2HA | GLY | 63 | 94.259 | 92.418 | 90.203 | 1.00 | 0.00 | H |
| ATOM | 200 | N | PHE | 64 | 96.503 | 94.806 | 90.594 | 0.00 | 0.00 | N |
| ATOM | 201 | CA | PHE | 64 | 97.650 | 95.340 | 91.383 | 0.00 | 0.00 | C |
| ATOM | 202 | C | PHE | 64 | 97.219 | 96.766 | 91.873 | 0.00 | 0.00 | C |
| ATOM | 203 | O | PHE | 64 | 96.780 | 96.906 | 93.017 | 0.00 | 0.00 | O |
| ATOM | 204 | CB | PHE | 64 | 98.957 | 95.211 | 90.531 | 0.00 | 0.00 | C |
| ATOM | 205 | CG | PHE | 64 | 100.322 | 95.180 | 91.262 | 0.00 | 0.00 | C |
| ATOM | 206 | CD1 | PHE | 64 | 101.339 | 94.374 | 90.729 | 0.00 | 0.00 | C |
| ATOM | 207 | CE1 | PHE | 64 | 102.589 | 94.313 | 91.339 | 0.00 | 0.00 | C |
| ATOM | 208 | CZ | PHE | 64 | 102.844 | 95.070 | 92.476 | 0.00 | 0.00 | C |
| ATOM | 209 | CE2 | PHE | 64 | 101.849 | 95.880 | 93.015 | 0.00 | 0.00 | C |
| ATOM | 210 | CD2 | PHE | 64 | 100.597 | 95.943 | 92.407 | 0.00 | 0.00 | C |
| ATOM | 211 | H | PHE | 64 | 96.013 | 95.366 | 89.888 | 0.00 | 0.00 | H |
| ATOM | 212 | HA | PHE | 64 | 97.815 | 94.744 | 92.306 | 0.00 | 0.00 | H |
| ATOM | 213 | 1HB | PHE | 64 | 98.995 | 95.988 | 89.745 | 0.00 | 0.00 | H |
| ATOM | 214 | 2HB | PHE | 64 | 98.885 | 94.272 | 89.943 | 0.00 | 0.00 | H |
| ATOM | 215 | HD1 | PHE | 64 | 101.175 | 93.786 | 89.834 | 0.00 | 0.00 | H |
| ATOM | 216 | HE1 | PHE | 64 | 103.371 | 93.687 | 90.923 | 0.00 | 0.00 | H |
| ATOM | 217 | HZ | PHE | 64 | 103.822 | 95.022 | 92.935 | 0.00 | 0.00 | H |
| ATOM | 218 | HE2 | PHE | 64 | 102.058 | 96.458 | 93.903 | 0.00 | 0.00 | H |
| ATOM | 219 | HD2 | PHE | 64 | 99.845 | 96.585 | 92.839 | 0.00 | 0.00 | H |
| ATOM | 220 | N | ALA | 65 | 97.243 | 97.795 | 90.990 | 1.00 | 0.00 | N |
| ATOM | 221 | CA | ALA | 65 | 96.401 | 99.013 | 91.144 | 1.00 | 0.00 | C |
| ATOM | 222 | C | ALA | 65 | 96.155 | 99.807 | 89.814 | 1.00 | 0.00 | C |
| ATOM | 223 | O | ALA | 65 | 95.088 | 99.660 | 89.208 | 1.00 | 0.00 | O |
| ATOM | 224 | CB | ALA | 65 | 96.915 | 99.890 | 92.311 | 1.00 | 0.00 | C |
| ATOM | 225 | H | ALA | 65 | 97.644 | 97.519 | 90.086 | 1.00 | 0.00 | H |
| ATOM | 226 | HA | ALA | 65 | 95.375 | 98.686 | 91.416 | 1.00 | 0.00 | H |
| ATOM | 227 | 2HB | ALA | 65 | 96.805 | 99.369 | 93.278 | 1.00 | 0.00 | H |
| ATOM | 228 | 3HB | ALA | 65 | 97.986 | 100.139 | 92.197 | 1.00 | 0.00 | H |
| ATOM | 229 | 1HB | ALA | 65 | 96.357 | 100.841 | 92.391 | 1.00 | 0.00 | H |
| ATOM | 230 | N | LYS | 66 | 97.056 | 100.720 | 89.405 | 1.00 | 0.00 | N |
| ATOM | 231 | CA | LYS | 66 | 96.846 | 101.620 | 88.233 | 1.00 | 0.00 | C |
| ATOM | 232 | C | LYS | 66 | 98.200 | 102.188 | 87.702 | 1.00 | 0.00 | C |
| ATOM | 233 | O | LYS | 66 | 98.947 | 102.808 | 88.469 | 1.00 | 0.00 | O |
| ATOM | 234 | CB | LYS | 66 | 95.815 | 102.752 | 88.564 | 1.00 | 0.00 | C |
| ATOM | 235 | CG | LYS | 66 | 96.230 | 103.839 | 89.590 | 1.00 | 0.00 | C |
| ATOM | 236 | CD | LYS | 66 | 95.060 | 104.754 | 89.991 | 1.00 | 0.00 | C |
| ATOM | 237 | CE | LYS | 66 | 95.529 | 105.928 | 90.862 | 1.00 | 0.00 | C |
| ATOM | 238 | NZ | LYS | 66 | 94.373 | 106.785 | 91.189 | 1.00 | 0.00 | N |
| ATOM | 239 | 1HZ | LYS | 66 | 94.684 | 107.574 | 91.773 | 1.00 | 0.00 | H |
| ATOM | 240 | 2HZ | LYS | 66 | 93.956 | 107.146 | 90.319 | 1.00 | 0.00 | H |
| ATOM | 241 | 3HZ | LYS | 66 | 93.670 | 106.234 | 91.702 | 1.00 | 0.00 | H |
| ATOM | 242 | H | LYS | 66 | 97.952 | 100.685 | 89.906 | 1.00 | 0.00 | H |
| ATOM | 243 | HA | LYS | 66 | 96.407 | 101.006 | 87.418 | 1.00 | 0.00 | H |
| ATOM | 244 | 1HB | LYS | 66 | 95.539 | 103.250 | 87.615 | 1.00 | 0.00 | H |
| ATOM | 245 | 2HB | LYS | 66 | 94.875 | 102.272 | 88.900 | 1.00 | 0.00 | H |
| ATOM | 246 | 1HG | LYS | 66 | 96.657 | 103.368 | 90.497 | 1.00 | 0.00 | H |
| ATOM | 247 | 2HG | LYS | 66 | 97.055 | 104.444 | 89.163 | 1.00 | 0.00 | H |
| ATOM | 248 | 1HD | LYS | 66 | 94.549 | 105.136 | 89.086 | 1.00 | 0.00 | H |
| ATOM | 249 | 2HD | LYS | 66 | 94.294 | 104.158 | 90.526 | 1.00 | 0.00 | H |
| ATOM | 250 | 1HE | LYS | 66 | 96.013 | 105.558 | 91.789 | 1.00 | 0.00 | H |
| ATOM | 251 | 2HE | LYS | 66 | 96.299 | 106.524 | 90.330 | 1.00 | 0.00 | H |
| ATOM | 252 | N | CYS | 67 | 98.478 | 102.033 | 86.400 | 1.00 | 0.00 | N |
| ATOM | 253 | CA | CYS | 67 | 99.654 | 102.670 | 85.746 | 1.00 | 0.00 | C |
| ATOM | 254 | C | CYS | 67 | 99.278 | 103.971 | 84.964 | 1.00 | 0.00 | C |
| ATOM | 255 | O | CYS | 67 | 98.198 | 104.091 | 84.372 | 1.00 | 0.00 | O |
| ATOM | 256 | CB | CYS | 67 | 100.379 | 101.592 | 84.922 | 1.00 | 0.00 | C |
| ATOM | 257 | SG | CYS | 67 | 99.311 | 100.811 | 83.669 | 1.00 | 0.00 | S |
| ATOM | 258 | H | CYS | 67 | 97.759 | 101.563 | 85.832 | 1.00 | 0.00 | H |
| ATOM | 259 | HA | CYS | 67 | 100.393 | 102.962 | 86.518 | 1.00 | 0.00 | H |
| ATOM | 260 | 1HB | CYS | 67 | 101.271 | 102.013 | 84.418 | 1.00 | 0.00 | H |
| ATOM | 261 | 2HB | CYS | 67 | 100.767 | 100.803 | 85.593 | 1.00 | 0.00 | H |
| ATOM | 262 | HG | CYS | 67 | 100.236 | 100.000 | 83.152 | 1.00 | 0.00 | H |
| ATOM | 263 | N | PHE | 68 | 100.157 | 104.979 | 85.038 | 1.00 | 0.00 | N |
| ATOM | 264 | CA | PHE | 68 | 99.953 | 106.316 | 84.412 | 1.00 | 0.00 | C |
| ATOM | 265 | C | PHE | 68 | 101.159 | 106.726 | 83.511 | 1.00 | 0.00 | C |
| ATOM | 266 | O | PHE | 68 | 102.314 | 106.458 | 83.840 | 1.00 | 0.00 | O |
| ATOM | 267 | CB | PHE | 68 | 99.753 | 107.364 | 85.554 | 1.00 | 0.00 | C |
| ATOM | 268 | CG | PHE | 68 | 98.298 | 107.699 | 85.907 | 1.00 | 0.00 | C |
| ATOM | 269 | CD1 | PHE | 68 | 97.740 | 108.912 | 85.487 | 1.00 | 0.00 | C |
| ATOM | 270 | CE1 | PHE | 68 | 96.435 | 109.246 | 85.843 | 1.00 | 0.00 | C |
| ATOM | 271 | CZ | PHE | 68 | 95.682 | 108.371 | 86.620 | 1.00 | 0.00 | C |
| ATOM | 272 | CE2 | PHE | 68 | 96.228 | 107.159 | 87.035 | 1.00 | 0.00 | C |
| ATOM | 273 | CD2 | PHE | 68 | 97.532 | 106.823 | 86.682 | 1.00 | 0.00 | C |
| ATOM | 274 | H | PHE | 68 | 101.038 | 104.758 | 85.528 | 1.00 | 0.00 | H |
| ATOM | 275 | HA | PHE | 68 | 99.054 | 106.312 | 83.764 | 1.00 | 0.00 | H |
| ATOM | 276 | 1HB | PHE | 68 | 100.300 | 107.066 | 86.470 | 1.00 | 0.00 | H |
| ATOM | 277 | 2HB | PHE | 68 | 100.257 | 108.312 | 85.283 | 1.00 | 0.00 | H |
| ATOM | 278 | HD1 | PHE | 68 | 98.312 | 109.598 | 84.877 | 1.00 | 0.00 | H |
| ATOM | 279 | HE1 | PHE | 68 | 96.009 | 110.183 | 85.512 | 1.00 | 0.00 | H |
| ATOM | 280 | HZ | PHE | 68 | 94.669 | 108.628 | 86.896 | 1.00 | 0.00 | H |
| ATOM | 281 | HE2 | PHE | 68 | 95.640 | 106.480 | 87.633 | 1.00 | 0.00 | H |
| ATOM | 282 | HD2 | PHE | 68 | 97.947 | 105.882 | 87.013 | 1.00 | 0.00 | H |
| ATOM | 283 | N | GLU | 69 | 100.875 | 107.502 | 82.456 | 1.00 | 0.00 | N |
| ATOM | 284 | CA | GLU | 69 | 101.904 | 108.317 | 81.744 | 1.00 | 0.00 | C |
| ATOM | 285 | C | GLU | 69 | 102.269 | 109.587 | 82.591 | 1.00 | 0.00 | C |
| ATOM | 286 | O | GLU | 69 | 101.475 | 110.527 | 82.695 | 1.00 | 0.00 | O |
| ATOM | 287 | CB | GLU | 69 | 101.313 | 108.631 | 80.346 | 1.00 | 0.00 | C |
| ATOM | 288 | CG | GLU | 69 | 102.257 | 109.425 | 79.404 | 1.00 | 0.00 | C |
| ATOM | 289 | CD | GLU | 69 | 101.724 | 109.581 | 77.983 | 1.00 | 0.00 | C |
| ATOM | 290 | OE1 | GLU | 69 | 101.463 | 108.612 | 77.239 | 1.00 | 0.00 | O |
| ATOM | 291 | OE2 | GLU | 69 | 101.433 | 110.872 | 77.674 | 1.00 | 0.00 | O |
| ATOM | 292 | H | GLU | 69 | 99.870 | 107.688 | 82.316 | 1.00 | 0.00 | H |
| ATOM | 293 | HA | GLU | 69 | 102.816 | 107.705 | 81.581 | 1.00 | 0.00 | H |
| ATOM | 294 | 1HB | GLU | 69 | 101.047 | 107.679 | 79.841 | 1.00 | 0.00 | H |
| ATOM | 295 | 2HB | GLU | 69 | 100.356 | 109.179 | 80.447 | 1.00 | 0.00 | H |
| ATOM | 296 | 1HG | GLU | 69 | 102.471 | 110.425 | 79.828 | 1.00 | 0.00 | H |
| ATOM | 297 | 2HG | GLU | 69 | 103.236 | 108.929 | 79.306 | 1.00 | 0.00 | H |
| ATOM | 298 | N | ILE | 70 | 103.448 | 109.579 | 83.229 | 1.00 | 0.00 | N |
| ATOM | 299 | CA | ILE | 70 | 103.873 | 110.642 | 84.193 | 1.00 | 0.00 | C |
| ATOM | 300 | C | ILE | 70 | 105.179 | 111.309 | 83.637 | 1.00 | 0.00 | C |
| ATOM | 301 | O | ILE | 70 | 106.208 | 110.650 | 83.454 | 1.00 | 0.00 | O |
| ATOM | 302 | CB | ILE | 70 | 104.059 | 110.093 | 85.662 | 1.00 | 0.00 | C |
| ATOM | 303 | CG1 | ILE | 70 | 102.877 | 109.225 | 86.195 | 1.00 | 0.00 | C |
| ATOM | 304 | CG2 | ILE | 70 | 104.317 | 111.255 | 86.658 | 1.00 | 0.00 | C |
| ATOM | 305 | CD1 | ILE | 70 | 103.026 | 108.633 | 87.611 | 1.00 | 0.00 | C |
| ATOM | 306 | H | ILE | 70 | 103.997 | 108.715 | 83.099 | 1.00 | 0.00 | H |
| ATOM | 307 | HA | ILE | 70 | 103.089 | 111.422 | 84.256 | 1.00 | 0.00 | H |
| ATOM | 308 | HB | ILE | 70 | 104.960 | 109.444 | 85.657 | 1.00 | 0.00 | H |
| ATOM | 309 | 1HG1 | ILE | 70 | 101.931 | 109.795 | 86.130 | 1.00 | 0.00 | H |
| ATOM | 310 | 2HG1 | ILE | 70 | 102.737 | 108.371 | 85.507 | 1.00 | 0.00 | H |
| ATOM | 311 | 2HG2 | ILE | 70 | 105.106 | 111.947 | 86.313 | 1.00 | 0.00 | H |
| ATOM | 312 | 3HG2 | ILE | 70 | 103.412 | 111.865 | 86.831 | 1.00 | 0.00 | H |
| ATOM | 313 | 1HG2 | ILE | 70 | 104.649 | 110.880 | 87.643 | 1.00 | 0.00 | H |
| ATOM | 314 | 2HD1 | ILE | 70 | 104.015 | 108.167 | 87.762 | 1.00 | 0.00 | H |
| ATOM | 315 | 3HD1 | ILE | 70 | 102.906 | 109.405 | 88.395 | 1.00 | 0.00 | H |
| ATOM | 316 | 1HD1 | ILE | 70 | 102.263 | 107.858 | 87.810 | 1.00 | 0.00 | H |
| ATOM | 317 | N | SER | 71 | 105.152 | 112.631 | 83.413 | 1.00 | 0.00 | N |
| ATOM | 318 | CA | SER | 71 | 106.376 | 113.412 | 83.098 | 1.00 | 0.00 | C |
| ATOM | 319 | C | SER | 71 | 107.154 | 113.834 | 84.385 | 1.00 | 0.00 | C |
| ATOM | 320 | O | SER | 71 | 106.586 | 114.421 | 85.309 | 1.00 | 0.00 | O |
| ATOM | 321 | CB | SER | 71 | 105.960 | 114.645 | 82.265 | 1.00 | 0.00 | C |
| ATOM | 322 | OG | SER | 71 | 107.110 | 115.366 | 81.807 | 1.00 | 0.00 | O |
| ATOM | 323 | H | SER | 71 | 104.293 | 113.093 | 83.746 | 1.00 | 0.00 | H |
| ATOM | 324 | HA | SER | 71 | 107.043 | 112.810 | 82.450 | 1.00 | 0.00 | H |
| ATOM | 325 | 1HB | SER | 71 | 105.366 | 114.332 | 81.383 | 1.00 | 0.00 | H |
| ATOM | 326 | 2HB | SER | 71 | 105.297 | 115.316 | 82.847 | 1.00 | 0.00 | H |
| ATOM | 327 | HG | SER | 71 | 107.585 | 115.699 | 82.578 | 1.00 | 0.00 | H |
| ATOM | 328 | N | ASP | 72 | 108.478 | 113.631 | 84.406 | 1.00 | 0.00 | N |
| ATOM | 329 | CA | ASP | 72 | 109.394 | 114.350 | 85.338 | 1.00 | 0.00 | C |
| ATOM | 330 | C | ASP | 72 | 109.438 | 115.878 | 84.976 | 1.00 | 0.00 | C |
| ATOM | 331 | O | ASP | 72 | 109.784 | 116.245 | 83.851 | 1.00 | 0.00 | O |
| ATOM | 332 | CB | ASP | 72 | 110.803 | 113.684 | 85.270 | 1.00 | 0.00 | C |
| ATOM | 333 | CG | ASP | 72 | 111.439 | 113.276 | 86.597 | 1.00 | 0.00 | C |
| ATOM | 334 | OD1 | ASP | 72 | 111.932 | 112.171 | 86.785 | 1.00 | 0.00 | O |
| ATOM | 335 | OD2 | ASP | 72 | 111.409 | 114.257 | 87.539 | 1.00 | 0.00 | O |
| ATOM | 336 | H | ASP | 72 | 108.829 | 113.054 | 83.626 | 1.00 | 0.00 | H |
| ATOM | 337 | HA | ASP | 72 | 108.993 | 114.230 | 86.367 | 1.00 | 0.00 | H |
| ATOM | 338 | 1HB | ASP | 72 | 110.779 | 112.765 | 84.660 | 1.00 | 0.00 | H |
| ATOM | 339 | 2HB | ASP | 72 | 111.527 | 114.326 | 84.736 | 1.00 | 0.00 | H |
| ATOM | 340 | N | ALA | 73 | 109.010 | 116.750 | 85.893 | 1.00 | 0.00 | N |
| ATOM | 341 | CA | ALA | 73 | 108.809 | 118.199 | 85.618 | 1.00 | 0.00 | C |
| ATOM | 342 | C | ALA | 73 | 110.090 | 119.067 | 85.378 | 1.00 | 0.00 | C |
| ATOM | 343 | O | ALA | 73 | 110.112 | 119.894 | 84.466 | 1.00 | 0.00 | O |
| ATOM | 344 | CB | ALA | 73 | 107.957 | 118.712 | 86.796 | 1.00 | 0.00 | C |
| ATOM | 345 | H | ALA | 73 | 108.846 | 116.340 | 86.825 | 1.00 | 0.00 | H |
| ATOM | 346 | HA | ALA | 73 | 108.197 | 118.298 | 84.699 | 1.00 | 0.00 | H |
| ATOM | 347 | 2HB | ALA | 73 | 107.046 | 118.105 | 86.956 | 1.00 | 0.00 | H |
| ATOM | 348 | 3HB | ALA | 73 | 108.520 | 118.720 | 87.749 | 1.00 | 0.00 | H |
| ATOM | 349 | 1HB | ALA | 73 | 107.622 | 119.748 | 86.618 | 1.00 | 0.00 | H |
| ATOM | 350 | N | ASP | 74 | 111.144 | 118.878 | 86.187 | 1.00 | 0.00 | N |
| ATOM | 351 | CA | ASP | 74 | 112.426 | 119.637 | 86.060 | 1.00 | 0.00 | C |
| ATOM | 352 | C | ASP | 74 | 113.439 | 119.098 | 84.983 | 1.00 | 0.00 | C |
| ATOM | 353 | O | ASP | 74 | 114.168 | 119.901 | 84.399 | 1.00 | 0.00 | O |
| ATOM | 354 | CB | ASP | 74 | 113.039 | 119.767 | 87.483 | 1.00 | 0.00 | C |
| ATOM | 355 | CG | ASP | 74 | 112.259 | 120.707 | 88.409 | 1.00 | 0.00 | C |
| ATOM | 356 | OD1 | ASP | 74 | 112.543 | 121.888 | 88.568 | 1.00 | 0.00 | O |
| ATOM | 357 | OD2 | ASP | 74 | 111.176 | 120.106 | 88.975 | 1.00 | 0.00 | O |
| ATOM | 358 | H | ASP | 74 | 110.954 | 118.227 | 86.954 | 1.00 | 0.00 | H |
| ATOM | 359 | HA | ASP | 74 | 112.196 | 120.667 | 85.713 | 1.00 | 0.00 | H |
| ATOM | 360 | 1HB | ASP | 74 | 113.155 | 118.780 | 87.967 | 1.00 | 0.00 | H |
| ATOM | 361 | 2HB | ASP | 74 | 114.063 | 120.175 | 87.408 | 1.00 | 0.00 | H |
| ATOM | 362 | N | THR | 75 | 113.491 | 117.782 | 84.701 | 0.00 | 0.00 | N |
| ATOM | 363 | CA | THR | 75 | 114.274 | 117.208 | 83.552 | 0.00 | 0.00 | C |
| ATOM | 364 | C | THR | 75 | 113.505 | 117.002 | 82.190 | 0.00 | 0.00 | C |
| ATOM | 365 | O | THR | 75 | 114.162 | 116.797 | 81.167 | 0.00 | 0.00 | O |
| ATOM | 366 | CB | THR | 75 | 114.958 | 115.866 | 83.972 | 0.00 | 0.00 | C |
| ATOM | 367 | OG1 | THR | 75 | 114.001 | 114.890 | 84.377 | 0.00 | 0.00 | O |
| ATOM | 368 | CG2 | THR | 75 | 116.000 | 115.964 | 85.096 | 0.00 | 0.00 | C |
| ATOM | 369 | H | THR | 75 | 112.860 | 117.204 | 85.264 | 0.00 | 0.00 | H |
| ATOM | 370 | HA | THR | 75 | 115.099 | 117.901 | 83.291 | 0.00 | 0.00 | H |
| ATOM | 371 | HB | THR | 75 | 115.494 | 115.471 | 83.084 | 0.00 | 0.00 | H |
| ATOM | 372 | HG1 | THR | 75 | 114.468 | 114.041 | 84.362 | 0.00 | 0.00 | H |
| ATOM | 373 | 1HG2 | THR | 75 | 116.485 | 114.990 | 85.288 | 0.00 | 0.00 | H |
| ATOM | 374 | 2HG2 | THR | 75 | 116.800 | 116.685 | 84.847 | 0.00 | 0.00 | H |
| ATOM | 375 | 3HG2 | THR | 75 | 115.544 | 116.296 | 86.048 | 0.00 | 0.00 | H |
| ATOM | 376 | N | LYS | 76 | 112.156 | 117.002 | 82.157 | 1.00 | 0.00 | N |
| ATOM | 377 | CA | LYS | 76 | 111.326 | 116.626 | 80.966 | 1.00 | 0.00 | C |
| ATOM | 378 | C | LYS | 76 | 111.521 | 115.147 | 80.454 | 1.00 | 0.00 | C |
| ATOM | 379 | O | LYS | 76 | 111.955 | 114.899 | 79.326 | 1.00 | 0.00 | O |
| ATOM | 380 | CB | LYS | 76 | 111.377 | 117.750 | 79.890 | 1.00 | 0.00 | C |
| ATOM | 381 | CG | LYS | 76 | 110.215 | 117.715 | 78.869 | 1.00 | 0.00 | C |
| ATOM | 382 | CD | LYS | 76 | 110.269 | 118.893 | 77.878 | 1.00 | 0.00 | C |
| ATOM | 383 | CE | LYS | 76 | 109.083 | 118.874 | 76.902 | 1.00 | 0.00 | C |
| ATOM | 384 | NZ | LYS | 76 | 109.178 | 120.032 | 75.991 | 1.00 | 0.00 | N |
| ATOM | 385 | 1HZ | LYS | 76 | 108.383 | 120.020 | 75.336 | 1.00 | 0.00 | H |
| ATOM | 386 | 2HZ | LYS | 76 | 109.156 | 120.904 | 76.540 | 1.00 | 0.00 | H |
| ATOM | 387 | 3HZ | LYS | 76 | 110.059 | 119.982 | 75.461 | 1.00 | 0.00 | H |
| ATOM | 388 | H | LYS | 76 | 111.732 | 117.241 | 83.060 | 1.00 | 0.00 | H |
| ATOM | 389 | HA | LYS | 76 | 110.283 | 116.647 | 81.335 | 1.00 | 0.00 | H |
| ATOM | 390 | 1HB | LYS | 76 | 111.373 | 118.738 | 80.390 | 1.00 | 0.00 | H |
| ATOM | 391 | 2HB | LYS | 76 | 112.350 | 117.701 | 79.362 | 1.00 | 0.00 | H |
| ATOM | 392 | 1HG | LYS | 76 | 110.236 | 116.758 | 78.310 | 1.00 | 0.00 | H |
| ATOM | 393 | 2HG | LYS | 76 | 109.246 | 117.726 | 79.405 | 1.00 | 0.00 | H |
| ATOM | 394 | 1HD | LYS | 76 | 110.276 | 119.850 | 78.439 | 1.00 | 0.00 | H |
| ATOM | 395 | 2HD | LYS | 76 | 111.226 | 118.866 | 77.323 | 1.00 | 0.00 | H |
| ATOM | 396 | 1HE | LYS | 76 | 109.072 | 117.929 | 76.323 | 1.00 | 0.00 | H |
| ATOM | 397 | 2HE | LYS | 76 | 108.124 | 118.910 | 77.455 | 1.00 | 0.00 | H |
| ATOM | 398 | N | GLU | 77 | 111.182 | 114.160 | 81.304 | 1.00 | 0.00 | N |
| ATOM | 399 | CA | GLU | 77 | 111.388 | 112.710 | 81.010 | 1.00 | 0.00 | C |
| ATOM | 400 | C | GLU | 77 | 110.056 | 111.924 | 81.242 | 1.00 | 0.00 | C |
| ATOM | 401 | O | GLU | 77 | 109.498 | 111.953 | 82.341 | 1.00 | 0.00 | O |
| ATOM | 402 | CB | GLU | 77 | 112.526 | 112.150 | 81.907 | 1.00 | 0.00 | C |
| ATOM | 403 | CG | GLU | 77 | 113.952 | 112.687 | 81.620 | 1.00 | 0.00 | C |
| ATOM | 404 | CD | GLU | 77 | 115.006 | 112.124 | 82.569 | 1.00 | 0.00 | C |
| ATOM | 405 | OE1 | GLU | 77 | 115.199 | 112.545 | 83.706 | 1.00 | 0.00 | O |
| ATOM | 406 | OE2 | GLU | 77 | 115.704 | 111.096 | 82.017 | 1.00 | 0.00 | O |
| ATOM | 407 | H | GLU | 77 | 110.912 | 114.507 | 82.230 | 1.00 | 0.00 | H |
| ATOM | 408 | HA | GLU | 77 | 111.698 | 112.563 | 79.954 | 1.00 | 0.00 | H |
| ATOM | 409 | 1HB | GLU | 77 | 112.276 | 112.325 | 82.971 | 1.00 | 0.00 | H |
| ATOM | 410 | 2HB | GLU | 77 | 112.548 | 111.047 | 81.804 | 1.00 | 0.00 | H |
| ATOM | 411 | 1HG | GLU | 77 | 114.243 | 112.476 | 80.574 | 1.00 | 0.00 | H |
| ATOM | 412 | 2HG | GLU | 77 | 113.973 | 113.790 | 81.706 | 1.00 | 0.00 | H |
| ATOM | 413 | N | VAL | 78 | 109.538 | 111.228 | 80.218 | 1.00 | 0.00 | N |
| ATOM | 414 | CA | VAL | 78 | 108.198 | 110.559 | 80.279 | 1.00 | 0.00 | C |
| ATOM | 415 | C | VAL | 78 | 108.278 | 109.046 | 80.702 | 1.00 | 0.00 | C |
| ATOM | 416 | O | VAL | 78 | 109.020 | 108.243 | 80.129 | 1.00 | 0.00 | O |
| ATOM | 417 | CB | VAL | 78 | 107.372 | 110.787 | 78.964 | 1.00 | 0.00 | C |
| ATOM | 418 | CG1 | VAL | 78 | 106.959 | 112.261 | 78.749 | 1.00 | 0.00 | C |
| ATOM | 419 | CG2 | VAL | 78 | 108.015 | 110.259 | 77.661 | 1.00 | 0.00 | C |
| ATOM | 420 | H | VAL | 78 | 110.071 | 111.278 | 79.345 | 1.00 | 0.00 | H |
| ATOM | 421 | HA | VAL | 78 | 107.598 | 111.065 | 81.062 | 1.00 | 0.00 | H |
| ATOM | 422 | HB | VAL | 78 | 106.424 | 110.234 | 79.103 | 1.00 | 0.00 | H |
| ATOM | 423 | 1HG1 | VAL | 78 | 107.833 | 112.918 | 78.578 | 1.00 | 0.00 | H |
| ATOM | 424 | 2HG1 | VAL | 78 | 106.287 | 112.382 | 77.879 | 1.00 | 0.00 | H |
| ATOM | 425 | 3HG1 | VAL | 78 | 106.421 | 112.669 | 79.625 | 1.00 | 0.00 | H |
| ATOM | 426 | 2HG2 | VAL | 78 | 108.267 | 109.184 | 77.736 | 1.00 | 0.00 | H |
| ATOM | 427 | 3HG2 | VAL | 78 | 107.338 | 110.363 | 76.794 | 1.00 | 0.00 | H |
| ATOM | 428 | 1HG2 | VAL | 78 | 108.950 | 110.791 | 77.408 | 1.00 | 0.00 | H |
| ATOM | 429 | N | PHE | 79 | 107.458 | 108.656 | 81.701 | 1.00 | 0.00 | N |
| ATOM | 430 | CA | PHE | 79 | 107.559 | 107.335 | 82.387 | 1.00 | 0.00 | C |
| ATOM | 431 | C | PHE | 79 | 106.160 | 106.654 | 82.583 | 1.00 | 0.00 | C |
| ATOM | 432 | O | PHE | 79 | 105.163 | 107.290 | 82.943 | 1.00 | 0.00 | O |
| ATOM | 433 | CB | PHE | 79 | 108.193 | 107.551 | 83.793 | 1.00 | 0.00 | C |
| ATOM | 434 | CG | PHE | 79 | 109.667 | 107.986 | 83.837 | 1.00 | 0.00 | C |
| ATOM | 435 | CD1 | PHE | 79 | 110.007 | 109.280 | 84.244 | 1.00 | 0.00 | C |
| ATOM | 436 | CE1 | PHE | 79 | 111.344 | 109.666 | 84.309 | 1.00 | 0.00 | C |
| ATOM | 437 | CZ | PHE | 79 | 112.347 | 108.773 | 83.948 | 1.00 | 0.00 | C |
| ATOM | 438 | CE2 | PHE | 79 | 112.018 | 107.481 | 83.544 | 1.00 | 0.00 | C |
| ATOM | 439 | CD2 | PHE | 79 | 110.684 | 107.088 | 83.496 | 1.00 | 0.00 | C |
| ATOM | 440 | H | PHE | 79 | 106.996 | 109.437 | 82.183 | 1.00 | 0.00 | H |
| ATOM | 441 | HA | PHE | 79 | 108.209 | 106.643 | 81.810 | 1.00 | 0.00 | H |
| ATOM | 442 | 1HB | PHE | 79 | 107.558 | 108.258 | 84.363 | 1.00 | 0.00 | H |
| ATOM | 443 | 2HB | PHE | 79 | 108.124 | 106.607 | 84.368 | 1.00 | 0.00 | H |
| ATOM | 444 | HD1 | PHE | 79 | 109.236 | 109.992 | 84.503 | 1.00 | 0.00 | H |
| ATOM | 445 | HE1 | PHE | 79 | 111.609 | 110.656 | 84.642 | 1.00 | 0.00 | H |
| ATOM | 446 | HZ | PHE | 79 | 113.384 | 109.073 | 84.006 | 1.00 | 0.00 | H |
| ATOM | 447 | HE2 | PHE | 79 | 112.798 | 106.782 | 83.282 | 1.00 | 0.00 | H |
| ATOM | 448 | HD2 | PHE | 79 | 110.445 | 106.085 | 83.177 | 1.00 | 0.00 | H |
| ATOM | 449 | N | ALA | 80 | 106.110 | 105.319 | 82.471 | 0.00 | 0.00 | N |
| ATOM | 450 | CA | ALA | 80 | 104.990 | 104.508 | 83.001 | 0.00 | 0.00 | C |
| ATOM | 451 | C | ALA | 80 | 105.107 | 104.272 | 84.547 | 0.00 | 0.00 | C |
| ATOM | 452 | O | ALA | 80 | 105.868 | 103.428 | 85.035 | 0.00 | 0.00 | O |
| ATOM | 453 | CB | ALA | 80 | 105.012 | 103.194 | 82.218 | 0.00 | 0.00 | C |
| ATOM | 454 | H | ALA | 80 | 107.042 | 104.891 | 82.358 | 0.00 | 0.00 | H |
| ATOM | 455 | HA | ALA | 80 | 104.026 | 105.000 | 82.765 | 0.00 | 0.00 | H |
| ATOM | 456 | 1HB | ALA | 80 | 104.222 | 102.506 | 82.564 | 0.00 | 0.00 | H |
| ATOM | 457 | 2HB | ALA | 80 | 104.850 | 103.354 | 81.136 | 0.00 | 0.00 | H |
| ATOM | 458 | 3HB | ALA | 80 | 105.975 | 102.656 | 82.325 | 0.00 | 0.00 | H |
| ATOM | 459 | N | GLY | 81 | 104.345 | 105.047 | 85.329 | 1.00 | 0.00 | N |
| ATOM | 460 | CA | GLY | 81 | 104.419 | 105.001 | 86.811 | 1.00 | 0.00 | C |
| ATOM | 461 | C | GLY | 81 | 103.218 | 104.300 | 87.487 | 1.00 | 0.00 | C |
| ATOM | 462 | O | GLY | 81 | 102.070 | 104.747 | 87.385 | 1.00 | 0.00 | O |
| ATOM | 463 | H | GLY | 81 | 103.838 | 105.767 | 84.788 | 1.00 | 0.00 | H |
| ATOM | 464 | 1HA | GLY | 81 | 105.375 | 104.556 | 87.150 | 1.00 | 0.00 | H |
| ATOM | 465 | 2HA | GLY | 81 | 104.492 | 106.029 | 87.189 | 1.00 | 0.00 | H |
| ATOM | 466 | N | LYS | 82 | 103.500 | 103.207 | 88.205 | 0.00 | 0.00 | N |
| ATOM | 467 | CA | LYS | 82 | 102.483 | 102.410 | 88.951 | 0.00 | 0.00 | C |
| ATOM | 468 | C | LYS | 82 | 102.243 | 102.984 | 90.404 | 0.00 | 0.00 | C |
| ATOM | 469 | O | LYS | 82 | 103.163 | 102.982 | 91.231 | 0.00 | 0.00 | O |
| ATOM | 470 | CB | LYS | 82 | 102.939 | 100.916 | 88.804 | 0.00 | 0.00 | C |
| ATOM | 471 | CG | LYS | 82 | 102.310 | 99.855 | 89.746 | 0.00 | 0.00 | C |
| ATOM | 472 | CD | LYS | 82 | 102.506 | 98.366 | 89.344 | 0.00 | 0.00 | C |
| ATOM | 473 | CE | LYS | 82 | 103.964 | 97.854 | 89.290 | 0.00 | 0.00 | C |
| ATOM | 474 | NZ | LYS | 82 | 104.023 | 96.406 | 88.932 | 0.00 | 0.00 | N |
| ATOM | 475 | 1HZ | LYS | 82 | 103.258 | 95.811 | 89.258 | 1.00 | 0.00 | H |
| ATOM | 476 | 2HZ | LYS | 82 | 104.049 | 96.196 | 87.906 | 1.00 | 0.00 | H |
| ATOM | 477 | 3HZ | LYS | 82 | 104.877 | 95.915 | 89.296 | 1.00 | 0.00 | H |
| ATOM | 478 | H | LYS | 82 | 104.503 | 102.956 | 88.189 | 0.00 | 0.00 | H |
| ATOM | 479 | HA | LYS | 82 | 101.518 | 102.463 | 88.412 | 0.00 | 0.00 | H |
| ATOM | 480 | 1HB | LYS | 82 | 104.038 | 100.839 | 88.899 | 0.00 | 0.00 | H |
| ATOM | 481 | 2HB | LYS | 82 | 102.760 | 100.609 | 87.752 | 0.00 | 0.00 | H |
| ATOM | 482 | 1HG | LYS | 82 | 101.223 | 100.047 | 89.831 | 0.00 | 0.00 | H |
| ATOM | 483 | 2HG | LYS | 82 | 102.711 | 100.010 | 90.766 | 0.00 | 0.00 | H |
| ATOM | 484 | 1HD | LYS | 82 | 102.006 | 98.183 | 88.371 | 0.00 | 0.00 | H |
| ATOM | 485 | 2HD | LYS | 82 | 101.939 | 97.735 | 90.057 | 0.00 | 0.00 | H |
| ATOM | 486 | 1HE | LYS | 82 | 104.477 | 98.016 | 90.265 | 0.00 | 0.00 | H |
| ATOM | 487 | 2HE | LYS | 82 | 104.562 | 98.441 | 88.556 | 0.00 | 0.00 | H |
| ATOM | 488 | N | ILE | 83 | 101.013 | 103.425 | 90.715 | 0.00 | 0.00 | N |
| ATOM | 489 | CA | ILE | 83 | 100.653 | 103.996 | 92.050 | 0.00 | 0.00 | C |
| ATOM | 490 | C | ILE | 83 | 99.997 | 102.861 | 92.910 | 0.00 | 0.00 | C |
| ATOM | 491 | O | ILE | 83 | 98.864 | 102.450 | 92.636 | 0.00 | 0.00 | O |
| ATOM | 492 | CB | ILE | 83 | 99.731 | 105.267 | 91.908 | 0.00 | 0.00 | C |
| ATOM | 493 | CG2 | ILE | 83 | 99.435 | 105.908 | 93.291 | 0.00 | 0.00 | C |
| ATOM | 494 | CG1 | ILE | 83 | 100.274 | 106.395 | 90.972 | 0.00 | 0.00 | C |
| ATOM | 495 | CD1 | ILE | 83 | 99.796 | 106.303 | 89.513 | 0.00 | 0.00 | C |
| ATOM | 496 | H | ILE | 83 | 100.334 | 103.382 | 89.944 | 0.00 | 0.00 | H |
| ATOM | 497 | HA | ILE | 83 | 101.573 | 104.343 | 92.566 | 0.00 | 0.00 | H |
| ATOM | 498 | HB | ILE | 83 | 98.753 | 104.929 | 91.502 | 0.00 | 0.00 | H |
| ATOM | 499 | 1HG2 | ILE | 83 | 98.747 | 106.771 | 93.207 | 0.00 | 0.00 | H |
| ATOM | 500 | 2HG2 | ILE | 83 | 98.949 | 105.197 | 93.985 | 0.00 | 0.00 | H |
| ATOM | 501 | 3HG2 | ILE | 83 | 100.356 | 106.268 | 93.788 | 0.00 | 0.00 | H |
| ATOM | 502 | 1HG1 | ILE | 83 | 101.381 | 106.416 | 91.000 | 0.00 | 0.00 | H |
| ATOM | 503 | 2HG1 | ILE | 83 | 99.975 | 107.399 | 91.330 | 0.00 | 0.00 | H |
| ATOM | 504 | 1HD1 | ILE | 83 | 100.166 | 107.159 | 88.919 | 0.00 | 0.00 | H |
| ATOM | 505 | 2HD1 | ILE | 83 | 100.143 | 105.386 | 89.003 | 0.00 | 0.00 | H |
| ATOM | 506 | 3HD1 | ILE | 83 | 98.693 | 106.323 | 89.443 | 0.00 | 0.00 | H |
| ATOM | 507 | N | VAL | 84 | 100.698 | 102.366 | 93.944 | 1.00 | 0.00 | N |
| ATOM | 508 | CA | VAL | 84 | 100.244 | 101.190 | 94.755 | 1.00 | 0.00 | C |
| ATOM | 509 | C | VAL | 84 | 99.727 | 101.685 | 96.168 | 1.00 | 0.00 | C |
| ATOM | 510 | O | VAL | 84 | 100.568 | 101.985 | 97.027 | 1.00 | 0.00 | O |
| ATOM | 511 | CB | VAL | 84 | 101.388 | 100.119 | 94.872 | 1.00 | 0.00 | C |
| ATOM | 512 | CG1 | VAL | 84 | 100.985 | 98.872 | 95.691 | 1.00 | 0.00 | C |
| ATOM | 513 | CG2 | VAL | 84 | 101.915 | 99.594 | 93.519 | 1.00 | 0.00 | C |
| ATOM | 514 | H | VAL | 84 | 101.673 | 102.693 | 93.997 | 1.00 | 0.00 | H |
| ATOM | 515 | HA | VAL | 84 | 99.421 | 100.666 | 94.236 | 1.00 | 0.00 | H |
| ATOM | 516 | HB | VAL | 84 | 102.238 | 100.600 | 95.394 | 1.00 | 0.00 | H |
| ATOM | 517 | 1HG1 | VAL | 84 | 100.133 | 98.329 | 95.239 | 1.00 | 0.00 | H |
| ATOM | 518 | 2HG1 | VAL | 84 | 101.820 | 98.152 | 95.783 | 1.00 | 0.00 | H |
| ATOM | 519 | 3HG1 | VAL | 84 | 100.689 | 99.129 | 96.724 | 1.00 | 0.00 | H |
| ATOM | 520 | 2HG2 | VAL | 84 | 102.366 | 100.415 | 92.933 | 1.00 | 0.00 | H |
| ATOM | 521 | 3HG2 | VAL | 84 | 102.707 | 98.831 | 93.635 | 1.00 | 0.00 | H |
| ATOM | 522 | 1HG2 | VAL | 84 | 101.112 | 99.150 | 92.899 | 1.00 | 0.00 | H |
| ATOM | 523 | N | PRO | 85 | 98.394 | 101.783 | 96.473 | 1.00 | 0.00 | N |
| ATOM | 524 | CA | PRO | 85 | 97.898 | 102.296 | 97.780 | 1.00 | 0.00 | C |
| ATOM | 525 | C | PRO | 85 | 97.963 | 101.264 | 98.958 | 1.00 | 0.00 | C |
| ATOM | 526 | O | PRO | 85 | 97.609 | 100.091 | 98.822 | 1.00 | 0.00 | O |
| ATOM | 527 | CB | PRO | 85 | 96.465 | 102.731 | 97.410 | 1.00 | 0.00 | C |
| ATOM | 528 | CG | PRO | 85 | 96.034 | 101.769 | 96.303 | 1.00 | 0.00 | C |
| ATOM | 529 | CD | PRO | 85 | 97.312 | 101.509 | 95.509 | 1.00 | 0.00 | C |
| ATOM | 530 | HA | PRO | 85 | 98.468 | 103.206 | 98.052 | 1.00 | 0.00 | H |
| ATOM | 531 | 1HB | PRO | 85 | 95.769 | 102.729 | 98.270 | 1.00 | 0.00 | H |
| ATOM | 532 | 2HB | PRO | 85 | 96.477 | 103.769 | 97.020 | 1.00 | 0.00 | H |
| ATOM | 533 | 1HG | PRO | 85 | 95.682 | 100.819 | 96.743 | 1.00 | 0.00 | H |
| ATOM | 534 | 2HG | PRO | 85 | 95.213 | 102.164 | 95.675 | 1.00 | 0.00 | H |
| ATOM | 535 | 1HD | PRO | 85 | 97.341 | 100.464 | 95.147 | 1.00 | 0.00 | H |
| ATOM | 536 | 2HD | PRO | 85 | 97.377 | 102.183 | 94.633 | 1.00 | 0.00 | H |
| ATOM | 537 | N | LYS | 86 | 98.387 | 101.742 | 100.135 | 0.00 | 0.00 | N |
| ATOM | 538 | CA | LYS | 86 | 98.662 | 100.888 | 101.331 | 0.00 | 0.00 | C |
| ATOM | 539 | C | LYS | 86 | 97.517 | 99.968 | 101.889 | 0.00 | 0.00 | C |
| ATOM | 540 | O | LYS | 86 | 97.759 | 98.781 | 102.113 | 0.00 | 0.00 | O |
| ATOM | 541 | CB | LYS | 86 | 99.331 | 101.795 | 102.404 | 0.00 | 0.00 | C |
| ATOM | 542 | CG | LYS | 86 | 98.485 | 102.960 | 102.985 | 0.00 | 0.00 | C |
| ATOM | 543 | CD | LYS | 86 | 99.335 | 103.922 | 103.828 | 0.00 | 0.00 | C |
| ATOM | 544 | CE | LYS | 86 | 98.550 | 105.160 | 104.284 | 0.00 | 0.00 | C |
| ATOM | 545 | NZ | LYS | 86 | 99.460 | 106.049 | 105.030 | 0.00 | 0.00 | N |
| ATOM | 546 | 1HZ | LYS | 86 | 98.944 | 106.885 | 105.341 | 1.00 | 0.00 | H |
| ATOM | 547 | 2HZ | LYS | 86 | 99.832 | 105.550 | 105.851 | 1.00 | 0.00 | H |
| ATOM | 548 | 3HZ | LYS | 86 | 100.239 | 106.335 | 104.420 | 1.00 | 0.00 | H |
| ATOM | 549 | H | LYS | 86 | 98.689 | 102.718 | 100.099 | 0.00 | 0.00 | H |
| ATOM | 550 | HA | LYS | 86 | 99.450 | 100.171 | 101.022 | 0.00 | 0.00 | H |
| ATOM | 551 | 1HB | LYS | 86 | 100.269 | 102.203 | 101.974 | 0.00 | 0.00 | H |
| ATOM | 552 | 2HB | LYS | 86 | 99.682 | 101.158 | 103.240 | 0.00 | 0.00 | H |
| ATOM | 553 | 1HG | LYS | 86 | 97.650 | 102.564 | 103.596 | 0.00 | 0.00 | H |
| ATOM | 554 | 2HG | LYS | 86 | 98.007 | 103.533 | 102.165 | 0.00 | 0.00 | H |
| ATOM | 555 | 1HD | LYS | 86 | 100.211 | 104.244 | 103.232 | 0.00 | 0.00 | H |
| ATOM | 556 | 2HD | LYS | 86 | 99.741 | 103.384 | 104.706 | 0.00 | 0.00 | H |
| ATOM | 557 | 1HE | LYS | 86 | 97.685 | 104.879 | 104.916 | 0.00 | 0.00 | H |
| ATOM | 558 | 2HE | LYS | 86 | 98.133 | 105.705 | 103.413 | 0.00 | 0.00 | H |
| ATOM | 559 | N | SER | 87 | 96.287 | 100.474 | 102.092 | 1.00 | 0.00 | N |
| ATOM | 560 | CA | SER | 87 | 95.133 | 99.633 | 102.540 | 1.00 | 0.00 | C |
| ATOM | 561 | C | SER | 87 | 94.623 | 98.560 | 101.515 | 1.00 | 0.00 | C |
| ATOM | 562 | O | SER | 87 | 94.403 | 97.411 | 101.905 | 1.00 | 0.00 | O |
| ATOM | 563 | CB | SER | 87 | 94.007 | 100.572 | 103.026 | 1.00 | 0.00 | C |
| ATOM | 564 | OG | SER | 87 | 92.956 | 99.832 | 103.653 | 1.00 | 0.00 | O |
| ATOM | 565 | H | SER | 87 | 96.215 | 101.475 | 101.887 | 1.00 | 0.00 | H |
| ATOM | 566 | HA | SER | 87 | 95.461 | 99.059 | 103.431 | 1.00 | 0.00 | H |
| ATOM | 567 | 1HB | SER | 87 | 94.402 | 101.305 | 103.758 | 1.00 | 0.00 | H |
| ATOM | 568 | 2HB | SER | 87 | 93.596 | 101.174 | 102.191 | 1.00 | 0.00 | H |
| ATOM | 569 | HG | SER | 87 | 92.584 | 99.229 | 102.998 | 1.00 | 0.00 | H |
| ATOM | 570 | N | LEU | 88 | 94.447 | 98.902 | 100.227 | 1.00 | 0.00 | N |
| ATOM | 571 | CA | LEU | 88 | 94.175 | 97.890 | 99.154 | 1.00 | 0.00 | C |
| ATOM | 572 | C | LEU | 88 | 95.362 | 96.936 | 98.752 | 1.00 | 0.00 | C |
| ATOM | 573 | O | LEU | 88 | 95.100 | 95.892 | 98.156 | 1.00 | 0.00 | O |
| ATOM | 574 | CB | LEU | 88 | 93.567 | 98.609 | 97.916 | 1.00 | 0.00 | C |
| ATOM | 575 | CG | LEU | 88 | 92.166 | 99.261 | 98.079 | 1.00 | 0.00 | C |
| ATOM | 576 | CD1 | LEU | 88 | 91.833 | 100.106 | 96.839 | 1.00 | 0.00 | C |
| ATOM | 577 | CD2 | LEU | 88 | 91.053 | 98.218 | 98.282 | 1.00 | 0.00 | C |
| ATOM | 578 | H | LEU | 88 | 94.777 | 99.847 | 100.006 | 1.00 | 0.00 | H |
| ATOM | 579 | HA | LEU | 88 | 93.404 | 97.191 | 99.529 | 1.00 | 0.00 | H |
| ATOM | 580 | 1HB | LEU | 88 | 94.292 | 99.365 | 97.570 | 1.00 | 0.00 | H |
| ATOM | 581 | 2HB | LEU | 88 | 93.512 | 97.889 | 97.075 | 1.00 | 0.00 | H |
| ATOM | 582 | HG | LEU | 88 | 92.179 | 99.938 | 98.956 | 1.00 | 0.00 | H |
| ATOM | 583 | 2HD1 | LEU | 88 | 92.580 | 100.904 | 96.672 | 1.00 | 0.00 | H |
| ATOM | 584 | 3HD1 | LEU | 88 | 91.798 | 99.496 | 95.915 | 1.00 | 0.00 | H |
| ATOM | 585 | 1HD1 | LEU | 88 | 90.851 | 100.605 | 96.935 | 1.00 | 0.00 | H |
| ATOM | 586 | 2HD2 | LEU | 88 | 91.004 | 97.492 | 97.449 | 1.00 | 0.00 | H |
| ATOM | 587 | 3HD2 | LEU | 88 | 91.192 | 97.637 | 99.211 | 1.00 | 0.00 | H |
| ATOM | 588 | 1HD2 | LEU | 88 | 90.055 | 98.690 | 98.359 | 1.00 | 0.00 | H |
| ATOM | 589 | N | LEU | 89 | 96.626 | 97.224 | 99.109 | 1.00 | 0.00 | N |
| ATOM | 590 | CA | LEU | 89 | 97.699 | 96.192 | 99.214 | 1.00 | 0.00 | C |
| ATOM | 591 | C | LEU | 89 | 97.562 | 95.227 | 100.455 | 1.00 | 0.00 | C |
| ATOM | 592 | O | LEU | 89 | 97.664 | 94.008 | 100.298 | 1.00 | 0.00 | O |
| ATOM | 593 | CB | LEU | 89 | 99.055 | 96.960 | 99.166 | 1.00 | 0.00 | C |
| ATOM | 594 | CG | LEU | 89 | 100.327 | 96.091 | 98.994 | 1.00 | 0.00 | C |
| ATOM | 595 | CD1 | LEU | 89 | 100.467 | 95.560 | 97.558 | 1.00 | 0.00 | C |
| ATOM | 596 | CD2 | LEU | 89 | 101.587 | 96.896 | 99.355 | 1.00 | 0.00 | C |
| ATOM | 597 | H | LEU | 89 | 96.766 | 98.189 | 99.426 | 1.00 | 0.00 | H |
| ATOM | 598 | HA | LEU | 89 | 97.643 | 95.547 | 98.314 | 1.00 | 0.00 | H |
| ATOM | 599 | 1HB | LEU | 89 | 99.038 | 97.716 | 98.354 | 1.00 | 0.00 | H |
| ATOM | 600 | 2HB | LEU | 89 | 99.142 | 97.561 | 100.093 | 1.00 | 0.00 | H |
| ATOM | 601 | HG | LEU | 89 | 100.270 | 95.225 | 99.684 | 1.00 | 0.00 | H |
| ATOM | 602 | 2HD1 | LEU | 89 | 99.536 | 95.099 | 97.181 | 1.00 | 0.00 | H |
| ATOM | 603 | 3HD1 | LEU | 89 | 100.723 | 96.351 | 96.834 | 1.00 | 0.00 | H |
| ATOM | 604 | 1HD1 | LEU | 89 | 101.247 | 94.781 | 97.489 | 1.00 | 0.00 | H |
| ATOM | 605 | 2HD2 | LEU | 89 | 101.702 | 97.797 | 98.723 | 1.00 | 0.00 | H |
| ATOM | 606 | 3HD2 | LEU | 89 | 101.562 | 97.237 | 100.406 | 1.00 | 0.00 | H |
| ATOM | 607 | 1HD2 | LEU | 89 | 102.507 | 96.295 | 99.239 | 1.00 | 0.00 | H |
| ATOM | 608 | N | LEU | 90 | 97.330 | 95.750 | 101.676 | 1.00 | 0.00 | N |
| ATOM | 609 | CA | LEU | 90 | 97.169 | 94.928 | 102.915 | 1.00 | 0.00 | C |
| ATOM | 610 | C | LEU | 90 | 95.928 | 93.965 | 102.973 | 1.00 | 0.00 | C |
| ATOM | 611 | O | LEU | 90 | 96.098 | 92.800 | 103.340 | 1.00 | 0.00 | O |
| ATOM | 612 | CB | LEU | 90 | 97.194 | 95.890 | 104.141 | 1.00 | 0.00 | C |
| ATOM | 613 | CG | LEU | 90 | 98.552 | 96.556 | 104.496 | 1.00 | 0.00 | C |
| ATOM | 614 | CD1 | LEU | 90 | 98.334 | 97.715 | 105.480 | 1.00 | 0.00 | C |
| ATOM | 615 | CD2 | LEU | 90 | 99.545 | 95.556 | 105.115 | 1.00 | 0.00 | C |
| ATOM | 616 | H | LEU | 90 | 97.315 | 96.778 | 101.696 | 1.00 | 0.00 | H |
| ATOM | 617 | HA | LEU | 90 | 98.046 | 94.258 | 102.994 | 1.00 | 0.00 | H |
| ATOM | 618 | 1HB | LEU | 90 | 96.424 | 96.670 | 103.982 | 1.00 | 0.00 | H |
| ATOM | 619 | 2HB | LEU | 90 | 96.838 | 95.348 | 105.041 | 1.00 | 0.00 | H |
| ATOM | 620 | HG | LEU | 90 | 99.004 | 96.976 | 103.574 | 1.00 | 0.00 | H |
| ATOM | 621 | 2HD1 | LEU | 90 | 97.669 | 98.489 | 105.053 | 1.00 | 0.00 | H |
| ATOM | 622 | 3HD1 | LEU | 90 | 97.880 | 97.378 | 106.432 | 1.00 | 0.00 | H |
| ATOM | 623 | 1HD1 | LEU | 90 | 99.284 | 98.222 | 105.733 | 1.00 | 0.00 | H |
| ATOM | 624 | 2HD2 | LEU | 90 | 99.137 | 95.070 | 106.021 | 1.00 | 0.00 | H |
| ATOM | 625 | 3HD2 | LEU | 90 | 99.817 | 94.752 | 104.408 | 1.00 | 0.00 | H |
| ATOM | 626 | 1HD2 | LEU | 90 | 100.488 | 96.050 | 105.410 | 1.00 | 0.00 | H |
| ATOM | 627 | N | LYS | 91 | 94.703 | 94.420 | 102.640 | 0.00 | 0.00 | N |
| ATOM | 628 | CA | LYS | 91 | 93.473 | 93.563 | 102.662 | 0.00 | 0.00 | C |
| ATOM | 629 | C | LYS | 91 | 93.508 | 92.243 | 101.787 | 0.00 | 0.00 | C |
| ATOM | 630 | O | LYS | 91 | 93.266 | 91.185 | 102.376 | 0.00 | 0.00 | O |
| ATOM | 631 | CB | LYS | 91 | 92.218 | 94.452 | 102.398 | 0.00 | 0.00 | C |
| ATOM | 632 | CG | LYS | 91 | 91.542 | 95.092 | 103.637 | 0.00 | 0.00 | C |
| ATOM | 633 | CD | LYS | 91 | 92.338 | 96.229 | 104.304 | 0.00 | 0.00 | C |
| ATOM | 634 | CE | LYS | 91 | 91.582 | 96.846 | 105.488 | 0.00 | 0.00 | C |
| ATOM | 635 | NZ | LYS | 91 | 92.386 | 97.929 | 106.087 | 0.00 | 0.00 | N |
| ATOM | 636 | 1HZ | LYS | 91 | 91.873 | 98.339 | 106.881 | 1.00 | 0.00 | H |
| ATOM | 637 | 2HZ | LYS | 91 | 93.283 | 97.549 | 106.419 | 1.00 | 0.00 | H |
| ATOM | 638 | 3HZ | LYS | 91 | 92.562 | 98.657 | 105.380 | 1.00 | 0.00 | H |
| ATOM | 639 | H | LYS | 91 | 94.687 | 95.406 | 102.350 | 0.00 | 0.00 | H |
| ATOM | 640 | HA | LYS | 91 | 93.377 | 93.183 | 103.699 | 0.00 | 0.00 | H |
| ATOM | 641 | 1HB | LYS | 91 | 91.440 | 93.828 | 101.916 | 0.00 | 0.00 | H |
| ATOM | 642 | 2HB | LYS | 91 | 92.435 | 95.229 | 101.637 | 0.00 | 0.00 | H |
| ATOM | 643 | 1HG | LYS | 91 | 91.314 | 94.304 | 104.382 | 0.00 | 0.00 | H |
| ATOM | 644 | 2HG | LYS | 91 | 90.551 | 95.483 | 103.331 | 0.00 | 0.00 | H |
| ATOM | 645 | 1HD | LYS | 91 | 92.559 | 97.007 | 103.551 | 0.00 | 0.00 | H |
| ATOM | 646 | 2HD | LYS | 91 | 93.322 | 95.847 | 104.640 | 0.00 | 0.00 | H |
| ATOM | 647 | 1HE | LYS | 91 | 91.361 | 96.077 | 106.256 | 0.00 | 0.00 | H |
| ATOM | 648 | 2HE | LYS | 91 | 90.598 | 97.243 | 105.168 | 0.00 | 0.00 | H |
| ATOM | 649 | N | PRO | 92 | 93.817 | 92.198 | 100.453 | 1.00 | 0.00 | N |
| ATOM | 650 | CA | PRO | 92 | 94.036 | 90.912 | 99.718 | 1.00 | 0.00 | C |
| ATOM | 651 | C | PRO | 92 | 95.423 | 90.180 | 99.890 | 1.00 | 0.00 | C |
| ATOM | 652 | O | PRO | 92 | 95.793 | 89.369 | 99.038 | 1.00 | 0.00 | O |
| ATOM | 653 | CB | PRO | 92 | 93.768 | 91.398 | 98.277 | 1.00 | 0.00 | C |
| ATOM | 654 | CG | PRO | 92 | 94.319 | 92.824 | 98.235 | 1.00 | 0.00 | C |
| ATOM | 655 | CD | PRO | 92 | 93.992 | 93.397 | 99.612 | 1.00 | 0.00 | C |
| ATOM | 656 | HA | PRO | 92 | 93.260 | 90.170 | 99.994 | 1.00 | 0.00 | H |
| ATOM | 657 | 1HB | PRO | 92 | 94.220 | 90.748 | 97.504 | 1.00 | 0.00 | H |
| ATOM | 658 | 2HB | PRO | 92 | 92.679 | 91.403 | 98.076 | 1.00 | 0.00 | H |
| ATOM | 659 | 1HG | PRO | 92 | 95.417 | 92.815 | 98.087 | 1.00 | 0.00 | H |
| ATOM | 660 | 2HG | PRO | 92 | 93.897 | 93.431 | 97.411 | 1.00 | 0.00 | H |
| ATOM | 661 | 1HD | PRO | 92 | 94.812 | 94.049 | 99.968 | 1.00 | 0.00 | H |
| ATOM | 662 | 2HD | PRO | 92 | 93.067 | 94.003 | 99.577 | 1.00 | 0.00 | H |
| ATOM | 663 | N | HIS | 93 | 96.172 | 90.428 | 100.981 | 1.00 | 0.00 | N |
| ATOM | 664 | CA | HIS | 93 | 97.474 | 89.747 | 101.304 | 1.00 | 0.00 | C |
| ATOM | 665 | C | HIS | 93 | 98.649 | 89.921 | 100.264 | 1.00 | 0.00 | C |
| ATOM | 666 | O | HIS | 93 | 99.418 | 88.994 | 99.993 | 1.00 | 0.00 | O |
| ATOM | 667 | CB | HIS | 93 | 97.234 | 88.275 | 101.763 | 1.00 | 0.00 | C |
| ATOM | 668 | CG | HIS | 93 | 96.378 | 88.105 | 103.018 | 1.00 | 0.00 | C |
| ATOM | 669 | ND1 | HIS | 93 | 96.872 | 88.240 | 104.307 | 1.00 | 0.00 | N |
| ATOM | 670 | CE1 | HIS | 93 | 95.697 | 88.123 | 105.005 | 1.00 | 0.00 | C |
| ATOM | 671 | NE2 | HIS | 93 | 94.519 | 87.915 | 104.337 | 1.00 | 0.00 | N |
| ATOM | 672 | CD2 | HIS | 93 | 94.985 | 87.921 | 103.034 | 1.00 | 0.00 | C |
| ATOM | 673 | H | HIS | 93 | 95.706 | 91.058 | 101.647 | 1.00 | 0.00 | H |
| ATOM | 674 | HA | HIS | 93 | 97.865 | 90.269 | 102.198 | 1.00 | 0.00 | H |
| ATOM | 675 | 1HB | HIS | 93 | 96.801 | 87.691 | 100.929 | 1.00 | 0.00 | H |
| ATOM | 676 | 2HB | HIS | 93 | 98.208 | 87.786 | 101.958 | 1.00 | 0.00 | H |
| ATOM | 677 | HE1 | HIS | 93 | 95.706 | 88.228 | 106.083 | 1.00 | 0.00 | H |
| ATOM | 678 | HE2 | HIS | 93 | 93.557 | 87.930 | 104.690 | 1.00 | 0.00 | H |
| ATOM | 679 | HD2 | HIS | 93 | 94.367 | 87.869 | 102.148 | 1.00 | 0.00 | H |
| ATOM | 680 | N | GLN | 94 | 98.823 | 91.137 | 99.720 | 0.00 | 0.00 | N |
| ATOM | 681 | CA | GLN | 94 | 99.797 | 91.412 | 98.619 | 0.00 | 0.00 | C |
| ATOM | 682 | C | GLN | 94 | 101.180 | 92.046 | 99.017 | 0.00 | 0.00 | C |
| ATOM | 683 | O | GLN | 94 | 102.059 | 92.109 | 98.155 | 0.00 | 0.00 | O |
| ATOM | 684 | CB | GLN | 94 | 99.060 | 92.295 | 97.574 | 0.00 | 0.00 | C |
| ATOM | 685 | CG | GLN | 94 | 97.975 | 91.596 | 96.709 | 0.00 | 0.00 | C |
| ATOM | 686 | CD | GLN | 94 | 97.355 | 92.477 | 95.614 | 0.00 | 0.00 | C |
| ATOM | 687 | OE1 | GLN | 94 | 97.667 | 93.647 | 95.421 | 0.00 | 0.00 | O |
| ATOM | 688 | NE2 | GLN | 94 | 96.472 | 91.924 | 94.827 | 0.00 | 0.00 | N |
| ATOM | 689 | H | GLN | 94 | 98.058 | 91.789 | 99.942 | 0.00 | 0.00 | H |
| ATOM | 690 | HA | GLN | 94 | 100.067 | 90.467 | 98.106 | 0.00 | 0.00 | H |
| ATOM | 691 | 1HB | GLN | 94 | 99.805 | 92.722 | 96.876 | 0.00 | 0.00 | H |
| ATOM | 692 | 2HB | GLN | 94 | 98.619 | 93.181 | 98.071 | 0.00 | 0.00 | H |
| ATOM | 693 | 1HG | GLN | 94 | 97.166 | 91.217 | 97.361 | 0.00 | 0.00 | H |
| ATOM | 694 | 2HG | GLN | 94 | 98.414 | 90.700 | 96.231 | 0.00 | 0.00 | H |
| ATOM | 695 | 1HE2 | GLN | 94 | 96.379 | 90.910 | 94.915 | 0.00 | 0.00 | H |
| ATOM | 696 | 2HE2 | GLN | 94 | 96.271 | 92.498 | 94.001 | 0.00 | 0.00 | H |
| ATOM | 697 | N | ARG | 95 | 101.412 | 92.495 | 100.268 | 1.00 | 0.00 | N |
| ATOM | 698 | CA | ARG | 95 | 102.691 | 93.151 | 100.696 | 1.00 | 0.00 | C |
| ATOM | 699 | C | ARG | 95 | 104.026 | 92.358 | 100.429 | 1.00 | 0.00 | C |
| ATOM | 700 | O | ARG | 95 | 104.961 | 92.917 | 99.853 | 1.00 | 0.00 | O |
| ATOM | 701 | CB | ARG | 95 | 102.495 | 93.590 | 102.174 | 1.00 | 0.00 | C |
| ATOM | 702 | CG | ARG | 95 | 103.631 | 94.474 | 102.750 | 1.00 | 0.00 | C |
| ATOM | 703 | CD | ARG | 95 | 103.389 | 94.848 | 104.218 | 1.00 | 0.00 | C |
| ATOM | 704 | NE | ARG | 95 | 104.538 | 95.658 | 104.692 | 1.00 | 0.00 | N |
| ATOM | 705 | CZ | ARG | 95 | 104.723 | 96.079 | 105.937 | 1.00 | 0.00 | C |
| ATOM | 706 | NH1 | ARG | 95 | 103.907 | 95.845 | 106.921 | 1.00 | 0.00 | N |
| ATOM | 707 | NH2 | ARG | 95 | 105.786 | 96.762 | 106.176 | 1.00 | 0.00 | N |
| ATOM | 708 | HE | ARG | 95 | 105.252 | 95.916 | 103.996 | 1.00 | 0.00 | H |
| ATOM | 709 | H | ARG | 95 | 100.593 | 92.395 | 100.878 | 1.00 | 0.00 | H |
| ATOM | 710 | HA | ARG | 95 | 102.788 | 94.078 | 100.097 | 1.00 | 0.00 | H |
| ATOM | 711 | 1HB | ARG | 95 | 101.543 | 94.148 | 102.272 | 1.00 | 0.00 | H |
| ATOM | 712 | 2HB | ARG | 95 | 102.374 | 92.691 | 102.810 | 1.00 | 0.00 | H |
| ATOM | 713 | 1HG | ARG | 95 | 104.606 | 93.950 | 102.667 | 1.00 | 0.00 | H |
| ATOM | 714 | 2HG | ARG | 95 | 103.748 | 95.395 | 102.145 | 1.00 | 0.00 | H |
| ATOM | 715 | 1HD | ARG | 95 | 102.448 | 95.423 | 104.319 | 1.00 | 0.00 | H |
| ATOM | 716 | 2HD | ARG | 95 | 103.278 | 93.931 | 104.835 | 1.00 | 0.00 | H |
| ATOM | 717 | 2HH1 | ARG | 95 | 103.101 | 95.282 | 106.651 | 1.00 | 0.00 | H |
| ATOM | 718 | 1HH1 | ARG | 95 | 104.170 | 96.209 | 107.835 | 1.00 | 0.00 | H |
| ATOM | 719 | 1HH2 | ARG | 95 | 106.362 | 96.872 | 105.340 | 1.00 | 0.00 | H |
| ATOM | 720 | 2HH2 | ARG | 95 | 105.945 | 97.078 | 107.131 | 1.00 | 0.00 | H |
| ATOM | 721 | N | GLU | 96 | 104.096 | 91.080 | 100.833 | 0.00 | 0.00 | N |
| ATOM | 722 | CA | GLU | 96 | 105.189 | 90.147 | 100.412 | 0.00 | 0.00 | C |
| ATOM | 723 | C | GLU | 96 | 104.931 | 89.350 | 99.079 | 0.00 | 0.00 | C |
| ATOM | 724 | O | GLU | 96 | 105.894 | 88.989 | 98.402 | 0.00 | 0.00 | O |
| ATOM | 725 | CB | GLU | 96 | 105.463 | 89.164 | 101.587 | 0.00 | 0.00 | C |
| ATOM | 726 | CG | GLU | 96 | 106.066 | 89.793 | 102.872 | 0.00 | 0.00 | C |
| ATOM | 727 | CD | GLU | 96 | 106.233 | 88.802 | 104.017 | 0.00 | 0.00 | C |
| ATOM | 728 | OE1 | GLU | 96 | 105.377 | 88.590 | 104.869 | 0.00 | 0.00 | O |
| ATOM | 729 | OE2 | GLU | 96 | 107.438 | 88.174 | 103.990 | 0.00 | 0.00 | O |
| ATOM | 730 | H | GLU | 96 | 103.250 | 90.754 | 101.306 | 0.00 | 0.00 | H |
| ATOM | 731 | HA | GLU | 96 | 106.124 | 90.718 | 100.229 | 0.00 | 0.00 | H |
| ATOM | 732 | 1HB | GLU | 96 | 106.166 | 88.381 | 101.235 | 0.00 | 0.00 | H |
| ATOM | 733 | 2HB | GLU | 96 | 104.534 | 88.612 | 101.832 | 0.00 | 0.00 | H |
| ATOM | 734 | 1HG | GLU | 96 | 105.426 | 90.617 | 103.237 | 0.00 | 0.00 | H |
| ATOM | 735 | 2HG | GLU | 96 | 107.044 | 90.260 | 102.651 | 0.00 | 0.00 | H |
| ATOM | 736 | N | LYS | 97 | 103.671 | 89.057 | 98.688 | 0.00 | 0.00 | N |
| ATOM | 737 | CA | LYS | 97 | 103.353 | 88.216 | 97.488 | 0.00 | 0.00 | C |
| ATOM | 738 | C | LYS | 97 | 103.443 | 88.919 | 96.085 | 0.00 | 0.00 | C |
| ATOM | 739 | O | LYS | 97 | 104.022 | 88.356 | 95.154 | 0.00 | 0.00 | O |
| ATOM | 740 | CB | LYS | 97 | 101.948 | 87.561 | 97.656 | 0.00 | 0.00 | C |
| ATOM | 741 | CG | LYS | 97 | 101.708 | 86.653 | 98.883 | 0.00 | 0.00 | C |
| ATOM | 742 | CD | LYS | 97 | 100.329 | 85.964 | 98.808 | 0.00 | 0.00 | C |
| ATOM | 743 | CE | LYS | 97 | 99.974 | 85.222 | 100.102 | 0.00 | 0.00 | C |
| ATOM | 744 | NZ | LYS | 97 | 98.655 | 84.577 | 99.947 | 0.00 | 0.00 | N |
| ATOM | 745 | 1HZ | LYS | 97 | 98.413 | 84.076 | 100.815 | 1.00 | 0.00 | H |
| ATOM | 746 | 2HZ | LYS | 97 | 97.942 | 85.294 | 99.755 | 1.00 | 0.00 | H |
| ATOM | 747 | 3HZ | LYS | 97 | 98.690 | 83.908 | 99.164 | 1.00 | 0.00 | H |
| ATOM | 748 | H | LYS | 97 | 102.948 | 89.411 | 99.318 | 0.00 | 0.00 | H |
| ATOM | 749 | HA | LYS | 97 | 104.088 | 87.389 | 97.445 | 0.00 | 0.00 | H |
| ATOM | 750 | 1HB | LYS | 97 | 101.171 | 88.351 | 97.642 | 0.00 | 0.00 | H |
| ATOM | 751 | 2HB | LYS | 97 | 101.748 | 86.960 | 96.746 | 0.00 | 0.00 | H |
| ATOM | 752 | 1HG | LYS | 97 | 102.512 | 85.895 | 98.965 | 0.00 | 0.00 | H |
| ATOM | 753 | 2HG | LYS | 97 | 101.772 | 87.260 | 99.808 | 0.00 | 0.00 | H |
| ATOM | 754 | 1HD | LYS | 97 | 99.549 | 86.725 | 98.598 | 0.00 | 0.00 | H |
| ATOM | 755 | 2HD | LYS | 97 | 100.312 | 85.270 | 97.945 | 0.00 | 0.00 | H |
| ATOM | 756 | 1HE | LYS | 97 | 100.743 | 84.464 | 100.353 | 0.00 | 0.00 | H |
| ATOM | 757 | 2HE | LYS | 97 | 99.946 | 85.926 | 100.959 | 0.00 | 0.00 | H |
| ATOM | 758 | N | MET | 98 | 102.791 | 90.083 | 95.914 | 0.00 | 0.00 | N |
| ATOM | 759 | CA | MET | 98 | 102.786 | 90.853 | 94.632 | 0.00 | 0.00 | C |
| ATOM | 760 | C | MET | 98 | 103.722 | 92.109 | 94.646 | 0.00 | 0.00 | C |
| ATOM | 761 | O | MET | 98 | 104.487 | 92.313 | 93.699 | 0.00 | 0.00 | O |
| ATOM | 762 | CB | MET | 98 | 101.318 | 91.226 | 94.282 | 0.00 | 0.00 | C |
| ATOM | 763 | CG | MET | 98 | 100.475 | 90.087 | 93.674 | 0.00 | 0.00 | C |
| ATOM | 764 | SD | MET | 98 | 98.916 | 90.757 | 93.075 | 0.00 | 0.00 | S |
| ATOM | 765 | CE | MET | 98 | 98.221 | 89.275 | 92.335 | 0.00 | 0.00 | C |
| ATOM | 766 | H | MET | 98 | 102.511 | 90.501 | 96.807 | 0.00 | 0.00 | H |
| ATOM | 767 | HA | MET | 98 | 103.171 | 90.224 | 93.803 | 0.00 | 0.00 | H |
| ATOM | 768 | 1HB | MET | 98 | 100.800 | 91.647 | 95.163 | 0.00 | 0.00 | H |
| ATOM | 769 | 2HB | MET | 98 | 101.318 | 92.056 | 93.554 | 0.00 | 0.00 | H |
| ATOM | 770 | 1HG | MET | 98 | 101.006 | 89.617 | 92.825 | 0.00 | 0.00 | H |
| ATOM | 771 | 2HG | MET | 98 | 100.289 | 89.287 | 94.414 | 0.00 | 0.00 | H |
| ATOM | 772 | 1HE | MET | 98 | 98.818 | 88.972 | 91.457 | 0.00 | 0.00 | H |
| ATOM | 773 | 2HE | MET | 98 | 97.185 | 89.464 | 92.000 | 0.00 | 0.00 | H |
| ATOM | 774 | 3HE | MET | 98 | 98.208 | 88.439 | 93.057 | 0.00 | 0.00 | H |
| ATOM | 775 | N | SER | 99 | 103.680 | 92.944 | 95.705 | 0.00 | 0.00 | N |
| ATOM | 776 | CA | SER | 99 | 104.779 | 93.904 | 95.997 | 0.00 | 0.00 | C |
| ATOM | 777 | C | SER | 99 | 106.098 | 93.204 | 96.500 | 0.00 | 0.00 | C |
| ATOM | 778 | O | SER | 99 | 106.170 | 91.989 | 96.692 | 0.00 | 0.00 | O |
| ATOM | 779 | CB | SER | 99 | 104.199 | 94.965 | 96.962 | 0.00 | 0.00 | C |
| ATOM | 780 | OG | SER | 99 | 105.102 | 96.061 | 97.138 | 0.00 | 0.00 | O |
| ATOM | 781 | H | SER | 99 | 103.120 | 92.555 | 96.475 | 0.00 | 0.00 | H |
| ATOM | 782 | HA | SER | 99 | 105.037 | 94.431 | 95.057 | 0.00 | 0.00 | H |
| ATOM | 783 | 1HB | SER | 99 | 103.243 | 95.360 | 96.567 | 0.00 | 0.00 | H |
| ATOM | 784 | 2HB | SER | 99 | 103.965 | 94.501 | 97.940 | 0.00 | 0.00 | H |
| ATOM | 785 | HG | SER | 99 | 104.767 | 96.590 | 97.870 | 0.00 | 0.00 | H |
| ATOM | 786 | N | MET | 100 | 107.175 | 93.992 | 96.592 | 1.00 | 0.00 | N |
| ATOM | 787 | CA | MET | 100 | 108.594 | 93.499 | 96.645 | 1.00 | 0.00 | C |
| ATOM | 788 | C | MET | 100 | 109.244 | 93.032 | 95.286 | 1.00 | 0.00 | C |
| ATOM | 789 | O | MET | 100 | 110.469 | 92.898 | 95.226 | 1.00 | 0.00 | O |
| ATOM | 790 | CB | MET | 100 | 108.921 | 92.578 | 97.861 | 1.00 | 0.00 | C |
| ATOM | 791 | CG | MET | 100 | 108.700 | 93.223 | 99.246 | 1.00 | 0.00 | C |
| ATOM | 792 | SD | MET | 100 | 109.201 | 92.084 | 100.548 | 1.00 | 0.00 | S |
| ATOM | 793 | CE | MET | 100 | 108.896 | 93.143 | 101.971 | 1.00 | 0.00 | C |
| ATOM | 794 | H | MET | 100 | 106.918 | 94.984 | 96.637 | 1.00 | 0.00 | H |
| ATOM | 795 | HA | MET | 100 | 109.184 | 94.413 | 96.850 | 1.00 | 0.00 | H |
| ATOM | 796 | 1HB | MET | 100 | 108.342 | 91.636 | 97.792 | 1.00 | 0.00 | H |
| ATOM | 797 | 2HB | MET | 100 | 109.979 | 92.253 | 97.800 | 1.00 | 0.00 | H |
| ATOM | 798 | 1HG | MET | 100 | 109.273 | 94.163 | 99.344 | 1.00 | 0.00 | H |
| ATOM | 799 | 2HG | MET | 100 | 107.633 | 93.478 | 99.390 | 1.00 | 0.00 | H |
| ATOM | 800 | 1HE | MET | 100 | 109.507 | 94.061 | 101.915 | 1.00 | 0.00 | H |
| ATOM | 801 | 3HE | MET | 100 | 107.830 | 93.435 | 102.019 | 1.00 | 0.00 | H |
| ATOM | 802 | 2HE | MET | 100 | 109.152 | 92.615 | 102.907 | 1.00 | 0.00 | H |
| ATOM | 803 | N | GLU | 101 | 108.496 | 92.944 | 94.163 | 0.00 | 0.00 | N |
| ATOM | 804 | CA | GLU | 101 | 109.067 | 92.992 | 92.774 | 0.00 | 0.00 | C |
| ATOM | 805 | C | GLU | 101 | 110.071 | 94.151 | 92.406 | 0.00 | 0.00 | C |
| ATOM | 806 | O | GLU | 101 | 110.931 | 93.963 | 91.547 | 0.00 | 0.00 | O |
| ATOM | 807 | CB | GLU | 101 | 107.894 | 92.894 | 91.754 | 0.00 | 0.00 | C |
| ATOM | 808 | CG | GLU | 101 | 106.842 | 94.060 | 91.838 | 0.00 | 0.00 | C |
| ATOM | 809 | CD | GLU | 101 | 106.279 | 94.737 | 90.600 | 0.00 | 0.00 | C |
| ATOM | 810 | OE1 | GLU | 101 | 105.843 | 94.086 | 89.632 | 0.00 | 0.00 | O |
| ATOM | 811 | OE2 | GLU | 101 | 106.170 | 95.980 | 90.633 | 0.00 | 0.00 | O |
| ATOM | 812 | H | GLU | 101 | 107.491 | 92.835 | 94.351 | 0.00 | 0.00 | H |
| ATOM | 813 | HA | GLU | 101 | 109.663 | 92.066 | 92.653 | 0.00 | 0.00 | H |
| ATOM | 814 | 1HB | GLU | 101 | 107.339 | 91.952 | 91.938 | 0.00 | 0.00 | H |
| ATOM | 815 | 2HB | GLU | 101 | 108.294 | 92.806 | 90.725 | 0.00 | 0.00 | H |
| ATOM | 816 | 1HG | GLU | 101 | 107.241 | 94.883 | 92.455 | 0.00 | 0.00 | H |
| ATOM | 817 | 2HG | GLU | 101 | 105.963 | 93.712 | 92.397 | 0.00 | 0.00 | H |
| ATOM | 818 | N | ILE | 102 | 110.011 | 95.307 | 93.091 | 0.00 | 0.00 | N |
| ATOM | 819 | CA | ILE | 102 | 111.117 | 96.324 | 93.126 | 0.00 | 0.00 | C |
| ATOM | 820 | C | ILE | 102 | 112.564 | 95.800 | 93.464 | 0.00 | 0.00 | C |
| ATOM | 821 | O | ILE | 102 | 113.523 | 96.238 | 92.831 | 0.00 | 0.00 | O |
| ATOM | 822 | CB | ILE | 102 | 110.710 | 97.558 | 94.018 | 0.00 | 0.00 | C |
| ATOM | 823 | CG2 | ILE | 102 | 109.536 | 98.355 | 93.396 | 0.00 | 0.00 | C |
| ATOM | 824 | CG1 | ILE | 102 | 110.409 | 97.228 | 95.514 | 0.00 | 0.00 | C |
| ATOM | 825 | CD1 | ILE | 102 | 110.410 | 98.432 | 96.472 | 0.00 | 0.00 | C |
| ATOM | 826 | H | ILE | 102 | 109.270 | 95.297 | 93.797 | 0.00 | 0.00 | H |
| ATOM | 827 | HA | ILE | 102 | 111.220 | 96.704 | 92.090 | 0.00 | 0.00 | H |
| ATOM | 828 | HB | ILE | 102 | 111.585 | 98.242 | 94.004 | 0.00 | 0.00 | H |
| ATOM | 829 | 1HG2 | ILE | 102 | 109.385 | 99.330 | 93.894 | 0.00 | 0.00 | H |
| ATOM | 830 | 2HG2 | ILE | 102 | 109.710 | 98.570 | 92.327 | 0.00 | 0.00 | H |
| ATOM | 831 | 3HG2 | ILE | 102 | 108.578 | 97.804 | 93.456 | 0.00 | 0.00 | H |
| ATOM | 832 | 1HG1 | ILE | 102 | 111.165 | 96.515 | 95.895 | 0.00 | 0.00 | H |
| ATOM | 833 | 2HG1 | ILE | 102 | 109.448 | 96.687 | 95.601 | 0.00 | 0.00 | H |
| ATOM | 834 | 1HD1 | ILE | 102 | 110.241 | 98.116 | 97.517 | 0.00 | 0.00 | H |
| ATOM | 835 | 2HD1 | ILE | 102 | 111.381 | 98.963 | 96.454 | 0.00 | 0.00 | H |
| ATOM | 836 | 3HD1 | ILE | 102 | 109.623 | 99.168 | 96.227 | 0.00 | 0.00 | H |
| ATOM | 837 | N | SER | 103 | 112.731 | 94.840 | 94.395 | 1.00 | 0.00 | N |
| ATOM | 838 | CA | SER | 103 | 114.010 | 94.078 | 94.548 | 1.00 | 0.00 | C |
| ATOM | 839 | C | SER | 103 | 114.407 | 93.084 | 93.393 | 1.00 | 0.00 | C |
| ATOM | 840 | O | SER | 103 | 115.580 | 92.714 | 93.302 | 1.00 | 0.00 | O |
| ATOM | 841 | CB | SER | 103 | 113.946 | 93.347 | 95.908 | 1.00 | 0.00 | C |
| ATOM | 842 | OG | SER | 103 | 115.211 | 92.763 | 96.229 | 1.00 | 0.00 | O |
| ATOM | 843 | H | SER | 103 | 111.847 | 94.461 | 94.757 | 1.00 | 0.00 | H |
| ATOM | 844 | HA | SER | 103 | 114.842 | 94.808 | 94.614 | 1.00 | 0.00 | H |
| ATOM | 845 | 1HB | SER | 103 | 113.664 | 94.044 | 96.721 | 1.00 | 0.00 | H |
| ATOM | 846 | 2HB | SER | 103 | 113.161 | 92.564 | 95.899 | 1.00 | 0.00 | H |
| ATOM | 847 | HG | SER | 103 | 115.563 | 92.369 | 95.418 | 1.00 | 0.00 | H |
| ATOM | 848 | N | ILE | 104 | 113.466 | 92.652 | 92.534 | 0.00 | 0.00 | N |
| ATOM | 849 | CA | ILE | 104 | 113.770 | 91.892 | 91.283 | 0.00 | 0.00 | C |
| ATOM | 850 | C | ILE | 104 | 114.234 | 92.901 | 90.167 | 0.00 | 0.00 | C |
| ATOM | 851 | O | ILE | 104 | 115.419 | 92.936 | 89.833 | 0.00 | 0.00 | O |
| ATOM | 852 | CB | ILE | 104 | 112.573 | 90.937 | 90.894 | 0.00 | 0.00 | C |
| ATOM | 853 | CG2 | ILE | 104 | 112.868 | 90.156 | 89.586 | 0.00 | 0.00 | C |
| ATOM | 854 | CG1 | ILE | 104 | 112.175 | 89.917 | 92.006 | 0.00 | 0.00 | C |
| ATOM | 855 | CD1 | ILE | 104 | 110.818 | 89.209 | 91.806 | 0.00 | 0.00 | C |
| ATOM | 856 | H | ILE | 104 | 112.552 | 93.098 | 92.676 | 0.00 | 0.00 | H |
| ATOM | 857 | HA | ILE | 104 | 114.641 | 91.231 | 91.467 | 0.00 | 0.00 | H |
| ATOM | 858 | HB | ILE | 104 | 111.688 | 91.577 | 90.702 | 0.00 | 0.00 | H |
| ATOM | 859 | 1HG2 | ILE | 104 | 112.044 | 89.477 | 89.301 | 0.00 | 0.00 | H |
| ATOM | 860 | 2HG2 | ILE | 104 | 112.997 | 90.835 | 88.724 | 0.00 | 0.00 | H |
| ATOM | 861 | 3HG2 | ILE | 104 | 113.784 | 89.541 | 89.662 | 0.00 | 0.00 | H |
| ATOM | 862 | 1HG1 | ILE | 104 | 112.122 | 90.432 | 92.984 | 0.00 | 0.00 | H |
| ATOM | 863 | 2HG1 | ILE | 104 | 112.976 | 89.162 | 92.138 | 0.00 | 0.00 | H |
| ATOM | 864 | 1HD1 | ILE | 104 | 110.546 | 88.603 | 92.691 | 0.00 | 0.00 | H |
| ATOM | 865 | 2HD1 | ILE | 104 | 109.990 | 89.923 | 91.641 | 0.00 | 0.00 | H |
| ATOM | 866 | 3HD1 | ILE | 104 | 110.825 | 88.518 | 90.944 | 0.00 | 0.00 | H |
| ATOM | 867 | N | HIS | 105 | 113.344 | 93.746 | 89.616 | 1.00 | 0.00 | N |
| ATOM | 868 | CA | HIS | 105 | 113.695 | 94.702 | 88.517 | 1.00 | 0.00 | C |
| ATOM | 869 | C | HIS | 105 | 114.587 | 95.965 | 88.831 | 1.00 | 0.00 | C |
| ATOM | 870 | O | HIS | 105 | 114.998 | 96.646 | 87.890 | 1.00 | 0.00 | O |
| ATOM | 871 | CB | HIS | 105 | 112.403 | 95.005 | 87.708 | 1.00 | 0.00 | C |
| ATOM | 872 | CG | HIS | 105 | 111.208 | 95.654 | 88.402 | 1.00 | 0.00 | C |
| ATOM | 873 | ND1 | HIS | 105 | 109.950 | 95.079 | 88.433 | 1.00 | 0.00 | N |
| ATOM | 874 | CE1 | HIS | 105 | 109.254 | 96.094 | 89.029 | 1.00 | 0.00 | C |
| ATOM | 875 | NE2 | HIS | 105 | 109.900 | 97.248 | 89.378 | 1.00 | 0.00 | N |
| ATOM | 876 | CD2 | HIS | 105 | 111.175 | 96.940 | 88.946 | 1.00 | 0.00 | C |
| ATOM | 877 | H | HIS | 105 | 112.395 | 93.691 | 90.011 | 1.00 | 0.00 | H |
| ATOM | 878 | HA | HIS | 105 | 114.335 | 94.144 | 87.803 | 1.00 | 0.00 | H |
| ATOM | 879 | 1HB | HIS | 105 | 112.660 | 95.634 | 86.835 | 1.00 | 0.00 | H |
| ATOM | 880 | 2HB | HIS | 105 | 112.066 | 94.058 | 87.248 | 1.00 | 0.00 | H |
| ATOM | 881 | HE1 | HIS | 105 | 108.185 | 95.999 | 89.157 | 1.00 | 0.00 | H |
| ATOM | 882 | HE2 | HIS | 105 | 109.511 | 98.124 | 89.737 | 1.00 | 0.00 | H |
| ATOM | 883 | HD2 | HIS | 105 | 112.024 | 97.602 | 88.960 | 1.00 | 0.00 | H |
| ATOM | 884 | N | ARG | 106 | 114.967 | 96.248 | 90.091 | 1.00 | 0.00 | N |
| ATOM | 885 | CA | ARG | 106 | 116.188 | 97.062 | 90.403 | 1.00 | 0.00 | C |
| ATOM | 886 | C | ARG | 106 | 117.561 | 96.302 | 90.234 | 1.00 | 0.00 | C |
| ATOM | 887 | O | ARG | 106 | 118.534 | 96.905 | 89.777 | 1.00 | 0.00 | O |
| ATOM | 888 | CB | ARG | 106 | 116.016 | 97.669 | 91.825 | 1.00 | 0.00 | C |
| ATOM | 889 | CG | ARG | 106 | 116.944 | 98.849 | 92.215 | 1.00 | 0.00 | C |
| ATOM | 890 | CD | ARG | 106 | 116.548 | 100.176 | 91.548 | 1.00 | 0.00 | C |
| ATOM | 891 | NE | ARG | 106 | 117.304 | 101.306 | 92.139 | 1.00 | 0.00 | N |
| ATOM | 892 | CZ | ARG | 106 | 117.196 | 102.573 | 91.754 | 1.00 | 0.00 | C |
| ATOM | 893 | NH1 | ARG | 106 | 116.436 | 102.985 | 90.782 | 1.00 | 0.00 | N |
| ATOM | 894 | NH2 | ARG | 106 | 117.895 | 103.444 | 92.389 | 1.00 | 0.00 | N |
| ATOM | 895 | HE | ARG | 106 | 117.960 | 101.093 | 92.904 | 1.00 | 0.00 | H |
| ATOM | 896 | H | ARG | 106 | 114.499 | 95.669 | 90.799 | 1.00 | 0.00 | H |
| ATOM | 897 | HA | ARG | 106 | 116.232 | 97.909 | 89.690 | 1.00 | 0.00 | H |
| ATOM | 898 | 1HB | ARG | 106 | 114.973 | 98.014 | 91.962 | 1.00 | 0.00 | H |
| ATOM | 899 | 2HB | ARG | 106 | 116.131 | 96.860 | 92.572 | 1.00 | 0.00 | H |
| ATOM | 900 | 1HG | ARG | 106 | 116.907 | 98.977 | 93.315 | 1.00 | 0.00 | H |
| ATOM | 901 | 2HG | ARG | 106 | 118.005 | 98.616 | 91.995 | 1.00 | 0.00 | H |
| ATOM | 902 | 1HD | ARG | 106 | 116.720 | 100.122 | 90.454 | 1.00 | 0.00 | H |
| ATOM | 903 | 2HD | ARG | 106 | 115.467 | 100.362 | 91.693 | 1.00 | 0.00 | H |
| ATOM | 904 | 2HH1 | ARG | 106 | 115.966 | 102.231 | 90.280 | 1.00 | 0.00 | H |
| ATOM | 905 | 1HH1 | ARG | 106 | 116.449 | 103.981 | 90.557 | 1.00 | 0.00 | H |
| ATOM | 906 | 1HH2 | ARG | 106 | 118.476 | 103.015 | 93.111 | 1.00 | 0.00 | H |
| ATOM | 907 | 2HH2 | ARG | 106 | 117.837 | 104.410 | 92.071 | 1.00 | 0.00 | H |
| ATOM | 908 | N | SER | 107 | 117.656 | 95.006 | 90.591 | 1.00 | 0.00 | N |
| ATOM | 909 | CA | SER | 107 | 118.898 | 94.191 | 90.412 | 1.00 | 0.00 | C |
| ATOM | 910 | C | SER | 107 | 119.231 | 93.611 | 88.983 | 1.00 | 0.00 | C |
| ATOM | 911 | O | SER | 107 | 120.313 | 93.038 | 88.811 | 1.00 | 0.00 | O |
| ATOM | 912 | CB | SER | 107 | 118.801 | 93.051 | 91.457 | 1.00 | 0.00 | C |
| ATOM | 913 | OG | SER | 107 | 120.020 | 92.306 | 91.515 | 1.00 | 0.00 | O |
| ATOM | 914 | H | SER | 107 | 116.758 | 94.573 | 90.835 | 1.00 | 0.00 | H |
| ATOM | 915 | HA | SER | 107 | 119.773 | 94.811 | 90.692 | 1.00 | 0.00 | H |
| ATOM | 916 | 1HB | SER | 107 | 118.583 | 93.450 | 92.468 | 1.00 | 0.00 | H |
| ATOM | 917 | 2HB | SER | 107 | 117.956 | 92.371 | 91.219 | 1.00 | 0.00 | H |
| ATOM | 918 | HG | SER | 107 | 120.314 | 92.177 | 90.601 | 1.00 | 0.00 | H |
| ATOM | 919 | N | LEU | 108 | 118.339 | 93.708 | 87.985 | 1.00 | 0.00 | N |
| ATOM | 920 | CA | LEU | 108 | 118.502 | 93.046 | 86.658 | 1.00 | 0.00 | C |
| ATOM | 921 | C | LEU | 108 | 119.119 | 94.006 | 85.584 | 1.00 | 0.00 | C |
| ATOM | 922 | O | LEU | 108 | 118.409 | 94.660 | 84.814 | 1.00 | 0.00 | O |
| ATOM | 923 | CB | LEU | 108 | 117.098 | 92.487 | 86.259 | 1.00 | 0.00 | C |
| ATOM | 924 | CG | LEU | 108 | 116.686 | 91.054 | 86.710 | 1.00 | 0.00 | C |
| ATOM | 925 | CD1 | LEU | 108 | 117.204 | 90.574 | 88.077 | 1.00 | 0.00 | C |
| ATOM | 926 | CD2 | LEU | 108 | 115.152 | 90.965 | 86.708 | 1.00 | 0.00 | C |
| ATOM | 927 | H | LEU | 108 | 117.463 | 94.155 | 88.273 | 1.00 | 0.00 | H |
| ATOM | 928 | HA | LEU | 108 | 119.184 | 92.174 | 86.733 | 1.00 | 0.00 | H |
| ATOM | 929 | 1HB | LEU | 108 | 116.324 | 93.229 | 86.542 | 1.00 | 0.00 | H |
| ATOM | 930 | 2HB | LEU | 108 | 117.022 | 92.480 | 85.156 | 1.00 | 0.00 | H |
| ATOM | 931 | HG | LEU | 108 | 117.076 | 90.334 | 85.960 | 1.00 | 0.00 | H |
| ATOM | 932 | 2HD1 | LEU | 108 | 118.307 | 90.517 | 88.100 | 1.00 | 0.00 | H |
| ATOM | 933 | 3HD1 | LEU | 108 | 116.898 | 91.239 | 88.903 | 1.00 | 0.00 | H |
| ATOM | 934 | 1HD1 | LEU | 108 | 116.842 | 89.558 | 88.321 | 1.00 | 0.00 | H |
| ATOM | 935 | 2HD2 | LEU | 108 | 114.700 | 91.680 | 87.419 | 1.00 | 0.00 | H |
| ATOM | 936 | 3HD2 | LEU | 108 | 114.728 | 91.191 | 85.712 | 1.00 | 0.00 | H |
| ATOM | 937 | 1HD2 | LEU | 108 | 114.791 | 89.961 | 86.993 | 1.00 | 0.00 | H |
| ATOM | 938 | N | ALA | 109 | 120.459 | 94.046 | 85.502 | 1.00 | 0.00 | N |
| ATOM | 939 | CA | ALA | 109 | 121.181 | 94.809 | 84.450 | 1.00 | 0.00 | C |
| ATOM | 940 | C | ALA | 109 | 121.276 | 94.026 | 83.092 | 1.00 | 0.00 | C |
| ATOM | 941 | O | ALA | 109 | 122.205 | 93.242 | 82.864 | 1.00 | 0.00 | O |
| ATOM | 942 | CB | ALA | 109 | 122.549 | 95.166 | 85.065 | 1.00 | 0.00 | C |
| ATOM | 943 | H | ALA | 109 | 120.937 | 93.517 | 86.238 | 1.00 | 0.00 | H |
| ATOM | 944 | HA | ALA | 109 | 120.661 | 95.770 | 84.261 | 1.00 | 0.00 | H |
| ATOM | 945 | 2HB | ALA | 109 | 122.442 | 95.760 | 85.993 | 1.00 | 0.00 | H |
| ATOM | 946 | 3HB | ALA | 109 | 123.147 | 94.269 | 85.313 | 1.00 | 0.00 | H |
| ATOM | 947 | 1HB | ALA | 109 | 123.155 | 95.773 | 84.367 | 1.00 | 0.00 | H |
| ATOM | 948 | N | HIS | 110 | 120.281 | 94.224 | 82.211 | 0.00 | 0.00 | N |
| ATOM | 949 | CA | HIS | 110 | 120.183 | 93.522 | 80.899 | 0.00 | 0.00 | C |
| ATOM | 950 | C | HIS | 110 | 119.536 | 94.431 | 79.796 | 0.00 | 0.00 | C |
| ATOM | 951 | O | HIS | 110 | 118.774 | 95.359 | 80.081 | 0.00 | 0.00 | O |
| ATOM | 952 | CB | HIS | 110 | 119.385 | 92.201 | 81.129 | 0.00 | 0.00 | C |
| ATOM | 953 | CG | HIS | 110 | 119.470 | 91.154 | 80.018 | 0.00 | 0.00 | C |
| ATOM | 954 | ND1 | HIS | 110 | 118.360 | 90.593 | 79.403 | 0.00 | 0.00 | N |
| ATOM | 955 | CE1 | HIS | 110 | 118.990 | 89.661 | 78.619 | 0.00 | 0.00 | C |
| ATOM | 956 | NE2 | HIS | 110 | 120.356 | 89.578 | 78.616 | 0.00 | 0.00 | N |
| ATOM | 957 | CD2 | HIS | 110 | 120.651 | 90.545 | 79.559 | 0.00 | 0.00 | C |
| ATOM | 958 | H | HIS | 110 | 119.499 | 94.777 | 82.586 | 0.00 | 0.00 | H |
| ATOM | 959 | HA | HIS | 110 | 121.208 | 93.272 | 80.555 | 0.00 | 0.00 | H |
| ATOM | 960 | 1HB | HIS | 110 | 118.324 | 92.438 | 81.344 | 0.00 | 0.00 | H |
| ATOM | 961 | 2HB | HIS | 110 | 119.738 | 91.705 | 82.050 | 0.00 | 0.00 | H |
| ATOM | 962 | HE1 | HIS | 110 | 118.402 | 88.948 | 78.058 | 0.00 | 0.00 | H |
| ATOM | 963 | HE2 | HIS | 110 | 120.958 | 88.868 | 78.185 | 0.00 | 0.00 | H |
| ATOM | 964 | HD2 | HIS | 110 | 121.637 | 90.755 | 79.950 | 0.00 | 0.00 | H |
| ATOM | 965 | N | GLN | 111 | 119.796 | 94.124 | 78.511 | 1.00 | 0.00 | N |
| ATOM | 966 | CA | GLN | 111 | 119.073 | 94.767 | 77.370 | 1.00 | 0.00 | C |
| ATOM | 967 | C | GLN | 111 | 117.522 | 94.518 | 77.294 | 1.00 | 0.00 | C |
| ATOM | 968 | O | GLN | 111 | 116.783 | 95.452 | 76.977 | 1.00 | 0.00 | O |
| ATOM | 969 | CB | GLN | 111 | 119.814 | 94.355 | 76.065 | 1.00 | 0.00 | C |
| ATOM | 970 | CG | GLN | 111 | 119.352 | 95.115 | 74.789 | 1.00 | 0.00 | C |
| ATOM | 971 | CD | GLN | 111 | 120.079 | 94.724 | 73.503 | 1.00 | 0.00 | C |
| ATOM | 972 | OE1 | GLN | 111 | 119.676 | 93.832 | 72.768 | 1.00 | 0.00 | O |
| ATOM | 973 | NE2 | GLN | 111 | 121.156 | 95.386 | 73.167 | 1.00 | 0.00 | N |
| ATOM | 974 | H | GLN | 111 | 120.352 | 93.273 | 78.391 | 1.00 | 0.00 | H |
| ATOM | 975 | HA | GLN | 111 | 119.191 | 95.864 | 77.481 | 1.00 | 0.00 | H |
| ATOM | 976 | 1HB | GLN | 111 | 120.904 | 94.513 | 76.190 | 1.00 | 0.00 | H |
| ATOM | 977 | 2HB | GLN | 111 | 119.695 | 93.264 | 75.901 | 1.00 | 0.00 | H |
| ATOM | 978 | 1HG | GLN | 111 | 118.276 | 94.920 | 74.613 | 1.00 | 0.00 | H |
| ATOM | 979 | 2HG | GLN | 111 | 119.413 | 96.208 | 74.943 | 1.00 | 0.00 | H |
| ATOM | 980 | 1HE2 | GLN | 111 | 121.424 | 96.166 | 73.769 | 1.00 | 0.00 | H |
| ATOM | 981 | 2HE2 | GLN | 111 | 121.540 | 95.113 | 72.259 | 1.00 | 0.00 | H |
| ATOM | 982 | N | HIS | 112 | 117.029 | 93.294 | 77.559 | 1.00 | 0.00 | N |
| ATOM | 983 | CA | HIS | 112 | 115.593 | 92.928 | 77.334 | 1.00 | 0.00 | C |
| ATOM | 984 | C | HIS | 112 | 114.665 | 93.099 | 78.602 | 1.00 | 0.00 | C |
| ATOM | 985 | O | HIS | 112 | 113.745 | 92.309 | 78.828 | 1.00 | 0.00 | O |
| ATOM | 986 | CB | HIS | 112 | 115.545 | 91.483 | 76.735 | 1.00 | 0.00 | C |
| ATOM | 987 | CG | HIS | 112 | 116.435 | 91.140 | 75.527 | 1.00 | 0.00 | C |
| ATOM | 988 | ND1 | HIS | 112 | 116.759 | 92.021 | 74.504 | 1.00 | 0.00 | N |
| ATOM | 989 | CE1 | HIS | 112 | 117.721 | 91.285 | 73.864 | 1.00 | 0.00 | C |
| ATOM | 990 | NE2 | HIS | 112 | 118.030 | 90.033 | 74.311 | 1.00 | 0.00 | N |
| ATOM | 991 | CD2 | HIS | 112 | 117.181 | 89.959 | 75.402 | 1.00 | 0.00 | C |
| ATOM | 992 | H | HIS | 112 | 117.723 | 92.612 | 77.883 | 1.00 | 0.00 | H |
| ATOM | 993 | HA | HIS | 112 | 115.158 | 93.595 | 76.563 | 1.00 | 0.00 | H |
| ATOM | 994 | 1HB | HIS | 112 | 115.756 | 90.764 | 77.550 | 1.00 | 0.00 | H |
| ATOM | 995 | 2HB | HIS | 112 | 114.502 | 91.264 | 76.442 | 1.00 | 0.00 | H |
| ATOM | 996 | HE1 | HIS | 112 | 118.248 | 91.719 | 73.024 | 1.00 | 0.00 | H |
| ATOM | 997 | HE2 | HIS | 112 | 118.792 | 89.412 | 74.014 | 1.00 | 0.00 | H |
| ATOM | 998 | HD2 | HIS | 112 | 117.131 | 89.137 | 76.100 | 1.00 | 0.00 | H |
| ATOM | 999 | N | VAL | 113 | 114.879 | 94.140 | 79.426 | 1.00 | 0.00 | N |
| ATOM | 1000 | CA | VAL | 113 | 114.177 | 94.363 | 80.732 | 1.00 | 0.00 | C |
| ATOM | 1001 | C | VAL | 113 | 113.876 | 95.899 | 80.839 | 1.00 | 0.00 | C |
| ATOM | 1002 | O | VAL | 113 | 114.762 | 96.727 | 80.603 | 1.00 | 0.00 | O |
| ATOM | 1003 | CB | VAL | 113 | 115.031 | 93.822 | 81.942 | 1.00 | 0.00 | C |
| ATOM | 1004 | CG1 | VAL | 113 | 114.422 | 94.136 | 83.330 | 1.00 | 0.00 | C |
| ATOM | 1005 | CG2 | VAL | 113 | 115.264 | 92.290 | 81.916 | 1.00 | 0.00 | C |
| ATOM | 1006 | H | VAL | 113 | 115.671 | 94.731 | 79.141 | 1.00 | 0.00 | H |
| ATOM | 1007 | HA | VAL | 113 | 113.208 | 93.826 | 80.741 | 1.00 | 0.00 | H |
| ATOM | 1008 | HB | VAL | 113 | 116.025 | 94.314 | 81.899 | 1.00 | 0.00 | H |
| ATOM | 1009 | 1HG1 | VAL | 113 | 113.405 | 93.722 | 83.446 | 1.00 | 0.00 | H |
| ATOM | 1010 | 2HG1 | VAL | 113 | 115.039 | 93.742 | 84.161 | 1.00 | 0.00 | H |
| ATOM | 1011 | 3HG1 | VAL | 113 | 114.345 | 95.227 | 83.509 | 1.00 | 0.00 | H |
| ATOM | 1012 | 2HG2 | VAL | 113 | 115.809 | 91.975 | 81.008 | 1.00 | 0.00 | H |
| ATOM | 1013 | 3HG2 | VAL | 113 | 115.874 | 91.945 | 82.773 | 1.00 | 0.00 | H |
| ATOM | 1014 | 1HG2 | VAL | 113 | 114.314 | 91.724 | 81.936 | 1.00 | 0.00 | H |
| ATOM | 1015 | N | VAL | 114 | 112.642 | 96.296 | 81.208 | 0.00 | 0.00 | N |
| ATOM | 1016 | CA | VAL | 114 | 112.230 | 97.744 | 81.262 | 0.00 | 0.00 | C |
| ATOM | 1017 | C | VAL | 114 | 113.120 | 98.550 | 82.289 | 0.00 | 0.00 | C |
| ATOM | 1018 | O | VAL | 114 | 113.304 | 98.124 | 83.436 | 0.00 | 0.00 | O |
| ATOM | 1019 | CB | VAL | 114 | 110.701 | 97.852 | 81.615 | 0.00 | 0.00 | C |
| ATOM | 1020 | CG1 | VAL | 114 | 110.200 | 99.308 | 81.734 | 0.00 | 0.00 | C |
| ATOM | 1021 | CG2 | VAL | 114 | 109.717 | 97.185 | 80.624 | 0.00 | 0.00 | C |
| ATOM | 1022 | H | VAL | 114 | 112.016 | 95.535 | 81.503 | 0.00 | 0.00 | H |
| ATOM | 1023 | HA | VAL | 114 | 112.370 | 98.187 | 80.256 | 0.00 | 0.00 | H |
| ATOM | 1024 | HB | VAL | 114 | 110.557 | 97.367 | 82.602 | 0.00 | 0.00 | H |
| ATOM | 1025 | 1HG1 | VAL | 114 | 110.767 | 99.897 | 82.479 | 0.00 | 0.00 | H |
| ATOM | 1026 | 2HG1 | VAL | 114 | 110.258 | 99.866 | 80.780 | 0.00 | 0.00 | H |
| ATOM | 1027 | 3HG1 | VAL | 114 | 109.150 | 99.339 | 82.069 | 0.00 | 0.00 | H |
| ATOM | 1028 | 1HG2 | VAL | 114 | 109.688 | 97.697 | 79.644 | 0.00 | 0.00 | H |
| ATOM | 1029 | 2HG2 | VAL | 114 | 109.962 | 96.125 | 80.438 | 0.00 | 0.00 | H |
| ATOM | 1030 | 3HG2 | VAL | 114 | 108.678 | 97.192 | 81.006 | 0.00 | 0.00 | H |
| ATOM | 1031 | N | GLY | 115 | 113.677 | 99.701 | 81.873 | 1.00 | 0.00 | N |
| ATOM | 1032 | CA | GLY | 115 | 114.555 | 100.527 | 82.744 | 1.00 | 0.00 | C |
| ATOM | 1033 | C | GLY | 115 | 113.860 | 101.190 | 83.956 | 1.00 | 0.00 | C |
| ATOM | 1034 | O | GLY | 115 | 112.941 | 101.999 | 83.811 | 1.00 | 0.00 | O |
| ATOM | 1035 | H | GLY | 115 | 113.367 | 100.067 | 80.961 | 1.00 | 0.00 | H |
| ATOM | 1036 | 1HA | GLY | 115 | 115.441 | 99.941 | 83.061 | 1.00 | 0.00 | H |
| ATOM | 1037 | 2HA | GLY | 115 | 114.975 | 101.342 | 82.130 | 1.00 | 0.00 | H |
| ATOM | 1038 | N | PHE | 116 | 114.284 | 100.805 | 85.163 | 1.00 | 0.00 | N |
| ATOM | 1039 | CA | PHE | 116 | 113.653 | 101.270 | 86.423 | 1.00 | 0.00 | C |
| ATOM | 1040 | C | PHE | 116 | 114.361 | 102.526 | 87.030 | 1.00 | 0.00 | C |
| ATOM | 1041 | O | PHE | 116 | 115.506 | 102.470 | 87.485 | 1.00 | 0.00 | O |
| ATOM | 1042 | CB | PHE | 116 | 113.598 | 100.040 | 87.373 | 1.00 | 0.00 | C |
| ATOM | 1043 | CG | PHE | 116 | 112.797 | 100.237 | 88.671 | 1.00 | 0.00 | C |
| ATOM | 1044 | CD1 | PHE | 116 | 111.508 | 100.777 | 88.644 | 1.00 | 0.00 | C |
| ATOM | 1045 | CE1 | PHE | 116 | 110.797 | 100.954 | 89.820 | 1.00 | 0.00 | C |
| ATOM | 1046 | CZ | PHE | 116 | 111.341 | 100.551 | 91.030 | 1.00 | 0.00 | C |
| ATOM | 1047 | CE2 | PHE | 116 | 112.610 | 99.980 | 91.072 | 1.00 | 0.00 | C |
| ATOM | 1048 | CD2 | PHE | 116 | 113.338 | 99.830 | 89.893 | 1.00 | 0.00 | C |
| ATOM | 1049 | H | PHE | 116 | 114.915 | 99.996 | 85.144 | 1.00 | 0.00 | H |
| ATOM | 1050 | HA | PHE | 116 | 112.602 | 101.542 | 86.204 | 1.00 | 0.00 | H |
| ATOM | 1051 | 1HB | PHE | 116 | 113.152 | 99.173 | 86.844 | 1.00 | 0.00 | H |
| ATOM | 1052 | 2HB | PHE | 116 | 114.633 | 99.711 | 87.601 | 1.00 | 0.00 | H |
| ATOM | 1053 | HD1 | PHE | 116 | 111.048 | 101.070 | 87.714 | 1.00 | 0.00 | H |
| ATOM | 1054 | HE1 | PHE | 116 | 109.818 | 101.392 | 89.786 | 1.00 | 0.00 | H |
| ATOM | 1055 | HZ | PHE | 116 | 110.760 | 100.687 | 91.925 | 1.00 | 0.00 | H |
| ATOM | 1056 | HE2 | PHE | 116 | 113.026 | 99.647 | 92.013 | 1.00 | 0.00 | H |
| ATOM | 1057 | HD2 | PHE | 116 | 114.313 | 99.368 | 89.922 | 1.00 | 0.00 | H |
| ATOM | 1058 | N | HIS | 117 | 113.642 | 103.657 | 87.050 | 1.00 | 0.00 | N |
| ATOM | 1059 | CA | HIS | 117 | 114.145 | 104.947 | 87.595 | 1.00 | 0.00 | C |
| ATOM | 1060 | C | HIS | 117 | 114.102 | 105.098 | 89.160 | 1.00 | 0.00 | C |
| ATOM | 1061 | O | HIS | 117 | 115.055 | 105.637 | 89.731 | 1.00 | 0.00 | O |
| ATOM | 1062 | CB | HIS | 117 | 113.346 | 106.045 | 86.836 | 1.00 | 0.00 | C |
| ATOM | 1063 | CG | HIS | 117 | 113.832 | 107.481 | 87.004 | 1.00 | 0.00 | C |
| ATOM | 1064 | ND1 | HIS | 117 | 113.032 | 108.504 | 87.493 | 1.00 | 0.00 | N |
| ATOM | 1065 | CE1 | HIS | 117 | 113.899 | 109.556 | 87.340 | 1.00 | 0.00 | C |
| ATOM | 1066 | NE2 | HIS | 117 | 115.144 | 109.346 | 86.812 | 1.00 | 0.00 | N |
| ATOM | 1067 | CD2 | HIS | 117 | 115.081 | 107.982 | 86.595 | 1.00 | 0.00 | C |
| ATOM | 1068 | H | HIS | 117 | 112.668 | 103.548 | 86.734 | 1.00 | 0.00 | H |
| ATOM | 1069 | HA | HIS | 117 | 115.213 | 105.053 | 87.312 | 1.00 | 0.00 | H |
| ATOM | 1070 | 1HB | HIS | 117 | 113.342 | 105.837 | 85.749 | 1.00 | 0.00 | H |
| ATOM | 1071 | 2HB | HIS | 117 | 112.280 | 105.988 | 87.128 | 1.00 | 0.00 | H |
| ATOM | 1072 | HE1 | HIS | 117 | 113.600 | 110.546 | 87.656 | 1.00 | 0.00 | H |
| ATOM | 1073 | HE2 | HIS | 117 | 115.907 | 110.015 | 86.667 | 1.00 | 0.00 | H |
| ATOM | 1074 | HD2 | HIS | 117 | 115.869 | 107.380 | 86.162 | 1.00 | 0.00 | H |
| ATOM | 1075 | N | GLY | 118 | 113.030 | 104.664 | 89.853 | 1.00 | 0.00 | N |
| ATOM | 1076 | CA | GLY | 118 | 112.920 | 104.847 | 91.324 | 1.00 | 0.00 | C |
| ATOM | 1077 | C | GLY | 118 | 111.772 | 104.081 | 92.021 | 1.00 | 0.00 | C |
| ATOM | 1078 | O | GLY | 118 | 110.634 | 104.043 | 91.544 | 1.00 | 0.00 | O |
| ATOM | 1079 | H | GLY | 118 | 112.330 | 104.175 | 89.282 | 1.00 | 0.00 | H |
| ATOM | 1080 | 1HA | GLY | 118 | 113.891 | 104.600 | 91.798 | 1.00 | 0.00 | H |
| ATOM | 1081 | 2HA | GLY | 118 | 112.763 | 105.919 | 91.540 | 1.00 | 0.00 | H |
| ATOM | 1082 | N | PHE | 119 | 112.086 | 103.527 | 93.199 | 1.00 | 0.00 | N |
| ATOM | 1083 | CA | PHE | 119 | 111.077 | 103.036 | 94.179 | 1.00 | 0.00 | C |
| ATOM | 1084 | C | PHE | 119 | 110.838 | 104.101 | 95.301 | 1.00 | 0.00 | C |
| ATOM | 1085 | O | PHE | 119 | 111.701 | 104.312 | 96.160 | 1.00 | 0.00 | O |
| ATOM | 1086 | CB | PHE | 119 | 111.485 | 101.633 | 94.732 | 1.00 | 0.00 | C |
| ATOM | 1087 | CG | PHE | 119 | 112.841 | 101.449 | 95.447 | 1.00 | 0.00 | C |
| ATOM | 1088 | CD1 | PHE | 119 | 112.914 | 101.471 | 96.843 | 1.00 | 0.00 | C |
| ATOM | 1089 | CE1 | PHE | 119 | 114.134 | 101.278 | 97.487 | 1.00 | 0.00 | C |
| ATOM | 1090 | CZ | PHE | 119 | 115.288 | 101.058 | 96.739 | 1.00 | 0.00 | C |
| ATOM | 1091 | CE2 | PHE | 119 | 115.225 | 101.031 | 95.348 | 1.00 | 0.00 | C |
| ATOM | 1092 | CD2 | PHE | 119 | 114.005 | 101.220 | 94.704 | 1.00 | 0.00 | C |
| ATOM | 1093 | H | PHE | 119 | 113.071 | 103.618 | 93.465 | 1.00 | 0.00 | H |
| ATOM | 1094 | HA | PHE | 119 | 110.110 | 102.870 | 93.664 | 1.00 | 0.00 | H |
| ATOM | 1095 | 1HB | PHE | 119 | 110.676 | 101.288 | 95.402 | 1.00 | 0.00 | H |
| ATOM | 1096 | 2HB | PHE | 119 | 111.447 | 100.900 | 93.913 | 1.00 | 0.00 | H |
| ATOM | 1097 | HD1 | PHE | 119 | 112.028 | 101.651 | 97.436 | 1.00 | 0.00 | H |
| ATOM | 1098 | HE1 | PHE | 119 | 114.185 | 101.302 | 98.566 | 1.00 | 0.00 | H |
| ATOM | 1099 | HZ | PHE | 119 | 116.236 | 100.912 | 97.237 | 1.00 | 0.00 | H |
| ATOM | 1100 | HE2 | PHE | 119 | 116.122 | 100.869 | 94.767 | 1.00 | 0.00 | H |
| ATOM | 1101 | HD2 | PHE | 119 | 113.975 | 101.198 | 93.624 | 1.00 | 0.00 | H |
| ATOM | 1102 | N | PHE | 120 | 109.682 | 104.788 | 95.304 | 1.00 | 0.00 | N |
| ATOM | 1103 | CA | PHE | 120 | 109.403 | 105.868 | 96.300 | 1.00 | 0.00 | C |
| ATOM | 1104 | C | PHE | 120 | 108.310 | 105.392 | 97.306 | 1.00 | 0.00 | C |
| ATOM | 1105 | O | PHE | 120 | 107.118 | 105.356 | 96.981 | 1.00 | 0.00 | O |
| ATOM | 1106 | CB | PHE | 120 | 109.042 | 107.233 | 95.639 | 1.00 | 0.00 | C |
| ATOM | 1107 | CG | PHE | 120 | 109.881 | 107.724 | 94.442 | 1.00 | 0.00 | C |
| ATOM | 1108 | CD1 | PHE | 120 | 109.235 | 108.097 | 93.260 | 1.00 | 0.00 | C |
| ATOM | 1109 | CE1 | PHE | 120 | 109.976 | 108.511 | 92.159 | 1.00 | 0.00 | C |
| ATOM | 1110 | CZ | PHE | 120 | 111.364 | 108.551 | 92.223 | 1.00 | 0.00 | C |
| ATOM | 1111 | CE2 | PHE | 120 | 112.017 | 108.195 | 93.399 | 1.00 | 0.00 | C |
| ATOM | 1112 | CD2 | PHE | 120 | 111.279 | 107.789 | 94.510 | 1.00 | 0.00 | C |
| ATOM | 1113 | H | PHE | 120 | 109.077 | 104.606 | 94.491 | 1.00 | 0.00 | H |
| ATOM | 1114 | HA | PHE | 120 | 110.320 | 106.093 | 96.884 | 1.00 | 0.00 | H |
| ATOM | 1115 | 1HB | PHE | 120 | 107.971 | 107.235 | 95.375 | 1.00 | 0.00 | H |
| ATOM | 1116 | 2HB | PHE | 120 | 109.108 | 108.011 | 96.421 | 1.00 | 0.00 | H |
| ATOM | 1117 | HD1 | PHE | 120 | 108.157 | 108.067 | 93.183 | 1.00 | 0.00 | H |
| ATOM | 1118 | HE1 | PHE | 120 | 109.473 | 108.812 | 91.255 | 1.00 | 0.00 | H |
| ATOM | 1119 | HZ | PHE | 120 | 111.928 | 108.856 | 91.355 | 1.00 | 0.00 | H |
| ATOM | 1120 | HE2 | PHE | 120 | 113.096 | 108.229 | 93.448 | 1.00 | 0.00 | H |
| ATOM | 1121 | HD2 | PHE | 120 | 111.798 | 107.499 | 95.411 | 1.00 | 0.00 | H |
| ATOM | 1122 | N | GLU | 121 | 108.716 | 105.034 | 98.534 | 1.00 | 0.00 | N |
| ATOM | 1123 | CA | GLU | 121 | 107.772 | 104.624 | 99.617 | 1.00 | 0.00 | C |
| ATOM | 1124 | C | GLU | 121 | 107.252 | 105.878 | 100.408 | 1.00 | 0.00 | C |
| ATOM | 1125 | O | GLU | 121 | 107.704 | 106.199 | 101.511 | 1.00 | 0.00 | O |
| ATOM | 1126 | CB | GLU | 121 | 108.493 | 103.563 | 100.497 | 1.00 | 0.00 | C |
| ATOM | 1127 | CG | GLU | 121 | 108.826 | 102.219 | 99.790 | 1.00 | 0.00 | C |
| ATOM | 1128 | CD | GLU | 121 | 109.594 | 101.222 | 100.647 | 1.00 | 0.00 | C |
| ATOM | 1129 | OE1 | GLU | 121 | 110.803 | 101.042 | 100.568 | 1.00 | 0.00 | O |
| ATOM | 1130 | OE2 | GLU | 121 | 108.785 | 100.544 | 101.503 | 1.00 | 0.00 | O |
| ATOM | 1131 | H | GLU | 121 | 109.733 | 105.042 | 98.666 | 1.00 | 0.00 | H |
| ATOM | 1132 | HA | GLU | 121 | 106.880 | 104.122 | 99.186 | 1.00 | 0.00 | H |
| ATOM | 1133 | 1HB | GLU | 121 | 109.416 | 104.005 | 100.925 | 1.00 | 0.00 | H |
| ATOM | 1134 | 2HB | GLU | 121 | 107.857 | 103.346 | 101.378 | 1.00 | 0.00 | H |
| ATOM | 1135 | 1HG | GLU | 121 | 107.903 | 101.733 | 99.430 | 1.00 | 0.00 | H |
| ATOM | 1136 | 2HG | GLU | 121 | 109.430 | 102.401 | 98.880 | 1.00 | 0.00 | H |
| ATOM | 1137 | N | ASP | 122 | 106.311 | 106.615 | 99.798 | 1.00 | 0.00 | N |
| ATOM | 1138 | CA | ASP | 122 | 105.920 | 107.981 | 100.241 | 1.00 | 0.00 | C |
| ATOM | 1139 | C | ASP | 122 | 104.687 | 107.918 | 101.192 | 1.00 | 0.00 | C |
| ATOM | 1140 | O | ASP | 122 | 103.546 | 107.784 | 100.747 | 1.00 | 0.00 | O |
| ATOM | 1141 | CB | ASP | 122 | 105.642 | 108.842 | 98.977 | 1.00 | 0.00 | C |
| ATOM | 1142 | CG | ASP | 122 | 106.847 | 109.209 | 98.112 | 1.00 | 0.00 | C |
| ATOM | 1143 | OD1 | ASP | 122 | 108.010 | 109.218 | 98.500 | 1.00 | 0.00 | O |
| ATOM | 1144 | OD2 | ASP | 122 | 106.480 | 109.557 | 96.851 | 1.00 | 0.00 | O |
| ATOM | 1145 | H | ASP | 122 | 105.914 | 106.174 | 98.957 | 1.00 | 0.00 | H |
| ATOM | 1146 | HA | ASP | 122 | 106.761 | 108.465 | 100.784 | 1.00 | 0.00 | H |
| ATOM | 1147 | 1HB | ASP | 122 | 104.884 | 108.341 | 98.348 | 1.00 | 0.00 | H |
| ATOM | 1148 | 2HB | ASP | 122 | 105.175 | 109.796 | 99.277 | 1.00 | 0.00 | H |
| ATOM | 1149 | N | ASN | 123 | 104.925 | 107.982 | 102.515 | 1.00 | 0.00 | N |
| ATOM | 1150 | CA | ASN | 123 | 103.886 | 107.675 | 103.557 | 1.00 | 0.00 | C |
| ATOM | 1151 | C | ASN | 123 | 103.339 | 106.194 | 103.514 | 1.00 | 0.00 | C |
| ATOM | 1152 | O | ASN | 123 | 102.127 | 105.962 | 103.542 | 1.00 | 0.00 | O |
| ATOM | 1153 | CB | ASN | 123 | 102.752 | 108.748 | 103.604 | 1.00 | 0.00 | C |
| ATOM | 1154 | CG | ASN | 123 | 103.189 | 110.195 | 103.819 | 1.00 | 0.00 | C |
| ATOM | 1155 | OD1 | ASN | 123 | 103.643 | 110.586 | 104.886 | 1.00 | 0.00 | O |
| ATOM | 1156 | ND2 | ASN | 123 | 103.070 | 111.034 | 102.823 | 1.00 | 0.00 | N |
| ATOM | 1157 | H | ASN | 123 | 105.924 | 108.049 | 102.740 | 1.00 | 0.00 | H |
| ATOM | 1158 | HA | ASN | 123 | 104.400 | 107.744 | 104.536 | 1.00 | 0.00 | H |
| ATOM | 1159 | 1HB | ASN | 123 | 102.123 | 108.658 | 102.700 | 1.00 | 0.00 | H |
| ATOM | 1160 | 2HB | ASN | 123 | 102.063 | 108.508 | 104.435 | 1.00 | 0.00 | H |
| ATOM | 1161 | 1HD2 | ASN | 123 | 103.425 | 111.971 | 103.025 | 1.00 | 0.00 | H |
| ATOM | 1162 | 2HD2 | ASN | 123 | 102.787 | 110.631 | 101.927 | 1.00 | 0.00 | H |
| ATOM | 1163 | N | ASP | 124 | 104.237 | 105.188 | 103.438 | 1.00 | 0.00 | N |
| ATOM | 1164 | CA | ASP | 124 | 103.882 | 103.736 | 103.266 | 1.00 | 0.00 | C |
| ATOM | 1165 | C | ASP | 124 | 103.351 | 103.261 | 101.859 | 1.00 | 0.00 | C |
| ATOM | 1166 | O | ASP | 124 | 103.606 | 102.112 | 101.488 | 1.00 | 0.00 | O |
| ATOM | 1167 | CB | ASP | 124 | 103.058 | 103.142 | 104.444 | 1.00 | 0.00 | C |
| ATOM | 1168 | CG | ASP | 124 | 103.715 | 103.228 | 105.818 | 1.00 | 0.00 | C |
| ATOM | 1169 | OD1 | ASP | 124 | 104.749 | 102.645 | 106.119 | 1.00 | 0.00 | O |
| ATOM | 1170 | OD2 | ASP | 124 | 103.021 | 104.022 | 106.677 | 1.00 | 0.00 | O |
| ATOM | 1171 | H | ASP | 124 | 105.196 | 105.526 | 103.305 | 1.00 | 0.00 | H |
| ATOM | 1172 | HA | ASP | 124 | 104.861 | 103.217 | 103.318 | 1.00 | 0.00 | H |
| ATOM | 1173 | 1HB | ASP | 124 | 102.065 | 103.615 | 104.486 | 1.00 | 0.00 | H |
| ATOM | 1174 | 2HB | ASP | 124 | 102.859 | 102.072 | 104.258 | 1.00 | 0.00 | H |
| ATOM | 1175 | N | PHE | 125 | 102.625 | 104.082 | 101.077 | 1.00 | 0.00 | N |
| ATOM | 1176 | CA | PHE | 125 | 102.218 | 103.745 | 99.679 | 1.00 | 0.00 | C |
| ATOM | 1177 | C | PHE | 125 | 103.367 | 103.892 | 98.614 | 1.00 | 0.00 | C |
| ATOM | 1178 | O | PHE | 125 | 104.244 | 104.757 | 98.716 | 1.00 | 0.00 | O |
| ATOM | 1179 | CB | PHE | 125 | 100.901 | 104.492 | 99.325 | 1.00 | 0.00 | C |
| ATOM | 1180 | CG | PHE | 125 | 100.837 | 106.028 | 99.414 | 1.00 | 0.00 | C |
| ATOM | 1181 | CD1 | PHE | 125 | 100.333 | 106.635 | 100.570 | 1.00 | 0.00 | C |
| ATOM | 1182 | CE1 | PHE | 125 | 100.190 | 108.019 | 100.631 | 1.00 | 0.00 | C |
| ATOM | 1183 | CZ | PHE | 125 | 100.550 | 108.805 | 99.539 | 1.00 | 0.00 | C |
| ATOM | 1184 | CE2 | PHE | 125 | 101.055 | 108.208 | 98.386 | 1.00 | 0.00 | C |
| ATOM | 1185 | CD2 | PHE | 125 | 101.200 | 106.824 | 98.323 | 1.00 | 0.00 | C |
| ATOM | 1186 | H | PHE | 125 | 102.548 | 105.039 | 101.444 | 1.00 | 0.00 | H |
| ATOM | 1187 | HA | PHE | 125 | 101.938 | 102.672 | 99.674 | 1.00 | 0.00 | H |
| ATOM | 1188 | 1HB | PHE | 125 | 100.599 | 104.191 | 98.304 | 1.00 | 0.00 | H |
| ATOM | 1189 | 2HB | PHE | 125 | 100.100 | 104.073 | 99.960 | 1.00 | 0.00 | H |
| ATOM | 1190 | HD1 | PHE | 125 | 100.052 | 106.040 | 101.426 | 1.00 | 0.00 | H |
| ATOM | 1191 | HE1 | PHE | 125 | 99.803 | 108.482 | 101.528 | 1.00 | 0.00 | H |
| ATOM | 1192 | HZ | PHE | 125 | 100.441 | 109.878 | 99.588 | 1.00 | 0.00 | H |
| ATOM | 1193 | HE2 | PHE | 125 | 101.326 | 108.821 | 97.541 | 1.00 | 0.00 | H |
| ATOM | 1194 | HD2 | PHE | 125 | 101.594 | 106.371 | 97.423 | 1.00 | 0.00 | H |
| ATOM | 1195 | N | VAL | 126 | 103.404 | 102.985 | 97.623 | 1.00 | 0.00 | N |
| ATOM | 1196 | CA | VAL | 126 | 104.648 | 102.689 | 96.843 | 1.00 | 0.00 | C |
| ATOM | 1197 | C | VAL | 126 | 104.499 | 103.180 | 95.362 | 1.00 | 0.00 | C |
| ATOM | 1198 | O | VAL | 126 | 103.635 | 102.708 | 94.614 | 1.00 | 0.00 | O |
| ATOM | 1199 | CB | VAL | 126 | 105.031 | 101.163 | 96.936 | 1.00 | 0.00 | C |
| ATOM | 1200 | CG1 | VAL | 126 | 106.404 | 100.850 | 96.290 | 1.00 | 0.00 | C |
| ATOM | 1201 | CG2 | VAL | 126 | 105.081 | 100.582 | 98.371 | 1.00 | 0.00 | C |
| ATOM | 1202 | H | VAL | 126 | 102.554 | 102.412 | 97.532 | 1.00 | 0.00 | H |
| ATOM | 1203 | HA | VAL | 126 | 105.504 | 103.229 | 97.296 | 1.00 | 0.00 | H |
| ATOM | 1204 | HB | VAL | 126 | 104.264 | 100.587 | 96.380 | 1.00 | 0.00 | H |
| ATOM | 1205 | 1HG1 | VAL | 126 | 107.235 | 101.379 | 96.795 | 1.00 | 0.00 | H |
| ATOM | 1206 | 2HG1 | VAL | 126 | 106.641 | 99.769 | 96.314 | 1.00 | 0.00 | H |
| ATOM | 1207 | 3HG1 | VAL | 126 | 106.439 | 101.147 | 95.225 | 1.00 | 0.00 | H |
| ATOM | 1208 | 2HG2 | VAL | 126 | 104.092 | 100.624 | 98.866 | 1.00 | 0.00 | H |
| ATOM | 1209 | 3HG2 | VAL | 126 | 105.391 | 99.521 | 98.392 | 1.00 | 0.00 | H |
| ATOM | 1210 | 1HG2 | VAL | 126 | 105.773 | 101.142 | 99.025 | 1.00 | 0.00 | H |
| ATOM | 1211 | N | PHE | 127 | 105.383 | 104.087 | 94.921 | 1.00 | 0.00 | N |
| ATOM | 1212 | CA | PHE | 127 | 105.474 | 104.515 | 93.496 | 1.00 | 0.00 | C |
| ATOM | 1213 | C | PHE | 127 | 106.590 | 103.719 | 92.746 | 1.00 | 0.00 | C |
| ATOM | 1214 | O | PHE | 127 | 107.765 | 103.756 | 93.129 | 1.00 | 0.00 | O |
| ATOM | 1215 | CB | PHE | 127 | 105.762 | 106.036 | 93.415 | 1.00 | 0.00 | C |
| ATOM | 1216 | CG | PHE | 127 | 104.615 | 106.959 | 93.846 | 1.00 | 0.00 | C |
| ATOM | 1217 | CD1 | PHE | 127 | 104.543 | 107.434 | 95.159 | 1.00 | 0.00 | C |
| ATOM | 1218 | CE1 | PHE | 127 | 103.508 | 108.284 | 95.539 | 1.00 | 0.00 | C |
| ATOM | 1219 | CZ | PHE | 127 | 102.537 | 108.658 | 94.615 | 1.00 | 0.00 | C |
| ATOM | 1220 | CE2 | PHE | 127 | 102.603 | 108.186 | 93.306 | 1.00 | 0.00 | C |
| ATOM | 1221 | CD2 | PHE | 127 | 103.636 | 107.337 | 92.922 | 1.00 | 0.00 | C |
| ATOM | 1222 | H | PHE | 127 | 105.994 | 104.480 | 95.651 | 1.00 | 0.00 | H |
| ATOM | 1223 | HA | PHE | 127 | 104.504 | 104.346 | 92.983 | 1.00 | 0.00 | H |
| ATOM | 1224 | 1HB | PHE | 127 | 106.677 | 106.262 | 93.988 | 1.00 | 0.00 | H |
| ATOM | 1225 | 2HB | PHE | 127 | 106.045 | 106.300 | 92.376 | 1.00 | 0.00 | H |
| ATOM | 1226 | HD1 | PHE | 127 | 105.287 | 107.144 | 95.888 | 1.00 | 0.00 | H |
| ATOM | 1227 | HE1 | PHE | 127 | 103.464 | 108.655 | 96.551 | 1.00 | 0.00 | H |
| ATOM | 1228 | HZ | PHE | 127 | 101.733 | 109.317 | 94.911 | 1.00 | 0.00 | H |
| ATOM | 1229 | HE2 | PHE | 127 | 101.855 | 108.475 | 92.583 | 1.00 | 0.00 | H |
| ATOM | 1230 | HD2 | PHE | 127 | 103.676 | 106.974 | 91.905 | 1.00 | 0.00 | H |
| ATOM | 1231 | N | VAL | 128 | 106.219 | 103.025 | 91.663 | 1.00 | 0.00 | N |
| ATOM | 1232 | CA | VAL | 128 | 107.158 | 102.171 | 90.872 | 1.00 | 0.00 | C |
| ATOM | 1233 | C | VAL | 128 | 107.331 | 102.863 | 89.474 | 1.00 | 0.00 | C |
| ATOM | 1234 | O | VAL | 128 | 106.446 | 102.756 | 88.615 | 1.00 | 0.00 | O |
| ATOM | 1235 | CB | VAL | 128 | 106.594 | 100.701 | 90.814 | 1.00 | 0.00 | C |
| ATOM | 1236 | CG1 | VAL | 128 | 107.465 | 99.721 | 89.999 | 1.00 | 0.00 | C |
| ATOM | 1237 | CG2 | VAL | 128 | 106.366 | 100.039 | 92.194 | 1.00 | 0.00 | C |
| ATOM | 1238 | H | VAL | 128 | 105.201 | 103.006 | 91.501 | 1.00 | 0.00 | H |
| ATOM | 1239 | HA | VAL | 128 | 108.153 | 102.117 | 91.363 | 1.00 | 0.00 | H |
| ATOM | 1240 | HB | VAL | 128 | 105.612 | 100.748 | 90.316 | 1.00 | 0.00 | H |
| ATOM | 1241 | 1HG1 | VAL | 128 | 107.653 | 100.079 | 88.969 | 1.00 | 0.00 | H |
| ATOM | 1242 | 2HG1 | VAL | 128 | 108.447 | 99.557 | 90.475 | 1.00 | 0.00 | H |
| ATOM | 1243 | 3HG1 | VAL | 128 | 106.997 | 98.722 | 89.899 | 1.00 | 0.00 | H |
| ATOM | 1244 | 2HG2 | VAL | 128 | 105.597 | 100.584 | 92.771 | 1.00 | 0.00 | H |
| ATOM | 1245 | 3HG2 | VAL | 128 | 106.020 | 98.990 | 92.117 | 1.00 | 0.00 | H |
| ATOM | 1246 | 1HG2 | VAL | 128 | 107.283 | 100.036 | 92.811 | 1.00 | 0.00 | H |
| ATOM | 1247 | N | VAL | 129 | 108.436 | 103.608 | 89.266 | 0.00 | 0.00 | N |
| ATOM | 1248 | CA | VAL | 129 | 108.635 | 104.444 | 88.036 | 0.00 | 0.00 | C |
| ATOM | 1249 | C | VAL | 129 | 109.469 | 103.667 | 86.957 | 0.00 | 0.00 | C |
| ATOM | 1250 | O | VAL | 129 | 110.682 | 103.484 | 87.095 | 0.00 | 0.00 | O |
| ATOM | 1251 | CB | VAL | 129 | 109.234 | 105.842 | 88.417 | 0.00 | 0.00 | C |
| ATOM | 1252 | CG1 | VAL | 129 | 109.436 | 106.777 | 87.201 | 0.00 | 0.00 | C |
| ATOM | 1253 | CG2 | VAL | 129 | 108.356 | 106.644 | 89.410 | 0.00 | 0.00 | C |
| ATOM | 1254 | H | VAL | 129 | 109.032 | 103.732 | 90.098 | 0.00 | 0.00 | H |
| ATOM | 1255 | HA | VAL | 129 | 107.648 | 104.677 | 87.586 | 0.00 | 0.00 | H |
| ATOM | 1256 | HB | VAL | 129 | 110.228 | 105.683 | 88.887 | 0.00 | 0.00 | H |
| ATOM | 1257 | 1HG1 | VAL | 129 | 109.949 | 107.717 | 87.483 | 0.00 | 0.00 | H |
| ATOM | 1258 | 2HG1 | VAL | 129 | 110.064 | 106.319 | 86.415 | 0.00 | 0.00 | H |
| ATOM | 1259 | 3HG1 | VAL | 129 | 108.480 | 107.059 | 86.723 | 0.00 | 0.00 | H |
| ATOM | 1260 | 1HG2 | VAL | 129 | 108.744 | 107.665 | 89.571 | 0.00 | 0.00 | H |
| ATOM | 1261 | 2HG2 | VAL | 129 | 107.313 | 106.749 | 89.057 | 0.00 | 0.00 | H |
| ATOM | 1262 | 3HG2 | VAL | 129 | 108.321 | 106.161 | 90.404 | 0.00 | 0.00 | H |
| ATOM | 1263 | N | LEU | 130 | 108.799 | 103.224 | 85.886 | 1.00 | 0.00 | N |
| ATOM | 1264 | CA | LEU | 130 | 109.415 | 102.478 | 84.746 | 1.00 | 0.00 | C |
| ATOM | 1265 | C | LEU | 130 | 109.327 | 103.300 | 83.403 | 1.00 | 0.00 | C |
| ATOM | 1266 | O | LEU | 130 | 108.558 | 104.257 | 83.303 | 1.00 | 0.00 | O |
| ATOM | 1267 | CB | LEU | 130 | 108.620 | 101.133 | 84.676 | 1.00 | 0.00 | C |
| ATOM | 1268 | CG | LEU | 130 | 108.913 | 100.080 | 85.784 | 1.00 | 0.00 | C |
| ATOM | 1269 | CD1 | LEU | 130 | 107.776 | 99.055 | 85.901 | 1.00 | 0.00 | C |
| ATOM | 1270 | CD2 | LEU | 130 | 110.226 | 99.320 | 85.529 | 1.00 | 0.00 | C |
| ATOM | 1271 | H | LEU | 130 | 107.802 | 103.474 | 85.837 | 1.00 | 0.00 | H |
| ATOM | 1272 | HA | LEU | 130 | 110.488 | 102.267 | 84.925 | 1.00 | 0.00 | H |
| ATOM | 1273 | 1HB | LEU | 130 | 107.535 | 101.361 | 84.647 | 1.00 | 0.00 | H |
| ATOM | 1274 | 2HB | LEU | 130 | 108.787 | 100.665 | 83.694 | 1.00 | 0.00 | H |
| ATOM | 1275 | HG | LEU | 130 | 108.983 | 100.603 | 86.758 | 1.00 | 0.00 | H |
| ATOM | 1276 | 2HD1 | LEU | 130 | 107.947 | 98.341 | 86.729 | 1.00 | 0.00 | H |
| ATOM | 1277 | 3HD1 | LEU | 130 | 106.801 | 99.541 | 86.090 | 1.00 | 0.00 | H |
| ATOM | 1278 | 1HD1 | LEU | 130 | 107.665 | 98.457 | 84.978 | 1.00 | 0.00 | H |
| ATOM | 1279 | 2HD2 | LEU | 130 | 110.487 | 98.655 | 86.373 | 1.00 | 0.00 | H |
| ATOM | 1280 | 3HD2 | LEU | 130 | 110.172 | 98.685 | 84.626 | 1.00 | 0.00 | H |
| ATOM | 1281 | 1HD2 | LEU | 130 | 111.082 | 100.001 | 85.379 | 1.00 | 0.00 | H |
| ATOM | 1282 | N | GLU | 131 | 110.080 | 102.933 | 82.351 | 0.00 | 0.00 | N |
| ATOM | 1283 | CA | GLU | 131 | 110.020 | 103.629 | 81.016 | 0.00 | 0.00 | C |
| ATOM | 1284 | C | GLU | 131 | 108.628 | 103.573 | 80.266 | 0.00 | 0.00 | C |
| ATOM | 1285 | O | GLU | 131 | 107.847 | 102.634 | 80.440 | 0.00 | 0.00 | O |
| ATOM | 1286 | CB | GLU | 131 | 111.158 | 103.061 | 80.122 | 0.00 | 0.00 | C |
| ATOM | 1287 | CG | GLU | 131 | 112.609 | 103.393 | 80.570 | 0.00 | 0.00 | C |
| ATOM | 1288 | CD | GLU | 131 | 113.670 | 102.993 | 79.570 | 0.00 | 0.00 | C |
| ATOM | 1289 | OE1 | GLU | 131 | 114.111 | 101.850 | 79.583 | 0.00 | 0.00 | O |
| ATOM | 1290 | OE2 | GLU | 131 | 114.051 | 103.935 | 78.666 | 0.00 | 0.00 | O |
| ATOM | 1291 | H | GLU | 131 | 110.871 | 102.320 | 82.581 | 0.00 | 0.00 | H |
| ATOM | 1292 | HA | GLU | 131 | 110.247 | 104.702 | 81.185 | 0.00 | 0.00 | H |
| ATOM | 1293 | 1HB | GLU | 131 | 111.036 | 103.469 | 79.097 | 0.00 | 0.00 | H |
| ATOM | 1294 | 2HB | GLU | 131 | 111.040 | 101.969 | 79.995 | 0.00 | 0.00 | H |
| ATOM | 1295 | 1HG | GLU | 131 | 112.861 | 102.929 | 81.533 | 0.00 | 0.00 | H |
| ATOM | 1296 | 2HG | GLU | 131 | 112.727 | 104.481 | 80.717 | 0.00 | 0.00 | H |
| ATOM | 1297 | N | LEU | 132 | 108.277 | 104.581 | 79.443 | 1.00 | 0.00 | N |
| ATOM | 1298 | CA | LEU | 132 | 106.903 | 104.705 | 78.848 | 1.00 | 0.00 | C |
| ATOM | 1299 | C | LEU | 132 | 106.653 | 103.852 | 77.552 | 1.00 | 0.00 | C |
| ATOM | 1300 | O | LEU | 132 | 106.574 | 104.373 | 76.440 | 1.00 | 0.00 | O |
| ATOM | 1301 | CB | LEU | 132 | 106.596 | 106.229 | 78.718 | 1.00 | 0.00 | C |
| ATOM | 1302 | CG | LEU | 132 | 105.145 | 106.739 | 78.479 | 1.00 | 0.00 | C |
| ATOM | 1303 | CD1 | LEU | 132 | 104.708 | 106.724 | 77.008 | 1.00 | 0.00 | C |
| ATOM | 1304 | CD2 | LEU | 132 | 104.071 | 106.043 | 79.333 | 1.00 | 0.00 | C |
| ATOM | 1305 | H | LEU | 132 | 109.018 | 105.267 | 79.254 | 1.00 | 0.00 | H |
| ATOM | 1306 | HA | LEU | 132 | 106.180 | 104.329 | 79.594 | 1.00 | 0.00 | H |
| ATOM | 1307 | 1HB | LEU | 132 | 106.924 | 106.718 | 79.651 | 1.00 | 0.00 | H |
| ATOM | 1308 | 2HB | LEU | 132 | 107.268 | 106.664 | 77.950 | 1.00 | 0.00 | H |
| ATOM | 1309 | HG | LEU | 132 | 105.158 | 107.808 | 78.772 | 1.00 | 0.00 | H |
| ATOM | 1310 | 2HD1 | LEU | 132 | 103.786 | 107.316 | 76.851 | 1.00 | 0.00 | H |
| ATOM | 1311 | 3HD1 | LEU | 132 | 105.484 | 107.156 | 76.347 | 1.00 | 0.00 | H |
| ATOM | 1312 | 1HD1 | LEU | 132 | 104.505 | 105.704 | 76.637 | 1.00 | 0.00 | H |
| ATOM | 1313 | 2HD2 | LEU | 132 | 103.075 | 106.495 | 79.174 | 1.00 | 0.00 | H |
| ATOM | 1314 | 3HD2 | LEU | 132 | 103.980 | 104.969 | 79.087 | 1.00 | 0.00 | H |
| ATOM | 1315 | 1HD2 | LEU | 132 | 104.296 | 106.123 | 80.410 | 1.00 | 0.00 | H |
| ATOM | 1316 | N | CYS | 133 | 106.472 | 102.527 | 77.702 | 1.00 | 0.00 | N |
| ATOM | 1317 | CA | CYS | 133 | 106.426 | 101.589 | 76.542 | 1.00 | 0.00 | C |
| ATOM | 1318 | C | CYS | 133 | 105.037 | 100.892 | 76.285 | 1.00 | 0.00 | C |
| ATOM | 1319 | O | CYS | 133 | 104.198 | 100.720 | 77.177 | 1.00 | 0.00 | O |
| ATOM | 1320 | CB | CYS | 133 | 107.598 | 100.603 | 76.742 | 1.00 | 0.00 | C |
| ATOM | 1321 | SG | CYS | 133 | 109.201 | 101.492 | 76.858 | 1.00 | 0.00 | S |
| ATOM | 1322 | H | CYS | 133 | 106.732 | 102.222 | 78.648 | 1.00 | 0.00 | H |
| ATOM | 1323 | HA | CYS | 133 | 106.666 | 102.141 | 75.607 | 1.00 | 0.00 | H |
| ATOM | 1324 | 1HB | CYS | 133 | 107.458 | 99.988 | 77.648 | 1.00 | 0.00 | H |
| ATOM | 1325 | 2HB | CYS | 133 | 107.663 | 99.900 | 75.893 | 1.00 | 0.00 | H |
| ATOM | 1326 | HG | CYS | 133 | 109.042 | 102.358 | 75.852 | 1.00 | 0.00 | H |
| ATOM | 1327 | N | ARG | 134 | 104.775 | 100.465 | 75.033 | 1.00 | 0.00 | N |
| ATOM | 1328 | CA | ARG | 134 | 103.490 | 99.798 | 74.659 | 1.00 | 0.00 | C |
| ATOM | 1329 | C | ARG | 134 | 103.590 | 98.225 | 74.735 | 1.00 | 0.00 | C |
| ATOM | 1330 | O | ARG | 134 | 104.065 | 97.571 | 73.813 | 1.00 | 0.00 | O |
| ATOM | 1331 | CB | ARG | 134 | 103.084 | 100.221 | 73.216 | 1.00 | 0.00 | C |
| ATOM | 1332 | CG | ARG | 134 | 102.443 | 101.619 | 73.038 | 1.00 | 0.00 | C |
| ATOM | 1333 | CD | ARG | 134 | 101.887 | 101.772 | 71.607 | 1.00 | 0.00 | C |
| ATOM | 1334 | NE | ARG | 134 | 101.161 | 103.064 | 71.452 | 1.00 | 0.00 | N |
| ATOM | 1335 | CZ | ARG | 134 | 100.461 | 103.419 | 70.376 | 1.00 | 0.00 | C |
| ATOM | 1336 | NH1 | ARG | 134 | 100.385 | 102.699 | 69.293 | 1.00 | 0.00 | N |
| ATOM | 1337 | NH2 | ARG | 134 | 99.837 | 104.556 | 70.389 | 1.00 | 0.00 | N |
| ATOM | 1338 | HE | ARG | 134 | 101.171 | 103.752 | 72.213 | 1.00 | 0.00 | H |
| ATOM | 1339 | H | ARG | 134 | 105.593 | 100.514 | 74.405 | 1.00 | 0.00 | H |
| ATOM | 1340 | HA | ARG | 134 | 102.674 | 100.125 | 75.335 | 1.00 | 0.00 | H |
| ATOM | 1341 | 1HB | ARG | 134 | 103.960 | 100.113 | 72.542 | 1.00 | 0.00 | H |
| ATOM | 1342 | 2HB | ARG | 134 | 102.359 | 99.472 | 72.836 | 1.00 | 0.00 | H |
| ATOM | 1343 | 1HG | ARG | 134 | 101.637 | 101.756 | 73.785 | 1.00 | 0.00 | H |
| ATOM | 1344 | 2HG | ARG | 134 | 103.197 | 102.403 | 73.263 | 1.00 | 0.00 | H |
| ATOM | 1345 | 1HD | ARG | 134 | 102.727 | 101.701 | 70.882 | 1.00 | 0.00 | H |
| ATOM | 1346 | 2HD | ARG | 134 | 101.205 | 100.921 | 71.383 | 1.00 | 0.00 | H |
| ATOM | 1347 | 2HH1 | ARG | 134 | 100.927 | 101.836 | 69.356 | 1.00 | 0.00 | H |
| ATOM | 1348 | 1HH1 | ARG | 134 | 99.983 | 103.155 | 68.477 | 1.00 | 0.00 | H |
| ATOM | 1349 | 1HH2 | ARG | 134 | 99.920 | 105.082 | 71.261 | 1.00 | 0.00 | H |
| ATOM | 1350 | 2HH2 | ARG | 134 | 99.221 | 104.761 | 69.596 | 1.00 | 0.00 | H |
| ATOM | 1351 | N | ARG | 135 | 103.087 | 97.624 | 75.813 | 1.00 | 0.00 | N |
| ATOM | 1352 | CA | ARG | 135 | 102.444 | 96.259 | 75.882 | 1.00 | 0.00 | C |
| ATOM | 1353 | C | ARG | 135 | 102.227 | 95.298 | 74.626 | 1.00 | 0.00 | C |
| ATOM | 1354 | O | ARG | 135 | 102.193 | 95.768 | 73.496 | 1.00 | 0.00 | O |
| ATOM | 1355 | CB | ARG | 135 | 101.170 | 96.500 | 76.763 | 1.00 | 0.00 | C |
| ATOM | 1356 | CG | ARG | 135 | 100.249 | 97.756 | 76.533 | 1.00 | 0.00 | C |
| ATOM | 1357 | CD | ARG | 135 | 99.239 | 98.054 | 77.662 | 1.00 | 0.00 | C |
| ATOM | 1358 | NE | ARG | 135 | 98.236 | 96.968 | 77.722 | 1.00 | 0.00 | N |
| ATOM | 1359 | CZ | ARG | 135 | 98.273 | 96.032 | 78.641 | 1.00 | 0.00 | C |
| ATOM | 1360 | NH1 | ARG | 135 | 98.483 | 96.230 | 79.894 | 1.00 | 0.00 | N |
| ATOM | 1361 | NH2 | ARG | 135 | 98.048 | 94.829 | 78.294 | 1.00 | 0.00 | N |
| ATOM | 1362 | HE | ARG | 135 | 97.982 | 96.480 | 76.853 | 1.00 | 0.00 | H |
| ATOM | 1363 | H | ARG | 135 | 102.981 | 98.280 | 76.595 | 1.00 | 0.00 | H |
| ATOM | 1364 | HA | ARG | 135 | 103.127 | 95.662 | 76.520 | 1.00 | 0.00 | H |
| ATOM | 1365 | 1HB | ARG | 135 | 100.547 | 95.585 | 76.749 | 1.00 | 0.00 | H |
| ATOM | 1366 | 2HB | ARG | 135 | 101.503 | 96.524 | 77.819 | 1.00 | 0.00 | H |
| ATOM | 1367 | 1HG | ARG | 135 | 100.861 | 98.670 | 76.445 | 1.00 | 0.00 | H |
| ATOM | 1368 | 2HG | ARG | 135 | 99.741 | 97.682 | 75.554 | 1.00 | 0.00 | H |
| ATOM | 1369 | 1HD | ARG | 135 | 99.775 | 98.187 | 78.627 | 1.00 | 0.00 | H |
| ATOM | 1370 | 2HD | ARG | 135 | 98.722 | 99.018 | 77.487 | 1.00 | 0.00 | H |
| ATOM | 1371 | 2HH1 | ARG | 135 | 97.841 | 96.966 | 80.275 | 1.00 | 0.00 | H |
| ATOM | 1372 | 1HH1 | ARG | 135 | 98.325 | 95.288 | 80.351 | 1.00 | 0.00 | H |
| ATOM | 1373 | 1HH2 | ARG | 135 | 97.606 | 94.599 | 77.401 | 1.00 | 0.00 | H |
| ATOM | 1374 | 2HH2 | ARG | 135 | 97.832 | 94.231 | 79.153 | 1.00 | 0.00 | H |
| ATOM | 1375 | N | ARG | 136 | 102.093 | 93.951 | 74.777 | 1.00 | 0.00 | N |
| ATOM | 1376 | CA | ARG | 136 | 102.057 | 92.967 | 73.624 | 1.00 | 0.00 | C |
| ATOM | 1377 | C | ARG | 136 | 100.688 | 92.376 | 73.061 | 1.00 | 0.00 | C |
| ATOM | 1378 | O | ARG | 136 | 100.739 | 91.506 | 72.193 | 1.00 | 0.00 | O |
| ATOM | 1379 | CB | ARG | 136 | 103.087 | 91.811 | 73.900 | 1.00 | 0.00 | C |
| ATOM | 1380 | CG | ARG | 136 | 104.158 | 91.580 | 72.810 | 1.00 | 0.00 | C |
| ATOM | 1381 | CD | ARG | 136 | 105.251 | 92.657 | 72.832 | 1.00 | 0.00 | C |
| ATOM | 1382 | NE | ARG | 136 | 106.206 | 92.383 | 71.732 | 1.00 | 0.00 | N |
| ATOM | 1383 | CZ | ARG | 136 | 107.389 | 92.960 | 71.582 | 1.00 | 0.00 | C |
| ATOM | 1384 | NH1 | ARG | 136 | 107.878 | 93.831 | 72.403 | 1.00 | 0.00 | N |
| ATOM | 1385 | NH2 | ARG | 136 | 108.094 | 92.643 | 70.557 | 1.00 | 0.00 | N |
| ATOM | 1386 | HE | ARG | 136 | 105.927 | 91.687 | 71.026 | 1.00 | 0.00 | H |
| ATOM | 1387 | H | ARG | 136 | 102.248 | 93.616 | 75.740 | 1.00 | 0.00 | H |
| ATOM | 1388 | HA | ARG | 136 | 102.443 | 93.480 | 72.718 | 1.00 | 0.00 | H |
| ATOM | 1389 | 1HB | ARG | 136 | 103.582 | 91.925 | 74.880 | 1.00 | 0.00 | H |
| ATOM | 1390 | 2HB | ARG | 136 | 102.546 | 90.859 | 74.040 | 1.00 | 0.00 | H |
| ATOM | 1391 | 1HG | ARG | 136 | 104.633 | 90.591 | 72.959 | 1.00 | 0.00 | H |
| ATOM | 1392 | 2HG | ARG | 136 | 103.686 | 91.522 | 71.809 | 1.00 | 0.00 | H |
| ATOM | 1393 | 1HD | ARG | 136 | 104.817 | 93.671 | 72.717 | 1.00 | 0.00 | H |
| ATOM | 1394 | 2HD | ARG | 136 | 105.766 | 92.652 | 73.815 | 1.00 | 0.00 | H |
| ATOM | 1395 | 2HH1 | ARG | 136 | 107.240 | 94.080 | 73.160 | 1.00 | 0.00 | H |
| ATOM | 1396 | 1HH1 | ARG | 136 | 108.786 | 94.233 | 72.170 | 1.00 | 0.00 | H |
| ATOM | 1397 | 1HH2 | ARG | 136 | 107.610 | 91.983 | 69.942 | 1.00 | 0.00 | H |
| ATOM | 1398 | 2HH2 | ARG | 136 | 108.989 | 93.111 | 70.433 | 1.00 | 0.00 | H |
| ATOM | 1399 | N | SER | 137 | 99.408 | 92.748 | 73.311 | 1.00 | 0.00 | N |
| ATOM | 1400 | CA | SER | 137 | 98.894 | 93.583 | 74.450 | 1.00 | 0.00 | C |
| ATOM | 1401 | C | SER | 137 | 98.599 | 95.136 | 74.259 | 1.00 | 0.00 | C |
| ATOM | 1402 | O | SER | 137 | 98.196 | 95.769 | 75.225 | 1.00 | 0.00 | O |
| ATOM | 1403 | CB | SER | 137 | 97.668 | 92.796 | 75.047 | 1.00 | 0.00 | C |
| ATOM | 1404 | OG | SER | 137 | 96.959 | 93.495 | 76.093 | 1.00 | 0.00 | O |
| ATOM | 1405 | H | SER | 137 | 98.778 | 92.393 | 72.582 | 1.00 | 0.00 | H |
| ATOM | 1406 | HA | SER | 137 | 99.623 | 93.559 | 75.279 | 1.00 | 0.00 | H |
| ATOM | 1407 | 1HB | SER | 137 | 96.937 | 92.555 | 74.252 | 1.00 | 0.00 | H |
| ATOM | 1408 | 2HB | SER | 137 | 97.996 | 91.822 | 75.453 | 1.00 | 0.00 | H |
| ATOM | 1409 | HG | SER | 137 | 96.783 | 92.949 | 76.949 | 1.00 | 0.00 | H |
| ATOM | 1410 | N | LEU | 138 | 98.732 | 96.006 | 73.240 | 1.00 | 0.00 | N |
| ATOM | 1411 | CA | LEU | 138 | 98.781 | 95.723 | 71.777 | 1.00 | 0.00 | C |
| ATOM | 1412 | C | LEU | 138 | 97.517 | 94.909 | 71.295 | 1.00 | 0.00 | C |
| ATOM | 1413 | O | LEU | 138 | 97.528 | 93.692 | 71.106 | 1.00 | 0.00 | O |
| ATOM | 1414 | CB | LEU | 138 | 100.246 | 95.409 | 71.362 | 1.00 | 0.00 | C |
| ATOM | 1415 | CG | LEU | 138 | 100.687 | 95.099 | 69.916 | 1.00 | 0.00 | C |
| ATOM | 1416 | CD1 | LEU | 138 | 102.198 | 94.813 | 69.905 | 1.00 | 0.00 | C |
| ATOM | 1417 | CD2 | LEU | 138 | 100.015 | 93.877 | 69.283 | 1.00 | 0.00 | C |
| ATOM | 1418 | H | LEU | 138 | 98.550 | 96.940 | 73.620 | 1.00 | 0.00 | H |
| ATOM | 1419 | HA | LEU | 138 | 98.653 | 96.705 | 71.281 | 1.00 | 0.00 | H |
| ATOM | 1420 | 1HB | LEU | 138 | 100.846 | 96.286 | 71.679 | 1.00 | 0.00 | H |
| ATOM | 1421 | 2HB | LEU | 138 | 100.635 | 94.628 | 72.003 | 1.00 | 0.00 | H |
| ATOM | 1422 | HG | LEU | 138 | 100.492 | 95.999 | 69.305 | 1.00 | 0.00 | H |
| ATOM | 1423 | 2HD1 | LEU | 138 | 102.762 | 95.565 | 70.484 | 1.00 | 0.00 | H |
| ATOM | 1424 | 3HD1 | LEU | 138 | 102.447 | 93.835 | 70.362 | 1.00 | 0.00 | H |
| ATOM | 1425 | 1HD1 | LEU | 138 | 102.610 | 94.808 | 68.881 | 1.00 | 0.00 | H |
| ATOM | 1426 | 2HD2 | LEU | 138 | 100.082 | 92.981 | 69.930 | 1.00 | 0.00 | H |
| ATOM | 1427 | 3HD2 | LEU | 138 | 98.945 | 94.055 | 69.092 | 1.00 | 0.00 | H |
| ATOM | 1428 | 1HD2 | LEU | 138 | 100.466 | 93.612 | 68.310 | 1.00 | 0.00 | H |
| ATOM | 1429 | N | LEU | 139 | 96.395 | 95.647 | 71.172 | 1.00 | 0.00 | N |
| ATOM | 1430 | CA | LEU | 139 | 95.028 | 95.086 | 71.001 | 1.00 | 0.00 | C |
| ATOM | 1431 | C | LEU | 139 | 94.092 | 95.753 | 69.895 | 1.00 | 0.00 | C |
| ATOM | 1432 | O | LEU | 139 | 92.912 | 95.417 | 69.861 | 1.00 | 0.00 | O |
| ATOM | 1433 | CB | LEU | 139 | 94.384 | 95.134 | 72.442 | 1.00 | 0.00 | C |
| ATOM | 1434 | CG | LEU | 139 | 93.658 | 93.861 | 72.954 | 1.00 | 0.00 | C |
| ATOM | 1435 | CD1 | LEU | 139 | 93.431 | 93.946 | 74.475 | 1.00 | 0.00 | C |
| ATOM | 1436 | CD2 | LEU | 139 | 92.298 | 93.624 | 72.287 | 1.00 | 0.00 | C |
| ATOM | 1437 | H | LEU | 139 | 96.525 | 96.607 | 71.496 | 1.00 | 0.00 | H |
| ATOM | 1438 | HA | LEU | 139 | 95.105 | 94.027 | 70.683 | 1.00 | 0.00 | H |
| ATOM | 1439 | 1HB | LEU | 139 | 95.156 | 95.360 | 73.209 | 1.00 | 0.00 | H |
| ATOM | 1440 | 2HB | LEU | 139 | 93.707 | 96.006 | 72.523 | 1.00 | 0.00 | H |
| ATOM | 1441 | HG | LEU | 139 | 94.301 | 92.982 | 72.746 | 1.00 | 0.00 | H |
| ATOM | 1442 | 2HD1 | LEU | 139 | 94.379 | 94.064 | 75.041 | 1.00 | 0.00 | H |
| ATOM | 1443 | 3HD1 | LEU | 139 | 92.776 | 94.796 | 74.748 | 1.00 | 0.00 | H |
| ATOM | 1444 | 1HD1 | LEU | 139 | 92.958 | 93.031 | 74.876 | 1.00 | 0.00 | H |
| ATOM | 1445 | 2HD2 | LEU | 139 | 91.625 | 94.498 | 72.380 | 1.00 | 0.00 | H |
| ATOM | 1446 | 3HD2 | LEU | 139 | 92.420 | 93.425 | 71.210 | 1.00 | 0.00 | H |
| ATOM | 1447 | 1HD2 | LEU | 139 | 91.766 | 92.749 | 72.704 | 1.00 | 0.00 | H |
| ATOM | 1448 | N | GLU | 140 | 94.311 | 96.661 | 68.919 | 1.00 | 0.00 | N |
| ATOM | 1449 | CA | GLU | 140 | 95.583 | 97.311 | 68.479 | 1.00 | 0.00 | C |
| ATOM | 1450 | C | GLU | 140 | 96.622 | 96.377 | 67.752 | 1.00 | 0.00 | C |
| ATOM | 1451 | O | GLU | 140 | 96.907 | 95.279 | 68.222 | 1.00 | 0.00 | O |
| ATOM | 1452 | CB | GLU | 140 | 96.197 | 98.284 | 69.534 | 1.00 | 0.00 | C |
| ATOM | 1453 | CG | GLU | 140 | 96.556 | 99.716 | 69.056 | 1.00 | 0.00 | C |
| ATOM | 1454 | CD | GLU | 140 | 97.614 | 100.407 | 69.926 | 1.00 | 0.00 | C |
| ATOM | 1455 | OE1 | GLU | 140 | 98.716 | 99.888 | 70.184 | 1.00 | 0.00 | O |
| ATOM | 1456 | OE2 | GLU | 140 | 97.295 | 101.695 | 70.221 | 1.00 | 0.00 | O |
| ATOM | 1457 | H | GLU | 140 | 93.439 | 96.792 | 68.391 | 1.00 | 0.00 | H |
| ATOM | 1458 | HA | GLU | 140 | 95.204 | 97.973 | 67.674 | 1.00 | 0.00 | H |
| ATOM | 1459 | 1HB | GLU | 140 | 95.535 | 98.373 | 70.417 | 1.00 | 0.00 | H |
| ATOM | 1460 | 2HB | GLU | 140 | 97.116 | 97.812 | 69.926 | 1.00 | 0.00 | H |
| ATOM | 1461 | 1HG | GLU | 140 | 96.948 | 99.703 | 68.025 | 1.00 | 0.00 | H |
| ATOM | 1462 | 2HG | GLU | 140 | 95.644 | 100.338 | 69.008 | 1.00 | 0.00 | H |
| ATOM | 1463 | N | LEU | 141 | 97.189 | 96.813 | 66.605 | 1.00 | 0.00 | N |
| ATOM | 1464 | CA | LEU | 141 | 98.215 | 96.059 | 65.806 | 1.00 | 0.00 | C |
| ATOM | 1465 | C | LEU | 141 | 97.869 | 94.561 | 65.465 | 1.00 | 0.00 | C |
| ATOM | 1466 | O | LEU | 141 | 97.231 | 94.319 | 64.446 | 1.00 | 0.00 | O |
| ATOM | 1467 | CB | LEU | 141 | 99.684 | 96.288 | 66.287 | 1.00 | 0.00 | C |
| ATOM | 1468 | CG | LEU | 141 | 100.294 | 97.716 | 66.239 | 1.00 | 0.00 | C |
| ATOM | 1469 | CD1 | LEU | 141 | 99.949 | 98.559 | 67.478 | 1.00 | 0.00 | C |
| ATOM | 1470 | CD2 | LEU | 141 | 101.828 | 97.644 | 66.150 | 1.00 | 0.00 | C |
| ATOM | 1471 | H | LEU | 141 | 96.894 | 97.753 | 66.330 | 1.00 | 0.00 | H |
| ATOM | 1472 | HA | LEU | 141 | 98.193 | 96.531 | 64.805 | 1.00 | 0.00 | H |
| ATOM | 1473 | 1HB | LEU | 141 | 99.800 | 95.860 | 67.293 | 1.00 | 0.00 | H |
| ATOM | 1474 | 2HB | LEU | 141 | 100.316 | 95.635 | 65.650 | 1.00 | 0.00 | H |
| ATOM | 1475 | HG | LEU | 141 | 99.924 | 98.234 | 65.331 | 1.00 | 0.00 | H |
| ATOM | 1476 | 2HD1 | LEU | 141 | 98.865 | 98.650 | 67.627 | 1.00 | 0.00 | H |
| ATOM | 1477 | 3HD1 | LEU | 141 | 100.359 | 98.128 | 68.411 | 1.00 | 0.00 | H |
| ATOM | 1478 | 1HD1 | LEU | 141 | 100.344 | 99.590 | 67.401 | 1.00 | 0.00 | H |
| ATOM | 1479 | 2HD2 | LEU | 141 | 102.277 | 97.137 | 67.025 | 1.00 | 0.00 | H |
| ATOM | 1480 | 3HD2 | LEU | 141 | 102.157 | 97.089 | 65.253 | 1.00 | 0.00 | H |
| ATOM | 1481 | 1HD2 | LEU | 141 | 102.288 | 98.647 | 66.082 | 1.00 | 0.00 | H |
| ATOM | 1482 | N | HIS | 142 | 98.239 | 93.574 | 66.305 | 1.00 | 0.00 | N |
| ATOM | 1483 | CA | HIS | 142 | 97.890 | 92.130 | 66.120 | 1.00 | 0.00 | C |
| ATOM | 1484 | C | HIS | 142 | 96.376 | 91.786 | 65.867 | 1.00 | 0.00 | C |
| ATOM | 1485 | O | HIS | 142 | 96.077 | 91.082 | 64.902 | 1.00 | 0.00 | O |
| ATOM | 1486 | CB | HIS | 142 | 98.520 | 91.384 | 67.333 | 1.00 | 0.00 | C |
| ATOM | 1487 | CG | HIS | 142 | 98.401 | 89.863 | 67.314 | 1.00 | 0.00 | C |
| ATOM | 1488 | ND1 | HIS | 142 | 99.032 | 89.064 | 66.378 | 1.00 | 0.00 | N |
| ATOM | 1489 | CE1 | HIS | 142 | 98.517 | 87.850 | 66.743 | 1.00 | 0.00 | C |
| ATOM | 1490 | NE2 | HIS | 142 | 97.673 | 87.762 | 67.818 | 1.00 | 0.00 | N |
| ATOM | 1491 | CD2 | HIS | 142 | 97.591 | 89.095 | 68.166 | 1.00 | 0.00 | C |
| ATOM | 1492 | H | HIS | 142 | 98.568 | 93.935 | 67.207 | 1.00 | 0.00 | H |
| ATOM | 1493 | HA | HIS | 142 | 98.425 | 91.776 | 65.215 | 1.00 | 0.00 | H |
| ATOM | 1494 | 1HB | HIS | 142 | 99.601 | 91.610 | 67.390 | 1.00 | 0.00 | H |
| ATOM | 1495 | 2HB | HIS | 142 | 98.095 | 91.771 | 68.280 | 1.00 | 0.00 | H |
| ATOM | 1496 | HE1 | HIS | 142 | 98.758 | 86.968 | 66.161 | 1.00 | 0.00 | H |
| ATOM | 1497 | HE2 | HIS | 142 | 97.139 | 86.941 | 68.140 | 1.00 | 0.00 | H |
| ATOM | 1498 | HD2 | HIS | 142 | 96.957 | 89.499 | 68.943 | 1.00 | 0.00 | H |
| ATOM | 1499 | N | LYS | 143 | 95.427 | 92.296 | 66.675 | 1.00 | 0.00 | N |
| ATOM | 1500 | CA | LYS | 143 | 93.966 | 92.168 | 66.366 | 1.00 | 0.00 | C |
| ATOM | 1501 | C | LYS | 143 | 93.456 | 92.911 | 65.071 | 1.00 | 0.00 | C |
| ATOM | 1502 | O | LYS | 143 | 92.547 | 92.416 | 64.401 | 1.00 | 0.00 | O |
| ATOM | 1503 | CB | LYS | 143 | 93.182 | 92.544 | 67.652 | 1.00 | 0.00 | C |
| ATOM | 1504 | CG | LYS | 143 | 91.694 | 92.119 | 67.639 | 1.00 | 0.00 | C |
| ATOM | 1505 | CD | LYS | 143 | 90.996 | 92.400 | 68.981 | 1.00 | 0.00 | C |
| ATOM | 1506 | CE | LYS | 143 | 89.522 | 91.976 | 68.981 | 1.00 | 0.00 | C |
| ATOM | 1507 | NZ | LYS | 143 | 88.936 | 92.262 | 70.305 | 1.00 | 0.00 | N |
| ATOM | 1508 | 1HZ | LYS | 143 | 87.946 | 91.979 | 70.312 | 1.00 | 0.00 | H |
| ATOM | 1509 | 2HZ | LYS | 143 | 89.448 | 91.737 | 71.029 | 1.00 | 0.00 | H |
| ATOM | 1510 | 3HZ | LYS | 143 | 89.005 | 93.271 | 70.501 | 1.00 | 0.00 | H |
| ATOM | 1511 | H | LYS | 143 | 95.796 | 92.914 | 67.405 | 1.00 | 0.00 | H |
| ATOM | 1512 | HA | LYS | 143 | 93.768 | 91.094 | 66.174 | 1.00 | 0.00 | H |
| ATOM | 1513 | 1HB | LYS | 143 | 93.663 | 92.071 | 68.532 | 1.00 | 0.00 | H |
| ATOM | 1514 | 2HB | LYS | 143 | 93.260 | 93.634 | 67.831 | 1.00 | 0.00 | H |
| ATOM | 1515 | 1HG | LYS | 143 | 91.157 | 92.643 | 66.822 | 1.00 | 0.00 | H |
| ATOM | 1516 | 2HG | LYS | 143 | 91.612 | 91.041 | 67.399 | 1.00 | 0.00 | H |
| ATOM | 1517 | 1HD | LYS | 143 | 91.533 | 91.864 | 69.788 | 1.00 | 0.00 | H |
| ATOM | 1518 | 2HD | LYS | 143 | 91.083 | 93.480 | 69.220 | 1.00 | 0.00 | H |
| ATOM | 1519 | 1HE | LYS | 143 | 88.963 | 92.511 | 68.187 | 1.00 | 0.00 | H |
| ATOM | 1520 | 2HE | LYS | 143 | 89.428 | 90.895 | 68.753 | 1.00 | 0.00 | H |
| ATOM | 1521 | N | ARG | 144 | 94.052 | 94.055 | 64.693 | 0.00 | 0.00 | N |
| ATOM | 1522 | CA | ARG | 144 | 93.818 | 94.707 | 63.364 | 0.00 | 0.00 | C |
| ATOM | 1523 | C | ARG | 144 | 94.430 | 93.932 | 62.133 | 0.00 | 0.00 | C |
| ATOM | 1524 | O | ARG | 144 | 93.771 | 93.765 | 61.107 | 0.00 | 0.00 | O |
| ATOM | 1525 | CB | ARG | 144 | 94.325 | 96.187 | 63.398 | 0.00 | 0.00 | C |
| ATOM | 1526 | CG | ARG | 144 | 94.048 | 97.079 | 64.638 | 0.00 | 0.00 | C |
| ATOM | 1527 | CD | ARG | 144 | 92.575 | 97.236 | 65.036 | 0.00 | 0.00 | C |
| ATOM | 1528 | NE | ARG | 144 | 92.517 | 98.058 | 66.275 | 0.00 | 0.00 | N |
| ATOM | 1529 | CZ | ARG | 144 | 91.428 | 98.276 | 67.001 | 0.00 | 0.00 | C |
| ATOM | 1530 | NH1 | ARG | 144 | 90.254 | 97.796 | 66.722 | 0.00 | 0.00 | N |
| ATOM | 1531 | NH2 | ARG | 144 | 91.551 | 99.010 | 68.049 | 0.00 | 0.00 | N |
| ATOM | 1532 | HE | ARG | 144 | 93.394 | 98.494 | 66.595 | 1.00 | 0.00 | H |
| ATOM | 1533 | H | ARG | 144 | 94.877 | 94.270 | 65.261 | 0.00 | 0.00 | H |
| ATOM | 1534 | HA | ARG | 144 | 92.724 | 94.742 | 63.188 | 0.00 | 0.00 | H |
| ATOM | 1535 | 1HB | ARG | 144 | 93.934 | 96.702 | 62.499 | 0.00 | 0.00 | H |
| ATOM | 1536 | 2HB | ARG | 144 | 95.422 | 96.182 | 63.249 | 0.00 | 0.00 | H |
| ATOM | 1537 | 1HG | ARG | 144 | 94.490 | 98.081 | 64.473 | 0.00 | 0.00 | H |
| ATOM | 1538 | 2HG | ARG | 144 | 94.603 | 96.661 | 65.499 | 0.00 | 0.00 | H |
| ATOM | 1539 | 1HD | ARG | 144 | 92.121 | 96.238 | 65.209 | 0.00 | 0.00 | H |
| ATOM | 1540 | 2HD | ARG | 144 | 91.997 | 97.713 | 64.219 | 0.00 | 0.00 | H |
| ATOM | 1541 | 1HH1 | ARG | 144 | 89.492 | 98.028 | 67.359 | 0.00 | 0.00 | H |
| ATOM | 1542 | 2HH1 | ARG | 144 | 90.243 | 97.220 | 65.880 | 0.00 | 0.00 | H |
| ATOM | 1543 | 1HH2 | ARG | 144 | 90.714 | 99.175 | 68.606 | 0.00 | 0.00 | H |
| ATOM | 1544 | 2HH2 | ARG | 144 | 92.504 | 99.350 | 68.179 | 0.00 | 0.00 | H |
| ATOM | 1545 | N | ARG | 145 | 95.685 | 93.464 | 62.247 | 1.00 | 0.00 | N |
| ATOM | 1546 | CA | ARG | 145 | 96.370 | 92.597 | 61.243 | 1.00 | 0.00 | C |
| ATOM | 1547 | C | ARG | 145 | 95.872 | 91.111 | 61.094 | 1.00 | 0.00 | C |
| ATOM | 1548 | O | ARG | 145 | 96.228 | 90.473 | 60.101 | 1.00 | 0.00 | O |
| ATOM | 1549 | CB | ARG | 145 | 97.877 | 92.563 | 61.650 | 1.00 | 0.00 | C |
| ATOM | 1550 | CG | ARG | 145 | 98.679 | 93.889 | 61.575 | 1.00 | 0.00 | C |
| ATOM | 1551 | CD | ARG | 145 | 100.051 | 93.757 | 62.256 | 1.00 | 0.00 | C |
| ATOM | 1552 | NE | ARG | 145 | 100.752 | 95.066 | 62.215 | 1.00 | 0.00 | N |
| ATOM | 1553 | CZ | ARG | 145 | 102.002 | 95.278 | 62.604 | 1.00 | 0.00 | C |
| ATOM | 1554 | NH1 | ARG | 145 | 102.785 | 94.365 | 63.098 | 1.00 | 0.00 | N |
| ATOM | 1555 | NH2 | ARG | 145 | 102.465 | 96.470 | 62.475 | 1.00 | 0.00 | N |
| ATOM | 1556 | HE | ARG | 145 | 100.224 | 95.874 | 61.856 | 1.00 | 0.00 | H |
| ATOM | 1557 | H | ARG | 145 | 96.135 | 93.688 | 63.146 | 1.00 | 0.00 | H |
| ATOM | 1558 | HA | ARG | 145 | 96.281 | 93.057 | 60.240 | 1.00 | 0.00 | H |
| ATOM | 1559 | 1HB | ARG | 145 | 97.951 | 92.141 | 62.674 | 1.00 | 0.00 | H |
| ATOM | 1560 | 2HB | ARG | 145 | 98.410 | 91.821 | 61.021 | 1.00 | 0.00 | H |
| ATOM | 1561 | 1HG | ARG | 145 | 98.805 | 94.204 | 60.520 | 1.00 | 0.00 | H |
| ATOM | 1562 | 2HG | ARG | 145 | 98.115 | 94.711 | 62.056 | 1.00 | 0.00 | H |
| ATOM | 1563 | 1HD | ARG | 145 | 99.923 | 93.427 | 63.309 | 1.00 | 0.00 | H |
| ATOM | 1564 | 2HD | ARG | 145 | 100.648 | 92.971 | 61.751 | 1.00 | 0.00 | H |
| ATOM | 1565 | 2HH1 | ARG | 145 | 102.341 | 93.449 | 63.156 | 1.00 | 0.00 | H |
| ATOM | 1566 | 1HH1 | ARG | 145 | 103.735 | 94.640 | 63.347 | 1.00 | 0.00 | H |
| ATOM | 1567 | 1HH2 | ARG | 145 | 101.776 | 97.099 | 62.057 | 1.00 | 0.00 | H |
| ATOM | 1568 | 2HH2 | ARG | 145 | 103.434 | 96.635 | 62.741 | 1.00 | 0.00 | H |
| ATOM | 1569 | N | LYS | 146 | 95.154 | 90.529 | 62.082 | 1.00 | 0.00 | N |
| ATOM | 1570 | CA | LYS | 146 | 94.909 | 89.056 | 62.215 | 1.00 | 0.00 | C |
| ATOM | 1571 | C | LYS | 146 | 96.148 | 88.227 | 62.721 | 1.00 | 0.00 | C |
| ATOM | 1572 | O | LYS | 146 | 96.076 | 87.587 | 63.775 | 1.00 | 0.00 | O |
| ATOM | 1573 | CB | LYS | 146 | 94.152 | 88.452 | 60.992 | 1.00 | 0.00 | C |
| ATOM | 1574 | CG | LYS | 146 | 93.601 | 87.019 | 61.176 | 1.00 | 0.00 | C |
| ATOM | 1575 | CD | LYS | 146 | 92.958 | 86.485 | 59.881 | 1.00 | 0.00 | C |
| ATOM | 1576 | CE | LYS | 146 | 92.463 | 85.041 | 60.031 | 1.00 | 0.00 | C |
| ATOM | 1577 | NZ | LYS | 146 | 91.884 | 84.588 | 58.751 | 1.00 | 0.00 | N |
| ATOM | 1578 | 1HZ | LYS | 146 | 91.550 | 83.618 | 58.848 | 1.00 | 0.00 | H |
| ATOM | 1579 | 2HZ | LYS | 146 | 91.094 | 85.197 | 58.495 | 1.00 | 0.00 | H |
| ATOM | 1580 | 3HZ | LYS | 146 | 92.603 | 84.630 | 58.014 | 1.00 | 0.00 | H |
| ATOM | 1581 | H | LYS | 146 | 95.036 | 91.156 | 62.887 | 1.00 | 0.00 | H |
| ATOM | 1582 | HA | LYS | 146 | 94.192 | 88.979 | 63.054 | 1.00 | 0.00 | H |
| ATOM | 1583 | 1HB | LYS | 146 | 93.317 | 89.127 | 60.718 | 1.00 | 0.00 | H |
| ATOM | 1584 | 2HB | LYS | 146 | 94.824 | 88.464 | 60.112 | 1.00 | 0.00 | H |
| ATOM | 1585 | 1HG | LYS | 146 | 94.419 | 86.340 | 61.489 | 1.00 | 0.00 | H |
| ATOM | 1586 | 2HG | LYS | 146 | 92.866 | 87.000 | 62.003 | 1.00 | 0.00 | H |
| ATOM | 1587 | 1HD | LYS | 146 | 92.118 | 87.145 | 59.582 | 1.00 | 0.00 | H |
| ATOM | 1588 | 2HD | LYS | 146 | 93.694 | 86.549 | 59.054 | 1.00 | 0.00 | H |
| ATOM | 1589 | 1HE | LYS | 146 | 93.296 | 84.375 | 60.333 | 1.00 | 0.00 | H |
| ATOM | 1590 | 2HE | LYS | 146 | 91.704 | 84.972 | 60.836 | 1.00 | 0.00 | H |
| ATOM | 1591 | N | ALA | 147 | 97.248 | 88.196 | 61.950 | 1.00 | 0.00 | N |
| ATOM | 1592 | CA | ALA | 147 | 98.402 | 87.294 | 62.187 | 1.00 | 0.00 | C |
| ATOM | 1593 | C | ALA | 147 | 99.779 | 87.983 | 61.895 | 1.00 | 0.00 | C |
| ATOM | 1594 | O | ALA | 147 | 99.933 | 88.722 | 60.915 | 1.00 | 0.00 | O |
| ATOM | 1595 | CB | ALA | 147 | 98.170 | 86.085 | 61.256 | 1.00 | 0.00 | C |
| ATOM | 1596 | H | ALA | 147 | 97.178 | 88.800 | 61.122 | 1.00 | 0.00 | H |
| ATOM | 1597 | HA | ALA | 147 | 98.408 | 86.934 | 63.237 | 1.00 | 0.00 | H |
| ATOM | 1598 | 2HB | ALA | 147 | 97.255 | 85.528 | 61.536 | 1.00 | 0.00 | H |
| ATOM | 1599 | 3HB | ALA | 147 | 98.071 | 86.380 | 60.195 | 1.00 | 0.00 | H |
| ATOM | 1600 | 1HB | ALA | 147 | 99.006 | 85.368 | 61.308 | 1.00 | 0.00 | H |
| ATOM | 1601 | N | LEU | 148 | 100.806 | 87.700 | 62.716 | 1.00 | 0.00 | N |
| ATOM | 1602 | CA | LEU | 148 | 102.207 | 88.129 | 62.439 | 1.00 | 0.00 | C |
| ATOM | 1603 | C | LEU | 148 | 102.986 | 87.003 | 61.680 | 1.00 | 0.00 | C |
| ATOM | 1604 | O | LEU | 148 | 103.014 | 85.843 | 62.105 | 1.00 | 0.00 | O |
| ATOM | 1605 | CB | LEU | 148 | 102.939 | 88.519 | 63.757 | 1.00 | 0.00 | C |
| ATOM | 1606 | CG | LEU | 148 | 102.326 | 89.637 | 64.641 | 1.00 | 0.00 | C |
| ATOM | 1607 | CD1 | LEU | 148 | 103.212 | 89.891 | 65.868 | 1.00 | 0.00 | C |
| ATOM | 1608 | CD2 | LEU | 148 | 102.098 | 90.967 | 63.907 | 1.00 | 0.00 | C |
| ATOM | 1609 | H | LEU | 148 | 100.590 | 87.057 | 63.490 | 1.00 | 0.00 | H |
| ATOM | 1610 | HA | LEU | 148 | 102.200 | 89.037 | 61.803 | 1.00 | 0.00 | H |
| ATOM | 1611 | 1HB | LEU | 148 | 103.042 | 87.607 | 64.378 | 1.00 | 0.00 | H |
| ATOM | 1612 | 2HB | LEU | 148 | 103.981 | 88.795 | 63.509 | 1.00 | 0.00 | H |
| ATOM | 1613 | HG | LEU | 148 | 101.343 | 89.275 | 65.004 | 1.00 | 0.00 | H |
| ATOM | 1614 | 2HD1 | LEU | 148 | 103.436 | 88.954 | 66.407 | 1.00 | 0.00 | H |
| ATOM | 1615 | 3HD1 | LEU | 148 | 104.185 | 90.351 | 65.609 | 1.00 | 0.00 | H |
| ATOM | 1616 | 1HD1 | LEU | 148 | 102.720 | 90.564 | 66.595 | 1.00 | 0.00 | H |
| ATOM | 1617 | 2HD2 | LEU | 148 | 103.034 | 91.381 | 63.491 | 1.00 | 0.00 | H |
| ATOM | 1618 | 3HD2 | LEU | 148 | 101.382 | 90.848 | 63.072 | 1.00 | 0.00 | H |
| ATOM | 1619 | 1HD2 | LEU | 148 | 101.664 | 91.730 | 64.580 | 1.00 | 0.00 | H |
| ATOM | 1620 | N | THR | 149 | 103.648 | 87.338 | 60.563 | 1.00 | 0.00 | N |
| ATOM | 1621 | CA | THR | 149 | 104.435 | 86.350 | 59.754 | 1.00 | 0.00 | C |
| ATOM | 1622 | C | THR | 149 | 105.682 | 85.746 | 60.498 | 1.00 | 0.00 | C |
| ATOM | 1623 | O | THR | 149 | 106.190 | 86.324 | 61.461 | 1.00 | 0.00 | O |
| ATOM | 1624 | CB | THR | 149 | 104.837 | 86.961 | 58.370 | 1.00 | 0.00 | C |
| ATOM | 1625 | OG1 | THR | 149 | 105.679 | 88.098 | 58.524 | 1.00 | 0.00 | O |
| ATOM | 1626 | CG2 | THR | 149 | 103.673 | 87.383 | 57.459 | 1.00 | 0.00 | C |
| ATOM | 1627 | H | THR | 149 | 103.553 | 88.318 | 60.285 | 1.00 | 0.00 | H |
| ATOM | 1628 | HA | THR | 149 | 103.758 | 85.496 | 59.545 | 1.00 | 0.00 | H |
| ATOM | 1629 | HB | THR | 149 | 105.407 | 86.192 | 57.810 | 1.00 | 0.00 | H |
| ATOM | 1630 | HG1 | THR | 149 | 105.422 | 88.711 | 57.831 | 1.00 | 0.00 | H |
| ATOM | 1631 | 1HG2 | THR | 149 | 104.024 | 87.738 | 56.472 | 1.00 | 0.00 | H |
| ATOM | 1632 | 2HG2 | THR | 149 | 102.982 | 86.539 | 57.266 | 1.00 | 0.00 | H |
| ATOM | 1633 | 3HG2 | THR | 149 | 103.065 | 88.194 | 57.904 | 1.00 | 0.00 | H |
| ATOM | 1634 | N | GLU | 150 | 106.193 | 84.586 | 60.038 | 0.00 | 0.00 | N |
| ATOM | 1635 | CA | GLU | 150 | 107.355 | 83.885 | 60.680 | 0.00 | 0.00 | C |
| ATOM | 1636 | C | GLU | 150 | 108.671 | 84.748 | 60.925 | 0.00 | 0.00 | C |
| ATOM | 1637 | O | GLU | 150 | 109.164 | 84.675 | 62.052 | 0.00 | 0.00 | O |
| ATOM | 1638 | CB | GLU | 150 | 107.587 | 82.517 | 59.960 | 0.00 | 0.00 | C |
| ATOM | 1639 | CG | GLU | 150 | 106.402 | 81.503 | 60.039 | 0.00 | 0.00 | C |
| ATOM | 1640 | CD | GLU | 150 | 106.509 | 80.210 | 59.224 | 0.00 | 0.00 | C |
| ATOM | 1641 | OE1 | GLU | 150 | 107.099 | 79.207 | 59.610 | 0.00 | 0.00 | O |
| ATOM | 1642 | OE2 | GLU | 150 | 105.835 | 80.283 | 58.040 | 0.00 | 0.00 | O |
| ATOM | 1643 | H | GLU | 150 | 105.612 | 84.128 | 59.330 | 0.00 | 0.00 | H |
| ATOM | 1644 | HA | GLU | 150 | 107.022 | 83.624 | 61.706 | 0.00 | 0.00 | H |
| ATOM | 1645 | 1HB | GLU | 150 | 108.487 | 82.034 | 60.389 | 0.00 | 0.00 | H |
| ATOM | 1646 | 2HB | GLU | 150 | 107.856 | 82.703 | 58.907 | 0.00 | 0.00 | H |
| ATOM | 1647 | 1HG | GLU | 150 | 105.461 | 81.993 | 59.736 | 0.00 | 0.00 | H |
| ATOM | 1648 | 2HG | GLU | 150 | 106.235 | 81.212 | 61.092 | 0.00 | 0.00 | H |
| ATOM | 1649 | N | PRO | 151 | 109.214 | 85.621 | 60.014 | 0.00 | 0.00 | N |
| ATOM | 1650 | CA | PRO | 151 | 110.199 | 86.694 | 60.384 | 0.00 | 0.00 | C |
| ATOM | 1651 | CD | PRO | 151 | 108.872 | 85.617 | 58.583 | 0.00 | 0.00 | C |
| ATOM | 1652 | C | PRO | 151 | 109.855 | 87.723 | 61.524 | 0.00 | 0.00 | C |
| ATOM | 1653 | O | PRO | 151 | 110.743 | 88.145 | 62.266 | 0.00 | 0.00 | O |
| ATOM | 1654 | CB | PRO | 151 | 110.413 | 87.422 | 59.037 | 0.00 | 0.00 | C |
| ATOM | 1655 | CG | PRO | 151 | 109.986 | 86.440 | 57.949 | 0.00 | 0.00 | C |
| ATOM | 1656 | HA | PRO | 151 | 111.150 | 86.203 | 60.668 | 0.00 | 0.00 | H |
| ATOM | 1657 | 1HD | PRO | 151 | 107.885 | 86.088 | 58.412 | 0.00 | 0.00 | H |
| ATOM | 1658 | 2HD | PRO | 151 | 108.856 | 84.598 | 58.157 | 0.00 | 0.00 | H |
| ATOM | 1659 | 1HB | PRO | 151 | 111.462 | 87.754 | 58.917 | 0.00 | 0.00 | H |
| ATOM | 1660 | 2HB | PRO | 151 | 109.787 | 88.335 | 58.968 | 0.00 | 0.00 | H |
| ATOM | 1661 | 1HG | PRO | 151 | 110.834 | 85.785 | 57.690 | 0.00 | 0.00 | H |
| ATOM | 1662 | 2HG | PRO | 151 | 109.669 | 86.944 | 57.018 | 0.00 | 0.00 | H |
| ATOM | 1663 | N | GLU | 152 | 108.587 | 88.146 | 61.648 | 1.00 | 0.00 | N |
| ATOM | 1664 | CA | GLU | 152 | 108.117 | 89.007 | 62.775 | 1.00 | 0.00 | C |
| ATOM | 1665 | C | GLU | 152 | 107.904 | 88.238 | 64.129 | 1.00 | 0.00 | C |
| ATOM | 1666 | O | GLU | 152 | 108.439 | 88.657 | 65.158 | 1.00 | 0.00 | O |
| ATOM | 1667 | CB | GLU | 152 | 106.855 | 89.756 | 62.250 | 1.00 | 0.00 | C |
| ATOM | 1668 | CG | GLU | 152 | 106.211 | 90.800 | 63.203 | 1.00 | 0.00 | C |
| ATOM | 1669 | CD | GLU | 152 | 106.987 | 92.086 | 63.492 | 1.00 | 0.00 | C |
| ATOM | 1670 | OE1 | GLU | 152 | 108.200 | 92.220 | 63.370 | 1.00 | 0.00 | O |
| ATOM | 1671 | OE2 | GLU | 152 | 106.172 | 93.078 | 63.939 | 1.00 | 0.00 | O |
| ATOM | 1672 | H | GLU | 152 | 107.932 | 87.605 | 61.072 | 1.00 | 0.00 | H |
| ATOM | 1673 | HA | GLU | 152 | 108.892 | 89.773 | 62.981 | 1.00 | 0.00 | H |
| ATOM | 1674 | 1HB | GLU | 152 | 107.096 | 90.261 | 61.293 | 1.00 | 0.00 | H |
| ATOM | 1675 | 2HB | GLU | 152 | 106.081 | 89.009 | 61.984 | 1.00 | 0.00 | H |
| ATOM | 1676 | 1HG | GLU | 152 | 105.230 | 91.093 | 62.786 | 1.00 | 0.00 | H |
| ATOM | 1677 | 2HG | GLU | 152 | 105.977 | 90.333 | 64.176 | 1.00 | 0.00 | H |
| ATOM | 1678 | N | ALA | 153 | 107.158 | 87.116 | 64.137 | 0.00 | 0.00 | N |
| ATOM | 1679 | CA | ALA | 153 | 107.037 | 86.226 | 65.325 | 0.00 | 0.00 | C |
| ATOM | 1680 | C | ALA | 153 | 108.359 | 85.596 | 65.902 | 0.00 | 0.00 | C |
| ATOM | 1681 | O | ALA | 153 | 108.492 | 85.516 | 67.125 | 0.00 | 0.00 | O |
| ATOM | 1682 | CB | ALA | 153 | 105.982 | 85.168 | 64.944 | 0.00 | 0.00 | C |
| ATOM | 1683 | H | ALA | 153 | 106.793 | 86.850 | 63.212 | 0.00 | 0.00 | H |
| ATOM | 1684 | HA | ALA | 153 | 106.608 | 86.829 | 66.151 | 0.00 | 0.00 | H |
| ATOM | 1685 | 1HB | ALA | 153 | 105.751 | 84.502 | 65.795 | 0.00 | 0.00 | H |
| ATOM | 1686 | 2HB | ALA | 153 | 105.021 | 85.624 | 64.636 | 0.00 | 0.00 | H |
| ATOM | 1687 | 3HB | ALA | 153 | 106.317 | 84.525 | 64.108 | 0.00 | 0.00 | H |
| ATOM | 1688 | N | ARG | 154 | 109.343 | 85.199 | 65.066 | 0.00 | 0.00 | N |
| ATOM | 1689 | CA | ARG | 154 | 110.722 | 84.852 | 65.539 | 0.00 | 0.00 | C |
| ATOM | 1690 | C | ARG | 154 | 111.530 | 85.995 | 66.258 | 0.00 | 0.00 | C |
| ATOM | 1691 | O | ARG | 154 | 112.158 | 85.741 | 67.286 | 0.00 | 0.00 | O |
| ATOM | 1692 | CB | ARG | 154 | 111.503 | 84.146 | 64.390 | 0.00 | 0.00 | C |
| ATOM | 1693 | CG | ARG | 154 | 112.107 | 85.065 | 63.296 | 0.00 | 0.00 | C |
| ATOM | 1694 | CD | ARG | 154 | 113.543 | 85.528 | 63.593 | 0.00 | 0.00 | C |
| ATOM | 1695 | NE | ARG | 154 | 113.880 | 86.656 | 62.687 | 0.00 | 0.00 | N |
| ATOM | 1696 | CZ | ARG | 154 | 114.866 | 87.527 | 62.870 | 0.00 | 0.00 | C |
| ATOM | 1697 | NH1 | ARG | 154 | 115.740 | 87.459 | 63.831 | 0.00 | 0.00 | N |
| ATOM | 1698 | NH2 | ARG | 154 | 114.962 | 88.493 | 62.030 | 0.00 | 0.00 | N |
| ATOM | 1699 | HE | ARG | 154 | 113.299 | 86.772 | 61.844 | 1.00 | 0.00 | H |
| ATOM | 1700 | H | ARG | 154 | 109.127 | 85.334 | 64.069 | 0.00 | 0.00 | H |
| ATOM | 1701 | HA | ARG | 154 | 110.592 | 84.074 | 66.310 | 0.00 | 0.00 | H |
| ATOM | 1702 | 1HB | ARG | 154 | 110.836 | 83.409 | 63.904 | 0.00 | 0.00 | H |
| ATOM | 1703 | 2HB | ARG | 154 | 112.305 | 83.522 | 64.828 | 0.00 | 0.00 | H |
| ATOM | 1704 | 1HG | ARG | 154 | 111.443 | 85.935 | 63.149 | 0.00 | 0.00 | H |
| ATOM | 1705 | 2HG | ARG | 154 | 112.090 | 84.564 | 62.315 | 0.00 | 0.00 | H |
| ATOM | 1706 | 1HD | ARG | 154 | 114.240 | 84.680 | 63.473 | 0.00 | 0.00 | H |
| ATOM | 1707 | 2HD | ARG | 154 | 113.654 | 85.843 | 64.648 | 0.00 | 0.00 | H |
| ATOM | 1708 | 1HH1 | ARG | 154 | 116.480 | 88.160 | 63.838 | 0.00 | 0.00 | H |
| ATOM | 1709 | 2HH1 | ARG | 154 | 115.646 | 86.618 | 64.402 | 0.00 | 0.00 | H |
| ATOM | 1710 | 1HH2 | ARG | 154 | 115.697 | 89.181 | 62.187 | 0.00 | 0.00 | H |
| ATOM | 1711 | 2HH2 | ARG | 154 | 114.203 | 88.476 | 61.347 | 0.00 | 0.00 | H |
| ATOM | 1712 | N | TYR | 155 | 111.514 | 87.235 | 65.733 | 1.00 | 0.00 | N |
| ATOM | 1713 | CA | TYR | 155 | 112.066 | 88.440 | 66.422 | 1.00 | 0.00 | C |
| ATOM | 1714 | C | TYR | 155 | 111.457 | 88.734 | 67.843 | 1.00 | 0.00 | C |
| ATOM | 1715 | O | TYR | 155 | 112.186 | 89.051 | 68.784 | 1.00 | 0.00 | O |
| ATOM | 1716 | CB | TYR | 155 | 111.895 | 89.611 | 65.411 | 1.00 | 0.00 | C |
| ATOM | 1717 | CG | TYR | 155 | 112.698 | 90.878 | 65.739 | 1.00 | 0.00 | C |
| ATOM | 1718 | CD1 | TYR | 155 | 113.979 | 91.045 | 65.204 | 1.00 | 0.00 | C |
| ATOM | 1719 | CD2 | TYR | 155 | 112.161 | 91.879 | 66.559 | 1.00 | 0.00 | C |
| ATOM | 1720 | CE1 | TYR | 155 | 114.708 | 92.198 | 65.479 | 1.00 | 0.00 | C |
| ATOM | 1721 | CE2 | TYR | 155 | 112.900 | 93.027 | 66.840 | 1.00 | 0.00 | C |
| ATOM | 1722 | CZ | TYR | 155 | 114.171 | 93.185 | 66.296 | 1.00 | 0.00 | C |
| ATOM | 1723 | OH | TYR | 155 | 114.904 | 94.310 | 66.558 | 1.00 | 0.00 | O |
| ATOM | 1724 | H | TYR | 155 | 111.095 | 87.361 | 64.800 | 1.00 | 0.00 | H |
| ATOM | 1725 | HA | TYR | 155 | 113.152 | 88.277 | 66.573 | 1.00 | 0.00 | H |
| ATOM | 1726 | 1HB | TYR | 155 | 112.182 | 89.280 | 64.391 | 1.00 | 0.00 | H |
| ATOM | 1727 | 2HB | TYR | 155 | 110.823 | 89.866 | 65.305 | 1.00 | 0.00 | H |
| ATOM | 1728 | HD1 | TYR | 155 | 114.411 | 90.280 | 64.573 | 1.00 | 0.00 | H |
| ATOM | 1729 | HD2 | TYR | 155 | 111.176 | 91.762 | 66.990 | 1.00 | 0.00 | H |
| ATOM | 1730 | HE1 | TYR | 155 | 115.698 | 92.323 | 65.073 | 1.00 | 0.00 | H |
| ATOM | 1731 | HE2 | TYR | 155 | 112.486 | 93.787 | 67.484 | 1.00 | 0.00 | H |
| ATOM | 1732 | HH | TYR | 155 | 114.330 | 94.967 | 66.971 | 1.00 | 0.00 | H |
| ATOM | 1733 | N | TYR | 156 | 110.132 | 88.574 | 67.992 | 0.00 | 0.00 | N |
| ATOM | 1734 | CA | TYR | 156 | 109.433 | 88.599 | 69.304 | 0.00 | 0.00 | C |
| ATOM | 1735 | C | TYR | 156 | 109.852 | 87.428 | 70.275 | 0.00 | 0.00 | C |
| ATOM | 1736 | O | TYR | 156 | 110.195 | 87.710 | 71.422 | 0.00 | 0.00 | O |
| ATOM | 1737 | CB | TYR | 156 | 107.898 | 88.642 | 69.018 | 0.00 | 0.00 | C |
| ATOM | 1738 | CG | TYR | 156 | 107.223 | 89.943 | 68.497 | 0.00 | 0.00 | C |
| ATOM | 1739 | CD1 | TYR | 156 | 107.798 | 90.774 | 67.520 | 0.00 | 0.00 | C |
| ATOM | 1740 | CE1 | TYR | 156 | 107.141 | 91.923 | 67.090 | 0.00 | 0.00 | C |
| ATOM | 1741 | CZ | TYR | 156 | 105.895 | 92.247 | 67.606 | 0.00 | 0.00 | C |
| ATOM | 1742 | OH | TYR | 156 | 105.263 | 93.392 | 67.205 | 0.00 | 0.00 | O |
| ATOM | 1743 | CE2 | TYR | 156 | 105.296 | 91.425 | 68.555 | 0.00 | 0.00 | C |
| ATOM | 1744 | CD2 | TYR | 156 | 105.959 | 90.281 | 68.997 | 0.00 | 0.00 | C |
| ATOM | 1745 | H | TYR | 156 | 109.565 | 88.424 | 67.145 | 1.00 | 0.00 | H |
| ATOM | 1746 | HA | TYR | 156 | 109.702 | 89.539 | 69.829 | 0.00 | 0.00 | H |
| ATOM | 1747 | 1HB | TYR | 156 | 107.396 | 88.358 | 69.964 | 0.00 | 0.00 | H |
| ATOM | 1748 | 2HB | TYR | 156 | 107.629 | 87.819 | 68.330 | 0.00 | 0.00 | H |
| ATOM | 1749 | HD1 | TYR | 156 | 108.758 | 90.552 | 67.084 | 0.00 | 0.00 | H |
| ATOM | 1750 | HE1 | TYR | 156 | 107.610 | 92.568 | 66.361 | 0.00 | 0.00 | H |
| ATOM | 1751 | HH | TYR | 156 | 104.508 | 93.523 | 67.781 | 0.00 | 0.00 | H |
| ATOM | 1752 | HE2 | TYR | 156 | 104.337 | 91.683 | 68.977 | 0.00 | 0.00 | H |
| ATOM | 1753 | HD2 | TYR | 156 | 105.486 | 89.679 | 69.760 | 0.00 | 0.00 | H |
| ATOM | 1754 | N | LEU | 157 | 109.860 | 86.143 | 69.853 | 1.00 | 0.00 | N |
| ATOM | 1755 | CA | LEU | 157 | 110.345 | 85.012 | 70.711 | 1.00 | 0.00 | C |
| ATOM | 1756 | C | LEU | 157 | 111.873 | 84.971 | 71.073 | 1.00 | 0.00 | C |
| ATOM | 1757 | O | LEU | 157 | 112.208 | 84.574 | 72.191 | 1.00 | 0.00 | O |
| ATOM | 1758 | CB | LEU | 157 | 109.771 | 83.662 | 70.181 | 1.00 | 0.00 | C |
| ATOM | 1759 | CG | LEU | 157 | 110.541 | 82.934 | 69.039 | 1.00 | 0.00 | C |
| ATOM | 1760 | CD1 | LEU | 157 | 111.558 | 81.907 | 69.572 | 1.00 | 0.00 | C |
| ATOM | 1761 | CD2 | LEU | 157 | 109.575 | 82.206 | 68.098 | 1.00 | 0.00 | C |
| ATOM | 1762 | H | LEU | 157 | 109.582 | 86.019 | 68.870 | 1.00 | 0.00 | H |
| ATOM | 1763 | HA | LEU | 157 | 109.856 | 85.147 | 71.693 | 1.00 | 0.00 | H |
| ATOM | 1764 | 1HB | LEU | 157 | 109.664 | 82.968 | 71.037 | 1.00 | 0.00 | H |
| ATOM | 1765 | 2HB | LEU | 157 | 108.718 | 83.834 | 69.881 | 1.00 | 0.00 | H |
| ATOM | 1766 | HG | LEU | 157 | 111.088 | 83.689 | 68.443 | 1.00 | 0.00 | H |
| ATOM | 1767 | 2HD1 | LEU | 157 | 112.317 | 82.369 | 70.227 | 1.00 | 0.00 | H |
| ATOM | 1768 | 3HD1 | LEU | 157 | 111.074 | 81.105 | 70.159 | 1.00 | 0.00 | H |
| ATOM | 1769 | 1HD1 | LEU | 157 | 112.116 | 81.418 | 68.750 | 1.00 | 0.00 | H |
| ATOM | 1770 | 2HD2 | LEU | 157 | 108.993 | 81.441 | 68.635 | 1.00 | 0.00 | H |
| ATOM | 1771 | 3HD2 | LEU | 157 | 108.848 | 82.902 | 67.638 | 1.00 | 0.00 | H |
| ATOM | 1772 | 1HD2 | LEU | 157 | 110.101 | 81.700 | 67.267 | 1.00 | 0.00 | H |
| ATOM | 1773 | N | ARG | 158 | 112.794 | 85.339 | 70.162 | 1.00 | 0.00 | N |
| ATOM | 1774 | CA | ARG | 158 | 114.267 | 85.286 | 70.417 | 1.00 | 0.00 | C |
| ATOM | 1775 | C | ARG | 158 | 114.818 | 86.178 | 71.582 | 1.00 | 0.00 | C |
| ATOM | 1776 | O | ARG | 158 | 115.619 | 85.706 | 72.392 | 1.00 | 0.00 | O |
| ATOM | 1777 | CB | ARG | 158 | 115.007 | 85.449 | 69.057 | 1.00 | 0.00 | C |
| ATOM | 1778 | CG | ARG | 158 | 115.175 | 86.874 | 68.458 | 1.00 | 0.00 | C |
| ATOM | 1779 | CD | ARG | 158 | 116.540 | 87.524 | 68.779 | 1.00 | 0.00 | C |
| ATOM | 1780 | NE | ARG | 158 | 116.446 | 89.000 | 68.662 | 1.00 | 0.00 | N |
| ATOM | 1781 | CZ | ARG | 158 | 117.045 | 89.775 | 67.769 | 1.00 | 0.00 | C |
| ATOM | 1782 | NH1 | ARG | 158 | 117.835 | 89.356 | 66.828 | 1.00 | 0.00 | N |
| ATOM | 1783 | NH2 | ARG | 158 | 116.819 | 91.036 | 67.853 | 1.00 | 0.00 | N |
| ATOM | 1784 | HE | ARG | 158 | 115.849 | 89.477 | 69.353 | 1.00 | 0.00 | H |
| ATOM | 1785 | H | ARG | 158 | 112.401 | 85.649 | 69.259 | 1.00 | 0.00 | H |
| ATOM | 1786 | HA | ARG | 158 | 114.482 | 84.249 | 70.742 | 1.00 | 0.00 | H |
| ATOM | 1787 | 1HB | ARG | 158 | 115.999 | 84.986 | 69.177 | 1.00 | 0.00 | H |
| ATOM | 1788 | 2HB | ARG | 158 | 114.521 | 84.802 | 68.299 | 1.00 | 0.00 | H |
| ATOM | 1789 | 1HG | ARG | 158 | 115.054 | 86.841 | 67.357 | 1.00 | 0.00 | H |
| ATOM | 1790 | 2HG | ARG | 158 | 114.337 | 87.520 | 68.789 | 1.00 | 0.00 | H |
| ATOM | 1791 | 1HD | ARG | 158 | 116.836 | 87.294 | 69.821 | 1.00 | 0.00 | H |
| ATOM | 1792 | 2HD | ARG | 158 | 117.337 | 87.084 | 68.149 | 1.00 | 0.00 | H |
| ATOM | 1793 | 2HH1 | ARG | 158 | 117.958 | 88.343 | 66.791 | 1.00 | 0.00 | H |
| ATOM | 1794 | 1HH1 | ARG | 158 | 118.205 | 90.060 | 66.191 | 1.00 | 0.00 | H |
| ATOM | 1795 | 1HH2 | ARG | 158 | 116.085 | 91.238 | 68.536 | 1.00 | 0.00 | H |
| ATOM | 1796 | 2HH2 | ARG | 158 | 117.170 | 91.627 | 67.101 | 1.00 | 0.00 | H |
| ATOM | 1797 | N | GLN | 159 | 114.342 | 87.426 | 71.705 | 0.00 | 0.00 | N |
| ATOM | 1798 | CA | GLN | 159 | 114.561 | 88.275 | 72.911 | 0.00 | 0.00 | C |
| ATOM | 1799 | C | GLN | 159 | 113.899 | 87.764 | 74.246 | 0.00 | 0.00 | C |
| ATOM | 1800 | O | GLN | 159 | 114.458 | 87.994 | 75.318 | 0.00 | 0.00 | O |
| ATOM | 1801 | CB | GLN | 159 | 114.096 | 89.718 | 72.581 | 0.00 | 0.00 | C |
| ATOM | 1802 | CG | GLN | 159 | 114.776 | 90.433 | 71.381 | 0.00 | 0.00 | C |
| ATOM | 1803 | CD | GLN | 159 | 114.427 | 91.912 | 71.236 | 0.00 | 0.00 | C |
| ATOM | 1804 | OE1 | GLN | 159 | 113.474 | 92.320 | 70.582 | 0.00 | 0.00 | O |
| ATOM | 1805 | NE2 | GLN | 159 | 115.171 | 92.782 | 71.861 | 0.00 | 0.00 | N |
| ATOM | 1806 | H | GLN | 159 | 113.734 | 87.719 | 70.929 | 0.00 | 0.00 | H |
| ATOM | 1807 | HA | GLN | 159 | 115.650 | 88.322 | 73.109 | 0.00 | 0.00 | H |
| ATOM | 1808 | 1HB | GLN | 159 | 114.249 | 90.337 | 73.487 | 0.00 | 0.00 | H |
| ATOM | 1809 | 2HB | GLN | 159 | 113.003 | 89.711 | 72.411 | 0.00 | 0.00 | H |
| ATOM | 1810 | 1HG | GLN | 159 | 114.475 | 89.939 | 70.438 | 0.00 | 0.00 | H |
| ATOM | 1811 | 2HG | GLN | 159 | 115.875 | 90.305 | 71.420 | 0.00 | 0.00 | H |
| ATOM | 1812 | 1HE2 | GLN | 159 | 115.731 | 92.400 | 72.632 | 0.00 | 0.00 | H |
| ATOM | 1813 | 2HE2 | GLN | 159 | 114.805 | 93.732 | 71.792 | 0.00 | 0.00 | H |
| ATOM | 1814 | N | ILE | 160 | 112.749 | 87.059 | 74.203 | 0.00 | 0.00 | N |
| ATOM | 1815 | CA | ILE | 160 | 112.178 | 86.320 | 75.382 | 0.00 | 0.00 | C |
| ATOM | 1816 | C | ILE | 160 | 113.065 | 85.090 | 75.820 | 0.00 | 0.00 | C |
| ATOM | 1817 | O | ILE | 160 | 113.349 | 84.959 | 77.011 | 0.00 | 0.00 | O |
| ATOM | 1818 | CB | ILE | 160 | 110.665 | 85.933 | 75.161 | 0.00 | 0.00 | C |
| ATOM | 1819 | CG2 | ILE | 160 | 110.033 | 85.314 | 76.441 | 0.00 | 0.00 | C |
| ATOM | 1820 | CG1 | ILE | 160 | 109.741 | 87.100 | 74.696 | 0.00 | 0.00 | C |
| ATOM | 1821 | CD1 | ILE | 160 | 108.408 | 86.662 | 74.063 | 0.00 | 0.00 | C |
| ATOM | 1822 | H | ILE | 160 | 112.391 | 86.927 | 73.248 | 0.00 | 0.00 | H |
| ATOM | 1823 | HA | ILE | 160 | 112.195 | 87.020 | 76.242 | 0.00 | 0.00 | H |
| ATOM | 1824 | HB | ILE | 160 | 110.651 | 85.163 | 74.364 | 0.00 | 0.00 | H |
| ATOM | 1825 | 1HG2 | ILE | 160 | 108.989 | 84.988 | 76.278 | 0.00 | 0.00 | H |
| ATOM | 1826 | 2HG2 | ILE | 160 | 110.580 | 84.418 | 76.788 | 0.00 | 0.00 | H |
| ATOM | 1827 | 3HG2 | ILE | 160 | 110.020 | 86.029 | 77.284 | 0.00 | 0.00 | H |
| ATOM | 1828 | 1HG1 | ILE | 160 | 110.259 | 87.714 | 73.936 | 0.00 | 0.00 | H |
| ATOM | 1829 | 2HG1 | ILE | 160 | 109.554 | 87.808 | 75.526 | 0.00 | 0.00 | H |
| ATOM | 1830 | 1HD1 | ILE | 160 | 107.902 | 87.513 | 73.570 | 0.00 | 0.00 | H |
| ATOM | 1831 | 2HD1 | ILE | 160 | 108.545 | 85.892 | 73.285 | 0.00 | 0.00 | H |
| ATOM | 1832 | 3HD1 | ILE | 160 | 107.708 | 86.253 | 74.812 | 0.00 | 0.00 | H |
| ATOM | 1833 | N | VAL | 161 | 113.521 | 84.213 | 74.899 | 0.00 | 0.00 | N |
| ATOM | 1834 | CA | VAL | 161 | 114.529 | 83.137 | 75.205 | 0.00 | 0.00 | C |
| ATOM | 1835 | C | VAL | 161 | 115.907 | 83.685 | 75.750 | 0.00 | 0.00 | C |
| ATOM | 1836 | O | VAL | 161 | 116.400 | 83.167 | 76.752 | 0.00 | 0.00 | O |
| ATOM | 1837 | CB | VAL | 161 | 114.681 | 82.153 | 73.984 | 0.00 | 0.00 | C |
| ATOM | 1838 | CG1 | VAL | 161 | 115.681 | 80.993 | 74.226 | 0.00 | 0.00 | C |
| ATOM | 1839 | CG2 | VAL | 161 | 113.363 | 81.472 | 73.541 | 0.00 | 0.00 | C |
| ATOM | 1840 | H | VAL | 161 | 113.202 | 84.416 | 73.940 | 0.00 | 0.00 | H |
| ATOM | 1841 | HA | VAL | 161 | 114.115 | 82.533 | 76.037 | 0.00 | 0.00 | H |
| ATOM | 1842 | HB | VAL | 161 | 115.060 | 82.747 | 73.126 | 0.00 | 0.00 | H |
| ATOM | 1843 | 1HG1 | VAL | 161 | 115.804 | 80.350 | 73.333 | 0.00 | 0.00 | H |
| ATOM | 1844 | 2HG1 | VAL | 161 | 116.693 | 81.358 | 74.477 | 0.00 | 0.00 | H |
| ATOM | 1845 | 3HG1 | VAL | 161 | 115.368 | 80.335 | 75.059 | 0.00 | 0.00 | H |
| ATOM | 1846 | 1HG2 | VAL | 161 | 113.504 | 80.827 | 72.653 | 0.00 | 0.00 | H |
| ATOM | 1847 | 2HG2 | VAL | 161 | 112.928 | 80.840 | 74.338 | 0.00 | 0.00 | H |
| ATOM | 1848 | 3HG2 | VAL | 161 | 112.588 | 82.210 | 73.260 | 0.00 | 0.00 | H |
| ATOM | 1849 | N | LEU | 162 | 116.504 | 84.730 | 75.145 | 0.00 | 0.00 | N |
| ATOM | 1850 | CA | LEU | 162 | 117.649 | 85.484 | 75.753 | 0.00 | 0.00 | C |
| ATOM | 1851 | C | LEU | 162 | 117.378 | 86.204 | 77.127 | 0.00 | 0.00 | C |
| ATOM | 1852 | O | LEU | 162 | 118.260 | 86.217 | 77.988 | 0.00 | 0.00 | O |
| ATOM | 1853 | CB | LEU | 162 | 118.193 | 86.481 | 74.690 | 0.00 | 0.00 | C |
| ATOM | 1854 | CG | LEU | 162 | 118.906 | 85.886 | 73.448 | 0.00 | 0.00 | C |
| ATOM | 1855 | CD1 | LEU | 162 | 119.135 | 86.980 | 72.393 | 0.00 | 0.00 | C |
| ATOM | 1856 | CD2 | LEU | 162 | 120.255 | 85.243 | 73.811 | 0.00 | 0.00 | C |
| ATOM | 1857 | H | LEU | 162 | 116.028 | 85.052 | 74.289 | 0.00 | 0.00 | H |
| ATOM | 1858 | HA | LEU | 162 | 118.451 | 84.756 | 75.972 | 0.00 | 0.00 | H |
| ATOM | 1859 | 1HB | LEU | 162 | 118.894 | 87.188 | 75.175 | 0.00 | 0.00 | H |
| ATOM | 1860 | 2HB | LEU | 162 | 117.350 | 87.120 | 74.357 | 0.00 | 0.00 | H |
| ATOM | 1861 | HG | LEU | 162 | 118.252 | 85.114 | 72.991 | 0.00 | 0.00 | H |
| ATOM | 1862 | 1HD1 | LEU | 162 | 119.596 | 86.571 | 71.476 | 0.00 | 0.00 | H |
| ATOM | 1863 | 2HD1 | LEU | 162 | 118.185 | 87.454 | 72.080 | 0.00 | 0.00 | H |
| ATOM | 1864 | 3HD1 | LEU | 162 | 119.801 | 87.785 | 72.757 | 0.00 | 0.00 | H |
| ATOM | 1865 | 1HD2 | LEU | 162 | 120.769 | 84.844 | 72.918 | 0.00 | 0.00 | H |
| ATOM | 1866 | 2HD2 | LEU | 162 | 120.944 | 85.963 | 74.291 | 0.00 | 0.00 | H |
| ATOM | 1867 | 3HD2 | LEU | 162 | 120.136 | 84.390 | 74.502 | 0.00 | 0.00 | H |
| ATOM | 1868 | N | GLY | 163 | 116.183 | 86.782 | 77.352 | 1.00 | 0.00 | N |
| ATOM | 1869 | CA | GLY | 163 | 115.710 | 87.181 | 78.711 | 1.00 | 0.00 | C |
| ATOM | 1870 | C | GLY | 163 | 115.582 | 86.064 | 79.778 | 1.00 | 0.00 | C |
| ATOM | 1871 | O | GLY | 163 | 116.190 | 86.183 | 80.837 | 1.00 | 0.00 | O |
| ATOM | 1872 | H | GLY | 163 | 115.552 | 86.757 | 76.537 | 1.00 | 0.00 | H |
| ATOM | 1873 | 1HA | GLY | 163 | 116.368 | 87.972 | 79.118 | 1.00 | 0.00 | H |
| ATOM | 1874 | 2HA | GLY | 163 | 114.724 | 87.667 | 78.616 | 1.00 | 0.00 | H |
| ATOM | 1875 | N | CYS | 164 | 114.850 | 84.974 | 79.497 | 1.00 | 0.00 | N |
| ATOM | 1876 | CA | CYS | 164 | 114.876 | 83.737 | 80.335 | 1.00 | 0.00 | C |
| ATOM | 1877 | C | CYS | 164 | 116.266 | 83.039 | 80.567 | 1.00 | 0.00 | C |
| ATOM | 1878 | O | CYS | 164 | 116.521 | 82.564 | 81.673 | 1.00 | 0.00 | O |
| ATOM | 1879 | CB | CYS | 164 | 113.829 | 82.778 | 79.729 | 1.00 | 0.00 | C |
| ATOM | 1880 | SG | CYS | 164 | 112.154 | 83.509 | 79.788 | 1.00 | 0.00 | S |
| ATOM | 1881 | H | CYS | 164 | 114.400 | 84.992 | 78.570 | 1.00 | 0.00 | H |
| ATOM | 1882 | HA | CYS | 164 | 114.518 | 84.010 | 81.346 | 1.00 | 0.00 | H |
| ATOM | 1883 | 1HB | CYS | 164 | 114.081 | 82.520 | 78.681 | 1.00 | 0.00 | H |
| ATOM | 1884 | 2HB | CYS | 164 | 113.813 | 81.822 | 80.285 | 1.00 | 0.00 | H |
| ATOM | 1885 | HG | CYS | 164 | 112.078 | 83.650 | 81.109 | 1.00 | 0.00 | H |
| ATOM | 1886 | N | GLN | 165 | 117.185 | 83.026 | 79.584 | 1.00 | 0.00 | N |
| ATOM | 1887 | CA | GLN | 165 | 118.630 | 82.717 | 79.812 | 1.00 | 0.00 | C |
| ATOM | 1888 | C | GLN | 165 | 119.377 | 83.597 | 80.884 | 1.00 | 0.00 | C |
| ATOM | 1889 | O | GLN | 165 | 120.137 | 83.048 | 81.686 | 1.00 | 0.00 | O |
| ATOM | 1890 | CB | GLN | 165 | 119.305 | 82.732 | 78.409 | 1.00 | 0.00 | C |
| ATOM | 1891 | CG | GLN | 165 | 120.812 | 82.361 | 78.337 | 1.00 | 0.00 | C |
| ATOM | 1892 | CD | GLN | 165 | 121.171 | 80.925 | 78.714 | 1.00 | 0.00 | C |
| ATOM | 1893 | OE1 | GLN | 165 | 121.176 | 80.021 | 77.889 | 1.00 | 0.00 | O |
| ATOM | 1894 | NE2 | GLN | 165 | 121.483 | 80.662 | 79.958 | 1.00 | 0.00 | N |
| ATOM | 1895 | H | GLN | 165 | 116.831 | 83.380 | 78.683 | 1.00 | 0.00 | H |
| ATOM | 1896 | HA | GLN | 165 | 118.685 | 81.679 | 80.192 | 1.00 | 0.00 | H |
| ATOM | 1897 | 1HB | GLN | 165 | 118.748 | 82.064 | 77.721 | 1.00 | 0.00 | H |
| ATOM | 1898 | 2HB | GLN | 165 | 119.186 | 83.742 | 77.971 | 1.00 | 0.00 | H |
| ATOM | 1899 | 1HG | GLN | 165 | 121.163 | 82.514 | 77.300 | 1.00 | 0.00 | H |
| ATOM | 1900 | 2HG | GLN | 165 | 121.416 | 83.072 | 78.933 | 1.00 | 0.00 | H |
| ATOM | 1901 | 1HE2 | GLN | 165 | 121.348 | 81.425 | 80.632 | 1.00 | 0.00 | H |
| ATOM | 1902 | 2HE2 | GLN | 165 | 121.686 | 79.679 | 80.146 | 1.00 | 0.00 | H |
| ATOM | 1903 | N | TYR | 166 | 119.158 | 84.923 | 80.915 | 0.00 | 0.00 | N |
| ATOM | 1904 | CA | TYR | 166 | 119.568 | 85.789 | 82.059 | 0.00 | 0.00 | C |
| ATOM | 1905 | C | TYR | 166 | 118.771 | 85.539 | 83.393 | 0.00 | 0.00 | C |
| ATOM | 1906 | O | TYR | 166 | 119.399 | 85.300 | 84.425 | 0.00 | 0.00 | O |
| ATOM | 1907 | CB | TYR | 166 | 119.502 | 87.256 | 81.545 | 0.00 | 0.00 | C |
| ATOM | 1908 | CG | TYR | 166 | 120.132 | 88.308 | 82.473 | 0.00 | 0.00 | C |
| ATOM | 1909 | CD1 | TYR | 166 | 121.487 | 88.633 | 82.352 | 0.00 | 0.00 | C |
| ATOM | 1910 | CE1 | TYR | 166 | 122.045 | 89.626 | 83.155 | 0.00 | 0.00 | C |
| ATOM | 1911 | CZ | TYR | 166 | 121.259 | 90.279 | 84.099 | 0.00 | 0.00 | C |
| ATOM | 1912 | OH | TYR | 166 | 121.795 | 91.289 | 84.845 | 0.00 | 0.00 | O |
| ATOM | 1913 | CE2 | TYR | 166 | 119.916 | 89.950 | 84.239 | 0.00 | 0.00 | C |
| ATOM | 1914 | CD2 | TYR | 166 | 119.349 | 88.973 | 83.422 | 0.00 | 0.00 | C |
| ATOM | 1915 | H | TYR | 166 | 118.685 | 85.361 | 80.112 | 1.00 | 0.00 | H |
| ATOM | 1916 | HA | TYR | 166 | 120.631 | 85.572 | 82.293 | 0.00 | 0.00 | H |
| ATOM | 1917 | 1HB | TYR | 166 | 118.451 | 87.533 | 81.332 | 0.00 | 0.00 | H |
| ATOM | 1918 | 2HB | TYR | 166 | 119.994 | 87.330 | 80.555 | 0.00 | 0.00 | H |
| ATOM | 1919 | HD1 | TYR | 166 | 122.106 | 88.133 | 81.622 | 0.00 | 0.00 | H |
| ATOM | 1920 | HE1 | TYR | 166 | 123.087 | 89.891 | 83.046 | 0.00 | 0.00 | H |
| ATOM | 1921 | HH | TYR | 166 | 122.244 | 91.892 | 84.240 | 0.00 | 0.00 | H |
| ATOM | 1922 | HE2 | TYR | 166 | 119.310 | 90.457 | 84.974 | 0.00 | 0.00 | H |
| ATOM | 1923 | HD2 | TYR | 166 | 118.302 | 88.727 | 83.543 | 0.00 | 0.00 | H |
| ATOM | 1924 | N | LEU | 167 | 117.424 | 85.573 | 83.383 | 0.00 | 0.00 | N |
| ATOM | 1925 | CA | LEU | 167 | 116.580 | 85.358 | 84.599 | 0.00 | 0.00 | C |
| ATOM | 1926 | C | LEU | 167 | 116.762 | 83.982 | 85.326 | 0.00 | 0.00 | C |
| ATOM | 1927 | O | LEU | 167 | 116.969 | 83.963 | 86.540 | 0.00 | 0.00 | O |
| ATOM | 1928 | CB | LEU | 167 | 115.083 | 85.622 | 84.256 | 0.00 | 0.00 | C |
| ATOM | 1929 | CG | LEU | 167 | 114.679 | 87.034 | 83.757 | 0.00 | 0.00 | C |
| ATOM | 1930 | CD1 | LEU | 167 | 113.194 | 87.063 | 83.364 | 0.00 | 0.00 | C |
| ATOM | 1931 | CD2 | LEU | 167 | 114.939 | 88.119 | 84.808 | 0.00 | 0.00 | C |
| ATOM | 1932 | H | LEU | 167 | 117.014 | 85.798 | 82.466 | 0.00 | 0.00 | H |
| ATOM | 1933 | HA | LEU | 167 | 116.884 | 86.113 | 85.350 | 0.00 | 0.00 | H |
| ATOM | 1934 | 1HB | LEU | 167 | 114.473 | 85.389 | 85.151 | 0.00 | 0.00 | H |
| ATOM | 1935 | 2HB | LEU | 167 | 114.764 | 84.870 | 83.510 | 0.00 | 0.00 | H |
| ATOM | 1936 | HG | LEU | 167 | 115.274 | 87.289 | 82.858 | 0.00 | 0.00 | H |
| ATOM | 1937 | 1HD1 | LEU | 167 | 112.894 | 88.053 | 82.971 | 0.00 | 0.00 | H |
| ATOM | 1938 | 2HD1 | LEU | 167 | 112.964 | 86.327 | 82.572 | 0.00 | 0.00 | H |
| ATOM | 1939 | 3HD1 | LEU | 167 | 112.532 | 86.847 | 84.222 | 0.00 | 0.00 | H |
| ATOM | 1940 | 1HD2 | LEU | 167 | 114.642 | 89.118 | 84.443 | 0.00 | 0.00 | H |
| ATOM | 1941 | 2HD2 | LEU | 167 | 114.388 | 87.929 | 85.748 | 0.00 | 0.00 | H |
| ATOM | 1942 | 3HD2 | LEU | 167 | 116.011 | 88.196 | 85.072 | 0.00 | 0.00 | H |
| ATOM | 1943 | N | HIS | 168 | 116.757 | 82.845 | 84.609 | 0.00 | 0.00 | N |
| ATOM | 1944 | CA | HIS | 168 | 117.105 | 81.512 | 85.190 | 0.00 | 0.00 | C |
| ATOM | 1945 | C | HIS | 168 | 118.576 | 81.396 | 85.749 | 0.00 | 0.00 | C |
| ATOM | 1946 | O | HIS | 168 | 118.773 | 80.818 | 86.819 | 0.00 | 0.00 | O |
| ATOM | 1947 | CB | HIS | 168 | 116.788 | 80.395 | 84.153 | 0.00 | 0.00 | C |
| ATOM | 1948 | CG | HIS | 168 | 115.353 | 80.235 | 83.617 | 0.00 | 0.00 | C |
| ATOM | 1949 | ND1 | HIS | 168 | 114.199 | 80.809 | 84.143 | 0.00 | 0.00 | N |
| ATOM | 1950 | CE1 | HIS | 168 | 113.278 | 80.323 | 83.255 | 0.00 | 0.00 | C |
| ATOM | 1951 | NE2 | HIS | 168 | 113.675 | 79.497 | 82.239 | 0.00 | 0.00 | N |
| ATOM | 1952 | CD2 | HIS | 168 | 115.034 | 79.451 | 82.498 | 0.00 | 0.00 | C |
| ATOM | 1953 | H | HIS | 168 | 116.578 | 82.972 | 83.603 | 0.00 | 0.00 | H |
| ATOM | 1954 | HA | HIS | 168 | 116.440 | 81.343 | 86.062 | 0.00 | 0.00 | H |
| ATOM | 1955 | 1HB | HIS | 168 | 117.074 | 79.422 | 84.594 | 0.00 | 0.00 | H |
| ATOM | 1956 | 2HB | HIS | 168 | 117.467 | 80.521 | 83.289 | 0.00 | 0.00 | H |
| ATOM | 1957 | HE1 | HIS | 168 | 112.235 | 80.561 | 83.392 | 0.00 | 0.00 | H |
| ATOM | 1958 | HE2 | HIS | 168 | 113.102 | 78.947 | 81.590 | 0.00 | 0.00 | H |
| ATOM | 1959 | HD2 | HIS | 168 | 115.756 | 78.876 | 81.940 | 0.00 | 0.00 | H |
| ATOM | 1960 | N | ARG | 169 | 119.587 | 81.991 | 85.080 | 1.00 | 0.00 | N |
| ATOM | 1961 | CA | ARG | 169 | 120.937 | 82.244 | 85.682 | 1.00 | 0.00 | C |
| ATOM | 1962 | C | ARG | 169 | 120.977 | 83.160 | 86.968 | 1.00 | 0.00 | C |
| ATOM | 1963 | O | ARG | 169 | 121.743 | 82.871 | 87.888 | 1.00 | 0.00 | O |
| ATOM | 1964 | CB | ARG | 169 | 121.828 | 82.766 | 84.521 | 1.00 | 0.00 | C |
| ATOM | 1965 | CG | ARG | 169 | 123.343 | 82.852 | 84.825 | 1.00 | 0.00 | C |
| ATOM | 1966 | CD | ARG | 169 | 124.142 | 83.372 | 83.619 | 1.00 | 0.00 | C |
| ATOM | 1967 | NE | ARG | 169 | 125.581 | 83.393 | 83.981 | 1.00 | 0.00 | N |
| ATOM | 1968 | CZ | ARG | 169 | 126.573 | 83.759 | 83.183 | 1.00 | 0.00 | C |
| ATOM | 1969 | NH1 | ARG | 169 | 126.421 | 84.150 | 81.954 | 1.00 | 0.00 | N |
| ATOM | 1970 | NH2 | ARG | 169 | 127.763 | 83.721 | 83.667 | 1.00 | 0.00 | N |
| ATOM | 1971 | HE | ARG | 169 | 125.830 | 83.099 | 84.936 | 1.00 | 0.00 | H |
| ATOM | 1972 | H | ARG | 169 | 119.258 | 82.535 | 84.276 | 1.00 | 0.00 | H |
| ATOM | 1973 | HA | ARG | 169 | 121.348 | 81.266 | 86.002 | 1.00 | 0.00 | H |
| ATOM | 1974 | 1HB | ARG | 169 | 121.700 | 82.114 | 83.635 | 1.00 | 0.00 | H |
| ATOM | 1975 | 2HB | ARG | 169 | 121.462 | 83.761 | 84.197 | 1.00 | 0.00 | H |
| ATOM | 1976 | 1HG | ARG | 169 | 123.525 | 83.511 | 85.698 | 1.00 | 0.00 | H |
| ATOM | 1977 | 2HG | ARG | 169 | 123.729 | 81.859 | 85.131 | 1.00 | 0.00 | H |
| ATOM | 1978 | 1HD | ARG | 169 | 123.973 | 82.723 | 82.737 | 1.00 | 0.00 | H |
| ATOM | 1979 | 2HD | ARG | 169 | 123.797 | 84.390 | 83.344 | 1.00 | 0.00 | H |
| ATOM | 1980 | 2HH1 | ARG | 169 | 125.445 | 84.158 | 81.655 | 1.00 | 0.00 | H |
| ATOM | 1981 | 1HH1 | ARG | 169 | 127.263 | 84.410 | 81.442 | 1.00 | 0.00 | H |
| ATOM | 1982 | 1HH2 | ARG | 169 | 127.763 | 83.411 | 84.641 | 1.00 | 0.00 | H |
| ATOM | 1983 | 2HH2 | ARG | 169 | 128.535 | 84.007 | 83.067 | 1.00 | 0.00 | H |
| ATOM | 1984 | N | ASN | 170 | 120.138 | 84.208 | 87.078 | 1.00 | 0.00 | N |
| ATOM | 1985 | CA | ASN | 170 | 119.868 | 84.914 | 88.374 | 1.00 | 0.00 | C |
| ATOM | 1986 | C | ASN | 170 | 119.114 | 84.109 | 89.511 | 1.00 | 0.00 | C |
| ATOM | 1987 | O | ASN | 170 | 118.897 | 84.666 | 90.590 | 1.00 | 0.00 | O |
| ATOM | 1988 | CB | ASN | 170 | 119.043 | 86.208 | 88.075 | 1.00 | 0.00 | C |
| ATOM | 1989 | CG | ASN | 170 | 119.476 | 87.253 | 87.043 | 1.00 | 0.00 | C |
| ATOM | 1990 | OD1 | ASN | 170 | 118.647 | 87.828 | 86.350 | 1.00 | 0.00 | O |
| ATOM | 1991 | ND2 | ASN | 170 | 120.734 | 87.585 | 86.923 | 1.00 | 0.00 | N |
| ATOM | 1992 | H | ASN | 170 | 119.600 | 84.408 | 86.222 | 1.00 | 0.00 | H |
| ATOM | 1993 | HA | ASN | 170 | 120.836 | 85.216 | 88.819 | 1.00 | 0.00 | H |
| ATOM | 1994 | 1HB | ASN | 170 | 118.016 | 85.911 | 87.792 | 1.00 | 0.00 | H |
| ATOM | 1995 | 2HB | ASN | 170 | 118.903 | 86.769 | 89.016 | 1.00 | 0.00 | H |
| ATOM | 1996 | 1HD2 | ASN | 170 | 120.898 | 88.227 | 86.142 | 1.00 | 0.00 | H |
| ATOM | 1997 | 2HD2 | ASN | 170 | 121.406 | 86.966 | 87.382 | 1.00 | 0.00 | H |
| ATOM | 1998 | N | ARG | 171 | 118.659 | 82.858 | 89.280 | 1.00 | 0.00 | N |
| ATOM | 1999 | CA | ARG | 171 | 117.640 | 82.151 | 90.127 | 1.00 | 0.00 | C |
| ATOM | 2000 | C | ARG | 171 | 116.210 | 82.824 | 90.209 | 1.00 | 0.00 | C |
| ATOM | 2001 | O | ARG | 171 | 115.514 | 82.730 | 91.223 | 1.00 | 0.00 | O |
| ATOM | 2002 | CB | ARG | 171 | 118.236 | 81.706 | 91.497 | 1.00 | 0.00 | C |
| ATOM | 2003 | CG | ARG | 171 | 119.399 | 80.683 | 91.421 | 1.00 | 0.00 | C |
| ATOM | 2004 | CD | ARG | 171 | 119.917 | 80.277 | 92.807 | 1.00 | 0.00 | C |
| ATOM | 2005 | NE | ARG | 171 | 120.996 | 79.272 | 92.633 | 1.00 | 0.00 | N |
| ATOM | 2006 | CZ | ARG | 171 | 121.728 | 78.749 | 93.607 | 1.00 | 0.00 | C |
| ATOM | 2007 | NH1 | ARG | 171 | 121.590 | 79.037 | 94.866 | 1.00 | 0.00 | N |
| ATOM | 2008 | NH2 | ARG | 171 | 122.632 | 77.900 | 93.271 | 1.00 | 0.00 | N |
| ATOM | 2009 | HE | ARG | 171 | 121.194 | 78.955 | 91.674 | 1.00 | 0.00 | H |
| ATOM | 2010 | H | ARG | 171 | 118.968 | 82.485 | 88.374 | 1.00 | 0.00 | H |
| ATOM | 2011 | HA | ARG | 171 | 117.420 | 81.210 | 89.588 | 1.00 | 0.00 | H |
| ATOM | 2012 | 1HB | ARG | 171 | 118.557 | 82.602 | 92.062 | 1.00 | 0.00 | H |
| ATOM | 2013 | 2HB | ARG | 171 | 117.428 | 81.265 | 92.113 | 1.00 | 0.00 | H |
| ATOM | 2014 | 1HG | ARG | 171 | 119.078 | 79.782 | 90.862 | 1.00 | 0.00 | H |
| ATOM | 2015 | 2HG | ARG | 171 | 120.235 | 81.107 | 90.828 | 1.00 | 0.00 | H |
| ATOM | 2016 | 1HD | ARG | 171 | 120.296 | 81.171 | 93.344 | 1.00 | 0.00 | H |
| ATOM | 2017 | 2HD | ARG | 171 | 119.093 | 79.858 | 93.421 | 1.00 | 0.00 | H |
| ATOM | 2018 | 2HH1 | ARG | 171 | 120.858 | 79.727 | 95.037 | 1.00 | 0.00 | H |
| ATOM | 2019 | 1HH1 | ARG | 171 | 122.219 | 78.576 | 95.520 | 1.00 | 0.00 | H |
| ATOM | 2020 | 1HH2 | ARG | 171 | 122.663 | 77.768 | 92.258 | 1.00 | 0.00 | H |
| ATOM | 2021 | 2HH2 | ARG | 171 | 123.214 | 77.504 | 94.006 | 1.00 | 0.00 | H |
| ATOM | 2022 | N | VAL | 172 | 115.741 | 83.442 | 89.110 | 1.00 | 0.00 | N |
| ATOM | 2023 | CA | VAL | 172 | 114.438 | 84.169 | 89.046 | 1.00 | 0.00 | C |
| ATOM | 2024 | C | VAL | 172 | 113.496 | 83.370 | 88.083 | 1.00 | 0.00 | C |
| ATOM | 2025 | O | VAL | 172 | 113.552 | 83.505 | 86.855 | 1.00 | 0.00 | O |
| ATOM | 2026 | CB | VAL | 172 | 114.661 | 85.671 | 88.631 | 1.00 | 0.00 | C |
| ATOM | 2027 | CG1 | VAL | 172 | 113.351 | 86.468 | 88.415 | 1.00 | 0.00 | C |
| ATOM | 2028 | CG2 | VAL | 172 | 115.491 | 86.480 | 89.657 | 1.00 | 0.00 | C |
| ATOM | 2029 | H | VAL | 172 | 116.426 | 83.513 | 88.344 | 1.00 | 0.00 | H |
| ATOM | 2030 | HA | VAL | 172 | 113.959 | 84.202 | 90.047 | 1.00 | 0.00 | H |
| ATOM | 2031 | HB | VAL | 172 | 115.214 | 85.677 | 87.669 | 1.00 | 0.00 | H |
| ATOM | 2032 | 1HG1 | VAL | 172 | 112.732 | 86.513 | 89.332 | 1.00 | 0.00 | H |
| ATOM | 2033 | 2HG1 | VAL | 172 | 113.547 | 87.510 | 88.101 | 1.00 | 0.00 | H |
| ATOM | 2034 | 3HG1 | VAL | 172 | 112.721 | 86.025 | 87.621 | 1.00 | 0.00 | H |
| ATOM | 2035 | 2HG2 | VAL | 172 | 116.478 | 86.016 | 89.846 | 1.00 | 0.00 | H |
| ATOM | 2036 | 3HG2 | VAL | 172 | 115.697 | 87.510 | 89.309 | 1.00 | 0.00 | H |
| ATOM | 2037 | 1HG2 | VAL | 172 | 114.986 | 86.556 | 90.638 | 1.00 | 0.00 | H |
| ATOM | 2038 | N | ILE | 173 | 112.575 | 82.575 | 88.653 | 0.00 | 0.00 | N |
| ATOM | 2039 | CA | ILE | 173 | 111.447 | 81.954 | 87.886 | 0.00 | 0.00 | C |
| ATOM | 2040 | C | ILE | 173 | 110.383 | 83.074 | 87.611 | 0.00 | 0.00 | C |
| ATOM | 2041 | O | ILE | 173 | 109.709 | 83.512 | 88.544 | 0.00 | 0.00 | O |
| ATOM | 2042 | CB | ILE | 173 | 110.844 | 80.716 | 88.658 | 0.00 | 0.00 | C |
| ATOM | 2043 | CG2 | ILE | 173 | 109.635 | 80.078 | 87.912 | 0.00 | 0.00 | C |
| ATOM | 2044 | CG1 | ILE | 173 | 111.881 | 79.595 | 88.975 | 0.00 | 0.00 | C |
| ATOM | 2045 | CD1 | ILE | 173 | 111.442 | 78.565 | 90.033 | 0.00 | 0.00 | C |
| ATOM | 2046 | H | ILE | 173 | 112.632 | 82.538 | 89.675 | 0.00 | 0.00 | H |
| ATOM | 2047 | HA | ILE | 173 | 111.836 | 81.577 | 86.918 | 0.00 | 0.00 | H |
| ATOM | 2048 | HB | ILE | 173 | 110.467 | 81.102 | 89.627 | 0.00 | 0.00 | H |
| ATOM | 2049 | 1HG2 | ILE | 173 | 109.151 | 79.289 | 88.517 | 0.00 | 0.00 | H |
| ATOM | 2050 | 2HG2 | ILE | 173 | 108.838 | 80.813 | 87.695 | 0.00 | 0.00 | H |
| ATOM | 2051 | 3HG2 | ILE | 173 | 109.929 | 79.606 | 86.958 | 0.00 | 0.00 | H |
| ATOM | 2052 | 1HG1 | ILE | 173 | 112.824 | 80.045 | 89.341 | 0.00 | 0.00 | H |
| ATOM | 2053 | 2HG1 | ILE | 173 | 112.172 | 79.077 | 88.044 | 0.00 | 0.00 | H |
| ATOM | 2054 | 1HD1 | ILE | 173 | 112.255 | 77.853 | 90.265 | 0.00 | 0.00 | H |
| ATOM | 2055 | 2HD1 | ILE | 173 | 111.160 | 79.050 | 90.988 | 0.00 | 0.00 | H |
| ATOM | 2056 | 3HD1 | ILE | 173 | 110.575 | 77.962 | 89.706 | 0.00 | 0.00 | H |
| ATOM | 2057 | N | HIS | 174 | 110.237 | 83.550 | 86.361 | 1.00 | 0.00 | N |
| ATOM | 2058 | CA | HIS | 174 | 109.317 | 84.693 | 86.059 | 1.00 | 0.00 | C |
| ATOM | 2059 | C | HIS | 174 | 107.793 | 84.449 | 86.376 | 1.00 | 0.00 | C |
| ATOM | 2060 | O | HIS | 174 | 107.197 | 85.215 | 87.138 | 1.00 | 0.00 | O |
| ATOM | 2061 | CB | HIS | 174 | 109.621 | 85.167 | 84.608 | 1.00 | 0.00 | C |
| ATOM | 2062 | CG | HIS | 174 | 108.991 | 86.510 | 84.245 | 1.00 | 0.00 | C |
| ATOM | 2063 | ND1 | HIS | 174 | 109.547 | 87.720 | 84.610 | 1.00 | 0.00 | N |
| ATOM | 2064 | CE1 | HIS | 174 | 108.528 | 88.561 | 84.240 | 1.00 | 0.00 | C |
| ATOM | 2065 | NE2 | HIS | 174 | 107.408 | 88.047 | 83.646 | 1.00 | 0.00 | N |
| ATOM | 2066 | CD2 | HIS | 174 | 107.722 | 86.700 | 83.677 | 1.00 | 0.00 | C |
| ATOM | 2067 | H | HIS | 174 | 111.089 | 83.401 | 85.808 | 1.00 | 0.00 | H |
| ATOM | 2068 | HA | HIS | 174 | 109.616 | 85.530 | 86.728 | 1.00 | 0.00 | H |
| ATOM | 2069 | 1HB | HIS | 174 | 110.714 | 85.272 | 84.465 | 1.00 | 0.00 | H |
| ATOM | 2070 | 2HB | HIS | 174 | 109.312 | 84.405 | 83.873 | 1.00 | 0.00 | H |
| ATOM | 2071 | HE1 | HIS | 174 | 108.594 | 89.618 | 84.467 | 1.00 | 0.00 | H |
| ATOM | 2072 | HE2 | HIS | 174 | 106.492 | 88.497 | 83.525 | 1.00 | 0.00 | H |
| ATOM | 2073 | HD2 | HIS | 174 | 107.043 | 85.901 | 83.421 | 1.00 | 0.00 | H |
| ATOM | 2074 | N | ARG | 175 | 107.160 | 83.405 | 85.801 | 0.00 | 0.00 | N |
| ATOM | 2075 | CA | ARG | 175 | 105.738 | 83.015 | 86.076 | 0.00 | 0.00 | C |
| ATOM | 2076 | C | ARG | 175 | 104.626 | 83.935 | 85.455 | 0.00 | 0.00 | C |
| ATOM | 2077 | O | ARG | 175 | 103.710 | 83.427 | 84.802 | 0.00 | 0.00 | O |
| ATOM | 2078 | CB | ARG | 175 | 105.530 | 82.631 | 87.571 | 0.00 | 0.00 | C |
| ATOM | 2079 | CG | ARG | 175 | 104.378 | 81.637 | 87.838 | 0.00 | 0.00 | C |
| ATOM | 2080 | CD | ARG | 175 | 104.296 | 81.222 | 89.315 | 0.00 | 0.00 | C |
| ATOM | 2081 | NE | ARG | 175 | 103.290 | 80.140 | 89.415 | 0.00 | 0.00 | N |
| ATOM | 2082 | CZ | ARG | 175 | 102.838 | 79.593 | 90.531 | 0.00 | 0.00 | C |
| ATOM | 2083 | NH1 | ARG | 175 | 103.214 | 79.931 | 91.729 | 0.00 | 0.00 | N |
| ATOM | 2084 | NH2 | ARG | 175 | 101.966 | 78.668 | 90.400 | 0.00 | 0.00 | N |
| ATOM | 2085 | HE | ARG | 175 | 102.905 | 79.778 | 88.530 | 1.00 | 0.00 | H |
| ATOM | 2086 | H | ARG | 175 | 107.738 | 82.919 | 85.104 | 0.00 | 0.00 | H |
| ATOM | 2087 | HA | ARG | 175 | 105.622 | 82.068 | 85.513 | 0.00 | 0.00 | H |
| ATOM | 2088 | 1HB | ARG | 175 | 105.397 | 83.551 | 88.171 | 0.00 | 0.00 | H |
| ATOM | 2089 | 2HB | ARG | 175 | 106.465 | 82.177 | 87.958 | 0.00 | 0.00 | H |
| ATOM | 2090 | 1HG | ARG | 175 | 104.517 | 80.733 | 87.210 | 0.00 | 0.00 | H |
| ATOM | 2091 | 2HG | ARG | 175 | 103.406 | 82.064 | 87.515 | 0.00 | 0.00 | H |
| ATOM | 2092 | 1HD | ARG | 175 | 104.009 | 82.089 | 89.943 | 0.00 | 0.00 | H |
| ATOM | 2093 | 2HD | ARG | 175 | 105.280 | 80.863 | 89.681 | 0.00 | 0.00 | H |
| ATOM | 2094 | 1HH1 | ARG | 175 | 102.744 | 79.451 | 92.498 | 0.00 | 0.00 | H |
| ATOM | 2095 | 2HH1 | ARG | 175 | 103.889 | 80.695 | 91.751 | 0.00 | 0.00 | H |
| ATOM | 2096 | 1HH2 | ARG | 175 | 101.643 | 78.167 | 91.237 | 0.00 | 0.00 | H |
| ATOM | 2097 | 2HH2 | ARG | 175 | 101.804 | 78.446 | 89.417 | 0.00 | 0.00 | H |
| ATOM | 2098 | N | ASP | 176 | 104.702 | 85.268 | 85.615 | 0.00 | 0.00 | N |
| ATOM | 2099 | CA | ASP | 176 | 103.764 | 86.234 | 84.968 | 0.00 | 0.00 | C |
| ATOM | 2100 | C | ASP | 176 | 104.080 | 86.497 | 83.442 | 0.00 | 0.00 | C |
| ATOM | 2101 | O | ASP | 176 | 104.454 | 87.605 | 83.049 | 0.00 | 0.00 | O |
| ATOM | 2102 | CB | ASP | 176 | 103.818 | 87.483 | 85.894 | 0.00 | 0.00 | C |
| ATOM | 2103 | CG | ASP | 176 | 102.699 | 88.484 | 85.653 | 0.00 | 0.00 | C |
| ATOM | 2104 | OD1 | ASP | 176 | 101.504 | 88.205 | 85.698 | 0.00 | 0.00 | O |
| ATOM | 2105 | OD2 | ASP | 176 | 103.054 | 89.672 | 85.637 | 0.00 | 0.00 | O |
| ATOM | 2106 | H | ASP | 176 | 105.525 | 85.562 | 86.163 | 0.00 | 0.00 | H |
| ATOM | 2107 | HA | ASP | 176 | 102.735 | 85.823 | 85.015 | 0.00 | 0.00 | H |
| ATOM | 2108 | 1HB | ASP | 176 | 104.797 | 87.993 | 85.798 | 0.00 | 0.00 | H |
| ATOM | 2109 | 2HB | ASP | 176 | 103.748 | 87.209 | 86.962 | 0.00 | 0.00 | H |
| ATOM | 2110 | N | LEU | 177 | 103.924 | 85.481 | 82.572 | 0.00 | 0.00 | N |
| ATOM | 2111 | CA | LEU | 177 | 104.341 | 85.556 | 81.137 | 0.00 | 0.00 | C |
| ATOM | 2112 | C | LEU | 177 | 103.112 | 85.411 | 80.177 | 0.00 | 0.00 | C |
| ATOM | 2113 | O | LEU | 177 | 102.648 | 84.308 | 79.872 | 0.00 | 0.00 | O |
| ATOM | 2114 | CB | LEU | 177 | 105.476 | 84.511 | 80.915 | 0.00 | 0.00 | C |
| ATOM | 2115 | CG | LEU | 177 | 106.235 | 84.562 | 79.560 | 0.00 | 0.00 | C |
| ATOM | 2116 | CD1 | LEU | 177 | 106.906 | 85.921 | 79.284 | 0.00 | 0.00 | C |
| ATOM | 2117 | CD2 | LEU | 177 | 107.333 | 83.485 | 79.529 | 0.00 | 0.00 | C |
| ATOM | 2118 | H | LEU | 177 | 103.702 | 84.587 | 83.030 | 0.00 | 0.00 | H |
| ATOM | 2119 | HA | LEU | 177 | 104.805 | 86.542 | 80.937 | 0.00 | 0.00 | H |
| ATOM | 2120 | 1HB | LEU | 177 | 105.054 | 83.496 | 81.051 | 0.00 | 0.00 | H |
| ATOM | 2121 | 2HB | LEU | 177 | 106.224 | 84.609 | 81.725 | 0.00 | 0.00 | H |
| ATOM | 2122 | HG | LEU | 177 | 105.517 | 84.348 | 78.741 | 0.00 | 0.00 | H |
| ATOM | 2123 | 1HD1 | LEU | 177 | 107.505 | 85.902 | 78.353 | 0.00 | 0.00 | H |
| ATOM | 2124 | 2HD1 | LEU | 177 | 106.167 | 86.732 | 79.150 | 0.00 | 0.00 | H |
| ATOM | 2125 | 3HD1 | LEU | 177 | 107.586 | 86.228 | 80.101 | 0.00 | 0.00 | H |
| ATOM | 2126 | 1HD2 | LEU | 177 | 107.855 | 83.463 | 78.556 | 0.00 | 0.00 | H |
| ATOM | 2127 | 2HD2 | LEU | 177 | 108.102 | 83.646 | 80.307 | 0.00 | 0.00 | H |
| ATOM | 2128 | 3HD2 | LEU | 177 | 106.926 | 82.471 | 79.685 | 0.00 | 0.00 | H |
| ATOM | 2129 | N | LYS | 178 | 102.583 | 86.554 | 79.709 | 0.00 | 0.00 | N |
| ATOM | 2130 | CA | LYS | 178 | 101.463 | 86.610 | 78.722 | 0.00 | 0.00 | C |
| ATOM | 2131 | C | LYS | 178 | 101.689 | 87.771 | 77.676 | 0.00 | 0.00 | C |
| ATOM | 2132 | O | LYS | 178 | 102.744 | 88.413 | 77.632 | 0.00 | 0.00 | O |
| ATOM | 2133 | CB | LYS | 178 | 100.114 | 86.647 | 79.527 | 0.00 | 0.00 | C |
| ATOM | 2134 | CG | LYS | 178 | 99.771 | 88.005 | 80.244 | 0.00 | 0.00 | C |
| ATOM | 2135 | CD | LYS | 178 | 98.457 | 88.773 | 79.928 | 0.00 | 0.00 | C |
| ATOM | 2136 | CE | LYS | 178 | 98.363 | 90.078 | 80.774 | 0.00 | 0.00 | C |
| ATOM | 2137 | NZ | LYS | 178 | 97.371 | 91.082 | 80.284 | 0.00 | 0.00 | N |
| ATOM | 2138 | 1HZ | LYS | 178 | 96.334 | 90.864 | 80.118 | 1.00 | 0.00 | H |
| ATOM | 2139 | 2HZ | LYS | 178 | 97.357 | 92.061 | 80.681 | 1.00 | 0.00 | H |
| ATOM | 2140 | 3HZ | LYS | 178 | 97.360 | 91.463 | 79.301 | 1.00 | 0.00 | H |
| ATOM | 2141 | H | LYS | 178 | 103.132 | 87.388 | 79.934 | 0.00 | 0.00 | H |
| ATOM | 2142 | HA | LYS | 178 | 101.469 | 85.678 | 78.119 | 0.00 | 0.00 | H |
| ATOM | 2143 | 1HB | LYS | 178 | 100.182 | 85.941 | 80.379 | 0.00 | 0.00 | H |
| ATOM | 2144 | 2HB | LYS | 178 | 99.318 | 86.209 | 78.895 | 0.00 | 0.00 | H |
| ATOM | 2145 | 1HG | LYS | 178 | 100.614 | 88.710 | 80.085 | 0.00 | 0.00 | H |
| ATOM | 2146 | 2HG | LYS | 178 | 99.828 | 87.850 | 81.343 | 0.00 | 0.00 | H |
| ATOM | 2147 | 1HD | LYS | 178 | 97.563 | 88.150 | 80.119 | 0.00 | 0.00 | H |
| ATOM | 2148 | 2HD | LYS | 178 | 98.407 | 89.015 | 78.850 | 0.00 | 0.00 | H |
| ATOM | 2149 | 1HE | LYS | 178 | 99.353 | 90.577 | 80.849 | 0.00 | 0.00 | H |
| ATOM | 2150 | 2HE | LYS | 178 | 98.140 | 89.833 | 81.834 | 0.00 | 0.00 | H |
| ATOM | 2151 | N | LEU | 179 | 100.665 | 88.092 | 76.867 | 1.00 | 0.00 | N |
| ATOM | 2152 | CA | LEU | 179 | 100.661 | 89.288 | 75.979 | 1.00 | 0.00 | C |
| ATOM | 2153 | C | LEU | 179 | 100.758 | 90.666 | 76.713 | 1.00 | 0.00 | C |
| ATOM | 2154 | O | LEU | 179 | 101.659 | 91.433 | 76.379 | 1.00 | 0.00 | O |
| ATOM | 2155 | CB | LEU | 179 | 99.451 | 89.228 | 75.007 | 1.00 | 0.00 | C |
| ATOM | 2156 | CG | LEU | 179 | 99.463 | 88.132 | 73.904 | 1.00 | 0.00 | C |
| ATOM | 2157 | CD1 | LEU | 179 | 98.968 | 86.763 | 74.404 | 1.00 | 0.00 | C |
| ATOM | 2158 | CD2 | LEU | 179 | 98.584 | 88.550 | 72.712 | 1.00 | 0.00 | C |
| ATOM | 2159 | H | LEU | 179 | 99.878 | 87.438 | 76.888 | 1.00 | 0.00 | H |
| ATOM | 2160 | HA | LEU | 179 | 101.577 | 89.250 | 75.358 | 1.00 | 0.00 | H |
| ATOM | 2161 | 1HB | LEU | 179 | 98.498 | 89.208 | 75.572 | 1.00 | 0.00 | H |
| ATOM | 2162 | 2HB | LEU | 179 | 99.425 | 90.204 | 74.488 | 1.00 | 0.00 | H |
| ATOM | 2163 | HG | LEU | 179 | 100.496 | 88.021 | 73.518 | 1.00 | 0.00 | H |
| ATOM | 2164 | 2HD1 | LEU | 179 | 99.607 | 86.339 | 75.196 | 1.00 | 0.00 | H |
| ATOM | 2165 | 3HD1 | LEU | 179 | 97.932 | 86.801 | 74.789 | 1.00 | 0.00 | H |
| ATOM | 2166 | 1HD1 | LEU | 179 | 98.988 | 86.014 | 73.593 | 1.00 | 0.00 | H |
| ATOM | 2167 | 2HD2 | LEU | 179 | 97.524 | 88.698 | 72.994 | 1.00 | 0.00 | H |
| ATOM | 2168 | 3HD2 | LEU | 179 | 98.936 | 89.497 | 72.259 | 1.00 | 0.00 | H |
| ATOM | 2169 | 1HD2 | LEU | 179 | 98.605 | 87.800 | 71.899 | 1.00 | 0.00 | H |
| ATOM | 2170 | N | GLY | 180 | 99.916 | 90.992 | 77.712 | 1.00 | 0.00 | N |
| ATOM | 2171 | CA | GLY | 180 | 100.099 | 92.232 | 78.527 | 1.00 | 0.00 | C |
| ATOM | 2172 | C | GLY | 180 | 101.529 | 92.588 | 79.036 | 1.00 | 0.00 | C |
| ATOM | 2173 | O | GLY | 180 | 102.069 | 93.647 | 78.710 | 1.00 | 0.00 | O |
| ATOM | 2174 | H | GLY | 180 | 99.039 | 90.461 | 77.744 | 1.00 | 0.00 | H |
| ATOM | 2175 | 1HA | GLY | 180 | 99.812 | 93.071 | 77.875 | 1.00 | 0.00 | H |
| ATOM | 2176 | 2HA | GLY | 180 | 99.394 | 92.319 | 79.367 | 1.00 | 0.00 | H |
| ATOM | 2177 | N | ASN | 181 | 102.174 | 91.645 | 79.727 | 0.00 | 0.00 | N |
| ATOM | 2178 | CA | ASN | 181 | 103.474 | 91.871 | 80.434 | 0.00 | 0.00 | C |
| ATOM | 2179 | C | ASN | 181 | 104.829 | 91.922 | 79.623 | 0.00 | 0.00 | C |
| ATOM | 2180 | O | ASN | 181 | 105.920 | 91.917 | 80.207 | 0.00 | 0.00 | O |
| ATOM | 2181 | CB | ASN | 181 | 103.567 | 90.754 | 81.519 | 0.00 | 0.00 | C |
| ATOM | 2182 | CG | ASN | 181 | 102.405 | 90.641 | 82.505 | 0.00 | 0.00 | C |
| ATOM | 2183 | OD1 | ASN | 181 | 101.450 | 89.904 | 82.283 | 0.00 | 0.00 | O |
| ATOM | 2184 | ND2 | ASN | 181 | 102.429 | 91.382 | 83.570 | 0.00 | 0.00 | N |
| ATOM | 2185 | H | ASN | 181 | 101.561 | 90.881 | 80.025 | 0.00 | 0.00 | H |
| ATOM | 2186 | HA | ASN | 181 | 103.420 | 92.840 | 80.969 | 0.00 | 0.00 | H |
| ATOM | 2187 | 1HB | ASN | 181 | 104.500 | 90.872 | 82.106 | 0.00 | 0.00 | H |
| ATOM | 2188 | 2HB | ASN | 181 | 103.685 | 89.766 | 81.039 | 0.00 | 0.00 | H |
| ATOM | 2189 | 1HD2 | ASN | 181 | 102.021 | 90.827 | 84.338 | 0.00 | 0.00 | H |
| ATOM | 2190 | 2HD2 | ASN | 181 | 103.337 | 91.837 | 83.699 | 0.00 | 0.00 | H |
| ATOM | 2191 | N | LEU | 182 | 104.794 | 92.001 | 78.289 | 0.00 | 0.00 | N |
| ATOM | 2192 | CA | LEU | 182 | 106.015 | 92.163 | 77.449 | 0.00 | 0.00 | C |
| ATOM | 2193 | C | LEU | 182 | 105.850 | 93.462 | 76.595 | 0.00 | 0.00 | C |
| ATOM | 2194 | O | LEU | 182 | 104.848 | 93.643 | 75.902 | 0.00 | 0.00 | O |
| ATOM | 2195 | CB | LEU | 182 | 106.188 | 90.902 | 76.560 | 0.00 | 0.00 | C |
| ATOM | 2196 | CG | LEU | 182 | 106.477 | 89.531 | 77.242 | 0.00 | 0.00 | C |
| ATOM | 2197 | CD1 | LEU | 182 | 106.212 | 88.418 | 76.223 | 0.00 | 0.00 | C |
| ATOM | 2198 | CD2 | LEU | 182 | 107.914 | 89.431 | 77.773 | 0.00 | 0.00 | C |
| ATOM | 2199 | H | LEU | 182 | 103.841 | 92.184 | 77.945 | 0.00 | 0.00 | H |
| ATOM | 2200 | HA | LEU | 182 | 106.927 | 92.272 | 78.072 | 0.00 | 0.00 | H |
| ATOM | 2201 | 1HB | LEU | 182 | 106.998 | 91.117 | 75.844 | 0.00 | 0.00 | H |
| ATOM | 2202 | 2HB | LEU | 182 | 105.275 | 90.809 | 75.941 | 0.00 | 0.00 | H |
| ATOM | 2203 | HG | LEU | 182 | 105.775 | 89.389 | 78.091 | 0.00 | 0.00 | H |
| ATOM | 2204 | 1HD1 | LEU | 182 | 106.449 | 87.416 | 76.624 | 0.00 | 0.00 | H |
| ATOM | 2205 | 2HD1 | LEU | 182 | 105.148 | 88.392 | 75.917 | 0.00 | 0.00 | H |
| ATOM | 2206 | 3HD1 | LEU | 182 | 106.808 | 88.561 | 75.304 | 0.00 | 0.00 | H |
| ATOM | 2207 | 1HD2 | LEU | 182 | 108.125 | 88.438 | 78.211 | 0.00 | 0.00 | H |
| ATOM | 2208 | 2HD2 | LEU | 182 | 108.666 | 89.603 | 76.980 | 0.00 | 0.00 | H |
| ATOM | 2209 | 3HD2 | LEU | 182 | 108.102 | 90.172 | 78.570 | 0.00 | 0.00 | H |
| ATOM | 2210 | N | PHE | 183 | 106.816 | 94.384 | 76.619 | 1.00 | 0.00 | N |
| ATOM | 2211 | CA | PHE | 183 | 106.566 | 95.801 | 76.213 | 1.00 | 0.00 | C |
| ATOM | 2212 | C | PHE | 183 | 107.378 | 96.162 | 74.934 | 1.00 | 0.00 | C |
| ATOM | 2213 | O | PHE | 183 | 108.602 | 96.004 | 74.910 | 1.00 | 0.00 | O |
| ATOM | 2214 | CB | PHE | 183 | 106.879 | 96.721 | 77.437 | 1.00 | 0.00 | C |
| ATOM | 2215 | CG | PHE | 183 | 105.873 | 96.583 | 78.598 | 1.00 | 0.00 | C |
| ATOM | 2216 | CD1 | PHE | 183 | 104.710 | 97.353 | 78.601 | 1.00 | 0.00 | C |
| ATOM | 2217 | CE1 | PHE | 183 | 103.664 | 97.046 | 79.467 | 1.00 | 0.00 | C |
| ATOM | 2218 | CZ | PHE | 183 | 103.797 | 96.002 | 80.375 | 1.00 | 0.00 | C |
| ATOM | 2219 | CE2 | PHE | 183 | 104.987 | 95.288 | 80.441 | 1.00 | 0.00 | C |
| ATOM | 2220 | CD2 | PHE | 183 | 106.024 | 95.575 | 79.557 | 1.00 | 0.00 | C |
| ATOM | 2221 | H | PHE | 183 | 107.542 | 94.195 | 77.325 | 1.00 | 0.00 | H |
| ATOM | 2222 | HA | PHE | 183 | 105.494 | 95.964 | 75.974 | 1.00 | 0.00 | H |
| ATOM | 2223 | 1HB | PHE | 183 | 107.909 | 96.554 | 77.805 | 1.00 | 0.00 | H |
| ATOM | 2224 | 2HB | PHE | 183 | 106.890 | 97.776 | 77.103 | 1.00 | 0.00 | H |
| ATOM | 2225 | HD1 | PHE | 183 | 104.614 | 98.187 | 77.924 | 1.00 | 0.00 | H |
| ATOM | 2226 | HE1 | PHE | 183 | 102.761 | 97.638 | 79.466 | 1.00 | 0.00 | H |
| ATOM | 2227 | HZ | PHE | 183 | 102.988 | 95.770 | 81.054 | 1.00 | 0.00 | H |
| ATOM | 2228 | HE2 | PHE | 183 | 105.094 | 94.496 | 81.163 | 1.00 | 0.00 | H |
| ATOM | 2229 | HD2 | PHE | 183 | 106.917 | 94.968 | 79.579 | 1.00 | 0.00 | H |
| ATOM | 2230 | N | LEU | 184 | 106.730 | 96.690 | 73.873 | 1.00 | 0.00 | N |
| ATOM | 2231 | CA | LEU | 184 | 107.416 | 97.309 | 72.691 | 1.00 | 0.00 | C |
| ATOM | 2232 | C | LEU | 184 | 108.064 | 98.692 | 73.028 | 1.00 | 0.00 | C |
| ATOM | 2233 | O | LEU | 184 | 107.333 | 99.660 | 73.283 | 1.00 | 0.00 | O |
| ATOM | 2234 | CB | LEU | 184 | 106.409 | 97.532 | 71.512 | 1.00 | 0.00 | C |
| ATOM | 2235 | CG | LEU | 184 | 106.138 | 96.389 | 70.506 | 1.00 | 0.00 | C |
| ATOM | 2236 | CD1 | LEU | 184 | 105.052 | 96.846 | 69.514 | 1.00 | 0.00 | C |
| ATOM | 2237 | CD2 | LEU | 184 | 107.372 | 96.012 | 69.667 | 1.00 | 0.00 | C |
| ATOM | 2238 | H | LEU | 184 | 105.739 | 96.918 | 74.050 | 1.00 | 0.00 | H |
| ATOM | 2239 | HA | LEU | 184 | 108.217 | 96.630 | 72.340 | 1.00 | 0.00 | H |
| ATOM | 2240 | 1HB | LEU | 184 | 105.456 | 97.912 | 71.919 | 1.00 | 0.00 | H |
| ATOM | 2241 | 2HB | LEU | 184 | 106.746 | 98.394 | 70.898 | 1.00 | 0.00 | H |
| ATOM | 2242 | HG | LEU | 184 | 105.777 | 95.497 | 71.059 | 1.00 | 0.00 | H |
| ATOM | 2243 | 2HD1 | LEU | 184 | 104.139 | 97.190 | 70.031 | 1.00 | 0.00 | H |
| ATOM | 2244 | 3HD1 | LEU | 184 | 105.390 | 97.690 | 68.883 | 1.00 | 0.00 | H |
| ATOM | 2245 | 1HD1 | LEU | 184 | 104.757 | 96.030 | 68.831 | 1.00 | 0.00 | H |
| ATOM | 2246 | 2HD2 | LEU | 184 | 107.705 | 96.837 | 69.013 | 1.00 | 0.00 | H |
| ATOM | 2247 | 3HD2 | LEU | 184 | 108.239 | 95.724 | 70.287 | 1.00 | 0.00 | H |
| ATOM | 2248 | 1HD2 | LEU | 184 | 107.163 | 95.145 | 69.011 | 1.00 | 0.00 | H |
| ATOM | 2249 | N | ASN | 185 | 109.403 | 98.774 | 73.058 | 1.00 | 0.00 | N |
| ATOM | 2250 | CA | ASN | 185 | 110.106 | 100.086 | 73.132 | 1.00 | 0.00 | C |
| ATOM | 2251 | C | ASN | 185 | 110.042 | 100.930 | 71.806 | 1.00 | 0.00 | C |
| ATOM | 2252 | O | ASN | 185 | 109.564 | 100.485 | 70.758 | 1.00 | 0.00 | O |
| ATOM | 2253 | CB | ASN | 185 | 111.522 | 99.846 | 73.732 | 1.00 | 0.00 | C |
| ATOM | 2254 | CG | ASN | 185 | 112.608 | 99.236 | 72.837 | 1.00 | 0.00 | C |
| ATOM | 2255 | OD1 | ASN | 185 | 112.593 | 99.298 | 71.615 | 1.00 | 0.00 | O |
| ATOM | 2256 | ND2 | ASN | 185 | 113.632 | 98.680 | 73.422 | 1.00 | 0.00 | N |
| ATOM | 2257 | H | ASN | 185 | 109.889 | 97.875 | 72.930 | 1.00 | 0.00 | H |
| ATOM | 2258 | HA | ASN | 185 | 109.576 | 100.716 | 73.879 | 1.00 | 0.00 | H |
| ATOM | 2259 | 1HB | ASN | 185 | 111.919 | 100.816 | 74.083 | 1.00 | 0.00 | H |
| ATOM | 2260 | 2HB | ASN | 185 | 111.419 | 99.249 | 74.657 | 1.00 | 0.00 | H |
| ATOM | 2261 | 1HD2 | ASN | 185 | 114.326 | 98.305 | 72.770 | 1.00 | 0.00 | H |
| ATOM | 2262 | 2HD2 | ASN | 185 | 113.566 | 98.525 | 74.431 | 1.00 | 0.00 | H |
| ATOM | 2263 | N | GLU | 186 | 110.562 | 102.162 | 71.853 | 1.00 | 0.00 | N |
| ATOM | 2264 | CA | GLU | 186 | 110.544 | 103.106 | 70.696 | 1.00 | 0.00 | C |
| ATOM | 2265 | C | GLU | 186 | 111.399 | 102.718 | 69.427 | 1.00 | 0.00 | C |
| ATOM | 2266 | O | GLU | 186 | 111.099 | 103.204 | 68.337 | 1.00 | 0.00 | O |
| ATOM | 2267 | CB | GLU | 186 | 110.887 | 104.518 | 71.262 | 1.00 | 0.00 | C |
| ATOM | 2268 | CG | GLU | 186 | 109.836 | 105.203 | 72.195 | 1.00 | 0.00 | C |
| ATOM | 2269 | CD | GLU | 186 | 109.642 | 104.655 | 73.619 | 1.00 | 0.00 | C |
| ATOM | 2270 | OE1 | GLU | 186 | 110.476 | 103.990 | 74.228 | 1.00 | 0.00 | O |
| ATOM | 2271 | OE2 | GLU | 186 | 108.515 | 104.880 | 74.100 | 1.00 | 0.00 | O |
| ATOM | 2272 | H | GLU | 186 | 110.799 | 102.491 | 72.797 | 1.00 | 0.00 | H |
| ATOM | 2273 | HA | GLU | 186 | 109.504 | 103.157 | 70.315 | 1.00 | 0.00 | H |
| ATOM | 2274 | 1HB | GLU | 186 | 111.880 | 104.501 | 71.757 | 1.00 | 0.00 | H |
| ATOM | 2275 | 2HB | GLU | 186 | 111.034 | 105.199 | 70.400 | 1.00 | 0.00 | H |
| ATOM | 2276 | 1HG | GLU | 186 | 110.104 | 106.268 | 72.311 | 1.00 | 0.00 | H |
| ATOM | 2277 | 2HG | GLU | 186 | 108.852 | 105.215 | 71.690 | 1.00 | 0.00 | H |
| ATOM | 2278 | N | ASP | 187 | 112.403 | 101.825 | 69.536 | 1.00 | 0.00 | N |
| ATOM | 2279 | CA | ASP | 187 | 112.999 | 101.111 | 68.358 | 1.00 | 0.00 | C |
| ATOM | 2280 | C | ASP | 187 | 112.360 | 99.707 | 67.989 | 1.00 | 0.00 | C |
| ATOM | 2281 | O | ASP | 187 | 112.928 | 98.972 | 67.175 | 1.00 | 0.00 | O |
| ATOM | 2282 | CB | ASP | 187 | 114.526 | 100.991 | 68.633 | 1.00 | 0.00 | C |
| ATOM | 2283 | CG | ASP | 187 | 115.313 | 102.299 | 68.593 | 1.00 | 0.00 | C |
| ATOM | 2284 | OD1 | ASP | 187 | 115.655 | 102.860 | 67.559 | 1.00 | 0.00 | O |
| ATOM | 2285 | OD2 | ASP | 187 | 115.596 | 102.774 | 69.835 | 1.00 | 0.00 | O |
| ATOM | 2286 | H | ASP | 187 | 112.486 | 101.438 | 70.483 | 1.00 | 0.00 | H |
| ATOM | 2287 | HA | ASP | 187 | 112.875 | 101.719 | 67.437 | 1.00 | 0.00 | H |
| ATOM | 2288 | 1HB | ASP | 187 | 114.712 | 100.468 | 69.590 | 1.00 | 0.00 | H |
| ATOM | 2289 | 2HB | ASP | 187 | 114.992 | 100.341 | 67.870 | 1.00 | 0.00 | H |
| ATOM | 2290 | N | LEU | 188 | 111.182 | 99.345 | 68.536 | 1.00 | 0.00 | N |
| ATOM | 2291 | CA | LEU | 188 | 110.435 | 98.074 | 68.258 | 1.00 | 0.00 | C |
| ATOM | 2292 | C | LEU | 188 | 111.031 | 96.740 | 68.850 | 1.00 | 0.00 | C |
| ATOM | 2293 | O | LEU | 188 | 110.984 | 95.685 | 68.210 | 1.00 | 0.00 | O |
| ATOM | 2294 | CB | LEU | 188 | 109.963 | 97.964 | 66.770 | 1.00 | 0.00 | C |
| ATOM | 2295 | CG | LEU | 188 | 109.099 | 99.113 | 66.185 | 1.00 | 0.00 | C |
| ATOM | 2296 | CD1 | LEU | 188 | 108.906 | 98.904 | 64.675 | 1.00 | 0.00 | C |
| ATOM | 2297 | CD2 | LEU | 188 | 107.720 | 99.221 | 66.860 | 1.00 | 0.00 | C |
| ATOM | 2298 | H | LEU | 188 | 110.807 | 100.045 | 69.191 | 1.00 | 0.00 | H |
| ATOM | 2299 | HA | LEU | 188 | 109.504 | 98.180 | 68.844 | 1.00 | 0.00 | H |
| ATOM | 2300 | 1HB | LEU | 188 | 110.866 | 97.831 | 66.144 | 1.00 | 0.00 | H |
| ATOM | 2301 | 2HB | LEU | 188 | 109.408 | 97.014 | 66.643 | 1.00 | 0.00 | H |
| ATOM | 2302 | HG | LEU | 188 | 109.638 | 100.073 | 66.327 | 1.00 | 0.00 | H |
| ATOM | 2303 | 2HD1 | LEU | 188 | 109.876 | 98.865 | 64.143 | 1.00 | 0.00 | H |
| ATOM | 2304 | 3HD1 | LEU | 188 | 108.372 | 97.962 | 64.446 | 1.00 | 0.00 | H |
| ATOM | 2305 | 1HD1 | LEU | 188 | 108.330 | 99.729 | 64.216 | 1.00 | 0.00 | H |
| ATOM | 2306 | 2HD2 | LEU | 188 | 107.141 | 98.282 | 66.782 | 1.00 | 0.00 | H |
| ATOM | 2307 | 3HD2 | LEU | 188 | 107.809 | 99.465 | 67.935 | 1.00 | 0.00 | H |
| ATOM | 2308 | 1HD2 | LEU | 188 | 107.108 | 100.025 | 66.411 | 1.00 | 0.00 | H |
| ATOM | 2309 | N | GLU | 189 | 111.538 | 96.755 | 70.097 | 1.00 | 0.00 | N |
| ATOM | 2310 | CA | GLU | 189 | 112.173 | 95.567 | 70.749 | 1.00 | 0.00 | C |
| ATOM | 2311 | C | GLU | 189 | 111.436 | 95.126 | 72.067 | 1.00 | 0.00 | C |
| ATOM | 2312 | O | GLU | 189 | 110.715 | 95.897 | 72.708 | 1.00 | 0.00 | O |
| ATOM | 2313 | CB | GLU | 189 | 113.678 | 95.893 | 71.008 | 1.00 | 0.00 | C |
| ATOM | 2314 | CG | GLU | 189 | 114.656 | 95.681 | 69.821 | 1.00 | 0.00 | C |
| ATOM | 2315 | CD | GLU | 189 | 114.692 | 96.837 | 68.832 | 1.00 | 0.00 | C |
| ATOM | 2316 | OE1 | GLU | 189 | 115.281 | 97.892 | 69.046 | 1.00 | 0.00 | O |
| ATOM | 2317 | OE2 | GLU | 189 | 113.970 | 96.585 | 67.707 | 1.00 | 0.00 | O |
| ATOM | 2318 | H | GLU | 189 | 111.640 | 97.704 | 70.480 | 1.00 | 0.00 | H |
| ATOM | 2319 | HA | GLU | 189 | 112.131 | 94.686 | 70.075 | 1.00 | 0.00 | H |
| ATOM | 2320 | 1HB | GLU | 189 | 113.790 | 96.916 | 71.405 | 1.00 | 0.00 | H |
| ATOM | 2321 | 2HB | GLU | 189 | 114.042 | 95.267 | 71.843 | 1.00 | 0.00 | H |
| ATOM | 2322 | 1HG | GLU | 189 | 115.685 | 95.563 | 70.206 | 1.00 | 0.00 | H |
| ATOM | 2323 | 2HG | GLU | 189 | 114.440 | 94.731 | 69.294 | 1.00 | 0.00 | H |
| ATOM | 2324 | N | VAL | 190 | 111.664 | 93.866 | 72.489 | 1.00 | 0.00 | N |
| ATOM | 2325 | CA | VAL | 190 | 111.100 | 93.293 | 73.754 | 1.00 | 0.00 | C |
| ATOM | 2326 | C | VAL | 190 | 111.790 | 93.877 | 75.036 | 1.00 | 0.00 | C |
| ATOM | 2327 | O | VAL | 190 | 112.974 | 93.637 | 75.298 | 1.00 | 0.00 | O |
| ATOM | 2328 | CB | VAL | 190 | 111.177 | 91.721 | 73.750 | 1.00 | 0.00 | C |
| ATOM | 2329 | CG1 | VAL | 190 | 110.578 | 91.047 | 75.010 | 1.00 | 0.00 | C |
| ATOM | 2330 | CG2 | VAL | 190 | 110.495 | 91.026 | 72.548 | 1.00 | 0.00 | C |
| ATOM | 2331 | H | VAL | 190 | 112.242 | 93.298 | 71.855 | 1.00 | 0.00 | H |
| ATOM | 2332 | HA | VAL | 190 | 110.025 | 93.542 | 73.801 | 1.00 | 0.00 | H |
| ATOM | 2333 | HB | VAL | 190 | 112.252 | 91.463 | 73.722 | 1.00 | 0.00 | H |
| ATOM | 2334 | 1HG1 | VAL | 190 | 109.496 | 91.252 | 75.118 | 1.00 | 0.00 | H |
| ATOM | 2335 | 2HG1 | VAL | 190 | 110.706 | 89.949 | 74.991 | 1.00 | 0.00 | H |
| ATOM | 2336 | 3HG1 | VAL | 190 | 111.068 | 91.390 | 75.941 | 1.00 | 0.00 | H |
| ATOM | 2337 | 2HG2 | VAL | 190 | 110.881 | 91.382 | 71.576 | 1.00 | 0.00 | H |
| ATOM | 2338 | 3HG2 | VAL | 190 | 110.658 | 89.933 | 72.556 | 1.00 | 0.00 | H |
| ATOM | 2339 | 1HG2 | VAL | 190 | 109.401 | 91.178 | 72.544 | 1.00 | 0.00 | H |
| ATOM | 2340 | N | LYS | 191 | 110.996 | 94.554 | 75.872 | 1.00 | 0.00 | N |
| ATOM | 2341 | CA | LYS | 191 | 111.369 | 94.885 | 77.269 | 1.00 | 0.00 | C |
| ATOM | 2342 | C | LYS | 191 | 110.423 | 94.113 | 78.258 | 1.00 | 0.00 | C |
| ATOM | 2343 | O | LYS | 191 | 109.215 | 94.364 | 78.306 | 1.00 | 0.00 | O |
| ATOM | 2344 | CB | LYS | 191 | 111.259 | 96.423 | 77.448 | 1.00 | 0.00 | C |
| ATOM | 2345 | CG | LYS | 191 | 112.257 | 97.343 | 76.706 | 1.00 | 0.00 | C |
| ATOM | 2346 | CD | LYS | 191 | 113.703 | 97.231 | 77.229 | 1.00 | 0.00 | C |
| ATOM | 2347 | CE | LYS | 191 | 114.545 | 98.495 | 76.998 | 1.00 | 0.00 | C |
| ATOM | 2348 | NZ | LYS | 191 | 115.860 | 98.307 | 77.636 | 1.00 | 0.00 | N |
| ATOM | 2349 | 1HZ | LYS | 191 | 116.437 | 99.148 | 77.489 | 1.00 | 0.00 | H |
| ATOM | 2350 | 2HZ | LYS | 191 | 115.733 | 98.151 | 78.646 | 1.00 | 0.00 | H |
| ATOM | 2351 | 3HZ | LYS | 191 | 116.331 | 97.491 | 77.220 | 1.00 | 0.00 | H |
| ATOM | 2352 | H | LYS | 191 | 110.119 | 94.893 | 75.451 | 1.00 | 0.00 | H |
| ATOM | 2353 | HA | LYS | 191 | 112.417 | 94.601 | 77.489 | 1.00 | 0.00 | H |
| ATOM | 2354 | 1HB | LYS | 191 | 110.229 | 96.745 | 77.190 | 1.00 | 0.00 | H |
| ATOM | 2355 | 2HB | LYS | 191 | 111.347 | 96.649 | 78.525 | 1.00 | 0.00 | H |
| ATOM | 2356 | 1HG | LYS | 191 | 112.222 | 97.146 | 75.616 | 1.00 | 0.00 | H |
| ATOM | 2357 | 2HG | LYS | 191 | 111.899 | 98.383 | 76.832 | 1.00 | 0.00 | H |
| ATOM | 2358 | 1HD | LYS | 191 | 113.684 | 97.025 | 78.314 | 1.00 | 0.00 | H |
| ATOM | 2359 | 2HD | LYS | 191 | 114.192 | 96.343 | 76.780 | 1.00 | 0.00 | H |
| ATOM | 2360 | 1HE | LYS | 191 | 114.675 | 98.701 | 75.921 | 1.00 | 0.00 | H |
| ATOM | 2361 | 2HE | LYS | 191 | 114.048 | 99.385 | 77.436 | 1.00 | 0.00 | H |
| ATOM | 2362 | N | ILE | 192 | 110.954 | 93.189 | 79.078 | 1.00 | 0.00 | N |
| ATOM | 2363 | CA | ILE | 192 | 110.156 | 92.448 | 80.114 | 1.00 | 0.00 | C |
| ATOM | 2364 | C | ILE | 192 | 109.807 | 93.417 | 81.306 | 1.00 | 0.00 | C |
| ATOM | 2365 | O | ILE | 192 | 110.706 | 93.999 | 81.924 | 1.00 | 0.00 | O |
| ATOM | 2366 | CB | ILE | 192 | 110.905 | 91.140 | 80.577 | 1.00 | 0.00 | C |
| ATOM | 2367 | CG1 | ILE | 192 | 111.266 | 90.151 | 79.424 | 1.00 | 0.00 | C |
| ATOM | 2368 | CG2 | ILE | 192 | 110.090 | 90.351 | 81.640 | 1.00 | 0.00 | C |
| ATOM | 2369 | CD1 | ILE | 192 | 112.362 | 89.124 | 79.760 | 1.00 | 0.00 | C |
| ATOM | 2370 | H | ILE | 192 | 111.953 | 92.986 | 78.923 | 1.00 | 0.00 | H |
| ATOM | 2371 | HA | ILE | 192 | 109.209 | 92.115 | 79.643 | 1.00 | 0.00 | H |
| ATOM | 2372 | HB | ILE | 192 | 111.853 | 91.469 | 81.050 | 1.00 | 0.00 | H |
| ATOM | 2373 | 1HG1 | ILE | 192 | 110.364 | 89.623 | 79.069 | 1.00 | 0.00 | H |
| ATOM | 2374 | 2HG1 | ILE | 192 | 111.620 | 90.711 | 78.536 | 1.00 | 0.00 | H |
| ATOM | 2375 | 2HG2 | ILE | 192 | 109.842 | 90.969 | 82.524 | 1.00 | 0.00 | H |
| ATOM | 2376 | 3HG2 | ILE | 192 | 109.133 | 89.964 | 81.241 | 1.00 | 0.00 | H |
| ATOM | 2377 | 1HG2 | ILE | 192 | 110.648 | 89.485 | 82.042 | 1.00 | 0.00 | H |
| ATOM | 2378 | 2HD1 | ILE | 192 | 113.309 | 89.619 | 80.049 | 1.00 | 0.00 | H |
| ATOM | 2379 | 3HD1 | ILE | 192 | 112.073 | 88.451 | 80.588 | 1.00 | 0.00 | H |
| ATOM | 2380 | 1HD1 | ILE | 192 | 112.580 | 88.482 | 78.887 | 1.00 | 0.00 | H |
| ATOM | 2381 | N | GLY | 193 | 108.501 | 93.593 | 81.560 | 1.00 | 0.00 | N |
| ATOM | 2382 | CA | GLY | 193 | 107.981 | 94.465 | 82.644 | 1.00 | 0.00 | C |
| ATOM | 2383 | C | GLY | 193 | 106.797 | 93.807 | 83.394 | 1.00 | 0.00 | C |
| ATOM | 2384 | O | GLY | 193 | 106.069 | 92.963 | 82.867 | 1.00 | 0.00 | O |
| ATOM | 2385 | H | GLY | 193 | 107.857 | 92.935 | 81.093 | 1.00 | 0.00 | H |
| ATOM | 2386 | 1HA | GLY | 193 | 108.780 | 94.742 | 83.361 | 1.00 | 0.00 | H |
| ATOM | 2387 | 2HA | GLY | 193 | 107.620 | 95.417 | 82.214 | 1.00 | 0.00 | H |
| ATOM | 2388 | N | ASP | 194 | 106.620 | 94.204 | 84.651 | 0.00 | 0.00 | N |
| ATOM | 2389 | CA | ASP | 194 | 105.779 | 93.470 | 85.637 | 0.00 | 0.00 | C |
| ATOM | 2390 | C | ASP | 194 | 106.338 | 92.050 | 86.040 | 0.00 | 0.00 | C |
| ATOM | 2391 | O | ASP | 194 | 106.282 | 91.059 | 85.311 | 0.00 | 0.00 | O |
| ATOM | 2392 | CB | ASP | 194 | 104.243 | 93.694 | 85.490 | 0.00 | 0.00 | C |
| ATOM | 2393 | CG | ASP | 194 | 103.710 | 94.969 | 86.165 | 0.00 | 0.00 | C |
| ATOM | 2394 | OD1 | ASP | 194 | 102.591 | 94.994 | 86.709 | 0.00 | 0.00 | O |
| ATOM | 2395 | OD2 | ASP | 194 | 104.420 | 95.994 | 86.207 | 0.00 | 0.00 | O |
| ATOM | 2396 | H | ASP | 194 | 107.243 | 94.956 | 84.952 | 0.00 | 0.00 | H |
| ATOM | 2397 | HA | ASP | 194 | 106.000 | 94.081 | 86.538 | 0.00 | 0.00 | H |
| ATOM | 2398 | 1HB | ASP | 194 | 103.702 | 92.835 | 85.929 | 0.00 | 0.00 | H |
| ATOM | 2399 | 2HB | ASP | 194 | 103.950 | 93.706 | 84.427 | 0.00 | 0.00 | H |
| ATOM | 2400 | N | PHE | 195 | 106.971 | 92.028 | 87.220 | 0.00 | 0.00 | N |
| ATOM | 2401 | CA | PHE | 195 | 107.659 | 90.844 | 87.809 | 0.00 | 0.00 | C |
| ATOM | 2402 | C | PHE | 195 | 106.955 | 90.346 | 89.118 | 0.00 | 0.00 | C |
| ATOM | 2403 | O | PHE | 195 | 107.603 | 89.677 | 89.928 | 0.00 | 0.00 | O |
| ATOM | 2404 | CB | PHE | 195 | 109.123 | 91.329 | 88.053 | 0.00 | 0.00 | C |
| ATOM | 2405 | CG | PHE | 195 | 110.056 | 91.346 | 86.828 | 0.00 | 0.00 | C |
| ATOM | 2406 | CD1 | PHE | 195 | 110.856 | 90.237 | 86.538 | 0.00 | 0.00 | C |
| ATOM | 2407 | CE1 | PHE | 195 | 111.733 | 90.267 | 85.457 | 0.00 | 0.00 | C |
| ATOM | 2408 | CZ | PHE | 195 | 111.821 | 91.406 | 84.661 | 0.00 | 0.00 | C |
| ATOM | 2409 | CE2 | PHE | 195 | 111.028 | 92.514 | 84.943 | 0.00 | 0.00 | C |
| ATOM | 2410 | CD2 | PHE | 195 | 110.140 | 92.480 | 86.015 | 0.00 | 0.00 | C |
| ATOM | 2411 | H | PHE | 195 | 106.824 | 92.881 | 87.779 | 0.00 | 0.00 | H |
| ATOM | 2412 | HA | PHE | 195 | 107.709 | 89.937 | 87.162 | 0.00 | 0.00 | H |
| ATOM | 2413 | 1HB | PHE | 195 | 109.585 | 90.688 | 88.817 | 0.00 | 0.00 | H |
| ATOM | 2414 | 2HB | PHE | 195 | 109.136 | 92.321 | 88.548 | 0.00 | 0.00 | H |
| ATOM | 2415 | HD1 | PHE | 195 | 110.792 | 89.343 | 87.141 | 0.00 | 0.00 | H |
| ATOM | 2416 | HE1 | PHE | 195 | 112.334 | 89.398 | 85.231 | 0.00 | 0.00 | H |
| ATOM | 2417 | HZ | PHE | 195 | 112.500 | 91.427 | 83.821 | 0.00 | 0.00 | H |
| ATOM | 2418 | HE2 | PHE | 195 | 111.092 | 93.402 | 84.334 | 0.00 | 0.00 | H |
| ATOM | 2419 | HD2 | PHE | 195 | 109.522 | 93.343 | 86.222 | 0.00 | 0.00 | H |
| ATOM | 2420 | N | GLY | 196 | 105.650 | 90.587 | 89.364 | 0.00 | 0.00 | N |
| ATOM | 2421 | CA | GLY | 196 | 105.014 | 90.327 | 90.689 | 0.00 | 0.00 | C |
| ATOM | 2422 | C | GLY | 196 | 104.105 | 89.088 | 90.820 | 0.00 | 0.00 | C |
| ATOM | 2423 | O | GLY | 196 | 102.957 | 89.227 | 91.239 | 0.00 | 0.00 | O |
| ATOM | 2424 | H | GLY | 196 | 105.226 | 91.180 | 88.638 | 0.00 | 0.00 | H |
| ATOM | 2425 | 1HA | GLY | 196 | 104.419 | 91.219 | 90.959 | 0.00 | 0.00 | H |
| ATOM | 2426 | 2HA | GLY | 196 | 105.762 | 90.262 | 91.504 | 0.00 | 0.00 | H |
| ATOM | 2427 | N | LEU | 197 | 104.634 | 87.889 | 90.543 | 1.00 | 0.00 | N |
| ATOM | 2428 | CA | LEU | 197 | 104.022 | 86.603 | 91.006 | 1.00 | 0.00 | C |
| ATOM | 2429 | C | LEU | 197 | 105.151 | 85.715 | 91.663 | 1.00 | 0.00 | C |
| ATOM | 2430 | O | LEU | 197 | 105.867 | 86.202 | 92.541 | 1.00 | 0.00 | O |
| ATOM | 2431 | CB | LEU | 197 | 103.184 | 85.943 | 89.856 | 1.00 | 0.00 | C |
| ATOM | 2432 | CG | LEU | 197 | 101.879 | 86.642 | 89.386 | 1.00 | 0.00 | C |
| ATOM | 2433 | CD1 | LEU | 197 | 101.258 | 85.871 | 88.209 | 1.00 | 0.00 | C |
| ATOM | 2434 | CD2 | LEU | 197 | 100.819 | 86.751 | 90.496 | 1.00 | 0.00 | C |
| ATOM | 2435 | H | LEU | 197 | 105.635 | 87.961 | 90.323 | 1.00 | 0.00 | H |
| ATOM | 2436 | HA | LEU | 197 | 103.337 | 86.799 | 91.855 | 1.00 | 0.00 | H |
| ATOM | 2437 | 1HB | LEU | 197 | 103.851 | 85.796 | 88.985 | 1.00 | 0.00 | H |
| ATOM | 2438 | 2HB | LEU | 197 | 102.903 | 84.921 | 90.170 | 1.00 | 0.00 | H |
| ATOM | 2439 | HG | LEU | 197 | 102.132 | 87.665 | 89.038 | 1.00 | 0.00 | H |
| ATOM | 2440 | 2HD1 | LEU | 197 | 101.973 | 85.727 | 87.381 | 1.00 | 0.00 | H |
| ATOM | 2441 | 3HD1 | LEU | 197 | 100.903 | 84.864 | 88.502 | 1.00 | 0.00 | H |
| ATOM | 2442 | 1HD1 | LEU | 197 | 100.391 | 86.412 | 87.783 | 1.00 | 0.00 | H |
| ATOM | 2443 | 2HD2 | LEU | 197 | 100.543 | 85.768 | 90.917 | 1.00 | 0.00 | H |
| ATOM | 2444 | 3HD2 | LEU | 197 | 101.167 | 87.379 | 91.337 | 1.00 | 0.00 | H |
| ATOM | 2445 | 1HD2 | LEU | 197 | 99.890 | 87.224 | 90.127 | 1.00 | 0.00 | H |
| ATOM | 2446 | N | ALA | 198 | 105.281 | 84.402 | 91.365 | 0.00 | 0.00 | N |
| ATOM | 2447 | CA | ALA | 198 | 106.339 | 83.495 | 91.934 | 0.00 | 0.00 | C |
| ATOM | 2448 | C | ALA | 198 | 106.806 | 83.637 | 93.440 | 0.00 | 0.00 | C |
| ATOM | 2449 | O | ALA | 198 | 108.002 | 83.748 | 93.727 | 0.00 | 0.00 | O |
| ATOM | 2450 | CB | ALA | 198 | 107.503 | 83.535 | 90.916 | 0.00 | 0.00 | C |
| ATOM | 2451 | H | ALA | 198 | 104.748 | 84.147 | 90.531 | 0.00 | 0.00 | H |
| ATOM | 2452 | HA | ALA | 198 | 105.931 | 82.469 | 91.885 | 0.00 | 0.00 | H |
| ATOM | 2453 | 1HB | ALA | 198 | 108.291 | 82.802 | 91.167 | 0.00 | 0.00 | H |
| ATOM | 2454 | 2HB | ALA | 198 | 107.191 | 83.322 | 89.879 | 0.00 | 0.00 | H |
| ATOM | 2455 | 3HB | ALA | 198 | 107.996 | 84.527 | 90.905 | 0.00 | 0.00 | H |
| ATOM | 2456 | N | THR | 199 | 105.868 | 83.616 | 94.406 | 0.00 | 0.00 | N |
| ATOM | 2457 | CA | THR | 199 | 106.156 | 84.018 | 95.827 | 0.00 | 0.00 | C |
| ATOM | 2458 | C | THR | 199 | 106.899 | 82.955 | 96.723 | 0.00 | 0.00 | C |
| ATOM | 2459 | O | THR | 199 | 106.343 | 82.424 | 97.690 | 0.00 | 0.00 | O |
| ATOM | 2460 | CB | THR | 199 | 104.846 | 84.607 | 96.453 | 0.00 | 0.00 | C |
| ATOM | 2461 | OG1 | THR | 199 | 105.108 | 85.041 | 97.781 | 0.00 | 0.00 | O |
| ATOM | 2462 | CG2 | THR | 199 | 103.621 | 83.679 | 96.562 | 0.00 | 0.00 | C |
| ATOM | 2463 | H | THR | 199 | 104.928 | 83.743 | 94.023 | 0.00 | 0.00 | H |
| ATOM | 2464 | HA | THR | 199 | 106.844 | 84.891 | 95.797 | 0.00 | 0.00 | H |
| ATOM | 2465 | HB | THR | 199 | 104.555 | 85.498 | 95.858 | 0.00 | 0.00 | H |
| ATOM | 2466 | HG1 | THR | 199 | 105.389 | 84.253 | 98.258 | 0.00 | 0.00 | H |
| ATOM | 2467 | 1HG2 | THR | 199 | 103.274 | 83.329 | 95.574 | 0.00 | 0.00 | H |
| ATOM | 2468 | 2HG2 | THR | 199 | 103.828 | 82.784 | 97.176 | 0.00 | 0.00 | H |
| ATOM | 2469 | 3HG2 | THR | 199 | 102.768 | 84.204 | 97.033 | 0.00 | 0.00 | H |
| ATOM | 2470 | N | LYS | 200 | 108.191 | 82.708 | 96.444 | 0.00 | 0.00 | N |
| ATOM | 2471 | CA | LYS | 200 | 109.045 | 81.751 | 97.211 | 0.00 | 0.00 | C |
| ATOM | 2472 | C | LYS | 200 | 110.269 | 82.493 | 97.854 | 0.00 | 0.00 | C |
| ATOM | 2473 | O | LYS | 200 | 110.965 | 83.267 | 97.191 | 0.00 | 0.00 | O |
| ATOM | 2474 | CB | LYS | 200 | 109.511 | 80.590 | 96.281 | 0.00 | 0.00 | C |
| ATOM | 2475 | CG | LYS | 200 | 108.392 | 79.733 | 95.634 | 0.00 | 0.00 | C |
| ATOM | 2476 | CD | LYS | 200 | 108.929 | 78.488 | 94.901 | 0.00 | 0.00 | C |
| ATOM | 2477 | CE | LYS | 200 | 107.840 | 77.788 | 94.074 | 0.00 | 0.00 | C |
| ATOM | 2478 | NZ | LYS | 200 | 108.399 | 76.594 | 93.408 | 0.00 | 0.00 | N |
| ATOM | 2479 | 1HZ | LYS | 200 | 107.662 | 76.132 | 92.856 | 1.00 | 0.00 | H |
| ATOM | 2480 | 2HZ | LYS | 200 | 109.168 | 76.877 | 92.784 | 1.00 | 0.00 | H |
| ATOM | 2481 | 3HZ | LYS | 200 | 108.757 | 75.940 | 94.118 | 1.00 | 0.00 | H |
| ATOM | 2482 | H | LYS | 200 | 108.518 | 83.167 | 95.581 | 0.00 | 0.00 | H |
| ATOM | 2483 | HA | LYS | 200 | 108.457 | 81.285 | 98.030 | 0.00 | 0.00 | H |
| ATOM | 2484 | 1HB | LYS | 200 | 110.160 | 81.001 | 95.481 | 0.00 | 0.00 | H |
| ATOM | 2485 | 2HB | LYS | 200 | 110.174 | 79.924 | 96.865 | 0.00 | 0.00 | H |
| ATOM | 2486 | 1HG | LYS | 200 | 107.653 | 79.425 | 96.400 | 0.00 | 0.00 | H |
| ATOM | 2487 | 2HG | LYS | 200 | 107.826 | 80.368 | 94.923 | 0.00 | 0.00 | H |
| ATOM | 2488 | 1HD | LYS | 200 | 109.766 | 78.777 | 94.234 | 0.00 | 0.00 | H |
| ATOM | 2489 | 2HD | LYS | 200 | 109.366 | 77.785 | 95.637 | 0.00 | 0.00 | H |
| ATOM | 2490 | 1HE | LYS | 200 | 106.978 | 77.501 | 94.711 | 0.00 | 0.00 | H |
| ATOM | 2491 | 2HE | LYS | 200 | 107.440 | 78.483 | 93.308 | 0.00 | 0.00 | H |
| ATOM | 2492 | N | VAL | 201 | 110.533 | 82.262 | 99.151 | 0.00 | 0.00 | N |
| ATOM | 2493 | CA | VAL | 201 | 111.582 | 83.002 | 99.926 | 0.00 | 0.00 | C |
| ATOM | 2494 | C | VAL | 201 | 112.931 | 82.205 | 100.056 | 0.00 | 0.00 | C |
| ATOM | 2495 | O | VAL | 201 | 112.932 | 80.973 | 100.172 | 0.00 | 0.00 | O |
| ATOM | 2496 | CB | VAL | 201 | 110.952 | 83.444 | 101.300 | 0.00 | 0.00 | C |
| ATOM | 2497 | CG1 | VAL | 201 | 110.769 | 82.319 | 102.349 | 0.00 | 0.00 | C |
| ATOM | 2498 | CG2 | VAL | 201 | 111.719 | 84.604 | 101.969 | 0.00 | 0.00 | C |
| ATOM | 2499 | H | VAL | 201 | 109.948 | 81.547 | 99.591 | 0.00 | 0.00 | H |
| ATOM | 2500 | HA | VAL | 201 | 111.822 | 83.938 | 99.379 | 0.00 | 0.00 | H |
| ATOM | 2501 | HB | VAL | 201 | 109.942 | 83.850 | 101.080 | 0.00 | 0.00 | H |
| ATOM | 2502 | 1HG1 | VAL | 201 | 111.738 | 81.929 | 102.713 | 0.00 | 0.00 | H |
| ATOM | 2503 | 2HG1 | VAL | 201 | 110.206 | 82.666 | 103.235 | 0.00 | 0.00 | H |
| ATOM | 2504 | 3HG1 | VAL | 201 | 110.214 | 81.455 | 101.938 | 0.00 | 0.00 | H |
| ATOM | 2505 | 1HG2 | VAL | 201 | 112.741 | 84.312 | 102.280 | 0.00 | 0.00 | H |
| ATOM | 2506 | 2HG2 | VAL | 201 | 111.820 | 85.473 | 101.292 | 0.00 | 0.00 | H |
| ATOM | 2507 | 3HG2 | VAL | 201 | 111.200 | 84.973 | 102.874 | 0.00 | 0.00 | H |
| ATOM | 2508 | N | GLU | 202 | 114.083 | 82.901 | 100.125 | 0.00 | 0.00 | N |
| ATOM | 2509 | CA | GLU | 202 | 115.425 | 82.250 | 100.286 | 0.00 | 0.00 | C |
| ATOM | 2510 | C | GLU | 202 | 115.779 | 81.725 | 101.739 | 0.00 | 0.00 | C |
| ATOM | 2511 | O | GLU | 202 | 116.881 | 81.934 | 102.253 | 0.00 | 0.00 | O |
| ATOM | 2512 | CB | GLU | 202 | 116.504 | 83.217 | 99.708 | 0.00 | 0.00 | C |
| ATOM | 2513 | CG | GLU | 202 | 116.458 | 83.535 | 98.184 | 0.00 | 0.00 | C |
| ATOM | 2514 | CD | GLU | 202 | 115.676 | 84.788 | 97.775 | 0.00 | 0.00 | C |
| ATOM | 2515 | OE1 | GLU | 202 | 114.539 | 85.053 | 98.156 | 0.00 | 0.00 | O |
| ATOM | 2516 | OE2 | GLU | 202 | 116.386 | 85.585 | 96.932 | 0.00 | 0.00 | O |
| ATOM | 2517 | H | GLU | 202 | 114.001 | 83.871 | 99.795 | 0.00 | 0.00 | H |
| ATOM | 2518 | HA | GLU | 202 | 115.455 | 81.342 | 99.651 | 0.00 | 0.00 | H |
| ATOM | 2519 | 1HB | GLU | 202 | 116.534 | 84.153 | 100.302 | 0.00 | 0.00 | H |
| ATOM | 2520 | 2HB | GLU | 202 | 117.493 | 82.758 | 99.902 | 0.00 | 0.00 | H |
| ATOM | 2521 | 1HG | GLU | 202 | 117.496 | 83.646 | 97.815 | 0.00 | 0.00 | H |
| ATOM | 2522 | 2HG | GLU | 202 | 116.054 | 82.676 | 97.616 | 0.00 | 0.00 | H |
| ATOM | 2523 | N | TYR | 203 | 114.863 | 80.962 | 102.354 | 0.00 | 0.00 | N |
| ATOM | 2524 | CA | TYR | 203 | 115.139 | 80.119 | 103.556 | 0.00 | 0.00 | C |
| ATOM | 2525 | C | TYR | 203 | 114.495 | 78.695 | 103.433 | 0.00 | 0.00 | C |
| ATOM | 2526 | O | TYR | 203 | 115.206 | 77.708 | 103.643 | 0.00 | 0.00 | O |
| ATOM | 2527 | CB | TYR | 203 | 114.729 | 80.840 | 104.875 | 0.00 | 0.00 | C |
| ATOM | 2528 | CG | TYR | 203 | 115.716 | 81.901 | 105.398 | 0.00 | 0.00 | C |
| ATOM | 2529 | CD1 | TYR | 203 | 116.989 | 81.520 | 105.840 | 0.00 | 0.00 | C |
| ATOM | 2530 | CE1 | TYR | 203 | 117.875 | 82.471 | 106.344 | 0.00 | 0.00 | C |
| ATOM | 2531 | CZ | TYR | 203 | 117.489 | 83.805 | 106.423 | 0.00 | 0.00 | C |
| ATOM | 2532 | OH | TYR | 203 | 118.352 | 84.738 | 106.929 | 0.00 | 0.00 | O |
| ATOM | 2533 | CE2 | TYR | 203 | 116.225 | 84.194 | 105.991 | 0.00 | 0.00 | C |
| ATOM | 2534 | CD2 | TYR | 203 | 115.340 | 83.246 | 105.477 | 0.00 | 0.00 | C |
| ATOM | 2535 | H | TYR | 203 | 113.907 | 80.956 | 101.972 | 1.00 | 0.00 | H |
| ATOM | 2536 | HA | TYR | 203 | 116.228 | 79.912 | 103.608 | 0.00 | 0.00 | H |
| ATOM | 2537 | 1HB | TYR | 203 | 113.711 | 81.261 | 104.769 | 0.00 | 0.00 | H |
| ATOM | 2538 | 2HB | TYR | 203 | 114.617 | 80.092 | 105.682 | 0.00 | 0.00 | H |
| ATOM | 2539 | HD1 | TYR | 203 | 117.300 | 80.485 | 105.787 | 0.00 | 0.00 | H |
| ATOM | 2540 | HE1 | TYR | 203 | 118.858 | 82.164 | 106.672 | 0.00 | 0.00 | H |
| ATOM | 2541 | HH | TYR | 203 | 119.183 | 84.305 | 107.133 | 0.00 | 0.00 | H |
| ATOM | 2542 | HE2 | TYR | 203 | 115.933 | 85.231 | 106.059 | 0.00 | 0.00 | H |
| ATOM | 2543 | HD2 | TYR | 203 | 114.361 | 83.561 | 105.145 | 0.00 | 0.00 | H |
| ATOM | 2544 | N | ASP | 204 | 113.189 | 78.546 | 103.110 | 0.00 | 0.00 | N |
| ATOM | 2545 | CA | ASP | 204 | 112.574 | 77.215 | 102.817 | 0.00 | 0.00 | C |
| ATOM | 2546 | C | ASP | 204 | 111.617 | 77.238 | 101.564 | 0.00 | 0.00 | C |
| ATOM | 2547 | O | ASP | 204 | 110.886 | 78.205 | 101.331 | 0.00 | 0.00 | O |
| ATOM | 2548 | CB | ASP | 204 | 111.973 | 76.596 | 104.114 | 0.00 | 0.00 | C |
| ATOM | 2549 | CG | ASP | 204 | 110.524 | 76.932 | 104.462 | 0.00 | 0.00 | C |
| ATOM | 2550 | OD1 | ASP | 204 | 110.164 | 77.963 | 105.017 | 0.00 | 0.00 | O |
| ATOM | 2551 | OD2 | ASP | 204 | 109.676 | 75.937 | 104.080 | 0.00 | 0.00 | O |
| ATOM | 2552 | H | ASP | 204 | 112.693 | 79.423 | 102.920 | 0.00 | 0.00 | H |
| ATOM | 2553 | HA | ASP | 204 | 113.401 | 76.532 | 102.528 | 0.00 | 0.00 | H |
| ATOM | 2554 | 1HB | ASP | 204 | 112.050 | 75.497 | 104.043 | 0.00 | 0.00 | H |
| ATOM | 2555 | 2HB | ASP | 204 | 112.599 | 76.844 | 104.991 | 0.00 | 0.00 | H |
| ATOM | 2556 | N | GLY | 205 | 111.602 | 76.142 | 100.783 | 0.00 | 0.00 | N |
| ATOM | 2557 | CA | GLY | 205 | 110.724 | 76.006 | 99.584 | 0.00 | 0.00 | C |
| ATOM | 2558 | C | GLY | 205 | 109.511 | 75.068 | 99.784 | 0.00 | 0.00 | C |
| ATOM | 2559 | O | GLY | 205 | 109.682 | 73.872 | 100.024 | 0.00 | 0.00 | O |
| ATOM | 2560 | H | GLY | 205 | 112.181 | 75.370 | 101.128 | 0.00 | 0.00 | H |
| ATOM | 2561 | 1HA | GLY | 205 | 110.407 | 76.994 | 99.196 | 0.00 | 0.00 | H |
| ATOM | 2562 | 2HA | GLY | 205 | 111.323 | 75.579 | 98.760 | 0.00 | 0.00 | H |
| ATOM | 2563 | N | GLU | 206 | 108.290 | 75.605 | 99.660 | 0.00 | 0.00 | N |
| ATOM | 2564 | CA | GLU | 206 | 107.034 | 74.858 | 99.972 | 0.00 | 0.00 | C |
| ATOM | 2565 | C | GLU | 206 | 105.858 | 75.267 | 99.020 | 0.00 | 0.00 | C |
| ATOM | 2566 | O | GLU | 206 | 105.527 | 76.451 | 98.906 | 0.00 | 0.00 | O |
| ATOM | 2567 | CB | GLU | 206 | 106.695 | 75.043 | 101.487 | 0.00 | 0.00 | C |
| ATOM | 2568 | CG | GLU | 206 | 106.381 | 76.487 | 101.985 | 0.00 | 0.00 | C |
| ATOM | 2569 | CD | GLU | 206 | 106.297 | 76.676 | 103.499 | 0.00 | 0.00 | C |
| ATOM | 2570 | OE1 | GLU | 206 | 107.039 | 76.129 | 104.309 | 0.00 | 0.00 | O |
| ATOM | 2571 | OE2 | GLU | 206 | 105.335 | 77.567 | 103.853 | 0.00 | 0.00 | O |
| ATOM | 2572 | H | GLU | 206 | 108.289 | 76.621 | 99.527 | 0.00 | 0.00 | H |
| ATOM | 2573 | HA | GLU | 206 | 107.213 | 73.774 | 99.821 | 0.00 | 0.00 | H |
| ATOM | 2574 | 1HB | GLU | 206 | 105.848 | 74.381 | 101.750 | 0.00 | 0.00 | H |
| ATOM | 2575 | 2HB | GLU | 206 | 107.545 | 74.637 | 102.075 | 0.00 | 0.00 | H |
| ATOM | 2576 | 1HG | GLU | 206 | 107.156 | 77.189 | 101.629 | 0.00 | 0.00 | H |
| ATOM | 2577 | 2HG | GLU | 206 | 105.439 | 76.843 | 101.526 | 0.00 | 0.00 | H |
| ATOM | 2578 | N | ARG | 207 | 105.178 | 74.299 | 98.371 | 0.00 | 0.00 | N |
| ATOM | 2579 | CA | ARG | 207 | 103.984 | 74.593 | 97.511 | 0.00 | 0.00 | C |
| ATOM | 2580 | C | ARG | 207 | 102.644 | 74.622 | 98.338 | 0.00 | 0.00 | C |
| ATOM | 2581 | O | ARG | 207 | 101.731 | 73.817 | 98.148 | 0.00 | 0.00 | O |
| ATOM | 2582 | CB | ARG | 207 | 103.948 | 73.615 | 96.295 | 0.00 | 0.00 | C |
| ATOM | 2583 | CG | ARG | 207 | 105.115 | 73.695 | 95.268 | 0.00 | 0.00 | C |
| ATOM | 2584 | CD | ARG | 207 | 106.268 | 72.718 | 95.557 | 0.00 | 0.00 | C |
| ATOM | 2585 | NE | ARG | 207 | 107.373 | 72.962 | 94.591 | 0.00 | 0.00 | N |
| ATOM | 2586 | CZ | ARG | 207 | 108.655 | 73.134 | 94.889 | 0.00 | 0.00 | C |
| ATOM | 2587 | NH1 | ARG | 207 | 109.140 | 73.144 | 96.096 | 0.00 | 0.00 | N |
| ATOM | 2588 | NH2 | ARG | 207 | 109.474 | 73.306 | 93.911 | 0.00 | 0.00 | N |
| ATOM | 2589 | HE | ARG | 207 | 107.118 | 73.002 | 93.594 | 1.00 | 0.00 | H |
| ATOM | 2590 | H | ARG | 207 | 105.521 | 73.344 | 98.513 | 0.00 | 0.00 | H |
| ATOM | 2591 | HA | ARG | 207 | 104.084 | 75.603 | 97.065 | 0.00 | 0.00 | H |
| ATOM | 2592 | 1HB | ARG | 207 | 103.801 | 72.574 | 96.640 | 0.00 | 0.00 | H |
| ATOM | 2593 | 2HB | ARG | 207 | 103.016 | 73.828 | 95.733 | 0.00 | 0.00 | H |
| ATOM | 2594 | 1HG | ARG | 207 | 104.731 | 73.468 | 94.252 | 0.00 | 0.00 | H |
| ATOM | 2595 | 2HG | ARG | 207 | 105.498 | 74.731 | 95.192 | 0.00 | 0.00 | H |
| ATOM | 2596 | 1HD | ARG | 207 | 106.593 | 72.797 | 96.609 | 0.00 | 0.00 | H |
| ATOM | 2597 | 2HD | ARG | 207 | 105.910 | 71.676 | 95.436 | 0.00 | 0.00 | H |
| ATOM | 2598 | 1HH1 | ARG | 207 | 110.147 | 73.271 | 96.191 | 0.00 | 0.00 | H |
| ATOM | 2599 | 2HH1 | ARG | 207 | 108.436 | 73.002 | 96.820 | 0.00 | 0.00 | H |
| ATOM | 2600 | 1HH2 | ARG | 207 | 109.015 | 73.300 | 92.994 | 0.00 | 0.00 | H |
| ATOM | 2601 | 2HH2 | ARG | 207 | 110.458 | 73.437 | 94.135 | 0.00 | 0.00 | H |
| ATOM | 2602 | N | LYS | 208 | 102.551 | 75.594 | 99.257 | 0.00 | 0.00 | N |
| ATOM | 2603 | CA | LYS | 208 | 101.367 | 75.800 | 100.154 | 0.00 | 0.00 | C |
| ATOM | 2604 | C | LYS | 208 | 100.318 | 76.879 | 99.671 | 0.00 | 0.00 | C |
| ATOM | 2605 | O | LYS | 208 | 99.196 | 76.904 | 100.180 | 0.00 | 0.00 | O |
| ATOM | 2606 | CB | LYS | 208 | 102.023 | 76.067 | 101.541 | 0.00 | 0.00 | C |
| ATOM | 2607 | CG | LYS | 208 | 101.078 | 76.180 | 102.758 | 0.00 | 0.00 | C |
| ATOM | 2608 | CD | LYS | 208 | 101.870 | 76.109 | 104.081 | 0.00 | 0.00 | C |
| ATOM | 2609 | CE | LYS | 208 | 101.036 | 76.510 | 105.304 | 0.00 | 0.00 | C |
| ATOM | 2610 | NZ | LYS | 208 | 101.838 | 76.307 | 106.527 | 0.00 | 0.00 | N |
| ATOM | 2611 | 1HZ | LYS | 208 | 101.282 | 76.576 | 107.351 | 1.00 | 0.00 | H |
| ATOM | 2612 | 2HZ | LYS | 208 | 102.106 | 75.315 | 106.602 | 1.00 | 0.00 | H |
| ATOM | 2613 | 3HZ | LYS | 208 | 102.687 | 76.889 | 106.482 | 1.00 | 0.00 | H |
| ATOM | 2614 | H | LYS | 208 | 103.435 | 76.102 | 99.387 | 0.00 | 0.00 | H |
| ATOM | 2615 | HA | LYS | 208 | 100.792 | 74.856 | 100.241 | 0.00 | 0.00 | H |
| ATOM | 2616 | 1HB | LYS | 208 | 102.739 | 75.245 | 101.756 | 0.00 | 0.00 | H |
| ATOM | 2617 | 2HB | LYS | 208 | 102.644 | 76.983 | 101.497 | 0.00 | 0.00 | H |
| ATOM | 2618 | 1HG | LYS | 208 | 100.505 | 77.126 | 102.695 | 0.00 | 0.00 | H |
| ATOM | 2619 | 2HG | LYS | 208 | 100.318 | 75.374 | 102.740 | 0.00 | 0.00 | H |
| ATOM | 2620 | 1HD | LYS | 208 | 102.260 | 75.079 | 104.211 | 0.00 | 0.00 | H |
| ATOM | 2621 | 2HD | LYS | 208 | 102.765 | 76.757 | 104.020 | 0.00 | 0.00 | H |
| ATOM | 2622 | 1HE | LYS | 208 | 100.722 | 77.570 | 105.221 | 0.00 | 0.00 | H |
| ATOM | 2623 | 2HE | LYS | 208 | 100.104 | 75.914 | 105.360 | 0.00 | 0.00 | H |
| ATOM | 2624 | N | LYS | 209 | 100.659 | 77.740 | 98.691 | 0.00 | 0.00 | N |
| ATOM | 2625 | CA | LYS | 209 | 99.705 | 78.666 | 97.997 | 0.00 | 0.00 | C |
| ATOM | 2626 | C | LYS | 209 | 99.374 | 78.284 | 96.498 | 0.00 | 0.00 | C |
| ATOM | 2627 | O | LYS | 209 | 98.226 | 78.420 | 96.065 | 0.00 | 0.00 | O |
| ATOM | 2628 | CB | LYS | 209 | 100.265 | 80.104 | 98.203 | 0.00 | 0.00 | C |
| ATOM | 2629 | CG | LYS | 209 | 99.332 | 81.261 | 97.765 | 0.00 | 0.00 | C |
| ATOM | 2630 | CD | LYS | 209 | 99.630 | 81.806 | 96.350 | 0.00 | 0.00 | C |
| ATOM | 2631 | CE | LYS | 209 | 98.518 | 82.713 | 95.793 | 0.00 | 0.00 | C |
| ATOM | 2632 | NZ | LYS | 209 | 97.422 | 81.903 | 95.221 | 0.00 | 0.00 | N |
| ATOM | 2633 | 1HZ | LYS | 209 | 96.689 | 82.527 | 94.854 | 1.00 | 0.00 | H |
| ATOM | 2634 | 2HZ | LYS | 209 | 97.789 | 81.319 | 94.457 | 1.00 | 0.00 | H |
| ATOM | 2635 | 3HZ | LYS | 209 | 97.023 | 81.299 | 95.954 | 1.00 | 0.00 | H |
| ATOM | 2636 | H | LYS | 209 | 101.624 | 77.598 | 98.384 | 0.00 | 0.00 | H |
| ATOM | 2637 | HA | LYS | 209 | 98.723 | 78.635 | 98.513 | 0.00 | 0.00 | H |
| ATOM | 2638 | 1HB | LYS | 209 | 100.457 | 80.245 | 99.286 | 0.00 | 0.00 | H |
| ATOM | 2639 | 2HB | LYS | 209 | 101.263 | 80.211 | 97.735 | 0.00 | 0.00 | H |
| ATOM | 2640 | 1HG | LYS | 209 | 98.273 | 80.948 | 97.853 | 0.00 | 0.00 | H |
| ATOM | 2641 | 2HG | LYS | 209 | 99.430 | 82.095 | 98.485 | 0.00 | 0.00 | H |
| ATOM | 2642 | 1HD | LYS | 209 | 100.572 | 82.388 | 96.396 | 0.00 | 0.00 | H |
| ATOM | 2643 | 2HD | LYS | 209 | 99.846 | 80.986 | 95.638 | 0.00 | 0.00 | H |
| ATOM | 2644 | 1HE | LYS | 209 | 98.125 | 83.390 | 96.578 | 0.00 | 0.00 | H |
| ATOM | 2645 | 2HE | LYS | 209 | 98.924 | 83.383 | 95.009 | 0.00 | 0.00 | H |
| ATOM | 2646 | N | THR | 210 | 100.341 | 77.767 | 95.710 | 0.00 | 0.00 | N |
| ATOM | 2647 | CA | THR | 210 | 100.077 | 76.742 | 94.640 | 0.00 | 0.00 | C |
| ATOM | 2648 | C | THR | 210 | 99.851 | 77.267 | 93.177 | 0.00 | 0.00 | C |
| ATOM | 2649 | O | THR | 210 | 100.677 | 76.956 | 92.318 | 0.00 | 0.00 | O |
| ATOM | 2650 | CB | THR | 210 | 99.149 | 75.559 | 95.084 | 0.00 | 0.00 | C |
| ATOM | 2651 | OG1 | THR | 210 | 99.601 | 75.041 | 96.330 | 0.00 | 0.00 | O |
| ATOM | 2652 | CG2 | THR | 210 | 99.114 | 74.344 | 94.145 | 0.00 | 0.00 | C |
| ATOM | 2653 | H | THR | 210 | 101.215 | 77.717 | 96.237 | 0.00 | 0.00 | H |
| ATOM | 2654 | HA | THR | 210 | 101.060 | 76.239 | 94.553 | 0.00 | 0.00 | H |
| ATOM | 2655 | HB | THR | 210 | 98.116 | 75.943 | 95.207 | 0.00 | 0.00 | H |
| ATOM | 2656 | HG1 | THR | 210 | 99.191 | 74.178 | 96.440 | 0.00 | 0.00 | H |
| ATOM | 2657 | 1HG2 | THR | 210 | 98.450 | 73.553 | 94.537 | 0.00 | 0.00 | H |
| ATOM | 2658 | 2HG2 | THR | 210 | 98.737 | 74.606 | 93.138 | 0.00 | 0.00 | H |
| ATOM | 2659 | 3HG2 | THR | 210 | 100.117 | 73.895 | 94.010 | 0.00 | 0.00 | H |
| ATOM | 2660 | N | LEU | 211 | 98.760 | 77.989 | 92.859 | 0.00 | 0.00 | N |
| ATOM | 2661 | CA | LEU | 211 | 98.390 | 78.383 | 91.462 | 0.00 | 0.00 | C |
| ATOM | 2662 | C | LEU | 211 | 98.152 | 79.921 | 91.268 | 0.00 | 0.00 | C |
| ATOM | 2663 | O | LEU | 211 | 97.299 | 80.524 | 91.926 | 0.00 | 0.00 | O |
| ATOM | 2664 | CB | LEU | 211 | 97.152 | 77.525 | 91.055 | 0.00 | 0.00 | C |
| ATOM | 2665 | CG | LEU | 211 | 96.653 | 77.648 | 89.589 | 0.00 | 0.00 | C |
| ATOM | 2666 | CD1 | LEU | 211 | 97.684 | 77.137 | 88.570 | 0.00 | 0.00 | C |
| ATOM | 2667 | CD2 | LEU | 211 | 95.344 | 76.862 | 89.403 | 0.00 | 0.00 | C |
| ATOM | 2668 | H | LEU | 211 | 98.174 | 78.215 | 93.673 | 1.00 | 0.00 | H |
| ATOM | 2669 | HA | LEU | 211 | 99.205 | 78.082 | 90.775 | 0.00 | 0.00 | H |
| ATOM | 2670 | 1HB | LEU | 211 | 96.317 | 77.777 | 91.739 | 0.00 | 0.00 | H |
| ATOM | 2671 | 2HB | LEU | 211 | 97.367 | 76.457 | 91.258 | 0.00 | 0.00 | H |
| ATOM | 2672 | HG | LEU | 211 | 96.437 | 78.714 | 89.370 | 0.00 | 0.00 | H |
| ATOM | 2673 | 1HD1 | LEU | 211 | 97.305 | 77.209 | 87.538 | 0.00 | 0.00 | H |
| ATOM | 2674 | 2HD1 | LEU | 211 | 98.623 | 77.717 | 88.593 | 0.00 | 0.00 | H |
| ATOM | 2675 | 3HD1 | LEU | 211 | 97.946 | 76.077 | 88.745 | 0.00 | 0.00 | H |
| ATOM | 2676 | 1HD2 | LEU | 211 | 94.944 | 76.959 | 88.377 | 0.00 | 0.00 | H |
| ATOM | 2677 | 2HD2 | LEU | 211 | 95.475 | 75.781 | 89.600 | 0.00 | 0.00 | H |
| ATOM | 2678 | 3HD2 | LEU | 211 | 94.551 | 77.222 | 90.084 | 0.00 | 0.00 | H |
| ATOM | 2679 | N | CYS | 212 | 98.844 | 80.530 | 90.287 | 0.00 | 0.00 | N |
| ATOM | 2680 | CA | CYS | 212 | 98.480 | 81.859 | 89.721 | 0.00 | 0.00 | C |
| ATOM | 2681 | C | CYS | 212 | 98.878 | 82.007 | 88.209 | 0.00 | 0.00 | C |
| ATOM | 2682 | O | CYS | 212 | 99.672 | 81.234 | 87.662 | 0.00 | 0.00 | O |
| ATOM | 2683 | CB | CYS | 212 | 99.121 | 82.952 | 90.615 | 0.00 | 0.00 | C |
| ATOM | 2684 | SG | CYS | 212 | 100.943 | 82.953 | 90.479 | 0.00 | 0.00 | S |
| ATOM | 2685 | H | CYS | 212 | 99.566 | 79.952 | 89.843 | 0.00 | 0.00 | H |
| ATOM | 2686 | HA | CYS | 212 | 97.378 | 81.976 | 89.772 | 0.00 | 0.00 | H |
| ATOM | 2687 | 1HB | CYS | 212 | 98.834 | 82.823 | 91.677 | 0.00 | 0.00 | H |
| ATOM | 2688 | 2HB | CYS | 212 | 98.750 | 83.954 | 90.330 | 0.00 | 0.00 | H |
| ATOM | 2689 | HG | CYS | 212 | 101.154 | 81.796 | 91.094 | 1.00 | 0.00 | H |
| ATOM | 2690 | N | GLY | 213 | 98.337 | 83.037 | 87.535 | 0.00 | 0.00 | N |
| ATOM | 2691 | CA | GLY | 213 | 98.800 | 83.453 | 86.181 | 0.00 | 0.00 | C |
| ATOM | 2692 | C | GLY | 213 | 97.663 | 83.738 | 85.183 | 0.00 | 0.00 | C |
| ATOM | 2693 | O | GLY | 213 | 96.866 | 84.657 | 85.383 | 0.00 | 0.00 | O |
| ATOM | 2694 | H | GLY | 213 | 97.707 | 83.619 | 88.099 | 0.00 | 0.00 | H |
| ATOM | 2695 | 1HA | GLY | 213 | 99.534 | 82.740 | 85.752 | 0.00 | 0.00 | H |
| ATOM | 2696 | 2HA | GLY | 213 | 99.374 | 84.392 | 86.282 | 0.00 | 0.00 | H |
| ATOM | 2697 | N | THR | 214 | 97.627 | 82.972 | 84.088 | 0.00 | 0.00 | N |
| ATOM | 2698 | CA | THR | 214 | 96.718 | 83.237 | 82.933 | 0.00 | 0.00 | C |
| ATOM | 2699 | C | THR | 214 | 96.128 | 81.867 | 82.441 | 0.00 | 0.00 | C |
| ATOM | 2700 | O | THR | 214 | 96.917 | 81.060 | 81.936 | 0.00 | 0.00 | O |
| ATOM | 2701 | CB | THR | 214 | 97.505 | 83.999 | 81.819 | 0.00 | 0.00 | C |
| ATOM | 2702 | OG1 | THR | 214 | 97.923 | 85.263 | 82.313 | 0.00 | 0.00 | O |
| ATOM | 2703 | CG2 | THR | 214 | 96.715 | 84.308 | 80.537 | 0.00 | 0.00 | C |
| ATOM | 2704 | H | THR | 214 | 98.341 | 82.239 | 84.062 | 0.00 | 0.00 | H |
| ATOM | 2705 | HA | THR | 214 | 95.895 | 83.909 | 83.235 | 0.00 | 0.00 | H |
| ATOM | 2706 | HB | THR | 214 | 98.407 | 83.413 | 81.557 | 0.00 | 0.00 | H |
| ATOM | 2707 | HG1 | THR | 214 | 98.390 | 85.696 | 81.594 | 0.00 | 0.00 | H |
| ATOM | 2708 | 1HG2 | THR | 214 | 97.329 | 84.855 | 79.799 | 0.00 | 0.00 | H |
| ATOM | 2709 | 2HG2 | THR | 214 | 96.367 | 83.386 | 80.036 | 0.00 | 0.00 | H |
| ATOM | 2710 | 3HG2 | THR | 214 | 95.823 | 84.928 | 80.747 | 0.00 | 0.00 | H |
| ATOM | 2711 | N | PRO | 215 | 94.799 | 81.546 | 82.525 | 0.00 | 0.00 | N |
| ATOM | 2712 | CA | PRO | 215 | 94.254 | 80.205 | 82.148 | 0.00 | 0.00 | C |
| ATOM | 2713 | CD | PRO | 215 | 93.794 | 82.405 | 83.185 | 0.00 | 0.00 | C |
| ATOM | 2714 | C | PRO | 215 | 94.668 | 79.504 | 80.814 | 0.00 | 0.00 | C |
| ATOM | 2715 | O | PRO | 215 | 94.961 | 78.311 | 80.835 | 0.00 | 0.00 | O |
| ATOM | 2716 | CB | PRO | 215 | 92.735 | 80.418 | 82.290 | 0.00 | 0.00 | C |
| ATOM | 2717 | CG | PRO | 215 | 92.599 | 81.474 | 83.388 | 0.00 | 0.00 | C |
| ATOM | 2718 | HA | PRO | 215 | 94.580 | 79.510 | 82.945 | 0.00 | 0.00 | H |
| ATOM | 2719 | 1HD | PRO | 215 | 93.527 | 83.261 | 82.535 | 0.00 | 0.00 | H |
| ATOM | 2720 | 2HD | PRO | 215 | 94.140 | 82.814 | 84.156 | 0.00 | 0.00 | H |
| ATOM | 2721 | 1HB | PRO | 215 | 92.203 | 79.478 | 82.536 | 0.00 | 0.00 | H |
| ATOM | 2722 | 2HB | PRO | 215 | 92.296 | 80.791 | 81.344 | 0.00 | 0.00 | H |
| ATOM | 2723 | 1HG | PRO | 215 | 92.652 | 80.997 | 84.386 | 0.00 | 0.00 | H |
| ATOM | 2724 | 2HG | PRO | 215 | 91.637 | 82.017 | 83.344 | 0.00 | 0.00 | H |
| ATOM | 2725 | N | ASN | 216 | 94.745 | 80.230 | 79.689 | 1.00 | 0.00 | N |
| ATOM | 2726 | CA | ASN | 216 | 95.298 | 79.690 | 78.401 | 1.00 | 0.00 | C |
| ATOM | 2727 | C | ASN | 216 | 96.866 | 79.462 | 78.324 | 1.00 | 0.00 | C |
| ATOM | 2728 | O | ASN | 216 | 97.340 | 78.817 | 77.387 | 1.00 | 0.00 | O |
| ATOM | 2729 | CB | ASN | 216 | 94.840 | 80.631 | 77.244 | 1.00 | 0.00 | C |
| ATOM | 2730 | CG | ASN | 216 | 93.338 | 80.908 | 77.057 | 1.00 | 0.00 | C |
| ATOM | 2731 | OD1 | ASN | 216 | 92.446 | 80.174 | 77.461 | 1.00 | 0.00 | O |
| ATOM | 2732 | ND2 | ASN | 216 | 92.998 | 82.007 | 76.437 | 1.00 | 0.00 | N |
| ATOM | 2733 | H | ASN | 216 | 94.425 | 81.195 | 79.805 | 1.00 | 0.00 | H |
| ATOM | 2734 | HA | ASN | 216 | 94.848 | 78.695 | 78.209 | 1.00 | 0.00 | H |
| ATOM | 2735 | 1HB | ASN | 216 | 95.381 | 81.591 | 77.334 | 1.00 | 0.00 | H |
| ATOM | 2736 | 2HB | ASN | 216 | 95.186 | 80.193 | 76.288 | 1.00 | 0.00 | H |
| ATOM | 2737 | 1HD2 | ASN | 216 | 91.991 | 82.076 | 76.264 | 1.00 | 0.00 | H |
| ATOM | 2738 | 2HD2 | ASN | 216 | 93.751 | 82.524 | 75.978 | 1.00 | 0.00 | H |
| ATOM | 2739 | N | TYR | 217 | 97.665 | 80.015 | 79.256 | 0.00 | 0.00 | N |
| ATOM | 2740 | CA | TYR | 217 | 99.167 | 79.977 | 79.236 | 0.00 | 0.00 | C |
| ATOM | 2741 | C | TYR | 217 | 99.882 | 79.104 | 80.338 | 0.00 | 0.00 | C |
| ATOM | 2742 | O | TYR | 217 | 101.110 | 78.969 | 80.279 | 0.00 | 0.00 | O |
| ATOM | 2743 | CB | TYR | 217 | 99.650 | 81.465 | 79.294 | 0.00 | 0.00 | C |
| ATOM | 2744 | CG | TYR | 217 | 99.775 | 82.174 | 77.933 | 0.00 | 0.00 | C |
| ATOM | 2745 | CD1 | TYR | 217 | 101.042 | 82.420 | 77.396 | 0.00 | 0.00 | C |
| ATOM | 2746 | CE1 | TYR | 217 | 101.172 | 82.951 | 76.118 | 0.00 | 0.00 | C |
| ATOM | 2747 | CZ | TYR | 217 | 100.038 | 83.236 | 75.364 | 0.00 | 0.00 | C |
| ATOM | 2748 | OH | TYR | 217 | 100.171 | 83.611 | 74.059 | 0.00 | 0.00 | O |
| ATOM | 2749 | CE2 | TYR | 217 | 98.772 | 83.046 | 75.907 | 0.00 | 0.00 | C |
| ATOM | 2750 | CD2 | TYR | 217 | 98.639 | 82.526 | 77.193 | 0.00 | 0.00 | C |
| ATOM | 2751 | H | TYR | 217 | 97.212 | 80.500 | 80.044 | 1.00 | 0.00 | H |
| ATOM | 2752 | HA | TYR | 217 | 99.528 | 79.534 | 78.286 | 0.00 | 0.00 | H |
| ATOM | 2753 | 1HB | TYR | 217 | 100.632 | 81.524 | 79.806 | 0.00 | 0.00 | H |
| ATOM | 2754 | 2HB | TYR | 217 | 99.005 | 82.062 | 79.960 | 0.00 | 0.00 | H |
| ATOM | 2755 | HD1 | TYR | 217 | 101.934 | 82.173 | 77.953 | 0.00 | 0.00 | H |
| ATOM | 2756 | HE1 | TYR | 217 | 102.158 | 83.101 | 75.707 | 0.00 | 0.00 | H |
| ATOM | 2757 | HH | TYR | 217 | 101.054 | 83.350 | 73.772 | 0.00 | 0.00 | H |
| ATOM | 2758 | HE2 | TYR | 217 | 97.896 | 83.266 | 75.315 | 0.00 | 0.00 | H |
| ATOM | 2759 | HD2 | TYR | 217 | 97.654 | 82.353 | 77.596 | 0.00 | 0.00 | H |
| ATOM | 2760 | N | ILE | 218 | 99.176 | 78.544 | 81.339 | 1.00 | 0.00 | N |
| ATOM | 2761 | CA | ILE | 218 | 99.789 | 77.778 | 82.473 | 1.00 | 0.00 | C |
| ATOM | 2762 | C | ILE | 218 | 100.234 | 76.315 | 82.106 | 1.00 | 0.00 | C |
| ATOM | 2763 | O | ILE | 218 | 99.593 | 75.634 | 81.302 | 1.00 | 0.00 | O |
| ATOM | 2764 | CB | ILE | 218 | 98.852 | 77.782 | 83.741 | 1.00 | 0.00 | C |
| ATOM | 2765 | CG1 | ILE | 218 | 97.441 | 77.159 | 83.512 | 1.00 | 0.00 | C |
| ATOM | 2766 | CG2 | ILE | 218 | 98.755 | 79.177 | 84.408 | 1.00 | 0.00 | C |
| ATOM | 2767 | CD1 | ILE | 218 | 96.717 | 76.713 | 84.792 | 1.00 | 0.00 | C |
| ATOM | 2768 | H | ILE | 218 | 98.162 | 78.681 | 81.261 | 1.00 | 0.00 | H |
| ATOM | 2769 | HA | ILE | 218 | 100.713 | 78.317 | 82.768 | 1.00 | 0.00 | H |
| ATOM | 2770 | HB | ILE | 218 | 99.357 | 77.140 | 84.489 | 1.00 | 0.00 | H |
| ATOM | 2771 | 1HG1 | ILE | 218 | 96.797 | 77.850 | 82.931 | 1.00 | 0.00 | H |
| ATOM | 2772 | 2HG1 | ILE | 218 | 97.535 | 76.264 | 82.866 | 1.00 | 0.00 | H |
| ATOM | 2773 | 2HG2 | ILE | 218 | 99.752 | 79.601 | 84.631 | 1.00 | 0.00 | H |
| ATOM | 2774 | 3HG2 | ILE | 218 | 98.227 | 79.903 | 83.765 | 1.00 | 0.00 | H |
| ATOM | 2775 | 1HG2 | ILE | 218 | 98.211 | 79.141 | 85.371 | 1.00 | 0.00 | H |
| ATOM | 2776 | 2HD1 | ILE | 218 | 97.360 | 76.074 | 85.427 | 1.00 | 0.00 | H |
| ATOM | 2777 | 3HD1 | ILE | 218 | 96.388 | 77.571 | 85.404 | 1.00 | 0.00 | H |
| ATOM | 2778 | 1HD1 | ILE | 218 | 95.818 | 76.116 | 84.556 | 1.00 | 0.00 | H |
| ATOM | 2779 | N | ALA | 219 | 101.320 | 75.825 | 82.731 | 0.00 | 0.00 | N |
| ATOM | 2780 | CA | ALA | 219 | 101.841 | 74.455 | 82.485 | 0.00 | 0.00 | C |
| ATOM | 2781 | C | ALA | 219 | 101.206 | 73.307 | 83.368 | 0.00 | 0.00 | C |
| ATOM | 2782 | O | ALA | 219 | 100.817 | 73.579 | 84.511 | 0.00 | 0.00 | O |
| ATOM | 2783 | CB | ALA | 219 | 103.364 | 74.552 | 82.711 | 0.00 | 0.00 | C |
| ATOM | 2784 | H | ALA | 219 | 101.770 | 76.471 | 83.382 | 0.00 | 0.00 | H |
| ATOM | 2785 | HA | ALA | 219 | 101.687 | 74.215 | 81.416 | 0.00 | 0.00 | H |
| ATOM | 2786 | 1HB | ALA | 219 | 103.872 | 73.623 | 82.397 | 0.00 | 0.00 | H |
| ATOM | 2787 | 2HB | ALA | 219 | 103.825 | 75.369 | 82.126 | 0.00 | 0.00 | H |
| ATOM | 2788 | 3HB | ALA | 219 | 103.616 | 74.720 | 83.775 | 0.00 | 0.00 | H |
| ATOM | 2789 | N | PRO | 220 | 101.158 | 72.005 | 82.943 | 0.00 | 0.00 | N |
| ATOM | 2790 | CA | PRO | 220 | 100.659 | 70.877 | 83.791 | 0.00 | 0.00 | C |
| ATOM | 2791 | CD | PRO | 220 | 101.394 | 71.597 | 81.543 | 0.00 | 0.00 | C |
| ATOM | 2792 | C | PRO | 220 | 101.187 | 70.651 | 85.247 | 0.00 | 0.00 | C |
| ATOM | 2793 | O | PRO | 220 | 100.407 | 70.199 | 86.082 | 0.00 | 0.00 | O |
| ATOM | 2794 | CB | PRO | 220 | 100.904 | 69.656 | 82.883 | 0.00 | 0.00 | C |
| ATOM | 2795 | CG | PRO | 220 | 100.785 | 70.200 | 81.462 | 0.00 | 0.00 | C |
| ATOM | 2796 | HA | PRO | 220 | 99.562 | 71.009 | 83.886 | 0.00 | 0.00 | H |
| ATOM | 2797 | 1HD | PRO | 220 | 102.476 | 71.581 | 81.306 | 0.00 | 0.00 | H |
| ATOM | 2798 | 2HD | PRO | 220 | 100.898 | 72.264 | 80.814 | 0.00 | 0.00 | H |
| ATOM | 2799 | 1HB | PRO | 220 | 100.186 | 68.836 | 83.080 | 0.00 | 0.00 | H |
| ATOM | 2800 | 2HB | PRO | 220 | 101.916 | 69.231 | 83.038 | 0.00 | 0.00 | H |
| ATOM | 2801 | 1HG | PRO | 220 | 99.721 | 70.265 | 81.158 | 0.00 | 0.00 | H |
| ATOM | 2802 | 2HG | PRO | 220 | 101.288 | 69.563 | 80.711 | 0.00 | 0.00 | H |
| ATOM | 2803 | N | GLU | 221 | 102.455 | 70.966 | 85.580 | 0.00 | 0.00 | N |
| ATOM | 2804 | CA | GLU | 221 | 102.945 | 70.910 | 86.995 | 0.00 | 0.00 | C |
| ATOM | 2805 | C | GLU | 221 | 102.260 | 71.890 | 88.015 | 0.00 | 0.00 | C |
| ATOM | 2806 | O | GLU | 221 | 101.936 | 71.471 | 89.129 | 0.00 | 0.00 | O |
| ATOM | 2807 | CB | GLU | 221 | 104.487 | 71.081 | 87.071 | 0.00 | 0.00 | C |
| ATOM | 2808 | CG | GLU | 221 | 105.359 | 69.952 | 86.464 | 0.00 | 0.00 | C |
| ATOM | 2809 | CD | GLU | 221 | 106.798 | 70.025 | 86.972 | 0.00 | 0.00 | C |
| ATOM | 2810 | OE1 | GLU | 221 | 107.633 | 70.826 | 86.560 | 0.00 | 0.00 | O |
| ATOM | 2811 | OE2 | GLU | 221 | 107.020 | 69.153 | 87.992 | 0.00 | 0.00 | O |
| ATOM | 2812 | H | GLU | 221 | 102.987 | 71.365 | 84.803 | 0.00 | 0.00 | H |
| ATOM | 2813 | HA | GLU | 221 | 102.712 | 69.898 | 87.380 | 0.00 | 0.00 | H |
| ATOM | 2814 | 1HB | GLU | 221 | 104.749 | 71.155 | 88.147 | 0.00 | 0.00 | H |
| ATOM | 2815 | 2HB | GLU | 221 | 104.783 | 72.059 | 86.645 | 0.00 | 0.00 | H |
| ATOM | 2816 | 1HG | GLU | 221 | 105.363 | 70.002 | 85.361 | 0.00 | 0.00 | H |
| ATOM | 2817 | 2HG | GLU | 221 | 104.941 | 68.959 | 86.717 | 0.00 | 0.00 | H |
| ATOM | 2818 | N | VAL | 222 | 102.046 | 73.170 | 87.658 | 1.00 | 0.00 | N |
| ATOM | 2819 | CA | VAL | 222 | 101.206 | 74.108 | 88.480 | 1.00 | 0.00 | C |
| ATOM | 2820 | C | VAL | 222 | 99.684 | 73.725 | 88.570 | 1.00 | 0.00 | C |
| ATOM | 2821 | O | VAL | 222 | 99.093 | 73.815 | 89.649 | 1.00 | 0.00 | O |
| ATOM | 2822 | CB | VAL | 222 | 101.425 | 75.616 | 88.090 | 1.00 | 0.00 | C |
| ATOM | 2823 | CG1 | VAL | 222 | 102.863 | 76.109 | 88.353 | 1.00 | 0.00 | C |
| ATOM | 2824 | CG2 | VAL | 222 | 101.043 | 76.020 | 86.649 | 1.00 | 0.00 | C |
| ATOM | 2825 | H | VAL | 222 | 102.203 | 73.339 | 86.661 | 1.00 | 0.00 | H |
| ATOM | 2826 | HA | VAL | 222 | 101.561 | 74.024 | 89.528 | 1.00 | 0.00 | H |
| ATOM | 2827 | HB | VAL | 222 | 100.774 | 76.205 | 88.767 | 1.00 | 0.00 | H |
| ATOM | 2828 | 1HG1 | VAL | 222 | 103.609 | 75.553 | 87.759 | 1.00 | 0.00 | H |
| ATOM | 2829 | 2HG1 | VAL | 222 | 102.997 | 77.183 | 88.122 | 1.00 | 0.00 | H |
| ATOM | 2830 | 3HG1 | VAL | 222 | 103.135 | 75.982 | 89.416 | 1.00 | 0.00 | H |
| ATOM | 2831 | 2HG2 | VAL | 222 | 100.005 | 75.734 | 86.400 | 1.00 | 0.00 | H |
| ATOM | 2832 | 3HG2 | VAL | 222 | 101.111 | 77.114 | 86.497 | 1.00 | 0.00 | H |
| ATOM | 2833 | 1HG2 | VAL | 222 | 101.699 | 75.546 | 85.896 | 1.00 | 0.00 | H |
| ATOM | 2834 | N | LEU | 223 | 99.058 | 73.284 | 87.463 | 1.00 | 0.00 | N |
| ATOM | 2835 | CA | LEU | 223 | 97.660 | 72.767 | 87.463 | 1.00 | 0.00 | C |
| ATOM | 2836 | C | LEU | 223 | 97.419 | 71.428 | 88.252 | 1.00 | 0.00 | C |
| ATOM | 2837 | O | LEU | 223 | 96.535 | 71.382 | 89.110 | 1.00 | 0.00 | O |
| ATOM | 2838 | CB | LEU | 223 | 97.223 | 72.719 | 85.970 | 1.00 | 0.00 | C |
| ATOM | 2839 | CG | LEU | 223 | 95.765 | 72.282 | 85.690 | 1.00 | 0.00 | C |
| ATOM | 2840 | CD1 | LEU | 223 | 94.728 | 73.218 | 86.333 | 1.00 | 0.00 | C |
| ATOM | 2841 | CD2 | LEU | 223 | 95.530 | 72.202 | 84.175 | 1.00 | 0.00 | C |
| ATOM | 2842 | H | LEU | 223 | 99.671 | 73.215 | 86.643 | 1.00 | 0.00 | H |
| ATOM | 2843 | HA | LEU | 223 | 97.031 | 73.525 | 87.968 | 1.00 | 0.00 | H |
| ATOM | 2844 | 1HB | LEU | 223 | 97.391 | 73.708 | 85.502 | 1.00 | 0.00 | H |
| ATOM | 2845 | 2HB | LEU | 223 | 97.903 | 72.033 | 85.428 | 1.00 | 0.00 | H |
| ATOM | 2846 | HG | LEU | 223 | 95.616 | 71.263 | 86.104 | 1.00 | 0.00 | H |
| ATOM | 2847 | 2HD1 | LEU | 223 | 94.851 | 73.284 | 87.429 | 1.00 | 0.00 | H |
| ATOM | 2848 | 3HD1 | LEU | 223 | 94.782 | 74.249 | 85.935 | 1.00 | 0.00 | H |
| ATOM | 2849 | 1HD1 | LEU | 223 | 93.699 | 72.854 | 86.179 | 1.00 | 0.00 | H |
| ATOM | 2850 | 2HD2 | LEU | 223 | 95.604 | 73.188 | 83.680 | 1.00 | 0.00 | H |
| ATOM | 2851 | 3HD2 | LEU | 223 | 96.256 | 71.532 | 83.677 | 1.00 | 0.00 | H |
| ATOM | 2852 | 1HD2 | LEU | 223 | 94.532 | 71.792 | 83.951 | 1.00 | 0.00 | H |
| ATOM | 2853 | N | SER | 224 | 98.187 | 70.356 | 87.989 | 1.00 | 0.00 | N |
| ATOM | 2854 | CA | SER | 224 | 98.075 | 69.064 | 88.736 | 1.00 | 0.00 | C |
| ATOM | 2855 | C | SER | 224 | 98.658 | 68.999 | 90.199 | 1.00 | 0.00 | C |
| ATOM | 2856 | O | SER | 224 | 98.658 | 67.921 | 90.799 | 1.00 | 0.00 | O |
| ATOM | 2857 | CB | SER | 224 | 98.741 | 67.985 | 87.840 | 1.00 | 0.00 | C |
| ATOM | 2858 | OG | SER | 224 | 98.135 | 67.870 | 86.550 | 1.00 | 0.00 | O |
| ATOM | 2859 | H | SER | 224 | 98.879 | 70.476 | 87.238 | 1.00 | 0.00 | H |
| ATOM | 2860 | HA | SER | 224 | 97.005 | 68.795 | 88.832 | 1.00 | 0.00 | H |
| ATOM | 2861 | 1HB | SER | 224 | 99.820 | 68.204 | 87.720 | 1.00 | 0.00 | H |
| ATOM | 2862 | 2HB | SER | 224 | 98.702 | 66.998 | 88.340 | 1.00 | 0.00 | H |
| ATOM | 2863 | HG | SER | 224 | 97.378 | 67.267 | 86.625 | 1.00 | 0.00 | H |
| ATOM | 2864 | N | LYS | 225 | 99.135 | 70.118 | 90.782 | 1.00 | 0.00 | N |
| ATOM | 2865 | CA | LYS | 225 | 99.694 | 70.188 | 92.172 | 1.00 | 0.00 | C |
| ATOM | 2866 | C | LYS | 225 | 101.005 | 69.347 | 92.400 | 1.00 | 0.00 | C |
| ATOM | 2867 | O | LYS | 225 | 101.078 | 68.465 | 93.259 | 1.00 | 0.00 | O |
| ATOM | 2868 | CB | LYS | 225 | 98.586 | 69.988 | 93.255 | 1.00 | 0.00 | C |
| ATOM | 2869 | CG | LYS | 225 | 97.368 | 70.940 | 93.162 | 1.00 | 0.00 | C |
| ATOM | 2870 | CD | LYS | 225 | 96.400 | 70.792 | 94.350 | 1.00 | 0.00 | C |
| ATOM | 2871 | CE | LYS | 225 | 95.179 | 71.713 | 94.203 | 1.00 | 0.00 | C |
| ATOM | 2872 | NZ | LYS | 225 | 94.285 | 71.539 | 95.364 | 1.00 | 0.00 | N |
| ATOM | 2873 | 1HZ | LYS | 225 | 93.467 | 72.156 | 95.264 | 1.00 | 0.00 | H |
| ATOM | 2874 | 2HZ | LYS | 225 | 93.970 | 70.559 | 95.412 | 1.00 | 0.00 | H |
| ATOM | 2875 | 3HZ | LYS | 225 | 94.792 | 71.779 | 96.228 | 1.00 | 0.00 | H |
| ATOM | 2876 | H | LYS | 225 | 98.995 | 70.950 | 90.199 | 1.00 | 0.00 | H |
| ATOM | 2877 | HA | LYS | 225 | 100.027 | 71.236 | 92.303 | 1.00 | 0.00 | H |
| ATOM | 2878 | 1HB | LYS | 225 | 98.236 | 68.938 | 93.219 | 1.00 | 0.00 | H |
| ATOM | 2879 | 2HB | LYS | 225 | 99.054 | 70.093 | 94.253 | 1.00 | 0.00 | H |
| ATOM | 2880 | 1HG | LYS | 225 | 97.711 | 71.987 | 93.077 | 1.00 | 0.00 | H |
| ATOM | 2881 | 2HG | LYS | 225 | 96.824 | 70.743 | 92.216 | 1.00 | 0.00 | H |
| ATOM | 2882 | 1HD | LYS | 225 | 96.066 | 69.738 | 94.425 | 1.00 | 0.00 | H |
| ATOM | 2883 | 2HD | LYS | 225 | 96.934 | 71.007 | 95.295 | 1.00 | 0.00 | H |
| ATOM | 2884 | 1HE | LYS | 225 | 95.494 | 72.772 | 94.114 | 1.00 | 0.00 | H |
| ATOM | 2885 | 2HE | LYS | 225 | 94.630 | 71.476 | 93.268 | 1.00 | 0.00 | H |
| ATOM | 2886 | N | LYS | 226 | 102.052 | 69.648 | 91.613 | 0.00 | 0.00 | N |
| ATOM | 2887 | CA | LYS | 226 | 103.328 | 68.872 | 91.587 | 0.00 | 0.00 | C |
| ATOM | 2888 | C | LYS | 226 | 104.564 | 69.752 | 92.003 | 0.00 | 0.00 | C |
| ATOM | 2889 | O | LYS | 226 | 104.453 | 70.949 | 92.291 | 0.00 | 0.00 | O |
| ATOM | 2890 | CB | LYS | 226 | 103.451 | 68.283 | 90.144 | 0.00 | 0.00 | C |
| ATOM | 2891 | CG | LYS | 226 | 102.481 | 67.124 | 89.805 | 0.00 | 0.00 | C |
| ATOM | 2892 | CD | LYS | 226 | 102.522 | 66.736 | 88.315 | 0.00 | 0.00 | C |
| ATOM | 2893 | CE | LYS | 226 | 101.637 | 65.515 | 88.023 | 0.00 | 0.00 | C |
| ATOM | 2894 | NZ | LYS | 226 | 101.527 | 65.311 | 86.566 | 0.00 | 0.00 | N |
| ATOM | 2895 | 1HZ | LYS | 226 | 100.932 | 64.491 | 86.378 | 1.00 | 0.00 | H |
| ATOM | 2896 | 2HZ | LYS | 226 | 101.107 | 66.146 | 86.133 | 1.00 | 0.00 | H |
| ATOM | 2897 | 3HZ | LYS | 226 | 102.464 | 65.153 | 86.169 | 1.00 | 0.00 | H |
| ATOM | 2898 | H | LYS | 226 | 101.835 | 70.349 | 90.892 | 0.00 | 0.00 | H |
| ATOM | 2899 | HA | LYS | 226 | 103.295 | 68.031 | 92.309 | 0.00 | 0.00 | H |
| ATOM | 2900 | 1HB | LYS | 226 | 104.478 | 67.911 | 89.973 | 0.00 | 0.00 | H |
| ATOM | 2901 | 2HB | LYS | 226 | 103.337 | 69.102 | 89.410 | 0.00 | 0.00 | H |
| ATOM | 2902 | 1HG | LYS | 226 | 101.442 | 67.407 | 90.074 | 0.00 | 0.00 | H |
| ATOM | 2903 | 2HG | LYS | 226 | 102.712 | 66.247 | 90.442 | 0.00 | 0.00 | H |
| ATOM | 2904 | 1HD | LYS | 226 | 103.564 | 66.527 | 88.001 | 0.00 | 0.00 | H |
| ATOM | 2905 | 2HD | LYS | 226 | 102.191 | 67.603 | 87.712 | 0.00 | 0.00 | H |
| ATOM | 2906 | 1HE | LYS | 226 | 100.625 | 65.645 | 88.456 | 0.00 | 0.00 | H |
| ATOM | 2907 | 2HE | LYS | 226 | 102.050 | 64.607 | 88.504 | 0.00 | 0.00 | H |
| ATOM | 2908 | N | GLY | 227 | 105.779 | 69.164 | 92.013 | 0.00 | 0.00 | N |
| ATOM | 2909 | CA | GLY | 227 | 107.043 | 69.942 | 92.196 | 0.00 | 0.00 | C |
| ATOM | 2910 | C | GLY | 227 | 107.474 | 70.781 | 90.969 | 0.00 | 0.00 | C |
| ATOM | 2911 | O | GLY | 227 | 108.252 | 70.317 | 90.129 | 0.00 | 0.00 | O |
| ATOM | 2912 | H | GLY | 227 | 105.763 | 68.170 | 91.767 | 0.00 | 0.00 | H |
| ATOM | 2913 | 1HA | GLY | 227 | 107.867 | 69.245 | 92.430 | 0.00 | 0.00 | H |
| ATOM | 2914 | 2HA | GLY | 227 | 106.975 | 70.593 | 93.090 | 0.00 | 0.00 | H |
| ATOM | 2915 | N | HIS | 228 | 106.938 | 72.001 | 90.862 | 1.00 | 0.00 | N |
| ATOM | 2916 | CA | HIS | 228 | 107.134 | 72.882 | 89.682 | 1.00 | 0.00 | C |
| ATOM | 2917 | C | HIS | 228 | 108.488 | 73.673 | 89.671 | 1.00 | 0.00 | C |
| ATOM | 2918 | O | HIS | 228 | 109.095 | 73.931 | 90.713 | 1.00 | 0.00 | O |
| ATOM | 2919 | CB | HIS | 228 | 105.849 | 73.745 | 89.522 | 1.00 | 0.00 | C |
| ATOM | 2920 | CG | HIS | 228 | 105.638 | 74.923 | 90.476 | 1.00 | 0.00 | C |
| ATOM | 2921 | ND1 | HIS | 228 | 104.839 | 74.852 | 91.605 | 1.00 | 0.00 | N |
| ATOM | 2922 | CE1 | HIS | 228 | 104.822 | 76.176 | 91.952 | 1.00 | 0.00 | C |
| ATOM | 2923 | NE2 | HIS | 228 | 105.504 | 77.090 | 91.195 | 1.00 | 0.00 | N |
| ATOM | 2924 | CD2 | HIS | 228 | 106.028 | 76.252 | 90.228 | 1.00 | 0.00 | C |
| ATOM | 2925 | H | HIS | 228 | 106.269 | 72.229 | 91.611 | 1.00 | 0.00 | H |
| ATOM | 2926 | HA | HIS | 228 | 107.156 | 72.227 | 88.789 | 1.00 | 0.00 | H |
| ATOM | 2927 | 1HB | HIS | 228 | 105.814 | 74.126 | 88.490 | 1.00 | 0.00 | H |
| ATOM | 2928 | 2HB | HIS | 228 | 104.952 | 73.095 | 89.578 | 1.00 | 0.00 | H |
| ATOM | 2929 | HE1 | HIS | 228 | 104.223 | 76.496 | 92.796 | 1.00 | 0.00 | H |
| ATOM | 2930 | HE2 | HIS | 228 | 105.433 | 78.113 | 91.204 | 1.00 | 0.00 | H |
| ATOM | 2931 | HD2 | HIS | 228 | 106.583 | 76.576 | 89.357 | 1.00 | 0.00 | H |
| ATOM | 2932 | N | SER | 229 | 108.982 | 74.034 | 88.477 | 1.00 | 0.00 | N |
| ATOM | 2933 | CA | SER | 229 | 110.344 | 74.627 | 88.307 | 1.00 | 0.00 | C |
| ATOM | 2934 | C | SER | 229 | 110.413 | 75.767 | 87.228 | 1.00 | 0.00 | C |
| ATOM | 2935 | O | SER | 229 | 109.414 | 76.160 | 86.618 | 1.00 | 0.00 | O |
| ATOM | 2936 | CB | SER | 229 | 111.307 | 73.436 | 88.019 | 1.00 | 0.00 | C |
| ATOM | 2937 | OG | SER | 229 | 111.082 | 72.858 | 86.728 | 1.00 | 0.00 | O |
| ATOM | 2938 | H | SER | 229 | 108.401 | 73.777 | 87.675 | 1.00 | 0.00 | H |
| ATOM | 2939 | HA | SER | 229 | 110.667 | 75.107 | 89.254 | 1.00 | 0.00 | H |
| ATOM | 2940 | 1HB | SER | 229 | 112.359 | 73.774 | 88.078 | 1.00 | 0.00 | H |
| ATOM | 2941 | 2HB | SER | 229 | 111.226 | 72.659 | 88.807 | 1.00 | 0.00 | H |
| ATOM | 2942 | HG | SER | 229 | 110.404 | 72.178 | 86.829 | 1.00 | 0.00 | H |
| ATOM | 2943 | N | PHE | 230 | 111.629 | 76.267 | 86.950 | 1.00 | 0.00 | N |
| ATOM | 2944 | CA | PHE | 230 | 111.937 | 77.099 | 85.741 | 1.00 | 0.00 | C |
| ATOM | 2945 | C | PHE | 230 | 111.471 | 76.593 | 84.316 | 1.00 | 0.00 | C |
| ATOM | 2946 | O | PHE | 230 | 111.222 | 77.406 | 83.423 | 1.00 | 0.00 | O |
| ATOM | 2947 | CB | PHE | 230 | 113.452 | 77.459 | 85.816 | 1.00 | 0.00 | C |
| ATOM | 2948 | CG | PHE | 230 | 114.486 | 76.333 | 85.620 | 1.00 | 0.00 | C |
| ATOM | 2949 | CD1 | PHE | 230 | 114.974 | 76.051 | 84.343 | 1.00 | 0.00 | C |
| ATOM | 2950 | CE1 | PHE | 230 | 115.897 | 75.025 | 84.148 | 1.00 | 0.00 | C |
| ATOM | 2951 | CZ | PHE | 230 | 116.347 | 74.284 | 85.237 | 1.00 | 0.00 | C |
| ATOM | 2952 | CE2 | PHE | 230 | 115.881 | 74.568 | 86.518 | 1.00 | 0.00 | C |
| ATOM | 2953 | CD2 | PHE | 230 | 114.956 | 75.593 | 86.710 | 1.00 | 0.00 | C |
| ATOM | 2954 | H | PHE | 230 | 112.386 | 75.821 | 87.479 | 1.00 | 0.00 | H |
| ATOM | 2955 | HA | PHE | 230 | 111.391 | 78.053 | 85.871 | 1.00 | 0.00 | H |
| ATOM | 2956 | 1HB | PHE | 230 | 113.651 | 78.247 | 85.071 | 1.00 | 0.00 | H |
| ATOM | 2957 | 2HB | PHE | 230 | 113.669 | 77.979 | 86.769 | 1.00 | 0.00 | H |
| ATOM | 2958 | HD1 | PHE | 230 | 114.632 | 76.633 | 83.501 | 1.00 | 0.00 | H |
| ATOM | 2959 | HE1 | PHE | 230 | 116.265 | 74.805 | 83.156 | 1.00 | 0.00 | H |
| ATOM | 2960 | HZ | PHE | 230 | 117.066 | 73.490 | 85.087 | 1.00 | 0.00 | H |
| ATOM | 2961 | HE2 | PHE | 230 | 116.243 | 73.999 | 87.361 | 1.00 | 0.00 | H |
| ATOM | 2962 | HD2 | PHE | 230 | 114.613 | 75.812 | 87.710 | 1.00 | 0.00 | H |
| ATOM | 2963 | N | GLU | 231 | 111.292 | 75.272 | 84.131 | 1.00 | 0.00 | N |
| ATOM | 2964 | CA | GLU | 231 | 110.562 | 74.677 | 82.969 | 1.00 | 0.00 | C |
| ATOM | 2965 | C | GLU | 231 | 109.060 | 75.098 | 82.737 | 1.00 | 0.00 | C |
| ATOM | 2966 | O | GLU | 231 | 108.584 | 75.005 | 81.604 | 1.00 | 0.00 | O |
| ATOM | 2967 | CB | GLU | 231 | 110.656 | 73.133 | 83.100 | 1.00 | 0.00 | C |
| ATOM | 2968 | CG | GLU | 231 | 112.078 | 72.518 | 83.008 | 1.00 | 0.00 | C |
| ATOM | 2969 | CD | GLU | 231 | 112.065 | 70.999 | 82.925 | 1.00 | 0.00 | C |
| ATOM | 2970 | OE1 | GLU | 231 | 112.137 | 70.254 | 83.897 | 1.00 | 0.00 | O |
| ATOM | 2971 | OE2 | GLU | 231 | 111.937 | 70.573 | 81.644 | 1.00 | 0.00 | O |
| ATOM | 2972 | H | GLU | 231 | 111.571 | 74.703 | 84.940 | 1.00 | 0.00 | H |
| ATOM | 2973 | HA | GLU | 231 | 111.097 | 74.967 | 82.041 | 1.00 | 0.00 | H |
| ATOM | 2974 | 1HB | GLU | 231 | 110.167 | 72.799 | 84.038 | 1.00 | 0.00 | H |
| ATOM | 2975 | 2HB | GLU | 231 | 110.045 | 72.676 | 82.297 | 1.00 | 0.00 | H |
| ATOM | 2976 | 1HG | GLU | 231 | 112.608 | 72.908 | 82.120 | 1.00 | 0.00 | H |
| ATOM | 2977 | 2HG | GLU | 231 | 112.696 | 72.813 | 83.876 | 1.00 | 0.00 | H |
| ATOM | 2978 | N | VAL | 232 | 108.315 | 75.563 | 83.762 | 0.00 | 0.00 | N |
| ATOM | 2979 | CA | VAL | 232 | 106.975 | 76.233 | 83.587 | 0.00 | 0.00 | C |
| ATOM | 2980 | C | VAL | 232 | 106.973 | 77.440 | 82.572 | 0.00 | 0.00 | C |
| ATOM | 2981 | O | VAL | 232 | 106.066 | 77.540 | 81.743 | 0.00 | 0.00 | O |
| ATOM | 2982 | CB | VAL | 232 | 106.397 | 76.618 | 85.002 | 0.00 | 0.00 | C |
| ATOM | 2983 | CG1 | VAL | 232 | 105.069 | 77.417 | 84.983 | 0.00 | 0.00 | C |
| ATOM | 2984 | CG2 | VAL | 232 | 106.142 | 75.406 | 85.927 | 0.00 | 0.00 | C |
| ATOM | 2985 | H | VAL | 232 | 108.833 | 75.616 | 84.649 | 0.00 | 0.00 | H |
| ATOM | 2986 | HA | VAL | 232 | 106.286 | 75.487 | 83.145 | 0.00 | 0.00 | H |
| ATOM | 2987 | HB | VAL | 232 | 107.150 | 77.263 | 85.501 | 0.00 | 0.00 | H |
| ATOM | 2988 | 1HG1 | VAL | 232 | 104.717 | 77.675 | 86.001 | 0.00 | 0.00 | H |
| ATOM | 2989 | 2HG1 | VAL | 232 | 105.171 | 78.380 | 84.448 | 0.00 | 0.00 | H |
| ATOM | 2990 | 3HG1 | VAL | 232 | 104.250 | 76.866 | 84.487 | 0.00 | 0.00 | H |
| ATOM | 2991 | 1HG2 | VAL | 232 | 105.825 | 75.734 | 86.933 | 0.00 | 0.00 | H |
| ATOM | 2992 | 2HG2 | VAL | 232 | 105.361 | 74.727 | 85.535 | 0.00 | 0.00 | H |
| ATOM | 2993 | 3HG2 | VAL | 232 | 107.054 | 74.800 | 86.076 | 0.00 | 0.00 | H |
| ATOM | 2994 | N | ASP | 233 | 107.988 | 78.323 | 82.624 | 0.00 | 0.00 | N |
| ATOM | 2995 | CA | ASP | 233 | 108.152 | 79.450 | 81.659 | 0.00 | 0.00 | C |
| ATOM | 2996 | C | ASP | 233 | 108.270 | 79.073 | 80.140 | 0.00 | 0.00 | C |
| ATOM | 2997 | O | ASP | 233 | 107.709 | 79.791 | 79.312 | 0.00 | 0.00 | O |
| ATOM | 2998 | CB | ASP | 233 | 109.365 | 80.306 | 82.112 | 0.00 | 0.00 | C |
| ATOM | 2999 | CG | ASP | 233 | 109.178 | 81.054 | 83.429 | 0.00 | 0.00 | C |
| ATOM | 3000 | OD1 | ASP | 233 | 108.462 | 82.039 | 83.552 | 0.00 | 0.00 | O |
| ATOM | 3001 | OD2 | ASP | 233 | 109.888 | 80.506 | 84.452 | 0.00 | 0.00 | O |
| ATOM | 3002 | H | ASP | 233 | 108.728 | 78.048 | 83.281 | 0.00 | 0.00 | H |
| ATOM | 3003 | HA | ASP | 233 | 107.242 | 80.084 | 81.725 | 0.00 | 0.00 | H |
| ATOM | 3004 | 1HB | ASP | 233 | 109.579 | 81.077 | 81.350 | 0.00 | 0.00 | H |
| ATOM | 3005 | 2HB | ASP | 233 | 110.280 | 79.686 | 82.156 | 0.00 | 0.00 | H |
| ATOM | 3006 | N | VAL | 234 | 108.966 | 77.984 | 79.753 | 1.00 | 0.00 | N |
| ATOM | 3007 | CA | VAL | 234 | 109.051 | 77.551 | 78.315 | 1.00 | 0.00 | C |
| ATOM | 3008 | C | VAL | 234 | 107.716 | 77.024 | 77.672 | 1.00 | 0.00 | C |
| ATOM | 3009 | O | VAL | 234 | 107.491 | 77.285 | 76.490 | 1.00 | 0.00 | O |
| ATOM | 3010 | CB | VAL | 234 | 110.287 | 76.640 | 77.996 | 1.00 | 0.00 | C |
| ATOM | 3011 | CG1 | VAL | 234 | 111.637 | 77.212 | 78.478 | 1.00 | 0.00 | C |
| ATOM | 3012 | CG2 | VAL | 234 | 110.179 | 75.177 | 78.463 | 1.00 | 0.00 | C |
| ATOM | 3013 | H | VAL | 234 | 109.295 | 77.390 | 80.522 | 1.00 | 0.00 | H |
| ATOM | 3014 | HA | VAL | 234 | 109.256 | 78.472 | 77.736 | 1.00 | 0.00 | H |
| ATOM | 3015 | HB | VAL | 234 | 110.352 | 76.595 | 76.889 | 1.00 | 0.00 | H |
| ATOM | 3016 | 1HG1 | VAL | 234 | 111.830 | 78.217 | 78.064 | 1.00 | 0.00 | H |
| ATOM | 3017 | 2HG1 | VAL | 234 | 111.679 | 77.294 | 79.581 | 1.00 | 0.00 | H |
| ATOM | 3018 | 3HG1 | VAL | 234 | 112.485 | 76.569 | 78.180 | 1.00 | 0.00 | H |
| ATOM | 3019 | 2HG2 | VAL | 234 | 109.275 | 74.691 | 78.054 | 1.00 | 0.00 | H |
| ATOM | 3020 | 3HG2 | VAL | 234 | 111.032 | 74.567 | 78.108 | 1.00 | 0.00 | H |
| ATOM | 3021 | 1HG2 | VAL | 234 | 110.144 | 75.080 | 79.563 | 1.00 | 0.00 | H |
| ATOM | 3022 | N | TRP | 235 | 106.805 | 76.370 | 78.426 | 0.00 | 0.00 | N |
| ATOM | 3023 | CA | TRP | 235 | 105.377 | 76.190 | 78.008 | 0.00 | 0.00 | C |
| ATOM | 3024 | C | TRP | 235 | 104.631 | 77.524 | 77.617 | 0.00 | 0.00 | C |
| ATOM | 3025 | O | TRP | 235 | 104.044 | 77.605 | 76.537 | 0.00 | 0.00 | O |
| ATOM | 3026 | CB | TRP | 235 | 104.678 | 75.370 | 79.134 | 0.00 | 0.00 | C |
| ATOM | 3027 | CG | TRP | 235 | 103.237 | 74.907 | 78.840 | 0.00 | 0.00 | C |
| ATOM | 3028 | CD1 | TRP | 235 | 102.087 | 75.732 | 78.847 | 0.00 | 0.00 | C |
| ATOM | 3029 | NE1 | TRP | 235 | 100.919 | 75.016 | 78.528 | 0.00 | 0.00 | N |
| ATOM | 3030 | CE2 | TRP | 235 | 101.363 | 73.721 | 78.340 | 0.00 | 0.00 | C |
| ATOM | 3031 | CD2 | TRP | 235 | 102.767 | 73.636 | 78.544 | 0.00 | 0.00 | C |
| ATOM | 3032 | CE3 | TRP | 235 | 103.427 | 72.383 | 78.450 | 0.00 | 0.00 | C |
| ATOM | 3033 | CZ3 | TRP | 235 | 102.666 | 71.256 | 78.137 | 0.00 | 0.00 | C |
| ATOM | 3034 | CH2 | TRP | 235 | 101.286 | 71.342 | 77.920 | 0.00 | 0.00 | C |
| ATOM | 3035 | CZ2 | TRP | 235 | 100.618 | 72.563 | 78.027 | 0.00 | 0.00 | C |
| ATOM | 3036 | H | TRP | 235 | 107.125 | 76.193 | 79.384 | 0.00 | 0.00 | H |
| ATOM | 3037 | HA | TRP | 235 | 105.383 | 75.559 | 77.096 | 0.00 | 0.00 | H |
| ATOM | 3038 | 1HB | TRP | 235 | 104.675 | 75.961 | 80.068 | 0.00 | 0.00 | H |
| ATOM | 3039 | 2HB | TRP | 235 | 105.288 | 74.475 | 79.371 | 0.00 | 0.00 | H |
| ATOM | 3040 | HD1 | TRP | 235 | 102.100 | 76.787 | 79.085 | 0.00 | 0.00 | H |
| ATOM | 3041 | HE1 | TRP | 235 | 99.945 | 75.330 | 78.617 | 0.00 | 0.00 | H |
| ATOM | 3042 | HE3 | TRP | 235 | 104.496 | 72.298 | 78.604 | 0.00 | 0.00 | H |
| ATOM | 3043 | HZ3 | TRP | 235 | 103.151 | 70.298 | 78.052 | 0.00 | 0.00 | H |
| ATOM | 3044 | HH2 | TRP | 235 | 100.726 | 70.451 | 77.675 | 0.00 | 0.00 | H |
| ATOM | 3045 | HZ2 | TRP | 235 | 99.552 | 72.617 | 77.872 | 0.00 | 0.00 | H |
| ATOM | 3046 | N | SER | 236 | 104.693 | 78.574 | 78.457 | 1.00 | 0.00 | N |
| ATOM | 3047 | CA | SER | 236 | 104.260 | 79.951 | 78.074 | 1.00 | 0.00 | C |
| ATOM | 3048 | C | SER | 236 | 105.007 | 80.630 | 76.859 | 1.00 | 0.00 | C |
| ATOM | 3049 | O | SER | 236 | 104.369 | 81.372 | 76.112 | 1.00 | 0.00 | O |
| ATOM | 3050 | CB | SER | 236 | 104.309 | 80.831 | 79.347 | 1.00 | 0.00 | C |
| ATOM | 3051 | OG | SER | 236 | 103.493 | 80.340 | 80.418 | 1.00 | 0.00 | O |
| ATOM | 3052 | H | SER | 236 | 105.222 | 78.388 | 79.317 | 1.00 | 0.00 | H |
| ATOM | 3053 | HA | SER | 236 | 103.196 | 79.893 | 77.768 | 1.00 | 0.00 | H |
| ATOM | 3054 | 1HB | SER | 236 | 105.351 | 80.918 | 79.709 | 1.00 | 0.00 | H |
| ATOM | 3055 | 2HB | SER | 236 | 103.998 | 81.867 | 79.110 | 1.00 | 0.00 | H |
| ATOM | 3056 | HG | SER | 236 | 102.778 | 79.795 | 80.055 | 1.00 | 0.00 | H |
| ATOM | 3057 | N | ILE | 237 | 106.309 | 80.357 | 76.616 | 1.00 | 0.00 | N |
| ATOM | 3058 | CA | ILE | 237 | 107.003 | 80.705 | 75.321 | 1.00 | 0.00 | C |
| ATOM | 3059 | C | ILE | 237 | 106.456 | 79.903 | 74.072 | 1.00 | 0.00 | C |
| ATOM | 3060 | O | ILE | 237 | 106.248 | 80.500 | 73.014 | 1.00 | 0.00 | O |
| ATOM | 3061 | CB | ILE | 237 | 108.578 | 80.663 | 75.438 | 1.00 | 0.00 | C |
| ATOM | 3062 | CG1 | ILE | 237 | 109.157 | 81.505 | 76.618 | 1.00 | 0.00 | C |
| ATOM | 3063 | CG2 | ILE | 237 | 109.275 | 81.142 | 74.129 | 1.00 | 0.00 | C |
| ATOM | 3064 | CD1 | ILE | 237 | 110.646 | 81.287 | 76.949 | 1.00 | 0.00 | C |
| ATOM | 3065 | H | ILE | 237 | 106.740 | 79.761 | 77.333 | 1.00 | 0.00 | H |
| ATOM | 3066 | HA | ILE | 237 | 106.758 | 81.763 | 75.117 | 1.00 | 0.00 | H |
| ATOM | 3067 | HB | ILE | 237 | 108.860 | 79.603 | 75.604 | 1.00 | 0.00 | H |
| ATOM | 3068 | 1HG1 | ILE | 237 | 108.966 | 82.579 | 76.437 | 1.00 | 0.00 | H |
| ATOM | 3069 | 2HG1 | ILE | 237 | 108.591 | 81.285 | 77.540 | 1.00 | 0.00 | H |
| ATOM | 3070 | 2HG2 | ILE | 237 | 108.960 | 80.560 | 73.244 | 1.00 | 0.00 | H |
| ATOM | 3071 | 3HG2 | ILE | 237 | 109.062 | 82.205 | 73.906 | 1.00 | 0.00 | H |
| ATOM | 3072 | 1HG2 | ILE | 237 | 110.374 | 81.034 | 74.173 | 1.00 | 0.00 | H |
| ATOM | 3073 | 2HD1 | ILE | 237 | 110.884 | 80.218 | 77.100 | 1.00 | 0.00 | H |
| ATOM | 3074 | 3HD1 | ILE | 237 | 111.312 | 81.668 | 76.152 | 1.00 | 0.00 | H |
| ATOM | 3075 | 1HD1 | ILE | 237 | 110.933 | 81.818 | 77.875 | 1.00 | 0.00 | H |
| ATOM | 3076 | N | GLY | 238 | 106.200 | 78.583 | 74.167 | 0.00 | 0.00 | N |
| ATOM | 3077 | CA | GLY | 238 | 105.387 | 77.841 | 73.149 | 0.00 | 0.00 | C |
| ATOM | 3078 | C | GLY | 238 | 103.976 | 78.385 | 72.795 | 0.00 | 0.00 | C |
| ATOM | 3079 | O | GLY | 238 | 103.633 | 78.481 | 71.615 | 0.00 | 0.00 | O |
| ATOM | 3080 | H | GLY | 238 | 106.452 | 78.180 | 75.081 | 0.00 | 0.00 | H |
| ATOM | 3081 | 1HA | GLY | 238 | 105.261 | 76.799 | 73.491 | 0.00 | 0.00 | H |
| ATOM | 3082 | 2HA | GLY | 238 | 105.967 | 77.757 | 72.212 | 0.00 | 0.00 | H |
| ATOM | 3083 | N | CYS | 239 | 103.189 | 78.784 | 73.803 | 1.00 | 0.00 | N |
| ATOM | 3084 | CA | CYS | 239 | 101.955 | 79.588 | 73.598 | 1.00 | 0.00 | C |
| ATOM | 3085 | C | CYS | 239 | 102.130 | 81.037 | 73.017 | 1.00 | 0.00 | C |
| ATOM | 3086 | O | CYS | 239 | 101.320 | 81.424 | 72.177 | 1.00 | 0.00 | O |
| ATOM | 3087 | CB | CYS | 239 | 101.175 | 79.555 | 74.929 | 1.00 | 0.00 | C |
| ATOM | 3088 | SG | CYS | 239 | 100.684 | 77.847 | 75.353 | 1.00 | 0.00 | S |
| ATOM | 3089 | H | CYS | 239 | 103.559 | 78.560 | 74.737 | 1.00 | 0.00 | H |
| ATOM | 3090 | HA | CYS | 239 | 101.333 | 79.056 | 72.854 | 1.00 | 0.00 | H |
| ATOM | 3091 | 1HB | CYS | 239 | 101.770 | 79.984 | 75.756 | 1.00 | 0.00 | H |
| ATOM | 3092 | 2HB | CYS | 239 | 100.257 | 80.168 | 74.857 | 1.00 | 0.00 | H |
| ATOM | 3093 | HG | CYS | 239 | 99.879 | 78.149 | 76.369 | 1.00 | 0.00 | H |
| ATOM | 3094 | N | ILE | 240 | 103.144 | 81.846 | 73.401 | 1.00 | 0.00 | N |
| ATOM | 3095 | CA | ILE | 240 | 103.446 | 83.157 | 72.720 | 1.00 | 0.00 | C |
| ATOM | 3096 | C | ILE | 240 | 103.857 | 83.041 | 71.205 | 1.00 | 0.00 | C |
| ATOM | 3097 | O | ILE | 240 | 103.322 | 83.776 | 70.376 | 1.00 | 0.00 | O |
| ATOM | 3098 | CB | ILE | 240 | 104.363 | 84.074 | 73.623 | 1.00 | 0.00 | C |
| ATOM | 3099 | CG1 | ILE | 240 | 104.030 | 85.592 | 73.537 | 1.00 | 0.00 | C |
| ATOM | 3100 | CG2 | ILE | 240 | 105.884 | 83.930 | 73.375 | 1.00 | 0.00 | C |
| ATOM | 3101 | CD1 | ILE | 240 | 102.718 | 86.012 | 74.219 | 1.00 | 0.00 | C |
| ATOM | 3102 | H | ILE | 240 | 103.768 | 81.425 | 74.103 | 1.00 | 0.00 | H |
| ATOM | 3103 | HA | ILE | 240 | 102.475 | 83.683 | 72.661 | 1.00 | 0.00 | H |
| ATOM | 3104 | HB | ILE | 240 | 104.204 | 83.784 | 74.683 | 1.00 | 0.00 | H |
| ATOM | 3105 | 1HG1 | ILE | 240 | 104.839 | 86.186 | 74.008 | 1.00 | 0.00 | H |
| ATOM | 3106 | 2HG1 | ILE | 240 | 104.027 | 85.921 | 72.479 | 1.00 | 0.00 | H |
| ATOM | 3107 | 2HG2 | ILE | 240 | 106.194 | 82.878 | 73.307 | 1.00 | 0.00 | H |
| ATOM | 3108 | 3HG2 | ILE | 240 | 106.204 | 84.406 | 72.428 | 1.00 | 0.00 | H |
| ATOM | 3109 | 1HG2 | ILE | 240 | 106.480 | 84.382 | 74.189 | 1.00 | 0.00 | H |
| ATOM | 3110 | 2HD1 | ILE | 240 | 101.825 | 85.624 | 73.696 | 1.00 | 0.00 | H |
| ATOM | 3111 | 3HD1 | ILE | 240 | 102.667 | 85.675 | 75.270 | 1.00 | 0.00 | H |
| ATOM | 3112 | 1HD1 | ILE | 240 | 102.619 | 87.114 | 74.233 | 1.00 | 0.00 | H |
| ATOM | 3113 | N | MET | 241 | 104.735 | 82.087 | 70.837 | 1.00 | 0.00 | N |
| ATOM | 3114 | CA | MET | 241 | 104.976 | 81.681 | 69.418 | 1.00 | 0.00 | C |
| ATOM | 3115 | C | MET | 241 | 103.699 | 81.313 | 68.578 | 1.00 | 0.00 | C |
| ATOM | 3116 | O | MET | 241 | 103.509 | 81.859 | 67.490 | 1.00 | 0.00 | O |
| ATOM | 3117 | CB | MET | 241 | 106.015 | 80.527 | 69.488 | 1.00 | 0.00 | C |
| ATOM | 3118 | CG | MET | 241 | 106.567 | 80.031 | 68.129 | 1.00 | 0.00 | C |
| ATOM | 3119 | SD | MET | 241 | 107.621 | 78.555 | 68.239 | 1.00 | 0.00 | S |
| ATOM | 3120 | CE | MET | 241 | 108.499 | 78.673 | 69.810 | 1.00 | 0.00 | C |
| ATOM | 3121 | H | MET | 241 | 105.091 | 81.530 | 71.626 | 1.00 | 0.00 | H |
| ATOM | 3122 | HA | MET | 241 | 105.453 | 82.535 | 68.899 | 1.00 | 0.00 | H |
| ATOM | 3123 | 1HB | MET | 241 | 106.874 | 80.850 | 70.110 | 1.00 | 0.00 | H |
| ATOM | 3124 | 2HB | MET | 241 | 105.577 | 79.664 | 70.029 | 1.00 | 0.00 | H |
| ATOM | 3125 | 1HG | MET | 241 | 105.735 | 79.767 | 67.450 | 1.00 | 0.00 | H |
| ATOM | 3126 | 2HG | MET | 241 | 107.116 | 80.834 | 67.606 | 1.00 | 0.00 | H |
| ATOM | 3127 | 1HE | MET | 241 | 109.111 | 79.588 | 69.866 | 1.00 | 0.00 | H |
| ATOM | 3128 | 3HE | MET | 241 | 107.793 | 78.671 | 70.659 | 1.00 | 0.00 | H |
| ATOM | 3129 | 2HE | MET | 241 | 109.169 | 77.807 | 69.936 | 1.00 | 0.00 | H |
| ATOM | 3130 | N | TYR | 242 | 102.832 | 80.420 | 69.086 | 0.00 | 0.00 | N |
| ATOM | 3131 | CA | TYR | 242 | 101.524 | 80.095 | 68.447 | 0.00 | 0.00 | C |
| ATOM | 3132 | C | TYR | 242 | 100.518 | 81.301 | 68.359 | 0.00 | 0.00 | C |
| ATOM | 3133 | O | TYR | 242 | 100.074 | 81.616 | 67.254 | 0.00 | 0.00 | O |
| ATOM | 3134 | CB | TYR | 242 | 100.977 | 78.843 | 69.192 | 0.00 | 0.00 | C |
| ATOM | 3135 | CG | TYR | 242 | 99.848 | 78.067 | 68.487 | 0.00 | 0.00 | C |
| ATOM | 3136 | CD1 | TYR | 242 | 98.549 | 78.587 | 68.430 | 0.00 | 0.00 | C |
| ATOM | 3137 | CE1 | TYR | 242 | 97.505 | 77.821 | 67.920 | 0.00 | 0.00 | C |
| ATOM | 3138 | CZ | TYR | 242 | 97.747 | 76.525 | 67.476 | 0.00 | 0.00 | C |
| ATOM | 3139 | OH | TYR | 242 | 96.709 | 75.742 | 67.055 | 0.00 | 0.00 | O |
| ATOM | 3140 | CE2 | TYR | 242 | 99.037 | 76.005 | 67.506 | 0.00 | 0.00 | C |
| ATOM | 3141 | CD2 | TYR | 242 | 100.086 | 76.773 | 68.008 | 0.00 | 0.00 | C |
| ATOM | 3142 | H | TYR | 242 | 103.082 | 79.937 | 69.961 | 1.00 | 0.00 | H |
| ATOM | 3143 | HA | TYR | 242 | 101.733 | 79.791 | 67.401 | 0.00 | 0.00 | H |
| ATOM | 3144 | 1HB | TYR | 242 | 100.640 | 79.136 | 70.204 | 0.00 | 0.00 | H |
| ATOM | 3145 | 2HB | TYR | 242 | 101.809 | 78.139 | 69.399 | 0.00 | 0.00 | H |
| ATOM | 3146 | HD1 | TYR | 242 | 98.325 | 79.574 | 68.813 | 0.00 | 0.00 | H |
| ATOM | 3147 | HE1 | TYR | 242 | 96.504 | 78.227 | 67.911 | 0.00 | 0.00 | H |
| ATOM | 3148 | HH | TYR | 242 | 95.880 | 76.166 | 67.285 | 0.00 | 0.00 | H |
| ATOM | 3149 | HE2 | TYR | 242 | 99.213 | 74.992 | 67.173 | 0.00 | 0.00 | H |
| ATOM | 3150 | HD2 | TYR | 242 | 101.076 | 76.342 | 68.058 | 0.00 | 0.00 | H |
| ATOM | 3151 | N | THR | 243 | 100.184 | 81.981 | 69.478 | 1.00 | 0.00 | N |
| ATOM | 3152 | CA | THR | 243 | 99.285 | 83.185 | 69.481 | 1.00 | 0.00 | C |
| ATOM | 3153 | C | THR | 243 | 99.713 | 84.342 | 68.514 | 1.00 | 0.00 | C |
| ATOM | 3154 | O | THR | 243 | 98.888 | 84.807 | 67.726 | 1.00 | 0.00 | O |
| ATOM | 3155 | CB | THR | 243 | 99.090 | 83.743 | 70.932 | 1.00 | 0.00 | C |
| ATOM | 3156 | OG1 | THR | 243 | 98.832 | 82.718 | 71.884 | 1.00 | 0.00 | O |
| ATOM | 3157 | CG2 | THR | 243 | 97.910 | 84.721 | 71.059 | 1.00 | 0.00 | C |
| ATOM | 3158 | H | THR | 243 | 100.655 | 81.660 | 70.333 | 1.00 | 0.00 | H |
| ATOM | 3159 | HA | THR | 243 | 98.293 | 82.839 | 69.129 | 1.00 | 0.00 | H |
| ATOM | 3160 | HB | THR | 243 | 100.024 | 84.260 | 71.238 | 1.00 | 0.00 | H |
| ATOM | 3161 | HG1 | THR | 243 | 99.139 | 83.061 | 72.734 | 1.00 | 0.00 | H |
| ATOM | 3162 | 1HG2 | THR | 243 | 97.704 | 85.005 | 72.105 | 1.00 | 0.00 | H |
| ATOM | 3163 | 2HG2 | THR | 243 | 98.085 | 85.655 | 70.491 | 1.00 | 0.00 | H |
| ATOM | 3164 | 3HG2 | THR | 243 | 96.979 | 84.281 | 70.658 | 1.00 | 0.00 | H |
| ATOM | 3165 | N | LEU | 244 | 100.985 | 84.781 | 68.545 | 1.00 | 0.00 | N |
| ATOM | 3166 | CA | LEU | 244 | 101.545 | 85.737 | 67.545 | 1.00 | 0.00 | C |
| ATOM | 3167 | C | LEU | 244 | 101.478 | 85.258 | 66.049 | 1.00 | 0.00 | C |
| ATOM | 3168 | O | LEU | 244 | 101.053 | 86.030 | 65.189 | 1.00 | 0.00 | O |
| ATOM | 3169 | CB | LEU | 244 | 102.996 | 86.108 | 67.971 | 1.00 | 0.00 | C |
| ATOM | 3170 | CG | LEU | 244 | 103.197 | 86.828 | 69.336 | 1.00 | 0.00 | C |
| ATOM | 3171 | CD1 | LEU | 244 | 104.693 | 86.857 | 69.681 | 1.00 | 0.00 | C |
| ATOM | 3172 | CD2 | LEU | 244 | 102.634 | 88.258 | 69.364 | 1.00 | 0.00 | C |
| ATOM | 3173 | H | LEU | 244 | 101.595 | 84.257 | 69.185 | 1.00 | 0.00 | H |
| ATOM | 3174 | HA | LEU | 244 | 100.944 | 86.664 | 67.584 | 1.00 | 0.00 | H |
| ATOM | 3175 | 1HB | LEU | 244 | 103.598 | 85.177 | 67.962 | 1.00 | 0.00 | H |
| ATOM | 3176 | 2HB | LEU | 244 | 103.453 | 86.732 | 67.179 | 1.00 | 0.00 | H |
| ATOM | 3177 | HG | LEU | 244 | 102.682 | 86.247 | 70.127 | 1.00 | 0.00 | H |
| ATOM | 3178 | 2HD1 | LEU | 244 | 105.106 | 85.835 | 69.783 | 1.00 | 0.00 | H |
| ATOM | 3179 | 3HD1 | LEU | 244 | 105.282 | 87.366 | 68.897 | 1.00 | 0.00 | H |
| ATOM | 3180 | 1HD1 | LEU | 244 | 104.891 | 87.376 | 70.636 | 1.00 | 0.00 | H |
| ATOM | 3181 | 2HD2 | LEU | 244 | 103.144 | 88.924 | 68.647 | 1.00 | 0.00 | H |
| ATOM | 3182 | 3HD2 | LEU | 244 | 101.554 | 88.286 | 69.124 | 1.00 | 0.00 | H |
| ATOM | 3183 | 1HD2 | LEU | 244 | 102.744 | 88.721 | 70.362 | 1.00 | 0.00 | H |
| ATOM | 3184 | N | LEU | 245 | 101.858 | 84.002 | 65.739 | 1.00 | 0.00 | N |
| ATOM | 3185 | CA | LEU | 245 | 101.796 | 83.446 | 64.357 | 1.00 | 0.00 | C |
| ATOM | 3186 | C | LEU | 245 | 100.365 | 83.227 | 63.747 | 1.00 | 0.00 | C |
| ATOM | 3187 | O | LEU | 245 | 100.130 | 83.686 | 62.629 | 1.00 | 0.00 | O |
| ATOM | 3188 | CB | LEU | 245 | 102.732 | 82.203 | 64.331 | 1.00 | 0.00 | C |
| ATOM | 3189 | CG | LEU | 245 | 102.919 | 81.454 | 62.985 | 1.00 | 0.00 | C |
| ATOM | 3190 | CD1 | LEU | 245 | 103.344 | 82.362 | 61.820 | 1.00 | 0.00 | C |
| ATOM | 3191 | CD2 | LEU | 245 | 103.963 | 80.340 | 63.152 | 1.00 | 0.00 | C |
| ATOM | 3192 | H | LEU | 245 | 102.133 | 83.433 | 66.548 | 1.00 | 0.00 | H |
| ATOM | 3193 | HA | LEU | 245 | 102.271 | 84.194 | 63.697 | 1.00 | 0.00 | H |
| ATOM | 3194 | 1HB | LEU | 245 | 103.730 | 82.513 | 64.700 | 1.00 | 0.00 | H |
| ATOM | 3195 | 2HB | LEU | 245 | 102.371 | 81.475 | 65.084 | 1.00 | 0.00 | H |
| ATOM | 3196 | HG | LEU | 245 | 101.953 | 80.980 | 62.714 | 1.00 | 0.00 | H |
| ATOM | 3197 | 2HD1 | LEU | 245 | 102.592 | 83.146 | 61.614 | 1.00 | 0.00 | H |
| ATOM | 3198 | 3HD1 | LEU | 245 | 104.301 | 82.881 | 62.016 | 1.00 | 0.00 | H |
| ATOM | 3199 | 1HD1 | LEU | 245 | 103.446 | 81.789 | 60.880 | 1.00 | 0.00 | H |
| ATOM | 3200 | 2HD2 | LEU | 245 | 104.967 | 80.740 | 63.387 | 1.00 | 0.00 | H |
| ATOM | 3201 | 3HD2 | LEU | 245 | 103.690 | 79.650 | 63.972 | 1.00 | 0.00 | H |
| ATOM | 3202 | 1HD2 | LEU | 245 | 104.055 | 79.729 | 62.234 | 1.00 | 0.00 | H |
| ATOM | 3203 | N | VAL | 246 | 99.420 | 82.532 | 64.412 | 1.00 | 0.00 | N |
| ATOM | 3204 | CA | VAL | 246 | 98.018 | 82.373 | 63.887 | 1.00 | 0.00 | C |
| ATOM | 3205 | C | VAL | 246 | 97.012 | 83.542 | 64.197 | 1.00 | 0.00 | C |
| ATOM | 3206 | O | VAL | 246 | 96.113 | 83.787 | 63.387 | 1.00 | 0.00 | O |
| ATOM | 3207 | CB | VAL | 246 | 97.466 | 80.939 | 64.220 | 1.00 | 0.00 | C |
| ATOM | 3208 | CG1 | VAL | 246 | 97.060 | 80.705 | 65.691 | 1.00 | 0.00 | C |
| ATOM | 3209 | CG2 | VAL | 246 | 96.256 | 80.541 | 63.340 | 1.00 | 0.00 | C |
| ATOM | 3210 | H | VAL | 246 | 99.698 | 82.253 | 65.364 | 1.00 | 0.00 | H |
| ATOM | 3211 | HA | VAL | 246 | 98.082 | 82.391 | 62.781 | 1.00 | 0.00 | H |
| ATOM | 3212 | HB | VAL | 246 | 98.274 | 80.217 | 63.987 | 1.00 | 0.00 | H |
| ATOM | 3213 | 1HG1 | VAL | 246 | 96.219 | 81.348 | 66.008 | 1.00 | 0.00 | H |
| ATOM | 3214 | 2HG1 | VAL | 246 | 96.751 | 79.661 | 65.873 | 1.00 | 0.00 | H |
| ATOM | 3215 | 3HG1 | VAL | 246 | 97.889 | 80.912 | 66.389 | 1.00 | 0.00 | H |
| ATOM | 3216 | 2HG2 | VAL | 246 | 96.503 | 80.566 | 62.265 | 1.00 | 0.00 | H |
| ATOM | 3217 | 3HG2 | VAL | 246 | 95.894 | 79.519 | 63.559 | 1.00 | 0.00 | H |
| ATOM | 3218 | 1HG2 | VAL | 246 | 95.393 | 81.219 | 63.488 | 1.00 | 0.00 | H |
| ATOM | 3219 | N | GLY | 247 | 97.099 | 84.206 | 65.360 | 0.00 | 0.00 | N |
| ATOM | 3220 | CA | GLY | 247 | 96.020 | 85.100 | 65.871 | 0.00 | 0.00 | C |
| ATOM | 3221 | C | GLY | 247 | 95.492 | 84.695 | 67.263 | 0.00 | 0.00 | C |
| ATOM | 3222 | O | GLY | 247 | 95.697 | 85.401 | 68.254 | 0.00 | 0.00 | O |
| ATOM | 3223 | H | GLY | 247 | 97.934 | 83.977 | 65.917 | 0.00 | 0.00 | H |
| ATOM | 3224 | 1HA | GLY | 247 | 95.163 | 85.180 | 65.173 | 0.00 | 0.00 | H |
| ATOM | 3225 | 2HA | GLY | 247 | 96.403 | 86.132 | 65.927 | 0.00 | 0.00 | H |
| ATOM | 3226 | N | LYS | 248 | 94.784 | 83.561 | 67.320 | 1.00 | 0.00 | N |
| ATOM | 3227 | CA | LYS | 248 | 94.201 | 83.016 | 68.579 | 1.00 | 0.00 | C |
| ATOM | 3228 | C | LYS | 248 | 95.191 | 82.099 | 69.402 | 1.00 | 0.00 | C |
| ATOM | 3229 | O | LYS | 248 | 95.994 | 81.382 | 68.791 | 1.00 | 0.00 | O |
| ATOM | 3230 | CB | LYS | 248 | 92.912 | 82.231 | 68.204 | 1.00 | 0.00 | C |
| ATOM | 3231 | CG | LYS | 248 | 91.705 | 83.103 | 67.777 | 1.00 | 0.00 | C |
| ATOM | 3232 | CD | LYS | 248 | 90.498 | 82.257 | 67.331 | 1.00 | 0.00 | C |
| ATOM | 3233 | CE | LYS | 248 | 89.278 | 83.127 | 66.999 | 1.00 | 0.00 | C |
| ATOM | 3234 | NZ | LYS | 248 | 88.159 | 82.262 | 66.577 | 1.00 | 0.00 | N |
| ATOM | 3235 | 1HZ | LYS | 248 | 87.339 | 82.845 | 66.354 | 1.00 | 0.00 | H |
| ATOM | 3236 | 2HZ | LYS | 248 | 88.436 | 81.725 | 65.743 | 1.00 | 0.00 | H |
| ATOM | 3237 | 3HZ | LYS | 248 | 87.920 | 81.612 | 67.339 | 1.00 | 0.00 | H |
| ATOM | 3238 | H | LYS | 248 | 94.798 | 83.034 | 66.443 | 1.00 | 0.00 | H |
| ATOM | 3239 | HA | LYS | 248 | 93.904 | 83.868 | 69.219 | 1.00 | 0.00 | H |
| ATOM | 3240 | 1HB | LYS | 248 | 93.145 | 81.487 | 67.415 | 1.00 | 0.00 | H |
| ATOM | 3241 | 2HB | LYS | 248 | 92.596 | 81.613 | 69.067 | 1.00 | 0.00 | H |
| ATOM | 3242 | 1HG | LYS | 248 | 91.419 | 83.774 | 68.610 | 1.00 | 0.00 | H |
| ATOM | 3243 | 2HG | LYS | 248 | 92.002 | 83.776 | 66.948 | 1.00 | 0.00 | H |
| ATOM | 3244 | 1HD | LYS | 248 | 90.780 | 81.656 | 66.443 | 1.00 | 0.00 | H |
| ATOM | 3245 | 2HD | LYS | 248 | 90.239 | 81.516 | 68.112 | 1.00 | 0.00 | H |
| ATOM | 3246 | 1HE | LYS | 248 | 88.979 | 83.741 | 67.873 | 1.00 | 0.00 | H |
| ATOM | 3247 | 2HE | LYS | 248 | 89.524 | 83.843 | 66.188 | 1.00 | 0.00 | H |
| ATOM | 3248 | N | PRO | 249 | 95.154 | 82.039 | 70.771 | 0.00 | 0.00 | N |
| ATOM | 3249 | CA | PRO | 249 | 96.020 | 81.112 | 71.555 | 0.00 | 0.00 | C |
| ATOM | 3250 | CD | PRO | 249 | 94.324 | 82.923 | 71.609 | 0.00 | 0.00 | C |
| ATOM | 3251 | C | PRO | 249 | 95.717 | 79.571 | 71.421 | 0.00 | 0.00 | C |
| ATOM | 3252 | O | PRO | 249 | 94.594 | 79.206 | 71.051 | 0.00 | 0.00 | O |
| ATOM | 3253 | CB | PRO | 249 | 95.856 | 81.670 | 72.986 | 0.00 | 0.00 | C |
| ATOM | 3254 | CG | PRO | 249 | 94.475 | 82.324 | 73.005 | 0.00 | 0.00 | C |
| ATOM | 3255 | HA | PRO | 249 | 97.059 | 81.260 | 71.214 | 0.00 | 0.00 | H |
| ATOM | 3256 | 1HD | PRO | 249 | 93.266 | 82.947 | 71.289 | 0.00 | 0.00 | H |
| ATOM | 3257 | 2HD | PRO | 249 | 94.709 | 83.962 | 71.575 | 0.00 | 0.00 | H |
| ATOM | 3258 | 1HB | PRO | 249 | 96.635 | 82.431 | 73.179 | 0.00 | 0.00 | H |
| ATOM | 3259 | 2HB | PRO | 249 | 95.969 | 80.898 | 73.772 | 0.00 | 0.00 | H |
| ATOM | 3260 | 1HG | PRO | 249 | 94.358 | 83.079 | 73.801 | 0.00 | 0.00 | H |
| ATOM | 3261 | 2HG | PRO | 249 | 93.697 | 81.554 | 73.164 | 0.00 | 0.00 | H |
| ATOM | 3262 | N | PRO | 250 | 96.669 | 78.630 | 71.710 | 0.00 | 0.00 | N |
| ATOM | 3263 | CA | PRO | 250 | 96.501 | 77.190 | 71.345 | 0.00 | 0.00 | C |
| ATOM | 3264 | CD | PRO | 250 | 98.027 | 78.957 | 72.199 | 0.00 | 0.00 | C |
| ATOM | 3265 | C | PRO | 250 | 95.405 | 76.338 | 72.063 | 0.00 | 0.00 | C |
| ATOM | 3266 | O | PRO | 250 | 94.769 | 75.510 | 71.408 | 0.00 | 0.00 | O |
| ATOM | 3267 | CB | PRO | 250 | 97.937 | 76.661 | 71.500 | 0.00 | 0.00 | C |
| ATOM | 3268 | CG | PRO | 250 | 98.615 | 77.582 | 72.511 | 0.00 | 0.00 | C |
| ATOM | 3269 | HA | PRO | 250 | 96.236 | 77.132 | 70.273 | 0.00 | 0.00 | H |
| ATOM | 3270 | 1HD | PRO | 250 | 98.022 | 79.609 | 73.093 | 0.00 | 0.00 | H |
| ATOM | 3271 | 2HD | PRO | 250 | 98.612 | 79.473 | 71.412 | 0.00 | 0.00 | H |
| ATOM | 3272 | 1HB | PRO | 250 | 98.465 | 76.713 | 70.531 | 0.00 | 0.00 | H |
| ATOM | 3273 | 2HB | PRO | 250 | 97.983 | 75.602 | 71.782 | 0.00 | 0.00 | H |
| ATOM | 3274 | 1HG | PRO | 250 | 99.717 | 77.552 | 72.427 | 0.00 | 0.00 | H |
| ATOM | 3275 | 2HG | PRO | 250 | 98.359 | 77.276 | 73.544 | 0.00 | 0.00 | H |
| ATOM | 3276 | N | PHE | 251 | 95.171 | 76.536 | 73.369 | 0.00 | 0.00 | N |
| ATOM | 3277 | CA | PHE | 251 | 93.963 | 75.999 | 74.056 | 0.00 | 0.00 | C |
| ATOM | 3278 | C | PHE | 251 | 93.134 | 77.170 | 74.676 | 0.00 | 0.00 | C |
| ATOM | 3279 | O | PHE | 251 | 93.535 | 77.784 | 75.669 | 0.00 | 0.00 | O |
| ATOM | 3280 | CB | PHE | 251 | 94.359 | 74.924 | 75.110 | 0.00 | 0.00 | C |
| ATOM | 3281 | CG | PHE | 251 | 94.855 | 73.539 | 74.627 | 0.00 | 0.00 | C |
| ATOM | 3282 | CD1 | PHE | 251 | 94.370 | 72.914 | 73.468 | 0.00 | 0.00 | C |
| ATOM | 3283 | CE1 | PHE | 251 | 94.778 | 71.624 | 73.136 | 0.00 | 0.00 | C |
| ATOM | 3284 | CZ | PHE | 251 | 95.658 | 70.940 | 73.966 | 0.00 | 0.00 | C |
| ATOM | 3285 | CE2 | PHE | 251 | 96.139 | 71.542 | 75.122 | 0.00 | 0.00 | C |
| ATOM | 3286 | CD2 | PHE | 251 | 95.739 | 72.836 | 75.452 | 0.00 | 0.00 | C |
| ATOM | 3287 | H | PHE | 251 | 95.711 | 77.322 | 73.743 | 0.00 | 0.00 | H |
| ATOM | 3288 | HA | PHE | 251 | 93.295 | 75.495 | 73.329 | 0.00 | 0.00 | H |
| ATOM | 3289 | 1HB | PHE | 251 | 93.478 | 74.727 | 75.750 | 0.00 | 0.00 | H |
| ATOM | 3290 | 2HB | PHE | 251 | 95.092 | 75.375 | 75.806 | 0.00 | 0.00 | H |
| ATOM | 3291 | HD1 | PHE | 251 | 93.671 | 73.410 | 72.814 | 0.00 | 0.00 | H |
| ATOM | 3292 | HE1 | PHE | 251 | 94.410 | 71.151 | 72.236 | 0.00 | 0.00 | H |
| ATOM | 3293 | HZ | PHE | 251 | 95.972 | 69.941 | 73.707 | 0.00 | 0.00 | H |
| ATOM | 3294 | HE2 | PHE | 251 | 96.825 | 71.005 | 75.763 | 0.00 | 0.00 | H |
| ATOM | 3295 | HD2 | PHE | 251 | 96.115 | 73.283 | 76.358 | 0.00 | 0.00 | H |
| ATOM | 3296 | N | GLU | 252 | 91.963 | 77.457 | 74.083 | 1.00 | 0.00 | N |
| ATOM | 3297 | CA | GLU | 252 | 90.978 | 78.433 | 74.629 | 1.00 | 0.00 | C |
| ATOM | 3298 | C | GLU | 252 | 89.511 | 77.887 | 74.512 | 1.00 | 0.00 | C |
| ATOM | 3299 | O | GLU | 252 | 88.774 | 78.205 | 73.573 | 1.00 | 0.00 | O |
| ATOM | 3300 | CB | GLU | 252 | 91.191 | 79.843 | 74.006 | 1.00 | 0.00 | C |
| ATOM | 3301 | CG | GLU | 252 | 91.120 | 79.990 | 72.457 | 1.00 | 0.00 | C |
| ATOM | 3302 | CD | GLU | 252 | 90.880 | 81.410 | 71.943 | 1.00 | 0.00 | C |
| ATOM | 3303 | OE1 | GLU | 252 | 91.116 | 82.435 | 72.575 | 1.00 | 0.00 | O |
| ATOM | 3304 | OE2 | GLU | 252 | 90.369 | 81.410 | 70.683 | 1.00 | 0.00 | O |
| ATOM | 3305 | H | GLU | 252 | 91.798 | 76.958 | 73.202 | 1.00 | 0.00 | H |
| ATOM | 3306 | HA | GLU | 252 | 91.157 | 78.567 | 75.716 | 1.00 | 0.00 | H |
| ATOM | 3307 | 1HB | GLU | 252 | 90.450 | 80.523 | 74.472 | 1.00 | 0.00 | H |
| ATOM | 3308 | 2HB | GLU | 252 | 92.170 | 80.226 | 74.346 | 1.00 | 0.00 | H |
| ATOM | 3309 | 1HG | GLU | 252 | 92.052 | 79.611 | 71.998 | 1.00 | 0.00 | H |
| ATOM | 3310 | 2HG | GLU | 252 | 90.319 | 79.351 | 72.044 | 1.00 | 0.00 | H |
| ATOM | 3311 | N | THR | 253 | 89.078 | 77.064 | 75.479 | 1.00 | 0.00 | N |
| ATOM | 3312 | CA | THR | 253 | 87.671 | 76.555 | 75.551 | 1.00 | 0.00 | C |
| ATOM | 3313 | C | THR | 253 | 86.821 | 77.456 | 76.525 | 1.00 | 0.00 | C |
| ATOM | 3314 | O | THR | 253 | 87.348 | 78.092 | 77.442 | 1.00 | 0.00 | O |
| ATOM | 3315 | CB | THR | 253 | 87.705 | 75.025 | 75.879 | 1.00 | 0.00 | C |
| ATOM | 3316 | OG1 | THR | 253 | 88.536 | 74.338 | 74.942 | 1.00 | 0.00 | O |
| ATOM | 3317 | CG2 | THR | 253 | 86.347 | 74.314 | 75.773 | 1.00 | 0.00 | C |
| ATOM | 3318 | H | THR | 253 | 89.751 | 76.885 | 76.233 | 1.00 | 0.00 | H |
| ATOM | 3319 | HA | THR | 253 | 87.207 | 76.633 | 74.548 | 1.00 | 0.00 | H |
| ATOM | 3320 | HB | THR | 253 | 88.122 | 74.883 | 76.897 | 1.00 | 0.00 | H |
| ATOM | 3321 | HG1 | THR | 253 | 88.566 | 73.395 | 75.177 | 1.00 | 0.00 | H |
| ATOM | 3322 | 1HG2 | THR | 253 | 86.442 | 73.235 | 76.002 | 1.00 | 0.00 | H |
| ATOM | 3323 | 2HG2 | THR | 253 | 85.601 | 74.722 | 76.478 | 1.00 | 0.00 | H |
| ATOM | 3324 | 3HG2 | THR | 253 | 85.921 | 74.392 | 74.756 | 1.00 | 0.00 | H |
| ATOM | 3325 | N | SER | 254 | 85.496 | 77.557 | 76.310 | 1.00 | 0.00 | N |
| ATOM | 3326 | CA | SER | 254 | 84.656 | 78.676 | 76.858 | 1.00 | 0.00 | C |
| ATOM | 3327 | C | SER | 254 | 84.494 | 78.929 | 78.410 | 1.00 | 0.00 | C |
| ATOM | 3328 | O | SER | 254 | 83.812 | 79.888 | 78.786 | 1.00 | 0.00 | O |
| ATOM | 3329 | CB | SER | 254 | 83.275 | 78.531 | 76.169 | 1.00 | 0.00 | C |
| ATOM | 3330 | OG | SER | 254 | 82.460 | 79.682 | 76.403 | 1.00 | 0.00 | O |
| ATOM | 3331 | H | SER | 254 | 85.181 | 77.003 | 75.509 | 1.00 | 0.00 | H |
| ATOM | 3332 | HA | SER | 254 | 85.095 | 79.620 | 76.478 | 1.00 | 0.00 | H |
| ATOM | 3333 | 1HB | SER | 254 | 83.378 | 78.402 | 75.073 | 1.00 | 0.00 | H |
| ATOM | 3334 | 2HB | SER | 254 | 82.749 | 77.626 | 76.532 | 1.00 | 0.00 | H |
| ATOM | 3335 | HG | SER | 254 | 82.592 | 79.944 | 77.326 | 1.00 | 0.00 | H |
| ATOM | 3336 | N | CYS | 255 | 85.100 | 78.129 | 79.298 | 1.00 | 0.00 | N |
| ATOM | 3337 | CA | CYS | 255 | 85.274 | 78.483 | 80.735 | 1.00 | 0.00 | C |
| ATOM | 3338 | C | CYS | 255 | 86.653 | 77.980 | 81.291 | 1.00 | 0.00 | C |
| ATOM | 3339 | O | CYS | 255 | 87.287 | 77.081 | 80.727 | 1.00 | 0.00 | O |
| ATOM | 3340 | CB | CYS | 255 | 84.059 | 77.949 | 81.532 | 1.00 | 0.00 | C |
| ATOM | 3341 | SG | CYS | 255 | 83.991 | 76.125 | 81.530 | 1.00 | 0.00 | S |
| ATOM | 3342 | H | CYS | 255 | 85.801 | 77.541 | 78.829 | 1.00 | 0.00 | H |
| ATOM | 3343 | HA | CYS | 255 | 85.285 | 79.588 | 80.834 | 1.00 | 0.00 | H |
| ATOM | 3344 | 1HB | CYS | 255 | 84.098 | 78.298 | 82.579 | 1.00 | 0.00 | H |
| ATOM | 3345 | 2HB | CYS | 255 | 83.111 | 78.350 | 81.122 | 1.00 | 0.00 | H |
| ATOM | 3346 | HG | CYS | 255 | 83.576 | 75.982 | 80.275 | 1.00 | 0.00 | H |
| ATOM | 3347 | N | LEU | 256 | 87.110 | 78.539 | 82.429 | 1.00 | 0.00 | N |
| ATOM | 3348 | CA | LEU | 256 | 88.437 | 78.204 | 83.043 | 1.00 | 0.00 | C |
| ATOM | 3349 | C | LEU | 256 | 88.717 | 76.678 | 83.289 | 1.00 | 0.00 | C |
| ATOM | 3350 | O | LEU | 256 | 89.732 | 76.166 | 82.819 | 1.00 | 0.00 | O |
| ATOM | 3351 | CB | LEU | 256 | 88.641 | 79.036 | 84.347 | 1.00 | 0.00 | C |
| ATOM | 3352 | CG | LEU | 256 | 88.951 | 80.551 | 84.214 | 1.00 | 0.00 | C |
| ATOM | 3353 | CD1 | LEU | 256 | 87.723 | 81.413 | 83.867 | 1.00 | 0.00 | C |
| ATOM | 3354 | CD2 | LEU | 256 | 89.546 | 81.077 | 85.533 | 1.00 | 0.00 | C |
| ATOM | 3355 | H | LEU | 256 | 86.525 | 79.294 | 82.798 | 1.00 | 0.00 | H |
| ATOM | 3356 | HA | LEU | 256 | 89.222 | 78.522 | 82.328 | 1.00 | 0.00 | H |
| ATOM | 3357 | 1HB | LEU | 256 | 87.794 | 78.878 | 85.043 | 1.00 | 0.00 | H |
| ATOM | 3358 | 2HB | LEU | 256 | 89.502 | 78.580 | 84.880 | 1.00 | 0.00 | H |
| ATOM | 3359 | HG | LEU | 256 | 89.710 | 80.687 | 83.419 | 1.00 | 0.00 | H |
| ATOM | 3360 | 2HD1 | LEU | 256 | 87.335 | 81.198 | 82.857 | 1.00 | 0.00 | H |
| ATOM | 3361 | 3HD1 | LEU | 256 | 86.893 | 81.266 | 84.584 | 1.00 | 0.00 | H |
| ATOM | 3362 | 1HD1 | LEU | 256 | 87.965 | 82.492 | 83.872 | 1.00 | 0.00 | H |
| ATOM | 3363 | 2HD2 | LEU | 256 | 88.839 | 80.978 | 86.380 | 1.00 | 0.00 | H |
| ATOM | 3364 | 3HD2 | LEU | 256 | 90.465 | 80.530 | 85.818 | 1.00 | 0.00 | H |
| ATOM | 3365 | 1HD2 | LEU | 256 | 89.825 | 82.144 | 85.467 | 1.00 | 0.00 | H |
| ATOM | 3366 | N | LYS | 257 | 87.800 | 75.951 | 83.953 | 1.00 | 0.00 | N |
| ATOM | 3367 | CA | LYS | 257 | 87.873 | 74.462 | 84.089 | 1.00 | 0.00 | C |
| ATOM | 3368 | C | LYS | 257 | 87.861 | 73.614 | 82.764 | 1.00 | 0.00 | C |
| ATOM | 3369 | O | LYS | 257 | 88.490 | 72.555 | 82.727 | 1.00 | 0.00 | O |
| ATOM | 3370 | CB | LYS | 257 | 86.754 | 74.003 | 85.066 | 1.00 | 0.00 | C |
| ATOM | 3371 | CG | LYS | 257 | 86.922 | 74.460 | 86.537 | 1.00 | 0.00 | C |
| ATOM | 3372 | CD | LYS | 257 | 85.765 | 73.981 | 87.434 | 1.00 | 0.00 | C |
| ATOM | 3373 | CE | LYS | 257 | 85.926 | 74.466 | 88.881 | 1.00 | 0.00 | C |
| ATOM | 3374 | NZ | LYS | 257 | 84.781 | 73.996 | 89.685 | 1.00 | 0.00 | N |
| ATOM | 3375 | 1HZ | LYS | 257 | 84.887 | 74.321 | 90.657 | 1.00 | 0.00 | H |
| ATOM | 3376 | 2HZ | LYS | 257 | 83.906 | 74.372 | 89.292 | 1.00 | 0.00 | H |
| ATOM | 3377 | 3HZ | LYS | 257 | 84.749 | 72.967 | 89.670 | 1.00 | 0.00 | H |
| ATOM | 3378 | H | LYS | 257 | 86.991 | 76.492 | 84.272 | 1.00 | 0.00 | H |
| ATOM | 3379 | HA | LYS | 257 | 88.840 | 74.217 | 84.568 | 1.00 | 0.00 | H |
| ATOM | 3380 | 1HB | LYS | 257 | 85.766 | 74.319 | 84.678 | 1.00 | 0.00 | H |
| ATOM | 3381 | 2HB | LYS | 257 | 86.711 | 72.897 | 85.064 | 1.00 | 0.00 | H |
| ATOM | 3382 | 1HG | LYS | 257 | 87.885 | 74.087 | 86.937 | 1.00 | 0.00 | H |
| ATOM | 3383 | 2HG | LYS | 257 | 86.992 | 75.564 | 86.588 | 1.00 | 0.00 | H |
| ATOM | 3384 | 1HD | LYS | 257 | 84.800 | 74.343 | 87.025 | 1.00 | 0.00 | H |
| ATOM | 3385 | 2HD | LYS | 257 | 85.706 | 72.875 | 87.407 | 1.00 | 0.00 | H |
| ATOM | 3386 | 1HE | LYS | 257 | 86.877 | 74.097 | 89.316 | 1.00 | 0.00 | H |
| ATOM | 3387 | 2HE | LYS | 257 | 85.978 | 75.573 | 88.918 | 1.00 | 0.00 | H |
| ATOM | 3388 | N | GLU | 258 | 87.189 | 74.064 | 81.686 | 1.00 | 0.00 | N |
| ATOM | 3389 | CA | GLU | 258 | 87.332 | 73.448 | 80.332 | 1.00 | 0.00 | C |
| ATOM | 3390 | C | GLU | 258 | 88.705 | 73.711 | 79.619 | 1.00 | 0.00 | C |
| ATOM | 3391 | O | GLU | 258 | 89.293 | 72.754 | 79.114 | 1.00 | 0.00 | O |
| ATOM | 3392 | CB | GLU | 258 | 86.118 | 73.847 | 79.451 | 1.00 | 0.00 | C |
| ATOM | 3393 | CG | GLU | 258 | 84.750 | 73.213 | 79.823 | 1.00 | 0.00 | C |
| ATOM | 3394 | CD | GLU | 258 | 84.645 | 71.709 | 79.571 | 1.00 | 0.00 | C |
| ATOM | 3395 | OE1 | GLU | 258 | 84.362 | 71.217 | 78.485 | 1.00 | 0.00 | O |
| ATOM | 3396 | OE2 | GLU | 258 | 84.899 | 70.977 | 80.687 | 1.00 | 0.00 | O |
| ATOM | 3397 | H | GLU | 258 | 86.861 | 75.028 | 81.801 | 1.00 | 0.00 | H |
| ATOM | 3398 | HA | GLU | 258 | 87.289 | 72.346 | 80.451 | 1.00 | 0.00 | H |
| ATOM | 3399 | 1HB | GLU | 258 | 86.027 | 74.951 | 79.425 | 1.00 | 0.00 | H |
| ATOM | 3400 | 2HB | GLU | 258 | 86.339 | 73.566 | 78.406 | 1.00 | 0.00 | H |
| ATOM | 3401 | 1HG | GLU | 258 | 84.495 | 73.423 | 80.877 | 1.00 | 0.00 | H |
| ATOM | 3402 | 2HG | GLU | 258 | 83.951 | 73.695 | 79.230 | 1.00 | 0.00 | H |
| ATOM | 3403 | N | THR | 259 | 89.256 | 74.941 | 79.608 | 1.00 | 0.00 | N |
| ATOM | 3404 | CA | THR | 259 | 90.700 | 75.186 | 79.255 | 1.00 | 0.00 | C |
| ATOM | 3405 | C | THR | 259 | 91.743 | 74.348 | 80.089 | 1.00 | 0.00 | C |
| ATOM | 3406 | O | THR | 259 | 92.662 | 73.765 | 79.509 | 1.00 | 0.00 | O |
| ATOM | 3407 | CB | THR | 259 | 90.988 | 76.720 | 79.303 | 1.00 | 0.00 | C |
| ATOM | 3408 | OG1 | THR | 259 | 90.192 | 77.414 | 78.352 | 1.00 | 0.00 | O |
| ATOM | 3409 | CG2 | THR | 259 | 92.425 | 77.133 | 78.955 | 1.00 | 0.00 | C |
| ATOM | 3410 | H | THR | 259 | 88.665 | 75.681 | 80.011 | 1.00 | 0.00 | H |
| ATOM | 3411 | HA | THR | 259 | 90.841 | 74.870 | 78.203 | 1.00 | 0.00 | H |
| ATOM | 3412 | HB | THR | 259 | 90.750 | 77.097 | 80.320 | 1.00 | 0.00 | H |
| ATOM | 3413 | HG1 | THR | 259 | 90.577 | 78.292 | 78.280 | 1.00 | 0.00 | H |
| ATOM | 3414 | 1HG2 | THR | 259 | 92.562 | 78.226 | 79.020 | 1.00 | 0.00 | H |
| ATOM | 3415 | 2HG2 | THR | 259 | 93.166 | 76.690 | 79.646 | 1.00 | 0.00 | H |
| ATOM | 3416 | 3HG2 | THR | 259 | 92.711 | 76.823 | 77.933 | 1.00 | 0.00 | H |
| ATOM | 3417 | N | TYR | 260 | 91.586 | 74.262 | 81.423 | 0.00 | 0.00 | N |
| ATOM | 3418 | CA | TYR | 260 | 92.363 | 73.330 | 82.294 | 0.00 | 0.00 | C |
| ATOM | 3419 | C | TYR | 260 | 92.279 | 71.804 | 81.919 | 0.00 | 0.00 | C |
| ATOM | 3420 | O | TYR | 260 | 93.315 | 71.139 | 81.844 | 0.00 | 0.00 | O |
| ATOM | 3421 | CB | TYR | 260 | 91.924 | 73.570 | 83.770 | 0.00 | 0.00 | C |
| ATOM | 3422 | CG | TYR | 260 | 92.128 | 74.943 | 84.457 | 0.00 | 0.00 | C |
| ATOM | 3423 | CD1 | TYR | 260 | 92.987 | 75.934 | 83.963 | 0.00 | 0.00 | C |
| ATOM | 3424 | CE1 | TYR | 260 | 93.105 | 77.157 | 84.618 | 0.00 | 0.00 | C |
| ATOM | 3425 | CZ | TYR | 260 | 92.370 | 77.402 | 85.772 | 0.00 | 0.00 | C |
| ATOM | 3426 | OH | TYR | 260 | 92.494 | 78.601 | 86.417 | 0.00 | 0.00 | O |
| ATOM | 3427 | CE2 | TYR | 260 | 91.523 | 76.424 | 86.280 | 0.00 | 0.00 | C |
| ATOM | 3428 | CD2 | TYR | 260 | 91.408 | 75.196 | 85.631 | 0.00 | 0.00 | C |
| ATOM | 3429 | H | TYR | 260 | 90.890 | 74.876 | 81.869 | 1.00 | 0.00 | H |
| ATOM | 3430 | HA | TYR | 260 | 93.435 | 73.594 | 82.207 | 0.00 | 0.00 | H |
| ATOM | 3431 | 1HB | TYR | 260 | 92.431 | 72.818 | 84.399 | 0.00 | 0.00 | H |
| ATOM | 3432 | 2HB | TYR | 260 | 90.856 | 73.289 | 83.848 | 0.00 | 0.00 | H |
| ATOM | 3433 | HD1 | TYR | 260 | 93.564 | 75.768 | 83.063 | 0.00 | 0.00 | H |
| ATOM | 3434 | HE1 | TYR | 260 | 93.777 | 77.903 | 84.231 | 0.00 | 0.00 | H |
| ATOM | 3435 | HH | TYR | 260 | 93.285 | 79.038 | 86.094 | 0.00 | 0.00 | H |
| ATOM | 3436 | HE2 | TYR | 260 | 90.964 | 76.616 | 87.184 | 0.00 | 0.00 | H |
| ATOM | 3437 | HD2 | TYR | 260 | 90.752 | 74.444 | 86.046 | 0.00 | 0.00 | H |
| ATOM | 3438 | N | LEU | 261 | 91.075 | 71.265 | 81.640 | 1.00 | 0.00 | N |
| ATOM | 3439 | CA | LEU | 261 | 90.897 | 69.932 | 80.984 | 1.00 | 0.00 | C |
| ATOM | 3440 | C | LEU | 261 | 91.694 | 69.702 | 79.646 | 1.00 | 0.00 | C |
| ATOM | 3441 | O | LEU | 261 | 92.322 | 68.652 | 79.497 | 1.00 | 0.00 | O |
| ATOM | 3442 | CB | LEU | 261 | 89.358 | 69.723 | 80.849 | 1.00 | 0.00 | C |
| ATOM | 3443 | CG | LEU | 261 | 88.856 | 68.354 | 80.325 | 1.00 | 0.00 | C |
| ATOM | 3444 | CD1 | LEU | 261 | 89.165 | 67.203 | 81.298 | 1.00 | 0.00 | C |
| ATOM | 3445 | CD2 | LEU | 261 | 87.340 | 68.412 | 80.075 | 1.00 | 0.00 | C |
| ATOM | 3446 | H | LEU | 261 | 90.292 | 71.922 | 81.746 | 1.00 | 0.00 | H |
| ATOM | 3447 | HA | LEU | 261 | 91.277 | 69.171 | 81.692 | 1.00 | 0.00 | H |
| ATOM | 3448 | 1HB | LEU | 261 | 88.869 | 69.924 | 81.824 | 1.00 | 0.00 | H |
| ATOM | 3449 | 2HB | LEU | 261 | 88.967 | 70.510 | 80.178 | 1.00 | 0.00 | H |
| ATOM | 3450 | HG | LEU | 261 | 89.350 | 68.138 | 79.355 | 1.00 | 0.00 | H |
| ATOM | 3451 | 2HD1 | LEU | 261 | 90.254 | 67.069 | 81.437 | 1.00 | 0.00 | H |
| ATOM | 3452 | 3HD1 | LEU | 261 | 88.721 | 67.367 | 82.298 | 1.00 | 0.00 | H |
| ATOM | 3453 | 1HD1 | LEU | 261 | 88.781 | 66.236 | 80.922 | 1.00 | 0.00 | H |
| ATOM | 3454 | 2HD2 | LEU | 261 | 86.774 | 68.623 | 81.001 | 1.00 | 0.00 | H |
| ATOM | 3455 | 3HD2 | LEU | 261 | 87.082 | 69.198 | 79.340 | 1.00 | 0.00 | H |
| ATOM | 3456 | 1HD2 | LEU | 261 | 86.959 | 67.459 | 79.664 | 1.00 | 0.00 | H |
| ATOM | 3457 | N | ARG | 262 | 91.731 | 70.679 | 78.720 | 1.00 | 0.00 | N |
| ATOM | 3458 | CA | ARG | 262 | 92.648 | 70.647 | 77.542 | 1.00 | 0.00 | C |
| ATOM | 3459 | C | ARG | 262 | 94.188 | 70.612 | 77.880 | 1.00 | 0.00 | C |
| ATOM | 3460 | O | ARG | 262 | 94.905 | 69.763 | 77.348 | 1.00 | 0.00 | O |
| ATOM | 3461 | CB | ARG | 262 | 92.340 | 71.831 | 76.577 | 1.00 | 0.00 | C |
| ATOM | 3462 | CG | ARG | 262 | 90.899 | 72.128 | 76.094 | 1.00 | 0.00 | C |
| ATOM | 3463 | CD | ARG | 262 | 90.194 | 70.966 | 75.385 | 1.00 | 0.00 | C |
| ATOM | 3464 | NE | ARG | 262 | 88.899 | 71.458 | 74.845 | 1.00 | 0.00 | N |
| ATOM | 3465 | CZ | ARG | 262 | 88.061 | 70.748 | 74.099 | 1.00 | 0.00 | C |
| ATOM | 3466 | NH1 | ARG | 262 | 88.245 | 69.501 | 73.778 | 1.00 | 0.00 | N |
| ATOM | 3467 | NH2 | ARG | 262 | 87.005 | 71.337 | 73.662 | 1.00 | 0.00 | N |
| ATOM | 3468 | HE | ARG | 262 | 88.631 | 72.428 | 75.067 | 1.00 | 0.00 | H |
| ATOM | 3469 | H | ARG | 262 | 91.155 | 71.497 | 78.953 | 1.00 | 0.00 | H |
| ATOM | 3470 | HA | ARG | 262 | 92.440 | 69.712 | 76.984 | 1.00 | 0.00 | H |
| ATOM | 3471 | 1HB | ARG | 262 | 92.731 | 72.763 | 77.029 | 1.00 | 0.00 | H |
| ATOM | 3472 | 2HB | ARG | 262 | 92.961 | 71.684 | 75.673 | 1.00 | 0.00 | H |
| ATOM | 3473 | 1HG | ARG | 262 | 90.280 | 72.468 | 76.943 | 1.00 | 0.00 | H |
| ATOM | 3474 | 2HG | ARG | 262 | 90.939 | 73.002 | 75.413 | 1.00 | 0.00 | H |
| ATOM | 3475 | 1HD | ARG | 262 | 90.835 | 70.574 | 74.570 | 1.00 | 0.00 | H |
| ATOM | 3476 | 2HD | ARG | 262 | 90.030 | 70.128 | 76.095 | 1.00 | 0.00 | H |
| ATOM | 3477 | 2HH1 | ARG | 262 | 89.103 | 69.110 | 74.168 | 1.00 | 0.00 | H |
| ATOM | 3478 | 1HH1 | ARG | 262 | 87.536 | 69.062 | 73.194 | 1.00 | 0.00 | H |
| ATOM | 3479 | 1HH2 | ARG | 262 | 86.968 | 72.323 | 73.921 | 1.00 | 0.00 | H |
| ATOM | 3480 | 2HH2 | ARG | 262 | 86.383 | 70.796 | 73.065 | 1.00 | 0.00 | H |
| ATOM | 3481 | N | ILE | 263 | 94.699 | 71.521 | 78.739 | 0.00 | 0.00 | N |
| ATOM | 3482 | CA | ILE | 263 | 96.162 | 71.638 | 79.087 | 0.00 | 0.00 | C |
| ATOM | 3483 | C | ILE | 263 | 96.852 | 70.322 | 79.595 | 0.00 | 0.00 | C |
| ATOM | 3484 | O | ILE | 263 | 97.919 | 69.961 | 79.089 | 0.00 | 0.00 | O |
| ATOM | 3485 | CB | ILE | 263 | 96.358 | 72.915 | 79.998 | 0.00 | 0.00 | C |
| ATOM | 3486 | CG2 | ILE | 263 | 97.713 | 72.981 | 80.760 | 0.00 | 0.00 | C |
| ATOM | 3487 | CG1 | ILE | 263 | 96.180 | 74.229 | 79.176 | 0.00 | 0.00 | C |
| ATOM | 3488 | CD1 | ILE | 263 | 96.033 | 75.524 | 79.988 | 0.00 | 0.00 | C |
| ATOM | 3489 | H | ILE | 263 | 93.987 | 72.147 | 79.138 | 0.00 | 0.00 | H |
| ATOM | 3490 | HA | ILE | 263 | 96.703 | 71.848 | 78.143 | 0.00 | 0.00 | H |
| ATOM | 3491 | HB | ILE | 263 | 95.570 | 72.886 | 80.779 | 0.00 | 0.00 | H |
| ATOM | 3492 | 1HG2 | ILE | 263 | 97.758 | 73.841 | 81.453 | 0.00 | 0.00 | H |
| ATOM | 3493 | 2HG2 | ILE | 263 | 97.888 | 72.090 | 81.391 | 0.00 | 0.00 | H |
| ATOM | 3494 | 3HG2 | ILE | 263 | 98.576 | 73.073 | 80.078 | 0.00 | 0.00 | H |
| ATOM | 3495 | 1HG1 | ILE | 263 | 95.276 | 74.150 | 78.541 | 0.00 | 0.00 | H |
| ATOM | 3496 | 2HG1 | ILE | 263 | 97.018 | 74.342 | 78.460 | 0.00 | 0.00 | H |
| ATOM | 3497 | 1HD1 | ILE | 263 | 95.825 | 76.385 | 79.327 | 0.00 | 0.00 | H |
| ATOM | 3498 | 2HD1 | ILE | 263 | 95.201 | 75.466 | 80.716 | 0.00 | 0.00 | H |
| ATOM | 3499 | 3HD1 | ILE | 263 | 96.952 | 75.777 | 80.549 | 0.00 | 0.00 | H |
| ATOM | 3500 | N | LYS | 264 | 96.248 | 69.602 | 80.554 | 1.00 | 0.00 | N |
| ATOM | 3501 | CA | LYS | 264 | 96.726 | 68.247 | 80.970 | 1.00 | 0.00 | C |
| ATOM | 3502 | C | LYS | 264 | 96.706 | 67.106 | 79.876 | 1.00 | 0.00 | C |
| ATOM | 3503 | O | LYS | 264 | 97.548 | 66.207 | 79.928 | 1.00 | 0.00 | O |
| ATOM | 3504 | CB | LYS | 264 | 95.936 | 67.915 | 82.266 | 1.00 | 0.00 | C |
| ATOM | 3505 | CG | LYS | 264 | 96.507 | 66.746 | 83.102 | 1.00 | 0.00 | C |
| ATOM | 3506 | CD | LYS | 264 | 95.859 | 66.668 | 84.497 | 1.00 | 0.00 | C |
| ATOM | 3507 | CE | LYS | 264 | 96.455 | 65.540 | 85.352 | 1.00 | 0.00 | C |
| ATOM | 3508 | NZ | LYS | 264 | 95.990 | 65.686 | 86.747 | 1.00 | 0.00 | N |
| ATOM | 3509 | 1HZ | LYS | 264 | 96.389 | 64.930 | 87.323 | 1.00 | 0.00 | H |
| ATOM | 3510 | 2HZ | LYS | 264 | 96.296 | 66.597 | 87.118 | 1.00 | 0.00 | H |
| ATOM | 3511 | 3HZ | LYS | 264 | 94.962 | 65.631 | 86.775 | 1.00 | 0.00 | H |
| ATOM | 3512 | H | LYS | 264 | 95.332 | 69.978 | 80.830 | 1.00 | 0.00 | H |
| ATOM | 3513 | HA | LYS | 264 | 97.791 | 68.352 | 81.256 | 1.00 | 0.00 | H |
| ATOM | 3514 | 1HB | LYS | 264 | 95.915 | 68.811 | 82.921 | 1.00 | 0.00 | H |
| ATOM | 3515 | 2HB | LYS | 264 | 94.873 | 67.717 | 82.021 | 1.00 | 0.00 | H |
| ATOM | 3516 | 1HG | LYS | 264 | 96.375 | 65.790 | 82.559 | 1.00 | 0.00 | H |
| ATOM | 3517 | 2HG | LYS | 264 | 97.603 | 66.872 | 83.218 | 1.00 | 0.00 | H |
| ATOM | 3518 | 1HD | LYS | 264 | 95.990 | 67.646 | 85.005 | 1.00 | 0.00 | H |
| ATOM | 3519 | 2HD | LYS | 264 | 94.763 | 66.533 | 84.400 | 1.00 | 0.00 | H |
| ATOM | 3520 | 1HE | LYS | 264 | 96.164 | 64.551 | 84.944 | 1.00 | 0.00 | H |
| ATOM | 3521 | 2HE | LYS | 264 | 97.564 | 65.563 | 85.324 | 1.00 | 0.00 | H |
| ATOM | 3522 | N | LYS | 265 | 95.801 | 67.156 | 78.879 | 1.00 | 0.00 | N |
| ATOM | 3523 | CA | LYS | 265 | 95.810 | 66.235 | 77.699 | 1.00 | 0.00 | C |
| ATOM | 3524 | C | LYS | 265 | 96.949 | 66.440 | 76.629 | 1.00 | 0.00 | C |
| ATOM | 3525 | O | LYS | 265 | 97.292 | 65.473 | 75.946 | 1.00 | 0.00 | O |
| ATOM | 3526 | CB | LYS | 265 | 94.419 | 66.320 | 77.003 | 1.00 | 0.00 | C |
| ATOM | 3527 | CG | LYS | 265 | 93.209 | 65.795 | 77.809 | 1.00 | 0.00 | C |
| ATOM | 3528 | CD | LYS | 265 | 91.881 | 66.006 | 77.056 | 1.00 | 0.00 | C |
| ATOM | 3529 | CE | LYS | 265 | 90.672 | 65.560 | 77.889 | 1.00 | 0.00 | C |
| ATOM | 3530 | NZ | LYS | 265 | 89.430 | 65.791 | 77.125 | 1.00 | 0.00 | N |
| ATOM | 3531 | 1HZ | LYS | 265 | 88.620 | 65.492 | 77.686 | 1.00 | 0.00 | H |
| ATOM | 3532 | 2HZ | LYS | 265 | 89.343 | 66.794 | 76.905 | 1.00 | 0.00 | H |
| ATOM | 3533 | 3HZ | LYS | 265 | 89.460 | 65.250 | 76.249 | 1.00 | 0.00 | H |
| ATOM | 3534 | H | LYS | 265 | 95.178 | 67.973 | 78.921 | 1.00 | 0.00 | H |
| ATOM | 3535 | HA | LYS | 265 | 95.937 | 65.197 | 78.066 | 1.00 | 0.00 | H |
| ATOM | 3536 | 1HB | LYS | 265 | 94.232 | 67.367 | 76.691 | 1.00 | 0.00 | H |
| ATOM | 3537 | 2HB | LYS | 265 | 94.456 | 65.750 | 76.053 | 1.00 | 0.00 | H |
| ATOM | 3538 | 1HG | LYS | 265 | 93.348 | 64.723 | 78.049 | 1.00 | 0.00 | H |
| ATOM | 3539 | 2HG | LYS | 265 | 93.160 | 66.311 | 78.789 | 1.00 | 0.00 | H |
| ATOM | 3540 | 1HD | LYS | 265 | 91.772 | 67.079 | 76.801 | 1.00 | 0.00 | H |
| ATOM | 3541 | 2HD | LYS | 265 | 91.908 | 65.464 | 76.090 | 1.00 | 0.00 | H |
| ATOM | 3542 | 1HE | LYS | 265 | 90.760 | 64.492 | 78.170 | 1.00 | 0.00 | H |
| ATOM | 3543 | 2HE | LYS | 265 | 90.640 | 66.128 | 78.842 | 1.00 | 0.00 | H |
| ATOM | 3544 | N | ASN | 266 | 97.497 | 67.659 | 76.420 | 1.00 | 0.00 | N |
| ATOM | 3545 | CA | ASN | 266 | 98.622 | 67.927 | 75.454 | 1.00 | 0.00 | C |
| ATOM | 3546 | C | ASN | 266 | 98.354 | 67.785 | 73.900 | 1.00 | 0.00 | C |
| ATOM | 3547 | O | ASN | 266 | 99.262 | 68.052 | 73.108 | 1.00 | 0.00 | O |
| ATOM | 3548 | CB | ASN | 266 | 99.918 | 67.198 | 75.952 | 1.00 | 0.00 | C |
| ATOM | 3549 | CG | ASN | 266 | 101.331 | 67.657 | 75.554 | 1.00 | 0.00 | C |
| ATOM | 3550 | OD1 | ASN | 266 | 102.317 | 67.035 | 75.926 | 1.00 | 0.00 | O |
| ATOM | 3551 | ND2 | ASN | 266 | 101.533 | 68.726 | 74.836 | 1.00 | 0.00 | N |
| ATOM | 3552 | H | ASN | 266 | 97.146 | 68.365 | 77.079 | 1.00 | 0.00 | H |
| ATOM | 3553 | HA | ASN | 266 | 98.819 | 69.010 | 75.570 | 1.00 | 0.00 | H |
| ATOM | 3554 | 1HB | ASN | 266 | 99.928 | 67.201 | 77.057 | 1.00 | 0.00 | H |
| ATOM | 3555 | 2HB | ASN | 266 | 99.845 | 66.127 | 75.691 | 1.00 | 0.00 | H |
| ATOM | 3556 | 1HD2 | ASN | 266 | 102.525 | 68.892 | 74.616 | 1.00 | 0.00 | H |
| ATOM | 3557 | 2HD2 | ASN | 266 | 100.737 | 68.989 | 74.248 | 1.00 | 0.00 | H |
| ATOM | 3558 | N | GLU | 267 | 97.150 | 67.411 | 73.431 | 1.00 | 0.00 | N |
| ATOM | 3559 | CA | GLU | 267 | 96.876 | 67.153 | 71.985 | 1.00 | 0.00 | C |
| ATOM | 3560 | C | GLU | 267 | 96.696 | 68.462 | 71.129 | 1.00 | 0.00 | C |
| ATOM | 3561 | O | GLU | 267 | 95.588 | 68.985 | 70.971 | 1.00 | 0.00 | O |
| ATOM | 3562 | CB | GLU | 267 | 95.662 | 66.183 | 71.946 | 1.00 | 0.00 | C |
| ATOM | 3563 | CG | GLU | 267 | 95.357 | 65.583 | 70.549 | 1.00 | 0.00 | C |
| ATOM | 3564 | CD | GLU | 267 | 94.191 | 64.600 | 70.541 | 1.00 | 0.00 | C |
| ATOM | 3565 | OE1 | GLU | 267 | 94.308 | 63.397 | 70.740 | 1.00 | 0.00 | O |
| ATOM | 3566 | OE2 | GLU | 267 | 93.002 | 65.207 | 70.284 | 1.00 | 0.00 | O |
| ATOM | 3567 | H | GLU | 267 | 96.511 | 67.122 | 74.178 | 1.00 | 0.00 | H |
| ATOM | 3568 | HA | GLU | 267 | 97.734 | 66.589 | 71.560 | 1.00 | 0.00 | H |
| ATOM | 3569 | 1HB | GLU | 267 | 95.833 | 65.341 | 72.650 | 1.00 | 0.00 | H |
| ATOM | 3570 | 2HB | GLU | 267 | 94.761 | 66.704 | 72.329 | 1.00 | 0.00 | H |
| ATOM | 3571 | 1HG | GLU | 267 | 95.145 | 66.387 | 69.818 | 1.00 | 0.00 | H |
| ATOM | 3572 | 2HG | GLU | 267 | 96.246 | 65.055 | 70.155 | 1.00 | 0.00 | H |
| ATOM | 3573 | N | TYR | 268 | 97.801 | 68.977 | 70.566 | 1.00 | 0.00 | N |
| ATOM | 3574 | CA | TYR | 268 | 97.809 | 70.263 | 69.810 | 1.00 | 0.00 | C |
| ATOM | 3575 | C | TYR | 268 | 97.716 | 70.049 | 68.260 | 1.00 | 0.00 | C |
| ATOM | 3576 | O | TYR | 268 | 98.681 | 69.627 | 67.613 | 1.00 | 0.00 | O |
| ATOM | 3577 | CB | TYR | 268 | 99.087 | 71.069 | 70.197 | 1.00 | 0.00 | C |
| ATOM | 3578 | CG | TYR | 268 | 99.105 | 71.681 | 71.610 | 1.00 | 0.00 | C |
| ATOM | 3579 | CD1 | TYR | 268 | 99.934 | 71.160 | 72.610 | 1.00 | 0.00 | C |
| ATOM | 3580 | CD2 | TYR | 268 | 98.293 | 72.781 | 71.901 | 1.00 | 0.00 | C |
| ATOM | 3581 | CE1 | TYR | 268 | 99.936 | 71.723 | 73.886 | 1.00 | 0.00 | C |
| ATOM | 3582 | CE2 | TYR | 268 | 98.311 | 73.348 | 73.172 | 1.00 | 0.00 | C |
| ATOM | 3583 | CZ | TYR | 268 | 99.135 | 72.824 | 74.159 | 1.00 | 0.00 | C |
| ATOM | 3584 | OH | TYR | 268 | 99.129 | 73.384 | 75.404 | 1.00 | 0.00 | O |
| ATOM | 3585 | H | TYR | 268 | 98.686 | 68.459 | 70.662 | 1.00 | 0.00 | H |
| ATOM | 3586 | HA | TYR | 268 | 96.943 | 70.887 | 70.118 | 1.00 | 0.00 | H |
| ATOM | 3587 | 1HB | TYR | 268 | 99.988 | 70.447 | 70.028 | 1.00 | 0.00 | H |
| ATOM | 3588 | 2HB | TYR | 268 | 99.218 | 71.902 | 69.480 | 1.00 | 0.00 | H |
| ATOM | 3589 | HD1 | TYR | 268 | 100.574 | 70.315 | 72.401 | 1.00 | 0.00 | H |
| ATOM | 3590 | HD2 | TYR | 268 | 97.630 | 73.189 | 71.151 | 1.00 | 0.00 | H |
| ATOM | 3591 | HE1 | TYR | 268 | 100.564 | 71.324 | 74.667 | 1.00 | 0.00 | H |
| ATOM | 3592 | HE2 | TYR | 268 | 97.647 | 74.166 | 73.408 | 1.00 | 0.00 | H |
| ATOM | 3593 | HH | TYR | 268 | 98.543 | 74.141 | 75.387 | 1.00 | 0.00 | H |
| ATOM | 3594 | N | SER | 269 | 96.568 | 70.395 | 67.653 | 1.00 | 0.00 | N |
| ATOM | 3595 | CA | SER | 269 | 96.405 | 70.397 | 66.171 | 1.00 | 0.00 | C |
| ATOM | 3596 | C | SER | 269 | 96.971 | 71.702 | 65.513 | 1.00 | 0.00 | C |
| ATOM | 3597 | O | SER | 269 | 96.390 | 72.782 | 65.646 | 1.00 | 0.00 | O |
| ATOM | 3598 | CB | SER | 269 | 94.904 | 70.187 | 65.869 | 1.00 | 0.00 | C |
| ATOM | 3599 | OG | SER | 269 | 94.671 | 70.101 | 64.462 | 1.00 | 0.00 | O |
| ATOM | 3600 | H | SER | 269 | 95.820 | 70.687 | 68.289 | 1.00 | 0.00 | H |
| ATOM | 3601 | HA | SER | 269 | 96.930 | 69.519 | 65.741 | 1.00 | 0.00 | H |
| ATOM | 3602 | 1HB | SER | 269 | 94.538 | 69.257 | 66.349 | 1.00 | 0.00 | H |
| ATOM | 3603 | 2HB | SER | 269 | 94.294 | 71.008 | 66.298 | 1.00 | 0.00 | H |
| ATOM | 3604 | HG | SER | 269 | 94.931 | 70.946 | 64.075 | 1.00 | 0.00 | H |
| ATOM | 3605 | N | ILE | 270 | 98.101 | 71.600 | 64.796 | 1.00 | 0.00 | N |
| ATOM | 3606 | CA | ILE | 270 | 98.790 | 72.785 | 64.192 | 1.00 | 0.00 | C |
| ATOM | 3607 | C | ILE | 270 | 98.061 | 73.392 | 62.921 | 1.00 | 0.00 | C |
| ATOM | 3608 | O | ILE | 270 | 97.670 | 72.619 | 62.038 | 1.00 | 0.00 | O |
| ATOM | 3609 | CB | ILE | 270 | 100.313 | 72.516 | 63.902 | 1.00 | 0.00 | C |
| ATOM | 3610 | CG1 | ILE | 270 | 100.607 | 71.347 | 62.913 | 1.00 | 0.00 | C |
| ATOM | 3611 | CG2 | ILE | 270 | 101.142 | 72.357 | 65.205 | 1.00 | 0.00 | C |
| ATOM | 3612 | CD1 | ILE | 270 | 101.972 | 71.429 | 62.207 | 1.00 | 0.00 | C |
| ATOM | 3613 | H | ILE | 270 | 98.550 | 70.680 | 64.833 | 1.00 | 0.00 | H |
| ATOM | 3614 | HA | ILE | 270 | 98.791 | 73.563 | 64.976 | 1.00 | 0.00 | H |
| ATOM | 3615 | HB | ILE | 270 | 100.692 | 73.441 | 63.419 | 1.00 | 0.00 | H |
| ATOM | 3616 | 1HG1 | ILE | 270 | 100.504 | 70.372 | 63.427 | 1.00 | 0.00 | H |
| ATOM | 3617 | 2HG1 | ILE | 270 | 99.834 | 71.324 | 62.120 | 1.00 | 0.00 | H |
| ATOM | 3618 | 2HG2 | ILE | 270 | 101.005 | 73.213 | 65.890 | 1.00 | 0.00 | H |
| ATOM | 3619 | 3HG2 | ILE | 270 | 100.862 | 71.445 | 65.766 | 1.00 | 0.00 | H |
| ATOM | 3620 | 1HG2 | ILE | 270 | 102.227 | 72.289 | 65.001 | 1.00 | 0.00 | H |
| ATOM | 3621 | 2HD1 | ILE | 270 | 102.088 | 72.375 | 61.642 | 1.00 | 0.00 | H |
| ATOM | 3622 | 3HD1 | ILE | 270 | 102.809 | 71.363 | 62.922 | 1.00 | 0.00 | H |
| ATOM | 3623 | 1HD1 | ILE | 270 | 102.101 | 70.606 | 61.481 | 1.00 | 0.00 | H |
| ATOM | 3624 | N | PRO | 271 | 97.905 | 74.741 | 62.737 | 0.00 | 0.00 | N |
| ATOM | 3625 | CA | PRO | 271 | 97.452 | 75.329 | 61.445 | 0.00 | 0.00 | C |
| ATOM | 3626 | CD | PRO | 271 | 98.045 | 75.733 | 63.816 | 0.00 | 0.00 | C |
| ATOM | 3627 | C | PRO | 271 | 98.411 | 75.108 | 60.225 | 0.00 | 0.00 | C |
| ATOM | 3628 | O | PRO | 271 | 99.637 | 75.076 | 60.365 | 0.00 | 0.00 | O |
| ATOM | 3629 | CB | PRO | 271 | 97.285 | 76.826 | 61.795 | 0.00 | 0.00 | C |
| ATOM | 3630 | CG | PRO | 271 | 97.187 | 76.890 | 63.320 | 0.00 | 0.00 | C |
| ATOM | 3631 | HA | PRO | 271 | 96.457 | 74.900 | 61.214 | 0.00 | 0.00 | H |
| ATOM | 3632 | 1HD | PRO | 271 | 99.104 | 76.028 | 63.948 | 0.00 | 0.00 | H |
| ATOM | 3633 | 2HD | PRO | 271 | 97.663 | 75.374 | 64.789 | 0.00 | 0.00 | H |
| ATOM | 3634 | 1HB | PRO | 271 | 96.401 | 77.273 | 61.302 | 0.00 | 0.00 | H |
| ATOM | 3635 | 2HB | PRO | 271 | 98.155 | 77.425 | 61.460 | 0.00 | 0.00 | H |
| ATOM | 3636 | 1HG | PRO | 271 | 96.138 | 76.747 | 63.647 | 0.00 | 0.00 | H |
| ATOM | 3637 | 2HG | PRO | 271 | 97.527 | 77.855 | 63.736 | 0.00 | 0.00 | H |
| ATOM | 3638 | N | LYS | 272 | 97.848 | 75.012 | 59.012 | 1.00 | 0.00 | N |
| ATOM | 3639 | CA | LYS | 272 | 98.620 | 74.638 | 57.781 | 1.00 | 0.00 | C |
| ATOM | 3640 | C | LYS | 272 | 99.753 | 75.604 | 57.258 | 1.00 | 0.00 | C |
| ATOM | 3641 | O | LYS | 272 | 100.562 | 75.181 | 56.429 | 1.00 | 0.00 | O |
| ATOM | 3642 | CB | LYS | 272 | 97.590 | 74.311 | 56.660 | 1.00 | 0.00 | C |
| ATOM | 3643 | CG | LYS | 272 | 96.728 | 73.042 | 56.890 | 1.00 | 0.00 | C |
| ATOM | 3644 | CD | LYS | 272 | 95.734 | 72.788 | 55.741 | 1.00 | 0.00 | C |
| ATOM | 3645 | CE | LYS | 272 | 94.875 | 71.542 | 55.996 | 1.00 | 0.00 | C |
| ATOM | 3646 | NZ | LYS | 272 | 93.936 | 71.353 | 54.873 | 1.00 | 0.00 | N |
| ATOM | 3647 | 1HZ | LYS | 272 | 93.359 | 70.516 | 55.044 | 1.00 | 0.00 | H |
| ATOM | 3648 | 2HZ | LYS | 272 | 93.326 | 72.179 | 54.792 | 1.00 | 0.00 | H |
| ATOM | 3649 | 3HZ | LYS | 272 | 94.465 | 71.231 | 53.998 | 1.00 | 0.00 | H |
| ATOM | 3650 | H | LYS | 272 | 96.826 | 74.945 | 59.037 | 1.00 | 0.00 | H |
| ATOM | 3651 | HA | LYS | 272 | 99.166 | 73.697 | 58.001 | 1.00 | 0.00 | H |
| ATOM | 3652 | 1HB | LYS | 272 | 96.940 | 75.189 | 56.482 | 1.00 | 0.00 | H |
| ATOM | 3653 | 2HB | LYS | 272 | 98.135 | 74.172 | 55.706 | 1.00 | 0.00 | H |
| ATOM | 3654 | 1HG | LYS | 272 | 97.390 | 72.162 | 57.013 | 1.00 | 0.00 | H |
| ATOM | 3655 | 2HG | LYS | 272 | 96.173 | 73.124 | 57.844 | 1.00 | 0.00 | H |
| ATOM | 3656 | 1HD | LYS | 272 | 95.079 | 73.673 | 55.612 | 1.00 | 0.00 | H |
| ATOM | 3657 | 2HD | LYS | 272 | 96.288 | 72.683 | 54.786 | 1.00 | 0.00 | H |
| ATOM | 3658 | 1HE | LYS | 272 | 95.515 | 70.644 | 56.117 | 1.00 | 0.00 | H |
| ATOM | 3659 | 2HE | LYS | 272 | 94.310 | 71.646 | 56.944 | 1.00 | 0.00 | H |
| ATOM | 3660 | N | HIS | 273 | 99.852 | 76.859 | 57.732 | 0.00 | 0.00 | N |
| ATOM | 3661 | CA | HIS | 273 | 101.068 | 77.716 | 57.532 | 0.00 | 0.00 | C |
| ATOM | 3662 | C | HIS | 273 | 102.303 | 77.505 | 58.495 | 0.00 | 0.00 | C |
| ATOM | 3663 | O | HIS | 273 | 103.356 | 78.106 | 58.261 | 0.00 | 0.00 | O |
| ATOM | 3664 | CB | HIS | 273 | 100.609 | 79.199 | 57.426 | 0.00 | 0.00 | C |
| ATOM | 3665 | CG | HIS | 273 | 100.004 | 79.854 | 58.670 | 0.00 | 0.00 | C |
| ATOM | 3666 | ND1 | HIS | 273 | 100.759 | 80.491 | 59.641 | 0.00 | 0.00 | N |
| ATOM | 3667 | CE1 | HIS | 273 | 99.745 | 80.966 | 60.435 | 0.00 | 0.00 | C |
| ATOM | 3668 | NE2 | HIS | 273 | 98.441 | 80.711 | 60.103 | 0.00 | 0.00 | N |
| ATOM | 3669 | CD2 | HIS | 273 | 98.631 | 79.995 | 58.934 | 0.00 | 0.00 | C |
| ATOM | 3670 | H | HIS | 273 | 99.200 | 77.027 | 58.504 | 0.00 | 0.00 | H |
| ATOM | 3671 | HA | HIS | 273 | 101.491 | 77.482 | 56.535 | 0.00 | 0.00 | H |
| ATOM | 3672 | 1HB | HIS | 273 | 99.904 | 79.300 | 56.580 | 0.00 | 0.00 | H |
| ATOM | 3673 | 2HB | HIS | 273 | 101.478 | 79.808 | 57.111 | 0.00 | 0.00 | H |
| ATOM | 3674 | HE1 | HIS | 273 | 99.988 | 81.567 | 61.300 | 0.00 | 0.00 | H |
| ATOM | 3675 | HE2 | HIS | 273 | 97.590 | 81.135 | 60.489 | 0.00 | 0.00 | H |
| ATOM | 3676 | HD2 | HIS | 273 | 97.843 | 79.641 | 58.283 | 0.00 | 0.00 | H |
| ATOM | 3677 | N | ILE | 274 | 102.221 | 76.650 | 59.534 | 1.00 | 0.00 | N |
| ATOM | 3678 | CA | ILE | 274 | 103.380 | 76.285 | 60.408 | 1.00 | 0.00 | C |
| ATOM | 3679 | C | ILE | 274 | 104.287 | 75.247 | 59.646 | 1.00 | 0.00 | C |
| ATOM | 3680 | O | ILE | 274 | 103.846 | 74.144 | 59.306 | 1.00 | 0.00 | O |
| ATOM | 3681 | CB | ILE | 274 | 102.859 | 75.786 | 61.818 | 1.00 | 0.00 | C |
| ATOM | 3682 | CG1 | ILE | 274 | 102.352 | 76.893 | 62.794 | 1.00 | 0.00 | C |
| ATOM | 3683 | CG2 | ILE | 274 | 103.914 | 74.996 | 62.640 | 1.00 | 0.00 | C |
| ATOM | 3684 | CD1 | ILE | 274 | 101.144 | 77.724 | 62.340 | 1.00 | 0.00 | C |
| ATOM | 3685 | H | ILE | 274 | 101.319 | 76.162 | 59.609 | 1.00 | 0.00 | H |
| ATOM | 3686 | HA | ILE | 274 | 103.981 | 77.196 | 60.601 | 1.00 | 0.00 | H |
| ATOM | 3687 | HB | ILE | 274 | 102.021 | 75.081 | 61.635 | 1.00 | 0.00 | H |
| ATOM | 3688 | 1HG1 | ILE | 274 | 102.069 | 76.430 | 63.760 | 1.00 | 0.00 | H |
| ATOM | 3689 | 2HG1 | ILE | 274 | 103.185 | 77.575 | 63.045 | 1.00 | 0.00 | H |
| ATOM | 3690 | 2HG2 | ILE | 274 | 104.283 | 74.114 | 62.088 | 1.00 | 0.00 | H |
| ATOM | 3691 | 3HG2 | ILE | 274 | 104.802 | 75.606 | 62.892 | 1.00 | 0.00 | H |
| ATOM | 3692 | 1HG2 | ILE | 274 | 103.506 | 74.600 | 63.590 | 1.00 | 0.00 | H |
| ATOM | 3693 | 2HD1 | ILE | 274 | 101.395 | 78.386 | 61.491 | 1.00 | 0.00 | H |
| ATOM | 3694 | 3HD1 | ILE | 274 | 100.312 | 77.074 | 62.015 | 1.00 | 0.00 | H |
| ATOM | 3695 | 1HD1 | ILE | 274 | 100.765 | 78.375 | 63.149 | 1.00 | 0.00 | H |
| ATOM | 3696 | N | ASN | 275 | 105.570 | 75.583 | 59.420 | 1.00 | 0.00 | N |
| ATOM | 3697 | CA | ASN | 275 | 106.556 | 74.631 | 58.827 | 1.00 | 0.00 | C |
| ATOM | 3698 | C | ASN | 275 | 107.024 | 73.470 | 59.801 | 1.00 | 0.00 | C |
| ATOM | 3699 | O | ASN | 275 | 106.988 | 73.680 | 61.018 | 1.00 | 0.00 | O |
| ATOM | 3700 | CB | ASN | 275 | 107.707 | 75.475 | 58.199 | 1.00 | 0.00 | C |
| ATOM | 3701 | CG | ASN | 275 | 108.908 | 75.847 | 59.075 | 1.00 | 0.00 | C |
| ATOM | 3702 | OD1 | ASN | 275 | 109.770 | 75.027 | 59.362 | 1.00 | 0.00 | O |
| ATOM | 3703 | ND2 | ASN | 275 | 109.051 | 77.070 | 59.501 | 1.00 | 0.00 | N |
| ATOM | 3704 | H | ASN | 275 | 105.796 | 76.553 | 59.657 | 1.00 | 0.00 | H |
| ATOM | 3705 | HA | ASN | 275 | 106.038 | 74.137 | 57.980 | 1.00 | 0.00 | H |
| ATOM | 3706 | 1HB | ASN | 275 | 108.130 | 74.902 | 57.357 | 1.00 | 0.00 | H |
| ATOM | 3707 | 2HB | ASN | 275 | 107.304 | 76.377 | 57.702 | 1.00 | 0.00 | H |
| ATOM | 3708 | 1HD2 | ASN | 275 | 109.868 | 77.196 | 60.104 | 1.00 | 0.00 | H |
| ATOM | 3709 | 2HD2 | ASN | 275 | 108.261 | 77.716 | 59.370 | 1.00 | 0.00 | H |
| ATOM | 3710 | N | PRO | 276 | 107.529 | 72.281 | 59.351 | 1.00 | 0.00 | N |
| ATOM | 3711 | CA | PRO | 276 | 108.012 | 71.204 | 60.272 | 1.00 | 0.00 | C |
| ATOM | 3712 | C | PRO | 276 | 109.169 | 71.505 | 61.293 | 1.00 | 0.00 | C |
| ATOM | 3713 | O | PRO | 276 | 109.175 | 70.916 | 62.375 | 1.00 | 0.00 | O |
| ATOM | 3714 | CB | PRO | 276 | 108.317 | 70.048 | 59.295 | 1.00 | 0.00 | C |
| ATOM | 3715 | CG | PRO | 276 | 108.521 | 70.705 | 57.929 | 1.00 | 0.00 | C |
| ATOM | 3716 | CD | PRO | 276 | 107.552 | 71.883 | 57.930 | 1.00 | 0.00 | C |
| ATOM | 3717 | HA | PRO | 276 | 107.154 | 70.888 | 60.894 | 1.00 | 0.00 | H |
| ATOM | 3718 | 1HB | PRO | 276 | 109.183 | 69.429 | 59.600 | 1.00 | 0.00 | H |
| ATOM | 3719 | 2HB | PRO | 276 | 107.450 | 69.360 | 59.253 | 1.00 | 0.00 | H |
| ATOM | 3720 | 1HG | PRO | 276 | 109.564 | 71.066 | 57.829 | 1.00 | 0.00 | H |
| ATOM | 3721 | 2HG | PRO | 276 | 108.334 | 70.011 | 57.088 | 1.00 | 0.00 | H |
| ATOM | 3722 | 1HD | PRO | 276 | 107.894 | 72.676 | 57.242 | 1.00 | 0.00 | H |
| ATOM | 3723 | 2HD | PRO | 276 | 106.538 | 71.571 | 57.604 | 1.00 | 0.00 | H |
| ATOM | 3724 | N | VAL | 277 | 110.109 | 72.418 | 60.991 | 1.00 | 0.00 | N |
| ATOM | 3725 | CA | VAL | 277 | 111.112 | 72.927 | 61.992 | 1.00 | 0.00 | C |
| ATOM | 3726 | C | VAL | 277 | 110.471 | 73.844 | 63.108 | 1.00 | 0.00 | C |
| ATOM | 3727 | O | VAL | 277 | 110.775 | 73.669 | 64.292 | 1.00 | 0.00 | O |
| ATOM | 3728 | CB | VAL | 277 | 112.343 | 73.601 | 61.275 | 1.00 | 0.00 | C |
| ATOM | 3729 | CG1 | VAL | 277 | 113.500 | 73.923 | 62.250 | 1.00 | 0.00 | C |
| ATOM | 3730 | CG2 | VAL | 277 | 112.986 | 72.778 | 60.130 | 1.00 | 0.00 | C |
| ATOM | 3731 | H | VAL | 277 | 109.923 | 72.910 | 60.110 | 1.00 | 0.00 | H |
| ATOM | 3732 | HA | VAL | 277 | 111.515 | 72.046 | 62.533 | 1.00 | 0.00 | H |
| ATOM | 3733 | HB | VAL | 277 | 111.989 | 74.556 | 60.836 | 1.00 | 0.00 | H |
| ATOM | 3734 | 1HG1 | VAL | 277 | 113.946 | 73.012 | 62.689 | 1.00 | 0.00 | H |
| ATOM | 3735 | 2HG1 | VAL | 277 | 114.317 | 74.489 | 61.763 | 1.00 | 0.00 | H |
| ATOM | 3736 | 3HG1 | VAL | 277 | 113.158 | 74.543 | 63.096 | 1.00 | 0.00 | H |
| ATOM | 3737 | 2HG2 | VAL | 277 | 112.267 | 72.588 | 59.312 | 1.00 | 0.00 | H |
| ATOM | 3738 | 3HG2 | VAL | 277 | 113.844 | 73.303 | 59.667 | 1.00 | 0.00 | H |
| ATOM | 3739 | 1HG2 | VAL | 277 | 113.347 | 71.793 | 60.477 | 1.00 | 0.00 | H |
| ATOM | 3740 | N | ALA | 278 | 109.573 | 74.787 | 62.751 | 1.00 | 0.00 | N |
| ATOM | 3741 | CA | ALA | 278 | 108.679 | 75.464 | 63.733 | 1.00 | 0.00 | C |
| ATOM | 3742 | C | ALA | 278 | 107.712 | 74.540 | 64.557 | 1.00 | 0.00 | C |
| ATOM | 3743 | O | ALA | 278 | 107.630 | 74.703 | 65.775 | 1.00 | 0.00 | O |
| ATOM | 3744 | CB | ALA | 278 | 107.921 | 76.556 | 62.955 | 1.00 | 0.00 | C |
| ATOM | 3745 | H | ALA | 278 | 109.333 | 74.738 | 61.754 | 1.00 | 0.00 | H |
| ATOM | 3746 | HA | ALA | 278 | 109.320 | 75.978 | 64.478 | 1.00 | 0.00 | H |
| ATOM | 3747 | 2HB | ALA | 278 | 108.606 | 77.275 | 62.472 | 1.00 | 0.00 | H |
| ATOM | 3748 | 3HB | ALA | 278 | 107.273 | 76.132 | 62.163 | 1.00 | 0.00 | H |
| ATOM | 3749 | 1HB | ALA | 278 | 107.267 | 77.145 | 63.624 | 1.00 | 0.00 | H |
| ATOM | 3750 | N | ALA | 279 | 107.026 | 73.560 | 63.934 | 1.00 | 0.00 | N |
| ATOM | 3751 | CA | ALA | 279 | 106.300 | 72.479 | 64.659 | 1.00 | 0.00 | C |
| ATOM | 3752 | C | ALA | 279 | 107.139 | 71.631 | 65.676 | 1.00 | 0.00 | C |
| ATOM | 3753 | O | ALA | 279 | 106.714 | 71.487 | 66.822 | 1.00 | 0.00 | O |
| ATOM | 3754 | CB | ALA | 279 | 105.640 | 71.588 | 63.591 | 1.00 | 0.00 | C |
| ATOM | 3755 | H | ALA | 279 | 107.144 | 73.548 | 62.910 | 1.00 | 0.00 | H |
| ATOM | 3756 | HA | ALA | 279 | 105.486 | 72.953 | 65.245 | 1.00 | 0.00 | H |
| ATOM | 3757 | 2HB | ALA | 279 | 105.033 | 72.162 | 62.868 | 1.00 | 0.00 | H |
| ATOM | 3758 | 3HB | ALA | 279 | 106.390 | 71.021 | 63.011 | 1.00 | 0.00 | H |
| ATOM | 3759 | 1HB | ALA | 279 | 104.968 | 70.841 | 64.053 | 1.00 | 0.00 | H |
| ATOM | 3760 | N | SER | 280 | 108.335 | 71.141 | 65.295 | 1.00 | 0.00 | N |
| ATOM | 3761 | CA | SER | 280 | 109.313 | 70.538 | 66.246 | 1.00 | 0.00 | C |
| ATOM | 3762 | C | SER | 280 | 109.736 | 71.432 | 67.466 | 1.00 | 0.00 | C |
| ATOM | 3763 | O | SER | 280 | 109.698 | 70.949 | 68.596 | 1.00 | 0.00 | O |
| ATOM | 3764 | CB | SER | 280 | 110.530 | 70.068 | 65.414 | 1.00 | 0.00 | C |
| ATOM | 3765 | OG | SER | 280 | 111.472 | 69.355 | 66.219 | 1.00 | 0.00 | O |
| ATOM | 3766 | H | SER | 280 | 108.550 | 71.281 | 64.299 | 1.00 | 0.00 | H |
| ATOM | 3767 | HA | SER | 280 | 108.843 | 69.630 | 66.675 | 1.00 | 0.00 | H |
| ATOM | 3768 | 1HB | SER | 280 | 110.202 | 69.410 | 64.585 | 1.00 | 0.00 | H |
| ATOM | 3769 | 2HB | SER | 280 | 111.030 | 70.929 | 64.926 | 1.00 | 0.00 | H |
| ATOM | 3770 | HG | SER | 280 | 111.677 | 69.903 | 66.986 | 1.00 | 0.00 | H |
| ATOM | 3771 | N | LEU | 281 | 110.091 | 72.716 | 67.265 | 0.00 | 0.00 | N |
| ATOM | 3772 | CA | LEU | 281 | 110.282 | 73.690 | 68.385 | 0.00 | 0.00 | C |
| ATOM | 3773 | C | LEU | 281 | 109.019 | 73.976 | 69.287 | 0.00 | 0.00 | C |
| ATOM | 3774 | O | LEU | 281 | 109.153 | 74.008 | 70.512 | 0.00 | 0.00 | O |
| ATOM | 3775 | CB | LEU | 281 | 110.916 | 74.971 | 67.767 | 0.00 | 0.00 | C |
| ATOM | 3776 | CG | LEU | 281 | 111.466 | 76.022 | 68.769 | 0.00 | 0.00 | C |
| ATOM | 3777 | CD1 | LEU | 281 | 112.724 | 75.538 | 69.510 | 0.00 | 0.00 | C |
| ATOM | 3778 | CD2 | LEU | 281 | 111.794 | 77.333 | 68.038 | 0.00 | 0.00 | C |
| ATOM | 3779 | H | LEU | 281 | 110.044 | 73.008 | 66.280 | 0.00 | 0.00 | H |
| ATOM | 3780 | HA | LEU | 281 | 111.034 | 73.249 | 69.067 | 0.00 | 0.00 | H |
| ATOM | 3781 | 1HB | LEU | 281 | 110.155 | 75.447 | 67.118 | 0.00 | 0.00 | H |
| ATOM | 3782 | 2HB | LEU | 281 | 111.735 | 74.695 | 67.073 | 0.00 | 0.00 | H |
| ATOM | 3783 | HG | LEU | 281 | 110.686 | 76.238 | 69.526 | 0.00 | 0.00 | H |
| ATOM | 3784 | 1HD1 | LEU | 281 | 113.118 | 76.305 | 70.202 | 0.00 | 0.00 | H |
| ATOM | 3785 | 2HD1 | LEU | 281 | 112.523 | 74.639 | 70.123 | 0.00 | 0.00 | H |
| ATOM | 3786 | 3HD1 | LEU | 281 | 113.541 | 75.285 | 68.812 | 0.00 | 0.00 | H |
| ATOM | 3787 | 1HD2 | LEU | 281 | 112.150 | 78.116 | 68.736 | 0.00 | 0.00 | H |
| ATOM | 3788 | 2HD2 | LEU | 281 | 112.581 | 77.195 | 67.273 | 0.00 | 0.00 | H |
| ATOM | 3789 | 3HD2 | LEU | 281 | 110.909 | 77.749 | 67.522 | 0.00 | 0.00 | H |
| ATOM | 3790 | N | ILE | 282 | 107.811 | 74.141 | 68.710 | 1.00 | 0.00 | N |
| ATOM | 3791 | CA | ILE | 282 | 106.519 | 74.183 | 69.481 | 1.00 | 0.00 | C |
| ATOM | 3792 | C | ILE | 282 | 106.240 | 72.868 | 70.311 | 1.00 | 0.00 | C |
| ATOM | 3793 | O | ILE | 282 | 105.937 | 72.968 | 71.499 | 1.00 | 0.00 | O |
| ATOM | 3794 | CB | ILE | 282 | 105.333 | 74.624 | 68.532 | 1.00 | 0.00 | C |
| ATOM | 3795 | CG1 | ILE | 282 | 105.508 | 76.060 | 67.944 | 1.00 | 0.00 | C |
| ATOM | 3796 | CG2 | ILE | 282 | 103.939 | 74.566 | 69.215 | 1.00 | 0.00 | C |
| ATOM | 3797 | CD1 | ILE | 282 | 104.640 | 76.398 | 66.716 | 1.00 | 0.00 | C |
| ATOM | 3798 | H | ILE | 282 | 107.829 | 74.102 | 67.681 | 1.00 | 0.00 | H |
| ATOM | 3799 | HA | ILE | 282 | 106.618 | 74.980 | 70.244 | 1.00 | 0.00 | H |
| ATOM | 3800 | HB | ILE | 282 | 105.319 | 73.908 | 67.686 | 1.00 | 0.00 | H |
| ATOM | 3801 | 1HG1 | ILE | 282 | 105.354 | 76.820 | 68.735 | 1.00 | 0.00 | H |
| ATOM | 3802 | 2HG1 | ILE | 282 | 106.559 | 76.201 | 67.631 | 1.00 | 0.00 | H |
| ATOM | 3803 | 2HG2 | ILE | 282 | 103.703 | 73.553 | 69.590 | 1.00 | 0.00 | H |
| ATOM | 3804 | 3HG2 | ILE | 282 | 103.874 | 75.257 | 70.076 | 1.00 | 0.00 | H |
| ATOM | 3805 | 1HG2 | ILE | 282 | 103.116 | 74.826 | 68.525 | 1.00 | 0.00 | H |
| ATOM | 3806 | 2HD1 | ILE | 282 | 104.804 | 75.679 | 65.892 | 1.00 | 0.00 | H |
| ATOM | 3807 | 3HD1 | ILE | 282 | 103.559 | 76.401 | 66.950 | 1.00 | 0.00 | H |
| ATOM | 3808 | 1HD1 | ILE | 282 | 104.881 | 77.403 | 66.322 | 1.00 | 0.00 | H |
| ATOM | 3809 | N | GLN | 283 | 106.365 | 71.662 | 69.727 | 1.00 | 0.00 | N |
| ATOM | 3810 | CA | GLN | 283 | 106.324 | 70.366 | 70.478 | 1.00 | 0.00 | C |
| ATOM | 3811 | C | GLN | 283 | 107.407 | 70.164 | 71.602 | 1.00 | 0.00 | C |
| ATOM | 3812 | O | GLN | 283 | 107.077 | 69.647 | 72.669 | 1.00 | 0.00 | O |
| ATOM | 3813 | CB | GLN | 283 | 106.371 | 69.207 | 69.441 | 1.00 | 0.00 | C |
| ATOM | 3814 | CG | GLN | 283 | 105.123 | 69.066 | 68.524 | 1.00 | 0.00 | C |
| ATOM | 3815 | CD | GLN | 283 | 105.263 | 67.998 | 67.437 | 1.00 | 0.00 | C |
| ATOM | 3816 | OE1 | GLN | 283 | 105.841 | 68.208 | 66.378 | 1.00 | 0.00 | O |
| ATOM | 3817 | NE2 | GLN | 283 | 104.736 | 66.821 | 67.648 | 1.00 | 0.00 | N |
| ATOM | 3818 | H | GLN | 283 | 106.619 | 71.701 | 68.728 | 1.00 | 0.00 | H |
| ATOM | 3819 | HA | GLN | 283 | 105.351 | 70.309 | 71.006 | 1.00 | 0.00 | H |
| ATOM | 3820 | 1HB | GLN | 283 | 107.284 | 69.312 | 68.820 | 1.00 | 0.00 | H |
| ATOM | 3821 | 2HB | GLN | 283 | 106.509 | 68.248 | 69.979 | 1.00 | 0.00 | H |
| ATOM | 3822 | 1HG | GLN | 283 | 104.215 | 68.892 | 69.130 | 1.00 | 0.00 | H |
| ATOM | 3823 | 2HG | GLN | 283 | 104.931 | 70.020 | 68.000 | 1.00 | 0.00 | H |
| ATOM | 3824 | 1HE2 | GLN | 283 | 104.316 | 66.660 | 68.566 | 1.00 | 0.00 | H |
| ATOM | 3825 | 2HE2 | GLN | 283 | 104.911 | 66.152 | 66.894 | 1.00 | 0.00 | H |
| ATOM | 3826 | N | LYS | 284 | 108.670 | 70.582 | 71.395 | 1.00 | 0.00 | N |
| ATOM | 3827 | CA | LYS | 284 | 109.702 | 70.663 | 72.477 | 1.00 | 0.00 | C |
| ATOM | 3828 | C | LYS | 284 | 109.350 | 71.603 | 73.689 | 1.00 | 0.00 | C |
| ATOM | 3829 | O | LYS | 284 | 109.483 | 71.189 | 74.843 | 1.00 | 0.00 | O |
| ATOM | 3830 | CB | LYS | 284 | 111.052 | 71.077 | 71.820 | 1.00 | 0.00 | C |
| ATOM | 3831 | CG | LYS | 284 | 111.752 | 69.995 | 70.965 | 1.00 | 0.00 | C |
| ATOM | 3832 | CD | LYS | 284 | 112.939 | 70.569 | 70.164 | 1.00 | 0.00 | C |
| ATOM | 3833 | CE | LYS | 284 | 113.574 | 69.518 | 69.246 | 1.00 | 0.00 | C |
| ATOM | 3834 | NZ | LYS | 284 | 114.669 | 70.131 | 68.468 | 1.00 | 0.00 | N |
| ATOM | 3835 | 1HZ | LYS | 284 | 115.093 | 69.421 | 67.853 | 1.00 | 0.00 | H |
| ATOM | 3836 | 2HZ | LYS | 284 | 114.295 | 70.901 | 67.895 | 1.00 | 0.00 | H |
| ATOM | 3837 | 3HZ | LYS | 284 | 115.385 | 70.498 | 69.111 | 1.00 | 0.00 | H |
| ATOM | 3838 | H | LYS | 284 | 108.845 | 70.927 | 70.442 | 1.00 | 0.00 | H |
| ATOM | 3839 | HA | LYS | 284 | 109.825 | 69.652 | 72.914 | 1.00 | 0.00 | H |
| ATOM | 3840 | 1HB | LYS | 284 | 110.891 | 71.994 | 71.217 | 1.00 | 0.00 | H |
| ATOM | 3841 | 2HB | LYS | 284 | 111.762 | 71.394 | 72.606 | 1.00 | 0.00 | H |
| ATOM | 3842 | 1HG | LYS | 284 | 112.088 | 69.161 | 71.612 | 1.00 | 0.00 | H |
| ATOM | 3843 | 2HG | LYS | 284 | 111.023 | 69.541 | 70.266 | 1.00 | 0.00 | H |
| ATOM | 3844 | 1HD | LYS | 284 | 112.585 | 71.426 | 69.555 | 1.00 | 0.00 | H |
| ATOM | 3845 | 2HD | LYS | 284 | 113.695 | 70.985 | 70.860 | 1.00 | 0.00 | H |
| ATOM | 3846 | 1HE | LYS | 284 | 113.956 | 68.663 | 69.840 | 1.00 | 0.00 | H |
| ATOM | 3847 | 2HE | LYS | 284 | 112.813 | 69.091 | 68.562 | 1.00 | 0.00 | H |
| ATOM | 3848 | N | MET | 285 | 108.903 | 72.845 | 73.438 | 1.00 | 0.00 | N |
| ATOM | 3849 | CA | MET | 285 | 108.343 | 73.747 | 74.490 | 1.00 | 0.00 | C |
| ATOM | 3850 | C | MET | 285 | 107.007 | 73.268 | 75.172 | 1.00 | 0.00 | C |
| ATOM | 3851 | O | MET | 285 | 106.880 | 73.356 | 76.396 | 1.00 | 0.00 | O |
| ATOM | 3852 | CB | MET | 285 | 108.192 | 75.163 | 73.863 | 1.00 | 0.00 | C |
| ATOM | 3853 | CG | MET | 285 | 109.509 | 75.909 | 73.553 | 1.00 | 0.00 | C |
| ATOM | 3854 | SD | MET | 285 | 109.147 | 77.567 | 72.955 | 1.00 | 0.00 | S |
| ATOM | 3855 | CE | MET | 285 | 110.808 | 78.115 | 72.529 | 1.00 | 0.00 | C |
| ATOM | 3856 | H | MET | 285 | 108.820 | 73.061 | 72.436 | 1.00 | 0.00 | H |
| ATOM | 3857 | HA | MET | 285 | 109.080 | 73.822 | 75.315 | 1.00 | 0.00 | H |
| ATOM | 3858 | 1HB | MET | 285 | 107.573 | 75.105 | 72.945 | 1.00 | 0.00 | H |
| ATOM | 3859 | 2HB | MET | 285 | 107.603 | 75.796 | 74.550 | 1.00 | 0.00 | H |
| ATOM | 3860 | 1HG | MET | 285 | 110.149 | 75.976 | 74.452 | 1.00 | 0.00 | H |
| ATOM | 3861 | 2HG | MET | 285 | 110.091 | 75.364 | 72.785 | 1.00 | 0.00 | H |
| ATOM | 3862 | 1HE | MET | 285 | 111.438 | 78.200 | 73.430 | 1.00 | 0.00 | H |
| ATOM | 3863 | 3HE | MET | 285 | 111.288 | 77.407 | 71.829 | 1.00 | 0.00 | H |
| ATOM | 3864 | 2HE | MET | 285 | 110.774 | 79.105 | 72.043 | 1.00 | 0.00 | H |
| ATOM | 3865 | N | LEU | 286 | 106.023 | 72.769 | 74.403 | 0.00 | 0.00 | N |
| ATOM | 3866 | CA | LEU | 286 | 104.734 | 72.249 | 74.938 | 0.00 | 0.00 | C |
| ATOM | 3867 | C | LEU | 286 | 104.765 | 70.689 | 75.136 | 0.00 | 0.00 | C |
| ATOM | 3868 | O | LEU | 286 | 104.191 | 69.933 | 74.344 | 0.00 | 0.00 | O |
| ATOM | 3869 | CB | LEU | 286 | 103.593 | 72.713 | 73.979 | 0.00 | 0.00 | C |
| ATOM | 3870 | CG | LEU | 286 | 103.350 | 74.234 | 73.781 | 0.00 | 0.00 | C |
| ATOM | 3871 | CD1 | LEU | 286 | 102.345 | 74.488 | 72.647 | 0.00 | 0.00 | C |
| ATOM | 3872 | CD2 | LEU | 286 | 102.828 | 74.911 | 75.053 | 0.00 | 0.00 | C |
| ATOM | 3873 | H | LEU | 286 | 106.248 | 72.721 | 73.399 | 0.00 | 0.00 | H |
| ATOM | 3874 | HA | LEU | 286 | 104.520 | 72.696 | 75.927 | 0.00 | 0.00 | H |
| ATOM | 3875 | 1HB | LEU | 286 | 102.649 | 72.257 | 74.326 | 0.00 | 0.00 | H |
| ATOM | 3876 | 2HB | LEU | 286 | 103.772 | 72.247 | 72.989 | 0.00 | 0.00 | H |
| ATOM | 3877 | HG | LEU | 286 | 104.310 | 74.702 | 73.489 | 0.00 | 0.00 | H |
| ATOM | 3878 | 1HD1 | LEU | 286 | 102.251 | 75.564 | 72.415 | 0.00 | 0.00 | H |
| ATOM | 3879 | 2HD1 | LEU | 286 | 102.649 | 73.981 | 71.715 | 0.00 | 0.00 | H |
| ATOM | 3880 | 3HD1 | LEU | 286 | 101.334 | 74.117 | 72.893 | 0.00 | 0.00 | H |
| ATOM | 3881 | 1HD2 | LEU | 286 | 102.662 | 75.994 | 74.907 | 0.00 | 0.00 | H |
| ATOM | 3882 | 2HD2 | LEU | 286 | 101.871 | 74.475 | 75.399 | 0.00 | 0.00 | H |
| ATOM | 3883 | 3HD2 | LEU | 286 | 103.544 | 74.819 | 75.886 | 0.00 | 0.00 | H |
| ATOM | 3884 | N | GLN | 287 | 105.413 | 70.203 | 76.211 | 0.00 | 0.00 | N |
| ATOM | 3885 | CA | GLN | 287 | 105.589 | 68.743 | 76.475 | 0.00 | 0.00 | C |
| ATOM | 3886 | C | GLN | 287 | 105.173 | 68.375 | 77.939 | 0.00 | 0.00 | C |
| ATOM | 3887 | O | GLN | 287 | 105.882 | 68.685 | 78.895 | 0.00 | 0.00 | O |
| ATOM | 3888 | CB | GLN | 287 | 107.068 | 68.397 | 76.131 | 0.00 | 0.00 | C |
| ATOM | 3889 | CG | GLN | 287 | 107.540 | 66.935 | 76.360 | 0.00 | 0.00 | C |
| ATOM | 3890 | CD | GLN | 287 | 106.894 | 65.837 | 75.513 | 0.00 | 0.00 | C |
| ATOM | 3891 | OE1 | GLN | 287 | 107.388 | 65.437 | 74.467 | 0.00 | 0.00 | O |
| ATOM | 3892 | NE2 | GLN | 287 | 105.791 | 65.276 | 75.937 | 0.00 | 0.00 | N |
| ATOM | 3893 | H | GLN | 287 | 105.978 | 70.908 | 76.702 | 0.00 | 0.00 | H |
| ATOM | 3894 | HA | GLN | 287 | 104.957 | 68.147 | 75.785 | 0.00 | 0.00 | H |
| ATOM | 3895 | 1HB | GLN | 287 | 107.737 | 69.060 | 76.715 | 0.00 | 0.00 | H |
| ATOM | 3896 | 2HB | GLN | 287 | 107.271 | 68.671 | 75.076 | 0.00 | 0.00 | H |
| ATOM | 3897 | 1HG | GLN | 287 | 107.477 | 66.675 | 77.433 | 0.00 | 0.00 | H |
| ATOM | 3898 | 2HG | GLN | 287 | 108.623 | 66.896 | 76.145 | 0.00 | 0.00 | H |
| ATOM | 3899 | 1HE2 | GLN | 287 | 105.310 | 65.754 | 76.700 | 0.00 | 0.00 | H |
| ATOM | 3900 | 2HE2 | GLN | 287 | 105.393 | 64.634 | 75.247 | 0.00 | 0.00 | H |
| ATOM | 3901 | N | THR | 288 | 104.048 | 67.664 | 78.139 | 1.00 | 0.00 | N |
| ATOM | 3902 | CA | THR | 288 | 103.543 | 67.291 | 79.511 | 1.00 | 0.00 | C |
| ATOM | 3903 | C | THR | 288 | 104.436 | 66.358 | 80.418 | 1.00 | 0.00 | C |
| ATOM | 3904 | O | THR | 288 | 104.234 | 66.308 | 81.634 | 1.00 | 0.00 | O |
| ATOM | 3905 | CB | THR | 288 | 102.058 | 66.822 | 79.390 | 1.00 | 0.00 | C |
| ATOM | 3906 | OG1 | THR | 288 | 101.424 | 66.860 | 80.660 | 1.00 | 0.00 | O |
| ATOM | 3907 | CG2 | THR | 288 | 101.822 | 65.403 | 78.845 | 1.00 | 0.00 | C |
| ATOM | 3908 | H | THR | 288 | 103.483 | 67.504 | 77.290 | 1.00 | 0.00 | H |
| ATOM | 3909 | HA | THR | 288 | 103.495 | 68.235 | 80.088 | 1.00 | 0.00 | H |
| ATOM | 3910 | HB | THR | 288 | 101.518 | 67.539 | 78.739 | 1.00 | 0.00 | H |
| ATOM | 3911 | HG1 | THR | 288 | 100.655 | 66.284 | 80.589 | 1.00 | 0.00 | H |
| ATOM | 3912 | 1HG2 | THR | 288 | 100.743 | 65.181 | 78.732 | 1.00 | 0.00 | H |
| ATOM | 3913 | 2HG2 | THR | 288 | 102.281 | 65.264 | 77.849 | 1.00 | 0.00 | H |
| ATOM | 3914 | 3HG2 | THR | 288 | 102.250 | 64.629 | 79.511 | 1.00 | 0.00 | H |
| ATOM | 3915 | N | ASP | 289 | 105.434 | 65.659 | 79.851 | 0.00 | 0.00 | N |
| ATOM | 3916 | CA | ASP | 289 | 106.531 | 65.022 | 80.628 | 0.00 | 0.00 | C |
| ATOM | 3917 | C | ASP | 289 | 107.747 | 66.022 | 80.745 | 0.00 | 0.00 | C |
| ATOM | 3918 | O | ASP | 289 | 108.507 | 66.112 | 79.773 | 0.00 | 0.00 | O |
| ATOM | 3919 | CB | ASP | 289 | 106.933 | 63.698 | 79.922 | 0.00 | 0.00 | C |
| ATOM | 3920 | CG | ASP | 289 | 105.899 | 62.579 | 79.992 | 0.00 | 0.00 | C |
| ATOM | 3921 | OD1 | ASP | 289 | 105.743 | 61.857 | 80.970 | 0.00 | 0.00 | O |
| ATOM | 3922 | OD2 | ASP | 289 | 105.173 | 62.469 | 78.848 | 0.00 | 0.00 | O |
| ATOM | 3923 | H | ASP | 289 | 105.566 | 65.931 | 78.873 | 0.00 | 0.00 | H |
| ATOM | 3924 | HA | ASP | 289 | 106.183 | 64.747 | 81.643 | 0.00 | 0.00 | H |
| ATOM | 3925 | 1HB | ASP | 289 | 107.856 | 63.302 | 80.382 | 0.00 | 0.00 | H |
| ATOM | 3926 | 2HB | ASP | 289 | 107.206 | 63.884 | 78.866 | 0.00 | 0.00 | H |
| ATOM | 3927 | N | PRO | 290 | 108.009 | 66.769 | 81.862 | 1.00 | 0.00 | N |
| ATOM | 3928 | CA | PRO | 290 | 109.137 | 67.754 | 81.926 | 1.00 | 0.00 | C |
| ATOM | 3929 | C | PRO | 290 | 110.615 | 67.241 | 81.787 | 1.00 | 0.00 | C |
| ATOM | 3930 | O | PRO | 290 | 111.483 | 68.016 | 81.382 | 1.00 | 0.00 | O |
| ATOM | 3931 | CB | PRO | 290 | 108.847 | 68.505 | 83.240 | 1.00 | 0.00 | C |
| ATOM | 3932 | CG | PRO | 290 | 107.998 | 67.547 | 84.076 | 1.00 | 0.00 | C |
| ATOM | 3933 | CD | PRO | 290 | 107.135 | 66.813 | 83.051 | 1.00 | 0.00 | C |
| ATOM | 3934 | HA | PRO | 290 | 109.003 | 68.477 | 81.096 | 1.00 | 0.00 | H |
| ATOM | 3935 | 1HB | PRO | 290 | 109.761 | 68.838 | 83.770 | 1.00 | 0.00 | H |
| ATOM | 3936 | 2HB | PRO | 290 | 108.273 | 69.427 | 83.016 | 1.00 | 0.00 | H |
| ATOM | 3937 | 1HG | PRO | 290 | 108.650 | 66.830 | 84.612 | 1.00 | 0.00 | H |
| ATOM | 3938 | 2HG | PRO | 290 | 107.394 | 68.066 | 84.842 | 1.00 | 0.00 | H |
| ATOM | 3939 | 1HD | PRO | 290 | 106.837 | 65.816 | 83.426 | 1.00 | 0.00 | H |
| ATOM | 3940 | 2HD | PRO | 290 | 106.212 | 67.384 | 82.828 | 1.00 | 0.00 | H |
| ATOM | 3941 | N | THR | 291 | 110.902 | 65.954 | 82.033 | 0.00 | 0.00 | N |
| ATOM | 3942 | CA | THR | 291 | 112.170 | 65.287 | 81.572 | 0.00 | 0.00 | C |
| ATOM | 3943 | C | THR | 291 | 112.443 | 65.277 | 80.021 | 0.00 | 0.00 | C |
| ATOM | 3944 | O | THR | 291 | 113.599 | 65.396 | 79.608 | 0.00 | 0.00 | O |
| ATOM | 3945 | CB | THR | 291 | 112.272 | 63.838 | 82.150 | 0.00 | 0.00 | C |
| ATOM | 3946 | OG1 | THR | 291 | 111.144 | 63.050 | 81.782 | 0.00 | 0.00 | O |
| ATOM | 3947 | CG2 | THR | 291 | 112.401 | 63.745 | 83.679 | 0.00 | 0.00 | C |
| ATOM | 3948 | H | THR | 291 | 110.076 | 65.407 | 82.290 | 0.00 | 0.00 | H |
| ATOM | 3949 | HA | THR | 291 | 113.018 | 65.861 | 81.998 | 0.00 | 0.00 | H |
| ATOM | 3950 | HB | THR | 291 | 113.182 | 63.366 | 81.723 | 0.00 | 0.00 | H |
| ATOM | 3951 | HG1 | THR | 291 | 111.255 | 62.203 | 82.220 | 0.00 | 0.00 | H |
| ATOM | 3952 | 1HG2 | THR | 291 | 112.536 | 62.702 | 84.020 | 0.00 | 0.00 | H |
| ATOM | 3953 | 2HG2 | THR | 291 | 113.270 | 64.321 | 84.048 | 0.00 | 0.00 | H |
| ATOM | 3954 | 3HG2 | THR | 291 | 111.506 | 64.145 | 84.192 | 0.00 | 0.00 | H |
| ATOM | 3955 | N | ALA | 292 | 111.407 | 65.155 | 79.170 | 1.00 | 0.00 | N |
| ATOM | 3956 | CA | ALA | 292 | 111.517 | 65.430 | 77.707 | 1.00 | 0.00 | C |
| ATOM | 3957 | C | ALA | 292 | 111.351 | 66.921 | 77.217 | 1.00 | 0.00 | C |
| ATOM | 3958 | O | ALA | 292 | 111.419 | 67.163 | 76.008 | 1.00 | 0.00 | O |
| ATOM | 3959 | CB | ALA | 292 | 110.464 | 64.496 | 77.075 | 1.00 | 0.00 | C |
| ATOM | 3960 | H | ALA | 292 | 110.496 | 65.160 | 79.642 | 1.00 | 0.00 | H |
| ATOM | 3961 | HA | ALA | 292 | 112.511 | 65.107 | 77.339 | 1.00 | 0.00 | H |
| ATOM | 3962 | 2HB | ALA | 292 | 110.646 | 63.432 | 77.318 | 1.00 | 0.00 | H |
| ATOM | 3963 | 3HB | ALA | 292 | 109.436 | 64.743 | 77.400 | 1.00 | 0.00 | H |
| ATOM | 3964 | 1HB | ALA | 292 | 110.472 | 64.572 | 75.971 | 1.00 | 0.00 | H |
| ATOM | 3965 | N | ARG | 293 | 111.155 | 67.913 | 78.102 | 0.00 | 0.00 | N |
| ATOM | 3966 | CA | ARG | 293 | 111.035 | 69.349 | 77.730 | 0.00 | 0.00 | C |
| ATOM | 3967 | C | ARG | 293 | 112.431 | 70.079 | 77.848 | 0.00 | 0.00 | C |
| ATOM | 3968 | O | ARG | 293 | 112.874 | 70.307 | 78.980 | 0.00 | 0.00 | O |
| ATOM | 3969 | CB | ARG | 293 | 109.945 | 69.948 | 78.668 | 0.00 | 0.00 | C |
| ATOM | 3970 | CG | ARG | 293 | 109.355 | 71.322 | 78.250 | 0.00 | 0.00 | C |
| ATOM | 3971 | CD | ARG | 293 | 108.545 | 72.043 | 79.350 | 0.00 | 0.00 | C |
| ATOM | 3972 | NE | ARG | 293 | 107.377 | 71.246 | 79.800 | 0.00 | 0.00 | N |
| ATOM | 3973 | CZ | ARG | 293 | 106.613 | 71.500 | 80.849 | 0.00 | 0.00 | C |
| ATOM | 3974 | NH1 | ARG | 293 | 106.746 | 72.531 | 81.627 | 0.00 | 0.00 | N |
| ATOM | 3975 | NH2 | ARG | 293 | 105.679 | 70.655 | 81.106 | 0.00 | 0.00 | N |
| ATOM | 3976 | HE | ARG | 293 | 107.138 | 70.414 | 79.242 | 1.00 | 0.00 | H |
| ATOM | 3977 | H | ARG | 293 | 111.190 | 67.608 | 79.081 | 0.00 | 0.00 | H |
| ATOM | 3978 | HA | ARG | 293 | 110.638 | 69.440 | 76.702 | 0.00 | 0.00 | H |
| ATOM | 3979 | 1HB | ARG | 293 | 110.346 | 70.018 | 79.696 | 0.00 | 0.00 | H |
| ATOM | 3980 | 2HB | ARG | 293 | 109.087 | 69.250 | 78.757 | 0.00 | 0.00 | H |
| ATOM | 3981 | 1HG | ARG | 293 | 108.717 | 71.197 | 77.352 | 0.00 | 0.00 | H |
| ATOM | 3982 | 2HG | ARG | 293 | 110.172 | 71.994 | 77.921 | 0.00 | 0.00 | H |
| ATOM | 3983 | 1HD | ARG | 293 | 108.180 | 73.013 | 78.961 | 0.00 | 0.00 | H |
| ATOM | 3984 | 2HD | ARG | 293 | 109.216 | 72.282 | 80.197 | 0.00 | 0.00 | H |
| ATOM | 3985 | 1HH1 | ARG | 293 | 106.110 | 72.599 | 82.417 | 0.00 | 0.00 | H |
| ATOM | 3986 | 2HH1 | ARG | 293 | 107.501 | 73.161 | 81.338 | 0.00 | 0.00 | H |
| ATOM | 3987 | 1HH2 | ARG | 293 | 105.098 | 70.793 | 81.925 | 0.00 | 0.00 | H |
| ATOM | 3988 | 2HH2 | ARG | 293 | 105.711 | 69.884 | 80.425 | 0.00 | 0.00 | H |
| ATOM | 3989 | N | PRO | 294 | 113.176 | 70.471 | 76.769 | 1.00 | 0.00 | N |
| ATOM | 3990 | CA | PRO | 294 | 114.485 | 71.175 | 76.914 | 1.00 | 0.00 | C |
| ATOM | 3991 | C | PRO | 294 | 114.392 | 72.639 | 77.466 | 1.00 | 0.00 | C |
| ATOM | 3992 | O | PRO | 294 | 113.468 | 73.393 | 77.152 | 1.00 | 0.00 | O |
| ATOM | 3993 | CB | PRO | 294 | 115.049 | 71.081 | 75.481 | 1.00 | 0.00 | C |
| ATOM | 3994 | CG | PRO | 294 | 113.832 | 70.955 | 74.566 | 1.00 | 0.00 | C |
| ATOM | 3995 | CD | PRO | 294 | 112.832 | 70.136 | 75.375 | 1.00 | 0.00 | C |
| ATOM | 3996 | HA | PRO | 294 | 115.134 | 70.581 | 77.588 | 1.00 | 0.00 | H |
| ATOM | 3997 | 1HB | PRO | 294 | 115.680 | 71.944 | 75.202 | 1.00 | 0.00 | H |
| ATOM | 3998 | 2HB | PRO | 294 | 115.686 | 70.180 | 75.389 | 1.00 | 0.00 | H |
| ATOM | 3999 | 1HG | PRO | 294 | 113.411 | 71.958 | 74.363 | 1.00 | 0.00 | H |
| ATOM | 4000 | 2HG | PRO | 294 | 114.067 | 70.495 | 73.588 | 1.00 | 0.00 | H |
| ATOM | 4001 | 1HD | PRO | 294 | 111.795 | 70.403 | 75.105 | 1.00 | 0.00 | H |
| ATOM | 4002 | 2HD | PRO | 294 | 112.957 | 69.050 | 75.192 | 1.00 | 0.00 | H |
| ATOM | 4003 | N | THR | 295 | 115.339 | 73.037 | 78.328 | 1.00 | 0.00 | N |
| ATOM | 4004 | CA | THR | 295 | 115.353 | 74.386 | 78.981 | 1.00 | 0.00 | C |
| ATOM | 4005 | C | THR | 295 | 116.817 | 74.695 | 79.466 | 1.00 | 0.00 | C |
| ATOM | 4006 | O | THR | 295 | 117.387 | 73.917 | 80.231 | 1.00 | 0.00 | O |
| ATOM | 4007 | CB | THR | 295 | 114.333 | 74.468 | 80.166 | 1.00 | 0.00 | C |
| ATOM | 4008 | OG1 | THR | 295 | 113.010 | 74.194 | 79.725 | 1.00 | 0.00 | O |
| ATOM | 4009 | CG2 | THR | 295 | 114.231 | 75.846 | 80.832 | 1.00 | 0.00 | C |
| ATOM | 4010 | H | THR | 295 | 116.015 | 72.303 | 78.578 | 1.00 | 0.00 | H |
| ATOM | 4011 | HA | THR | 295 | 115.040 | 75.133 | 78.232 | 1.00 | 0.00 | H |
| ATOM | 4012 | HB | THR | 295 | 114.610 | 73.714 | 80.932 | 1.00 | 0.00 | H |
| ATOM | 4013 | HG1 | THR | 295 | 113.092 | 73.632 | 78.939 | 1.00 | 0.00 | H |
| ATOM | 4014 | 1HG2 | THR | 295 | 113.968 | 76.636 | 80.108 | 1.00 | 0.00 | H |
| ATOM | 4015 | 2HG2 | THR | 295 | 113.451 | 75.851 | 81.616 | 1.00 | 0.00 | H |
| ATOM | 4016 | 3HG2 | THR | 295 | 115.181 | 76.140 | 81.311 | 1.00 | 0.00 | H |
| ATOM | 4017 | N | ILE | 296 | 117.571 | 75.760 | 79.174 | 1.00 | 0.00 | N |
| ATOM | 4018 | CA | ILE | 296 | 117.200 | 76.943 | 78.320 | 1.00 | 0.00 | C |
| ATOM | 4019 | C | ILE | 296 | 118.198 | 77.193 | 77.130 | 1.00 | 0.00 | C |
| ATOM | 4020 | O | ILE | 296 | 117.754 | 77.424 | 76.002 | 1.00 | 0.00 | O |
| ATOM | 4021 | CB | ILE | 296 | 116.909 | 78.203 | 79.219 | 1.00 | 0.00 | C |
| ATOM | 4022 | CG1 | ILE | 296 | 116.285 | 79.421 | 78.478 | 1.00 | 0.00 | C |
| ATOM | 4023 | CG2 | ILE | 296 | 118.123 | 78.712 | 80.038 | 1.00 | 0.00 | C |
| ATOM | 4024 | CD1 | ILE | 296 | 114.886 | 79.182 | 77.886 | 1.00 | 0.00 | C |
| ATOM | 4025 | H | ILE | 296 | 118.460 | 75.689 | 79.683 | 1.00 | 0.00 | H |
| ATOM | 4026 | HA | ILE | 296 | 116.253 | 76.730 | 77.797 | 1.00 | 0.00 | H |
| ATOM | 4027 | HB | ILE | 296 | 116.153 | 77.880 | 79.960 | 1.00 | 0.00 | H |
| ATOM | 4028 | 1HG1 | ILE | 296 | 116.200 | 80.276 | 79.177 | 1.00 | 0.00 | H |
| ATOM | 4029 | 2HG1 | ILE | 296 | 116.970 | 79.783 | 77.684 | 1.00 | 0.00 | H |
| ATOM | 4030 | 2HG2 | ILE | 296 | 118.571 | 77.916 | 80.662 | 1.00 | 0.00 | H |
| ATOM | 4031 | 3HG2 | ILE | 296 | 118.925 | 79.109 | 79.388 | 1.00 | 0.00 | H |
| ATOM | 4032 | 1HG2 | ILE | 296 | 117.843 | 79.526 | 80.735 | 1.00 | 0.00 | H |
| ATOM | 4033 | 2HD1 | ILE | 296 | 114.897 | 78.440 | 77.066 | 1.00 | 0.00 | H |
| ATOM | 4034 | 3HD1 | ILE | 296 | 114.170 | 78.831 | 78.652 | 1.00 | 0.00 | H |
| ATOM | 4035 | 1HD1 | ILE | 296 | 114.472 | 80.115 | 77.461 | 1.00 | 0.00 | H |
| ATOM | 4036 | N | ASN | 297 | 119.524 | 77.082 | 77.342 | 1.00 | 0.00 | N |
| ATOM | 4037 | CA | ASN | 297 | 120.539 | 76.963 | 76.244 | 1.00 | 0.00 | C |
| ATOM | 4038 | C | ASN | 297 | 120.318 | 75.865 | 75.137 | 1.00 | 0.00 | C |
| ATOM | 4039 | O | ASN | 297 | 120.720 | 76.074 | 73.993 | 1.00 | 0.00 | O |
| ATOM | 4040 | CB | ASN | 297 | 121.955 | 76.877 | 76.885 | 1.00 | 0.00 | C |
| ATOM | 4041 | CG | ASN | 297 | 122.271 | 75.635 | 77.728 | 1.00 | 0.00 | C |
| ATOM | 4042 | OD1 | ASN | 297 | 121.591 | 75.318 | 78.696 | 1.00 | 0.00 | O |
| ATOM | 4043 | ND2 | ASN | 297 | 123.300 | 74.896 | 77.410 | 1.00 | 0.00 | N |
| ATOM | 4044 | H | ASN | 297 | 119.777 | 76.885 | 78.316 | 1.00 | 0.00 | H |
| ATOM | 4045 | HA | ASN | 297 | 120.509 | 77.926 | 75.696 | 1.00 | 0.00 | H |
| ATOM | 4046 | 1HB | ASN | 297 | 122.710 | 77.007 | 76.087 | 1.00 | 0.00 | H |
| ATOM | 4047 | 2HB | ASN | 297 | 122.116 | 77.751 | 77.540 | 1.00 | 0.00 | H |
| ATOM | 4048 | 1HD2 | ASN | 297 | 123.467 | 74.122 | 78.059 | 1.00 | 0.00 | H |
| ATOM | 4049 | 2HD2 | ASN | 297 | 123.883 | 75.208 | 76.631 | 1.00 | 0.00 | H |
| ATOM | 4050 | N | GLU | 298 | 119.665 | 74.735 | 75.458 | 1.00 | 0.00 | N |
| ATOM | 4051 | CA | GLU | 298 | 119.131 | 73.759 | 74.458 | 1.00 | 0.00 | C |
| ATOM | 4052 | C | GLU | 298 | 118.095 | 74.333 | 73.417 | 1.00 | 0.00 | C |
| ATOM | 4053 | O | GLU | 298 | 118.223 | 74.070 | 72.223 | 1.00 | 0.00 | O |
| ATOM | 4054 | CB | GLU | 298 | 118.500 | 72.576 | 75.246 | 1.00 | 0.00 | C |
| ATOM | 4055 | CG | GLU | 298 | 119.433 | 71.725 | 76.149 | 1.00 | 0.00 | C |
| ATOM | 4056 | CD | GLU | 298 | 118.675 | 70.676 | 76.962 | 1.00 | 0.00 | C |
| ATOM | 4057 | OE1 | GLU | 298 | 117.817 | 70.947 | 77.795 | 1.00 | 0.00 | O |
| ATOM | 4058 | OE2 | GLU | 298 | 119.052 | 69.407 | 76.656 | 1.00 | 0.00 | O |
| ATOM | 4059 | H | GLU | 298 | 119.435 | 74.674 | 76.456 | 1.00 | 0.00 | H |
| ATOM | 4060 | HA | GLU | 298 | 119.980 | 73.366 | 73.862 | 1.00 | 0.00 | H |
| ATOM | 4061 | 1HB | GLU | 298 | 117.661 | 72.964 | 75.858 | 1.00 | 0.00 | H |
| ATOM | 4062 | 2HB | GLU | 298 | 118.022 | 71.888 | 74.519 | 1.00 | 0.00 | H |
| ATOM | 4063 | 1HG | GLU | 298 | 120.219 | 71.239 | 75.542 | 1.00 | 0.00 | H |
| ATOM | 4064 | 2HG | GLU | 298 | 119.971 | 72.366 | 76.871 | 1.00 | 0.00 | H |
| ATOM | 4065 | N | LEU | 299 | 117.091 | 75.115 | 73.859 | 1.00 | 0.00 | H |
| ATOM | 4066 | CA | LEU | 299 | 116.186 | 75.882 | 72.942 | 1.00 | 0.00 | C |
| ATOM | 4067 | C | LEU | 299 | 116.861 | 77.050 | 72.136 | 1.00 | 0.00 | C |
| ATOM | 4068 | O | LEU | 299 | 116.532 | 77.249 | 70.965 | 1.00 | 0.00 | O |
| ATOM | 4069 | CB | LEU | 299 | 114.977 | 76.408 | 73.767 | 1.00 | 0.00 | C |
| ATOM | 4070 | CG | LEU | 299 | 113.984 | 75.356 | 74.328 | 1.00 | 0.00 | C |
| ATOM | 4071 | CD1 | LEU | 299 | 113.061 | 76.019 | 75.358 | 1.00 | 0.00 | C |
| ATOM | 4072 | CD2 | LEU | 299 | 113.127 | 74.708 | 73.229 | 1.00 | 0.00 | C |
| ATOM | 4073 | H | LEU | 299 | 117.153 | 75.335 | 74.857 | 1.00 | 0.00 | H |
| ATOM | 4074 | HA | LEU | 299 | 115.798 | 75.187 | 72.171 | 1.00 | 0.00 | H |
| ATOM | 4075 | 1HB | LEU | 299 | 115.370 | 77.032 | 74.594 | 1.00 | 0.00 | H |
| ATOM | 4076 | 2HB | LEU | 299 | 114.398 | 77.123 | 73.148 | 1.00 | 0.00 | H |
| ATOM | 4077 | HG | LEU | 299 | 114.545 | 74.556 | 74.851 | 1.00 | 0.00 | H |
| ATOM | 4078 | 2HD1 | LEU | 299 | 113.638 | 76.425 | 76.207 | 1.00 | 0.00 | H |
| ATOM | 4079 | 3HD1 | LEU | 299 | 112.472 | 76.850 | 74.931 | 1.00 | 0.00 | H |
| ATOM | 4080 | 1HD1 | LEU | 299 | 112.342 | 75.295 | 75.786 | 1.00 | 0.00 | H |
| ATOM | 4081 | 2HD2 | LEU | 299 | 112.564 | 75.457 | 72.642 | 1.00 | 0.00 | H |
| ATOM | 4082 | 3HD2 | LEU | 299 | 113.738 | 74.125 | 72.517 | 1.00 | 0.00 | H |
| ATOM | 4083 | 1HD2 | LEU | 299 | 112.380 | 74.008 | 73.649 | 1.00 | 0.00 | H |
| ATOM | 4084 | N | LEU | 300 | 117.800 | 77.804 | 72.742 | 0.00 | 0.00 | N |
| ATOM | 4085 | CA | LEU | 300 | 118.689 | 78.749 | 72.003 | 0.00 | 0.00 | C |
| ATOM | 4086 | C | LEU | 300 | 119.619 | 78.094 | 70.910 | 0.00 | 0.00 | C |
| ATOM | 4087 | O | LEU | 300 | 119.696 | 78.602 | 69.791 | 0.00 | 0.00 | O |
| ATOM | 4088 | CB | LEU | 300 | 119.475 | 79.548 | 73.087 | 0.00 | 0.00 | C |
| ATOM | 4089 | CG | LEU | 300 | 120.146 | 80.865 | 72.617 | 0.00 | 0.00 | C |
| ATOM | 4090 | CD1 | LEU | 300 | 119.115 | 81.989 | 72.419 | 0.00 | 0.00 | C |
| ATOM | 4091 | CD2 | LEU | 300 | 121.191 | 81.327 | 73.645 | 0.00 | 0.00 | C |
| ATOM | 4092 | H | LEU | 300 | 117.959 | 77.545 | 73.722 | 0.00 | 0.00 | H |
| ATOM | 4093 | HA | LEU | 300 | 118.035 | 79.456 | 71.458 | 0.00 | 0.00 | H |
| ATOM | 4094 | 1HB | LEU | 300 | 120.243 | 78.874 | 73.511 | 0.00 | 0.00 | H |
| ATOM | 4095 | 2HB | LEU | 300 | 118.821 | 79.790 | 73.950 | 0.00 | 0.00 | H |
| ATOM | 4096 | HG | LEU | 300 | 120.669 | 80.685 | 71.654 | 0.00 | 0.00 | H |
| ATOM | 4097 | 1HD1 | LEU | 300 | 119.592 | 82.917 | 72.055 | 0.00 | 0.00 | H |
| ATOM | 4098 | 2HD1 | LEU | 300 | 118.343 | 81.724 | 71.675 | 0.00 | 0.00 | H |
| ATOM | 4099 | 3HD1 | LEU | 300 | 118.589 | 82.249 | 73.357 | 0.00 | 0.00 | H |
| ATOM | 4100 | 1HD2 | LEU | 300 | 121.695 | 82.259 | 73.327 | 0.00 | 0.00 | H |
| ATOM | 4101 | 2HD2 | LEU | 300 | 120.746 | 81.517 | 74.640 | 0.00 | 0.00 | H |
| ATOM | 4102 | 3HD2 | LEU | 300 | 121.988 | 80.573 | 73.783 | 0.00 | 0.00 | H |
| ATOM | 4103 | N | ASN | 301 | 120.297 | 76.970 | 71.206 | 1.00 | 0.00 | N |
| ATOM | 4104 | CA | ASN | 301 | 121.104 | 76.208 | 70.204 | 1.00 | 0.00 | C |
| ATOM | 4105 | C | ASN | 301 | 120.227 | 75.154 | 69.432 | 1.00 | 0.00 | C |
| ATOM | 4106 | O | ASN | 301 | 120.271 | 73.949 | 69.696 | 1.00 | 0.00 | O |
| ATOM | 4107 | CB | ASN | 301 | 122.318 | 75.573 | 70.940 | 1.00 | 0.00 | C |
| ATOM | 4108 | CG | ASN | 301 | 123.381 | 76.548 | 71.451 | 1.00 | 0.00 | C |
| ATOM | 4109 | OD1 | ASN | 301 | 124.255 | 77.005 | 70.727 | 1.00 | 0.00 | O |
| ATOM | 4110 | ND2 | ASN | 301 | 123.349 | 76.909 | 72.706 | 1.00 | 0.00 | N |
| ATOM | 4111 | H | ASN | 301 | 120.089 | 76.589 | 72.137 | 1.00 | 0.00 | H |
| ATOM | 4112 | HA | ASN | 301 | 121.521 | 76.899 | 69.441 | 1.00 | 0.00 | H |
| ATOM | 4113 | 1HB | ASN | 301 | 121.973 | 74.907 | 71.753 | 1.00 | 0.00 | H |
| ATOM | 4114 | 2HB | ASN | 301 | 122.841 | 74.896 | 70.241 | 1.00 | 0.00 | H |
| ATOM | 4115 | 1HD2 | ASN | 301 | 124.007 | 77.663 | 72.912 | 1.00 | 0.00 | H |
| ATOM | 4116 | 2HD2 | ASN | 301 | 122.480 | 76.659 | 73.193 | 1.00 | 0.00 | H |
| ATOM | 4117 | N | ASP | 302 | 119.431 | 75.633 | 68.462 | 1.00 | 0.00 | N |
| ATOM | 4118 | CA | ASP | 302 | 118.470 | 74.800 | 67.682 | 1.00 | 0.00 | C |
| ATOM | 4119 | C | ASP | 302 | 118.558 | 75.124 | 66.146 | 1.00 | 0.00 | C |
| ATOM | 4120 | O | ASP | 302 | 118.888 | 76.244 | 65.746 | 1.00 | 0.00 | O |
| ATOM | 4121 | CB | ASP | 302 | 117.068 | 75.070 | 68.304 | 1.00 | 0.00 | C |
| ATOM | 4122 | CG | ASP | 302 | 115.930 | 74.205 | 67.769 | 1.00 | 0.00 | C |
| ATOM | 4123 | OD1 | ASP | 302 | 115.315 | 74.453 | 66.736 | 1.00 | 0.00 | O |
| ATOM | 4124 | OD2 | ASP | 302 | 115.684 | 73.122 | 68.555 | 1.00 | 0.00 | O |
| ATOM | 4125 | H | ASP | 302 | 119.450 | 76.657 | 68.388 | 1.00 | 0.00 | H |
| ATOM | 4126 | HA | ASP | 302 | 118.713 | 73.724 | 67.810 | 1.00 | 0.00 | H |
| ATOM | 4127 | 1HB | ASP | 302 | 117.108 | 74.932 | 69.403 | 1.00 | 0.00 | H |
| ATOM | 4128 | 2HB | ASP | 302 | 116.784 | 76.131 | 68.168 | 1.00 | 0.00 | H |
| ATOM | 4129 | N | GLU | 303 | 118.186 | 74.166 | 65.272 | 1.00 | 0.00 | N |
| ATOM | 4130 | CA | GLU | 303 | 118.106 | 74.393 | 63.789 | 1.00 | 0.00 | C |
| ATOM | 4131 | C | GLU | 303 | 117.247 | 75.612 | 63.272 | 1.00 | 0.00 | C |
| ATOM | 4132 | O | GLU | 303 | 117.653 | 76.272 | 62.311 | 1.00 | 0.00 | O |
| ATOM | 4133 | CB | GLU | 303 | 117.698 | 73.037 | 63.143 | 1.00 | 0.00 | C |
| ATOM | 4134 | CG | GLU | 303 | 117.791 | 72.950 | 61.594 | 1.00 | 0.00 | C |
| ATOM | 4135 | CD | GLU | 303 | 119.196 | 73.093 | 61.008 | 1.00 | 0.00 | C |
| ATOM | 4136 | OE1 | GLU | 303 | 120.002 | 72.174 | 60.934 | 1.00 | 0.00 | O |
| ATOM | 4137 | OE2 | GLU | 303 | 119.453 | 74.358 | 60.580 | 1.00 | 0.00 | O |
| ATOM | 4138 | H | GLU | 303 | 117.932 | 73.274 | 65.704 | 1.00 | 0.00 | H |
| ATOM | 4139 | HA | GLU | 303 | 119.138 | 74.609 | 63.453 | 1.00 | 0.00 | H |
| ATOM | 4140 | 1HB | GLU | 303 | 118.319 | 72.218 | 63.562 | 1.00 | 0.00 | H |
| ATOM | 4141 | 2HB | GLU | 303 | 116.662 | 72.789 | 63.449 | 1.00 | 0.00 | H |
| ATOM | 4142 | 1HG | GLU | 303 | 117.397 | 71.974 | 61.259 | 1.00 | 0.00 | H |
| ATOM | 4143 | 2HG | GLU | 303 | 117.126 | 73.698 | 61.122 | 1.00 | 0.00 | H |
| ATOM | 4144 | N | PHE | 304 | 116.123 | 75.957 | 63.930 | 1.00 | 0.00 | N |
| ATOM | 4145 | CA | PHE | 304 | 115.408 | 77.255 | 63.706 | 1.00 | 0.00 | C |
| ATOM | 4146 | C | PHE | 304 | 116.187 | 78.601 | 64.004 | 1.00 | 0.00 | C |
| ATOM | 4147 | O | PHE | 304 | 115.678 | 79.671 | 63.666 | 1.00 | 0.00 | O |
| ATOM | 4148 | CB | PHE | 304 | 114.086 | 77.155 | 64.532 | 1.00 | 0.00 | C |
| ATOM | 4149 | CG | PHE | 304 | 112.952 | 78.103 | 64.094 | 1.00 | 0.00 | C |
| ATOM | 4150 | CD1 | PHE | 304 | 112.209 | 77.828 | 62.941 | 1.00 | 0.00 | C |
| ATOM | 4151 | CE1 | PHE | 304 | 111.199 | 78.696 | 62.531 | 1.00 | 0.00 | C |
| ATOM | 4152 | CZ | PHE | 304 | 110.911 | 79.832 | 63.280 | 1.00 | 0.00 | C |
| ATOM | 4153 | CE2 | PHE | 304 | 111.637 | 80.110 | 64.436 | 1.00 | 0.00 | C |
| ATOM | 4154 | CD2 | PHE | 304 | 112.654 | 79.248 | 64.842 | 1.00 | 0.00 | C |
| ATOM | 4155 | H | PHE | 304 | 115.943 | 75.378 | 64.761 | 1.00 | 0.00 | H |
| ATOM | 4156 | HA | PHE | 304 | 115.142 | 77.303 | 62.631 | 1.00 | 0.00 | H |
| ATOM | 4157 | 1HB | PHE | 304 | 113.671 | 76.131 | 64.512 | 1.00 | 0.00 | H |
| ATOM | 4158 | 2HB | PHE | 304 | 114.322 | 77.303 | 65.605 | 1.00 | 0.00 | H |
| ATOM | 4159 | HD1 | PHE | 304 | 112.421 | 76.945 | 62.353 | 1.00 | 0.00 | H |
| ATOM | 4160 | HE1 | PHE | 304 | 110.639 | 78.489 | 61.632 | 1.00 | 0.00 | H |
| ATOM | 4161 | HZ | PHE | 304 | 110.124 | 80.503 | 62.962 | 1.00 | 0.00 | H |
| ATOM | 4162 | HE2 | PHE | 304 | 111.414 | 80.993 | 65.017 | 1.00 | 0.00 | H |
| ATOM | 4163 | HD2 | PHE | 304 | 113.223 | 79.482 | 65.731 | 1.00 | 0.00 | H |
| ATOM | 4164 | N | PHE | 305 | 117.381 | 78.573 | 64.630 | 1.00 | 0.00 | N |
| ATOM | 4165 | CA | PHE | 305 | 118.152 | 79.787 | 65.016 | 1.00 | 0.00 | C |
| ATOM | 4166 | C | PHE | 305 | 119.662 | 79.644 | 64.585 | 1.00 | 0.00 | C |
| ATOM | 4167 | O | PHE | 305 | 120.540 | 79.402 | 65.413 | 1.00 | 0.00 | O |
| ATOM | 4168 | CB | PHE | 305 | 117.850 | 80.000 | 66.535 | 1.00 | 0.00 | C |
| ATOM | 4169 | CG | PHE | 305 | 118.007 | 81.423 | 67.096 | 1.00 | 0.00 | C |
| ATOM | 4170 | CD1 | PHE | 305 | 117.364 | 82.517 | 66.501 | 1.00 | 0.00 | C |
| ATOM | 4171 | CE1 | PHE | 305 | 117.504 | 83.793 | 67.039 | 1.00 | 0.00 | C |
| ATOM | 4172 | CZ | PHE | 305 | 118.254 | 83.979 | 68.198 | 1.00 | 0.00 | C |
| ATOM | 4173 | CE2 | PHE | 305 | 118.865 | 82.894 | 68.816 | 1.00 | 0.00 | C |
| ATOM | 4174 | CD2 | PHE | 305 | 118.748 | 81.623 | 68.264 | 1.00 | 0.00 | C |
| ATOM | 4175 | H | PHE | 305 | 117.712 | 77.629 | 64.875 | 1.00 | 0.00 | H |
| ATOM | 4176 | HA | PHE | 305 | 117.765 | 80.671 | 64.481 | 1.00 | 0.00 | H |
| ATOM | 4177 | 1HB | PHE | 305 | 116.802 | 79.722 | 66.766 | 1.00 | 0.00 | H |
| ATOM | 4178 | 2HB | PHE | 305 | 118.447 | 79.271 | 67.120 | 1.00 | 0.00 | H |
| ATOM | 4179 | HD1 | PHE | 305 | 116.765 | 82.390 | 65.609 | 1.00 | 0.00 | H |
| ATOM | 4180 | HE1 | PHE | 305 | 117.030 | 84.635 | 66.552 | 1.00 | 0.00 | H |
| ATOM | 4181 | HZ | PHE | 305 | 118.367 | 84.968 | 68.618 | 1.00 | 0.00 | H |
| ATOM | 4182 | HE2 | PHE | 305 | 119.442 | 83.036 | 69.715 | 1.00 | 0.00 | H |
| ATOM | 4183 | HD2 | PHE | 305 | 119.245 | 80.795 | 68.747 | 1.00 | 0.00 | H |
| ATOM | 4184 | N | THR | 306 | 119.955 | 79.784 | 63.270 | 1.00 | 0.00 | N |
| ATOM | 4185 | CA | THR | 306 | 121.282 | 79.388 | 62.661 | 1.00 | 0.00 | C |
| ATOM | 4186 | C | THR | 306 | 122.021 | 80.408 | 61.698 | 1.00 | 0.00 | C |
| ATOM | 4187 | O | THR | 306 | 123.248 | 80.336 | 61.626 | 1.00 | 0.00 | O |
| ATOM | 4188 | CB | THR | 306 | 121.171 | 77.981 | 61.978 | 1.00 | 0.00 | C |
| ATOM | 4189 | OG1 | THR | 306 | 120.041 | 77.881 | 61.114 | 1.00 | 0.00 | O |
| ATOM | 4190 | CG2 | THR | 306 | 121.072 | 76.801 | 62.954 | 1.00 | 0.00 | C |
| ATOM | 4191 | H | THR | 306 | 119.106 | 79.752 | 62.694 | 1.00 | 0.00 | H |
| ATOM | 4192 | HA | THR | 306 | 122.024 | 79.272 | 63.477 | 1.00 | 0.00 | H |
| ATOM | 4193 | HB | THR | 306 | 122.083 | 77.809 | 61.368 | 1.00 | 0.00 | H |
| ATOM | 4194 | HG1 | THR | 306 | 119.374 | 77.379 | 61.601 | 1.00 | 0.00 | H |
| ATOM | 4195 | 1HG2 | THR | 306 | 121.035 | 75.832 | 62.425 | 1.00 | 0.00 | H |
| ATOM | 4196 | 2HG2 | THR | 306 | 121.942 | 76.765 | 63.637 | 1.00 | 0.00 | H |
| ATOM | 4197 | 3HG2 | THR | 306 | 120.171 | 76.867 | 63.591 | 1.00 | 0.00 | H |
| ATOM | 4198 | N | SER | 307 | 121.351 | 81.322 | 60.959 | 1.00 | 0.00 | N |
| ATOM | 4199 | CA | SER | 307 | 122.008 | 82.279 | 60.005 | 1.00 | 0.00 | C |
| ATOM | 4200 | C | SER | 307 | 121.336 | 83.709 | 59.965 | 1.00 | 0.00 | C |
| ATOM | 4201 | O | SER | 307 | 120.332 | 83.893 | 59.268 | 1.00 | 0.00 | O |
| ATOM | 4202 | CB | SER | 307 | 122.038 | 81.621 | 58.598 | 1.00 | 0.00 | C |
| ATOM | 4203 | OG | SER | 307 | 120.733 | 81.519 | 58.018 | 1.00 | 0.00 | O |
| ATOM | 4204 | H | SER | 307 | 120.334 | 81.201 | 60.982 | 1.00 | 0.00 | H |
| ATOM | 4205 | HA | SER | 307 | 123.069 | 82.426 | 60.287 | 1.00 | 0.00 | H |
| ATOM | 4206 | 1HB | SER | 307 | 122.689 | 82.210 | 57.925 | 1.00 | 0.00 | H |
| ATOM | 4207 | 2HB | SER | 307 | 122.506 | 80.618 | 58.645 | 1.00 | 0.00 | H |
| ATOM | 4208 | HG | SER | 307 | 120.294 | 82.369 | 58.183 | 1.00 | 0.00 | H |
| ATOM | 4209 | N | GLY | 308 | 121.864 | 84.730 | 60.673 | 0.00 | 0.00 | N |
| ATOM | 4210 | CA | GLY | 308 | 121.237 | 86.094 | 60.694 | 0.00 | 0.00 | C |
| ATOM | 4211 | C | GLY | 308 | 121.856 | 87.165 | 61.631 | 0.00 | 0.00 | C |
| ATOM | 4212 | OC | GLY | 308 | 121.218 | 87.503 | 62.866 | 1.00 | 0.00 | O |
| ATOM | 4213 | O | GLY | 308 | 122.904 | 87.719 | 61.299 | 0.00 | 0.00 | O |
| ATOM | 4214 | HC | GLY | 308 | 120.660 | 86.703 | 63.195 | 1.00 | 0.00 | H |
| ATOM | 4215 | H | GLY | 308 | 122.592 | 84.429 | 61.338 | 1.00 | 0.00 | H |
| ATOM | 4216 | 1HA | GLY | 308 | 121.270 | 86.515 | 59.672 | 0.00 | 0.00 | H |
| ATOM | 4217 | 2HA | GLY | 308 | 120.155 | 86.006 | 60.912 | 0.00 | 0.00 | H |
| TER | | | | | | | | | | |
| ATOM | 4218 | PG | ATP | 400H | 94.957 | 91.733 | 84.664 | 1.00 | 56.62 | P |
| ATOM | 4219 | O1G | ATP | 400H | 94.187 | 90.944 | 83.509 | 1.00 | 61.42 | O |
| ATOM | 4220 | PB | ATP | 400H | 95.915 | 93.870 | 83.671 | 1.00 | 53.17 | P |
| ATOM | 4221 | O1B | ATP | 400H | 96.089 | 93.010 | 82.330 | 1.00 | 54.96 | O |
| ATOM | 4222 | O1A | ATP | 400H | 96.187 | 97.690 | 82.708 | 1.00 | 34.47 | O |
| ATOM | 4223 | PA | ATP | 400H | 96.566 | 96.129 | 82.542 | 1.00 | 45.52 | P |
| ATOM | 4224 | O5 | ATP | 400H | 98.142 | 95.970 | 82.935 | 1.00 | 43.48 | O |
| ATOM | 4225 | O3B | ATP | 400H | 94.875 | 93.256 | 84.560 | 1.00 | 53.99 | O |
| ATOM | 4226 | O2G | ATP | 400H | 96.437 | 91.126 | 84.983 | 1.00 | 57.35 | O |
| ATOM | 4227 | O3G | ATP | 400H | 94.312 | 91.481 | 86.097 | 1.00 | 57.62 | O |
| ATOM | 4228 | O2A | ATP | 400H | 96.428 | 95.839 | 80.960 | 1.00 | 42.48 | O |
| ATOM | 4229 | O3A | ATP | 400H | 95.542 | 95.326 | 83.388 | 1.00 | 50.69 | O |
| ATOM | 4230 | O2B | ATP | 400H | 97.232 | 93.930 | 84.606 | 1.00 | 58.07 | O |
| ATOM | 4231 | C5A | ATP | 400H | 99.030 | 97.140 | 83.056 | 1.00 | 39.42 | C |
| ATOM | 4232 | C5 | ATP | 400H | 105.482 | 98.998 | 81.874 | 1.00 | 22.15 | C |
| ATOM | 4233 | O2 | ATP | 400H | 102.885 | 98.017 | 86.127 | 1.00 | 41.77 | O |
| ATOM | 4234 | C2A | ATP | 400H | 102.084 | 98.131 | 84.952 | 1.00 | 38.93 | C |
| ATOM | 4235 | C2 | ATP | 400H | 103.750 | 100.569 | 80.504 | 1.00 | 24.73 | C |
| ATOM | 4236 | O4 | ATP | 400H | 101.513 | 97.285 | 82.879 | 1.00 | 32.13 | O |
| ATOM | 4237 | C1 | ATP | 400H | 102.647 | 97.361 | 83.759 | 1.00 | 30.56 | C |
| ATOM | 4238 | N9 | ATP | 400H | 103.870 | 98.006 | 83.189 | 1.00 | 23.28 | N |
| ATOM | 4239 | C8 | ATP | 400H | 105.187 | 97.588 | 83.362 | 1.00 | 19.33 | C |
| ATOM | 4240 | N7 | ATP | 400H | 106.229 | 98.104 | 82.631 | 1.00 | 24.44 | N |
| ATOM | 4241 | C4 | ATP | 400H | 104.058 | 98.982 | 82.211 | 1.00 | 23.86 | C |
| ATOM | 4242 | C6 | ATP | 400H | 105.895 | 99.858 | 80.790 | 1.00 | 20.25 | C |
| ATOM | 4243 | N6 | ATP | 400H | 107.148 | 99.906 | 80.376 | 1.00 | 24.16 | N |
| ATOM | 4244 | N1 | ATP | 400H | 105.019 | 100.612 | 80.163 | 1.00 | 22.35 | N |
| ATOM | 4245 | N3 | ATP | 400H | 103.170 | 99.872 | 81.450 | 1.00 | 25.23 | N |
| ATOM | 4246 | O3 | ATP | 400H | 100.578 | 96.609 | 86.178 | 1.00 | 49.35 | O |
| ATOM | 4247 | C3 | ATP | 400H | 100.670 | 97.532 | 85.088 | 1.00 | 39.09 | C |
| ATOM | 4248 | C4A | ATP | 400H | 100.405 | 96.839 | 83.718 | 1.00 | 36.99 | C |
| ATOM | 4249 | 2H5 | ATP | 400H | 99.210 | 97.562 | 82.051 | 1.00 | 0.00 | H |
| ATOM | 4250 | 1H5 | ATP | 400H | 98.505 | 97.953 | 83.587 | 1.00 | 0.00 | H |
| ATOM | 4251 | H2A | ATP | 400H | 102.013 | 99.195 | 84.663 | 1.00 | 0.00 | H |
| ATOM | 4252 | H2 | ATP | 400H | 103.102 | 101.207 | 79.921 | 1.00 | 0.00 | H |
| ATOM | 4253 | H1 | ATP | 400H | 102.912 | 96.326 | 84.052 | 1.00 | 0.00 | H |
| ATOM | 4254 | H8 | ATP | 400H | 105.406 | 96.787 | 84.057 | 1.00 | 0.00 | H |
| ATOM | 4255 | 1H6 | ATP | 400H | 107.816 | 99.359 | 80.926 | 1.00 | 0.00 | H |
| ATOM | 4256 | 2H6 | ATP | 400H | 107.380 | 100.761 | 79.847 | 1.00 | 0.00 | H |
| ATOM | 4257 | HO2 | ATP | 400H | 103.375 | 97.170 | 86.050 | 1.00 | 0.00 | H |
| ATOM | 4258 | HO3 | ATP | 400H | 101.361 | 96.023 | 86.141 | 1.00 | 0.00 | H |
| ATOM | 4259 | H3 | ATP | 400H | 99.958 | 98.359 | 85.265 | 1.00 | 0.00 | H |
| ATOM | 4260 | H4 | ATP | 400H | 100.487 | 95.738 | 83.832 | 1.00 | 0.00 | H |
| END | | | | | | | | | | |

TABLE 3
Inhibition of PLK1 enzymatic activity by adenosine, thioadenosines, and
various thiol-reactive compounds in the presence or absence of
dithiothreitol (+DTT or −DTT); IC₅₀; concentration with
half-maximal inhibition.

IC₅₀ (μM)

| Compound | + DTT | − DTT |
|---|---|---|
| Thimerosal | >200 | 22 |
| N-ethylmaleimide | >200 | 55 |
| Iodoacetamide | >200 | 83 |
| Adenosine | >200 | >200 |
| 2′-Thioadenosine | >200 | 120 |
| 5′-Thioadenosine | >200 | 39 |

TABLE 4
PLK1 contact model (Maestro) for ATP.

| | | | |
|---|---|---|---|
| | | | Contact |
| PLK1 | ATP | Distance | cut-off |

| Residue | Atom | atom | (Å) | ratio |
|---|---|---|---|---|
| K178 | NZ | O1B | 3.1 | 1.0 |
| K178 | CE | O1B | 4.0 | 1.2 |
| R135 | NH1 | O1A | 3.9 | 1.2 |
| K61 | CA | O1A | 4.2 | 1.3 |
| K61 | N | O1A | 3.0 | 1.0 |
| G60 | N | O1A | 4.1 | 1.3 |
| G60 | C | O1A | 3.2 | 1.0 |
| R135 | NH1 | PA | 3.3 | 1.0 |
| R135 | CZ | PA | 4.3 | 1.2 |
| G60 | 3HD2 | PA | 4.4 | 1.3 |
| R135 | NH1 | O5 | 3.1 | 1.0 |
| G63 | N | O3G | 3.9 | 1.2 |
| R135 | NE | O2A | 3.9 | 1.2 |
| F135 | NH2 | O2A | 3.3 | 1.0 |
| R136 | CZ | O2A | 3.0 | 0.9 |
| F136 | NH1 | C5A | 3.3 | 1.0 |
| C67 | SG | C5A | 3.7 | 1.1 |
| F183 | CE2 | C5 | 4.0 | 1.1 |
| F183 | CZ | C5 | 3.8 | 1.1 |
| F183 | CE1 | C5 | 3.6 | 1.0 |
| F183 | CD1 | C5 | 3.7 | 1.1 |
| A80 | CB | C5 | 4.2 | 1.2 |
| F183 | CD2 | C5 | 4.2 | 1.2 |
| F183 | CG | C5 | 4.1 | 1.2 |
| D194 | OD1 | O2 | 3.1 | 1.0 |
| D194 | CG | O2 | 3.2 | 1.0 |
| K82 | NZ | O2 | 3.4 | 1.1 |
| K82 | CE | O2 | 3.3 | 1.0 |
| K82 | CD | O2 | 3.3 | 1.0 |
| K82 | CG | O2 | 4.1 | 1.3 |
| K82 | CB | O2 | 3.9 | 1.2 |
| C67 | SG | C2A | 4.1 | 1.2 |
| D194 | OD2 | C2A | 3.4 | 1.1 |
| D194 | OD1 | C2A | 3.6 | 1.1 |
| D194 | CG | C2A | 3.8 | 1.1 |
| K82 | CD | C2A | 4.4 | 1.3 |
| C67 | CB | C2A | 3.9 | 1.1 |
| F183 | CZ | C2 | 4.6 | 1.3 |
| F183 | CE1 | C2 | 3.7 | 1.0 |
| F183 | CD1 | C2 | 3.9 | 1.1 |
| C133 | O | C2 | 3.4 | 1.0 |
| C133 | C | C2 | 4.4 | 1.3 |
| A80 | CB | C2 | 3.4 | 1.0 |
| L59 | CD1 | C2 | 4.3 | 1.2 |
| L59 | CG | C2 | 4.4 | 1.3 |
| C67 | SG | O4 | 4.2 | 1.3 |
| F183 | CZ | O4 | 3.6 | 1.1 |
| F183 | CE1 | O4 | 4.0 | 1.2 |
| D194 | CB | C1 | 4.4 | 1.3 |
| F183 | CZ | C1 | 3.8 | 1.1 |
| F183 | CE1 | C1 | 4.4 | 1.3 |
| D194 | OD2 | C1 | 3.3 | 1.0 |
| D194 | OD1 | C1 | 3.8 | 1.1 |
| D194 | CG | C1 | 3.6 | 1.0 |
| F183 | CE2 | N9 | 4.0 | 1.2 |
| F183 | CZ | N9 | 3.5 | 1.0 |
| F183 | CE1 | N9 | 3.8 | 1.2 |
| D194 | OD2 | N9 | 3.7 | 1.2 |
| D194 | CG | N9 | 4.3 | 1.3 |
| D194 | CB | C8 | 4.5 | 1.3 |
| F183 | CE2 | C8 | 3.7 | 1.1 |
| F183 | CZ | C8 | 3.7 | 1.0 |
| F183 | CE1 | C8 | 4.2 | 1.2 |
| D194 | OD2 | C8 | 3.4 | 1.0 |
| D194 | CG | C8 | 4.1 | 1.2 |
| G193 | C | C8 | 4.1 | 1.2 |
| G193 | C | C8 | 4.3 | 1.2 |
| D194 | N | C8 | 3.9 | 1.2 |
| F183 | CD2 | C8 | 4.4 | 1.2 |
| L130 | CD1 | C8 | 3.9 | 1.1 |
| F183 | CE2 | N7 | 3.8 | 1.1 |
| F183 | CZ | N7 | 3.9 | 1.1 |
| F183 | CE1 | N7 | 4.2 | 1.2 |
| F183 | CD1 | N7 | 4.4 | 1.3 |
| G193 | C | N7 | 4.4 | 1.3 |
| G193 | C | N7 | 4.0 | 1.2 |
| F183 | CD2 | N7 | 4.0 | 1.2 |
| F183 | CG | N7 | 4.3 | 1.3 |
| V114 | CG2 | N7 | 4.1 | 1.2 |
| V114 | CG1 | N7 | 4.2 | 1.2 |
| L130 | CD1 | N7 | 3.7 | 1.1 |
| L130 | CB | N7 | 4.4 | 1.3 |
| F183 | CE2 | C4 | 4.2 | 1.2 |
| F183 | CZ | C4 | 3.5 | 1.0 |
| F183 | CE1 | C4 | 3.4 | 1.0 |
| F183 | CD1 | C4 | 4.0 | 1.1 |
| A80 | CB | C4 | 4.3 | 1.2 |
| F183 | CZ | C6 | 4.4 | 1.3 |
| F183 | CE1 | C6 | 3.8 | 1.1 |
| F183 | CD1 | C6 | 3.5 | 1.0 |
| C133 | O | C6 | 4.1 | 1.2 |
| A80 | CB | C6 | 3.7 | 1.1 |
| F183 | CD2 | C6 | 4.5 | 1.3 |
| F183 | CG | C6 | 3.9 | 1.1 |
| V114 | CG1 | C6 | 4.4 | 1.3 |
| C133 | CB | C6 | 4.5 | 1.3 |
| C133 | N | C6 | 4.1 | 1.2 |
| E131 | O | C6 | 3.4 | 1.0 |
| F183 | CD1 | N6 | 4.0 | 1.2 |
| F183 | CG | N6 | 4.0 | 1.2 |
| V114 | CG2 | N6 | 3.8 | 1.1 |
| V114 | CG1 | N6 | 3.4 | 1.0 |
| V114 | CB | N6 | 4.3 | 1.3 |
| C133 | SG | N6 | 4.4 | 1.3 |
| C133 | CB | N6 | 3.7 | 1.1 |
| C133 | CA | N6 | 4.2 | 1.3 |
| C133 | N | N6 | 3.8 | 1.2 |
| E131 | O | N6 | 2.8 | 0.9 |
| E131 | C | N6 | 4.0 | 1.2 |
| F183 | CE1 | N1 | 3.9 | 1.1 |
| F183 | CD1 | N1 | 3.6 | 1.1 |
| C133 | O | N1 | 3.1 | 1.0 |
| C133 | C | N1 | 3.9 | 1.1 |
| F183 | CD1 | N1 | 3.3 | 1.0 |
| F183 | CG | N1 | 4.4 | 1.3 |
| C133 | CB | N1 | 4.3 | 1.3 |
| C133 | CA | N1 | 4.0 | 1.2 |
| C133 | N | N1 | 3.4 | 1.1 |
| E131 | O | N1 | 3.5 | 1.1 |
| C67 | SG | N3 | 4.5 | 1.3 |
| F183 | CZ | N3 | 4.1 | 1.2 |
| F183 | CE1 | N3 | 3.5 | 1.0 |
| F183 | CD1 | N3 | 4.1 | 1.2 |
| F183 | CD1 | N3 | 3.9 | 1.1 |
| L59 | CD1 | N3 | 4.1 | 1.2 |
| D194 | OD2 | O3 | 3.9 | 1.3 |
| D194 | CG | O3 | 3.5 | 1.1 |
| K82 | CD | O3 | 4.1 | 1.3 |
| C67 | SG | C3 | 3.8 | 1.1 |
| D194 | OD1 | C3 | 3.6 | 1.1 |
| D194 | CG | C3 | 4.1 | 1.2 |
| C67 | CB | C3 | 4.1 | 1.2 |
| C67 | SG | C4A | 4.1 | 1.2 |
| D194 | OD1 | C4A | 4.1 | 1.2 |

TABLE 5
PLK1 contact model (Quanta) for ATP.

| | | Protein - |
|---|---|---|
| PLK1 | Residue | ligand atom |
| residue | atom | distance (Å) |
| L59 | HG | 3.5 |
| L59 | HD11 | 3.2 |
| L59 | HG | 2.6 |
| L59 | HD13 | 3.1 |
| G60 | CA | 2.8 |
| G60 | C | 3.2 |
| G60 | HA1 | 1.9 |
| G60 | HA2 | 3.1 |
| G60 | HA1 | 3.5 |
| G60 | HA1 | 3.5 |
| G60 | HA1 | 2.9 |
| K61 | N | 3.0 |
| K61 | H | 2.2 |
| G62 | HA1 | 3.1 |
| G63 | H | 2.9 |
| C67 | HG | 3.1 |
| C67 | HB2 | 3.0 |
| C67 | HG | 3.2 |
| C67 | HG | 3.0 |
| C67 | HG | 3.4 |
| C67 | HB2 | 3.3 |
| C67 | HG | 3.2 |
| C67 | HG | 3.2 |
| C67 | HG | 2.9 |
| C67 | SG | 3.0 |
| C67 | HG | 2.7 |
| C67 | CB | 2.9 |
| C67 | SG | 3.3 |
| C67 | HB1 | 2.9 |
| C67 | HB2 | 2.2 |
| C67 | HG | 2.5 |
| C67 | CB | 3.3 |
| C67 | SG | 3.0 |
| C67 | HB2 | 2.6 |
| C67 | HG | 2.7 |
| A80 | CB | 3.4 |
| A80 | HB1 | 2.9 |
| A80 | HB2 | 3.1 |
| A80 | HB3 | 3.2 |
| A80 | CB | 3.3 |
| A80 | HB1 | 3.2 |
| A80 | HB2 | 2.9 |
| A80 | HB3 | 3.1 |
| A80 | HB1 | 3.0 |
| A80 | HB1 | 3.2 |
| A80 | HB2 | 3.0 |
| A80 | HB3 | 3.4 |
| K82 | CD | 3.3 |
| K82 | CE | 3.3 |
| K82 | NZ | 3.4 |
| K82 | HZ2 | 2.8 |
| K82 | HB2 | 3.1 |
| K82 | HD1 | 2.4 |
| K82 | HE2 | 3.0 |
| K82 | HD1 | 3.4 |
| K82 | HD1 | 3.1 |
| K82 | HB2 | 3.5 |
| K82 | CE | 3.4 |
| K82 | NZ | 3.1 |
| K82 | HZ1 | 3.5 |
| K82 | HZ2 | 2.2 |
| K82 | HD1 | 2.9 |
| K82 | HE2 | 3.1 |
| K82 | HZ2 | 3.2 |
| K82 | HD1 | 3.2 |
| V114 | HG13 | 3.2 |
| V114 | HG23 | 3.1 |
| V114 | CG1 | 3.4 |
| V114 | HG12 | 3.1 |
| V114 | HG13 | 2.7 |
| V114 | HG21 | 3.4 |
| V114 | HG23 | 3.2 |
| V114 | CB | 3.3 |
| V114 | CG1 | 2.5 |
| V114 | CG2 | 2.9 |
| V114 | HG11 | 3.4 |
| V114 | HG12 | 2.5 |
| V114 | HG13 | 1.8 |
| V114 | HG21 | 2.8 |
| V114 | HG23 | 2.3 |
| V114 | HG12 | 3.2 |
| V114 | HG13 | 3.2 |
| L130 | HD11 | 3.1 |
| L130 | HD11 | 2.8 |
| L130 | HD11 | 3.0 |
| L130 | HB2 | 3.2 |
| E131 | O | 3.4 |
| E131 | O | 2.8 |
| E131 | O | 3.5 |
| E131 | O | 3.3 |
| E131 | C | 3.1 |
| E131 | O | 2.0 |
| C133 | O | 3.4 |
| C133 | H | 3.3 |
| C133 | H | 2.9 |
| C133 | HB1 | 2.7 |
| C133 | N | 3.4 |
| C133 | O | 3.1 |
| C133 | H | 2.8 |
| C133 | O | 3.0 |
| C133 | HB1 | 3.4 |
| C133 | N | 2.9 |
| C133 | CB | 3.1 |
| C133 | H | 2.0 |
| C133 | HB1 | 2.3 |
| R135 | HH12 | 3.0 |
| R135 | NH1 | 3.3 |
| R135 | HH12 | 2.7 |
| R135 | HH11 | 2.9 |
| R135 | NH1 | 3.1 |
| R135 | HH12 | 2.9 |
| R135 | HH11 | 2.7 |
| R135 | CZ | 3.0 |
| R135 | NH1 | 2.3 |
| R135 | NH2 | 3.3 |
| R135 | HH12 | 1.9 |
| R135 | HH11 | 2.1 |
| R135 | HH22 | 2.8 |
| R135 | NH1 | 3.3 |
| R135 | HH12 | 3.0 |
| R135 | HH11 | 3.4 |
| R135 | NH1 | 2.6 |
| R135 | HH12 | 2.3 |
| R135 | HH11 | 3.0 |
| K178 | NZ | 3.1 |
| K178 | HZ1 | 3.1 |
| K178 | HZ2 | 2.3 |
| F183 | HE1 | 3.3 |
| F183 | HZ | 2.8 |
| F183 | HZ | 3.2 |
| F183 | CZ | 3.5 |
| F183 | HZ | 3.2 |
| F183 | CE1 | 3.4 |
| F183 | HE1 | 3.3 |
| F183 | HD1 | 3.3 |
| F183 | CE1 | 3.5 |
| F183 | HE1 | 3.0 |
| F183 | HZ | 3.1 |
| G193 | HA2 | 3.5 |
| G193 | HA2 | 3.1 |
| G193 | C | 3.4 |
| G193 | HA2 | 3.2 |
| D194 | CG | 3.2 |
| D194 | OD1 | 3.1 |
| D194 | OD2 | 2.5 |
| D194 | OD2 | 3.4 |
| D194 | OD2 | 3.3 |
| D194 | OD2 | 3.4 |
| D194 | OD1 | 2.6 |
| D194 | CB | 3.3 |
| D194 | CG | 2.6 |
| D194 | OD1 | 3.0 |
| D194 | OD2 | 2.7 |
| D194 | HB2 | 2.8 |
| D194 | N | 2.9 |
| D194 | CG | 3.3 |
| D194 | OD2 | 2.5 |
| D194 | H | 2.7 |
| D194 | HB2 | 3.4 |
| D194 | CG | 2.2 |
| D194 | OD1 | 2.4 |
| D194 | OD2 | 1.6 |
| D194 | CG | 2.6 |
| D194 | OD1 | 1.7 |
| D194 | OD2 | 3.1 |

TABLE 6
PLK1 contact model (Maestro) for 5′-thioadenosine.

| | | | |
|---|---|---|---|
| | 5′-Thio | | Contact |
| PLK1 | adenosine | Distance | cut-off |

| Residue | Atom | atom | (Å) | ratio |
|---|---|---|---|---|
| G60 | CA | S5 | 4.0 | 1.1 |
| C67 | N | S5 | 3.3 | 1.0 |
| K66 | C | S5 | 3.9 | 1.1 |
| K66 | CA | S5 | 4.1 | 1.2 |
| K61 | CA | S5 | 4.3 | 1.2 |
| K61 | N | S5 | 3.9 | 1.2 |
| G60 | O | S5 | 3.4 | 1.0 |
| G60 | C | S5 | 3.5 | 1.0 |
| C67 | SG | S5 | 3.3 | 0.9 |
| C67 | CB | S5 | 3.6 | 1.0 |
| C67 | CA | S5 | 4.1 | 1.2 |
| R135 | NH2 | C5A | 3.7 | 1.2 |
| R135 | CZ | C5A | 4.0 | 1.2 |
| R135 | NH2 | C5A | 3.6 | 1.1 |
| C67 | SG | C5A | 3.6 | 1.0 |
| C67 | CB | C5A | 4.2 | 1.2 |
| F183 | CZ | C5 | 3.5 | 1.0 |
| F183 | CE1 | C5 | 3.5 | 1.0 |
| F183 | CD1 | C5 | 4.1 | 1.2 |
| A80 | CB | C5 | 3.8 | 1.1 |
| F183 | CE2 | C5 | 4.0 | 1.2 |
| D194 | OD1 | O2 | 3.6 | 1.2 |
| D194 | CG | O2 | 3.3 | 1.0 |
| K82 | NZ | O2 | 3.1 | 1.0 |
| K82 | CB | O2 | 3.8 | 1.2 |
| K82 | CE | O2 | 3.1 | 1.0 |
| K82 | CD1 | O2 | 3.2 | 1.0 |
| K82 | CG | O2 | 4.0 | 1.3 |
| D194 | OD2 | C2A | 3.4 | 1.0 |
| D194 | OD1 | C2A | 3.6 | 1.1 |
| D194 | CG | C2A | 3.8 | 1.1 |
| K82 | CB | C2A | 4.1 | 1.2 |
| C67 | CB | C2A | 3.9 | 1.2 |
| K82 | CE | C2A | 4.2 | 1.2 |
| K82 | CD | C2A | 3.9 | 1.1 |
| F183 | CZ | C2 | 4.2 | 1.2 |
| C133 | O | C2 | 3.5 | 1.1 |
| F183 | CE1 | C2 | 3.5 | 1.0 |
| F183 | CD1 | C2 | 4.0 | 1.2 |
| R135 | NH2 | C2 | 4.1 | 1.3 |
| L59 | CD1 | C2 | 3.8 | 1.1 |
| L59 | CG | C2 | 4.2 | 1.2 |
| A80 | CB | C2 | 3.4 | 1.0 |
| R135 | NH2 | O4 | 3.0 | 1.0 |
| R135 | CZ | O4 | 3.3 | 1.0 |
| D194 | OD1 | O4 | 3.8 | 1.3 |
| R135 | NH2 | O4 | 2.8 | 0.9 |
| C67 | SG | O4 | 3.6 | 1.1 |
| C67 | CB | O4 | 3.9 | 1.2 |
| F183 | CZ | C1 | 4.2 | 1.2 |
| R135 | NH2 | C1 | 3.7 | 1.2 |
| R135 | CZ | C1 | 4.3 | 1.3 |
| D194 | OD2 | C1 | 3.6 | 1.1 |
| D194 | OD1 | C1 | 3.3 | 1.0 |
| D194 | CG | C1 | 3.7 | 1.1 |
| R135 | NH2 | C1 | 3.9 | 1.2 |
| C67 | CB | C1 | 4.4 | 1.3 |
| F183 | CZ | N9 | 3.7 | 1.1 |
| F183 | CE1 | N9 | 4.2 | 1.3 |
| F183 | CZ | C8 | 3.8 | 1.1 |
| D194 | OD2 | C8 | 4.3 | 1.2 |
| F183 | CE2 | C8 | 4.1 | 1.2 |
| L130 | CD1 | C8 | 3.6 | 1.1 |
| F183 | CZ | N7 | 3.8 | 1.1 |
| F183 | CE1 | N7 | 4.2 | 1.3 |
| F183 | CE2 | N7 | 3.9 | 1.2 |
| L130 | CD1 | N7 | 3.6 | 1.1 |
| L130 | CB | N7 | 3.9 | 1.2 |
| F183 | CZ | C4 | 3.5 | 1.0 |
| F183 | CE1 | C4 | 3.6 | 1.0 |
| R135 | NH2 | C4 | 4.1 | 1.2 |
| A80 | CB | C4 | 4.0 | 1.2 |
| F183 | CE2 | C4 | 4.4 | 1.3 |
| F183 | CZ | C6 | 3.9 | 1.1 |
| C133 | O | C6 | 4.1 | 1.3 |
| F183 | CE1 | C6 | 3.4 | 1.0 |
| F183 | CD1 | C6 | 3.5 | 1.0 |
| A80 | CB | C6 | 3.5 | 1.0 |
| F183 | CD2 | C6 | 4.5 | 1.3 |
| F183 | CE2 | C6 | 4.4 | 1.3 |
| F183 | CG | C6 | 4.1 | 1.2 |
| C133 | N | C6 | 4.1 | 1.3 |
| E131 | O | C6 | 3.5 | 1.1 |
| F183 | CE1 | N6 | 4.0 | 1.2 |
| F183 | CD1 | N6 | 3.7 | 1.1 |
| A80 | CB | N6 | 4.1 | 1.3 |
| F183 | CG | N6 | 3.9 | 1.2 |
| C133 | SG | N6 | 4.3 | 1.3 |
| C133 | SB | N6 | 3.6 | 1.1 |
| F183 | CE1 | N6 | 4.1 | 1.3 |
| C133 | N | N6 | 3.7 | 1.2 |
| E131 | O | N6 | 2.9 | 1.0 |
| V114 | CG2 | N6 | 4.1 | 1.3 |
| V114 | CG1 | N6 | 3.7 | 1.1 |
| F125 | CE2 | N6 | 4.0 | 1.2 |
| F183 | CZ | N1 | 4.3 | 1.3 |
| C133 | O | N1 | 3.1 | 1.0 |
| C133 | C | N1 | 3.8 | 1.2 |
| F183 | CE1 | N1 | 3.4 | 1.0 |
| F183 | CD1 | N1 | 3.6 | 1.1 |
| A80 | CB | N1 | 3.3 | 1.0 |
| C133 | CA | N1 | 4.1 | 1.2 |
| C133 | N | N1 | 3.6 | 1.1 |
| E131 | O | N1 | 3.8 | 1.2 |
| F183 | CZ | N3 | 4.0 | 1.2 |
| F183 | CE1 | N3 | 3.6 | 1.1 |
| R135 | NH2 | N3 | 3.4 | 1.1 |
| C67 | SG | N3 | 4.0 | 1.2 |
| L59 | CD1 | N3 | 3.8 | 1.2 |
| A80 | CB | N3 | 3.8 | 1.1 |
| D194 | OD2 | O3 | 3.0 | 0.9 |
| D194 | CG | O3 | 3.2 | 1.0 |
| K82 | NZ | O3 | 3.7 | 1.2 |
| K82 | CE | O3 | 4.1 | 1.3 |
| K82 | CD | O3 | 3.5 | 1.1 |
| D194 | OD2 | C3 | 3.7 | 1.1 |
| D194 | OD1 | C3 | 3.3 | 1.0 |
| D194 | CG | C3 | 3.9 | 1.2 |
| C67 | SG | C3 | 4.5 | 1.3 |
| C67 | CB | C3 | 4.1 | 1.2 |
| K82 | CD | C3 | 3.8 | 1.1 |
| R135 | NH2 | C4A | 3.4 | 1.0 |
| R135 | CZ | C4A | 3.9 | 1.1 |
| D194 | OD2 | C4A | 4.4 | 1.3 |
| D194 | OD1 | C4A | 3.3 | 1.0 |
| D194 | CG | C4A | 4.2 | 1.2 |
| R135 | NH2 | C4A | 3.6 | 1.1 |
| C67 | SG | C4A | 4.0 | 1.1 |
| C67 | CB | C4A | 4.2 | 1.2 |

TABLE 7
PLK1 contact model (Quanta) for 5′-thioadenosine.

| | | Protein - |
|---|---|---|
| PLK1 | Residue | ligand atom |
| residue | atom | distance (Å) |
| L59 | HG | 3.4 |
| L59 | HD11 | 3.5 |
| L59 | HD13 | 3.3 |
| L59 | HD11 | 3.2 |
| L59 | CG | 3.2 |
| L59 | CD1 | 3.0 |
| L59 | HG | 2.5 |
| L59 | HD11 | 2.9 |
| L59 | HD13 | 2.5 |
| G60 | C | 3.2 |
| G60 | O | 3.2 |
| G60 | HA1 | 3.1 |
| G60 | HA1 | 3.3 |
| C67 | N | 3.1 |
| C67 | CB | 3.1 |
| C67 | SG | 2.0 |
| C67 | H | 2.7 |
| C67 | HB2 | 3.0 |
| C67 | SG | 3.2 |
| C67 | HB2 | 3.0 |
| C67 | SG | 3.4 |
| C67 | HB2 | 3.4 |
| C67 | HB2 | 3.1 |
| C67 | SG | 3.4 |
| C67 | CB | 3.1 |
| C67 | HB1 | 3.2 |
| C67 | HB2 | 2.1 |
| C67 | CB | 3.5 |
| C67 | HB2 | 2.6 |
| A80 | HB1 | 3.4 |
| A80 | HB3 | 3.2 |
| A80 | CB | 3.4 |
| A80 | HB1 | 2.8 |
| A80 | HB2 | 3.1 |
| A80 | HB1 | 3.2 |
| A80 | CB | 3.5 |
| A80 | HB1 | 3.3 |
| A80 | HB2 | 3.4 |
| A80 | HB3 | 3.0 |
| A80 | HB3 | 3.4 |
| A80 | CB | 3.3 |
| A80 | HB1 | 3.1 |
| A80 | HB2 | 2.9 |
| A80 | HB3 | 3.2 |
| A80 | HB1 | 2.9 |
| A80 | HB1 | 3.3 |
| A80 | HB2 | 3.3 |
| K82 | CD | 3.2 |
| K82 | CE | 3.1 |
| K82 | NZ | 3.1 |
| K82 | HZ2 | 2.5 |
| K82 | HB2 | 3.0 |
| K82 | HD1 | 2.5 |
| K82 | HE2 | 2.6 |
| K82 | HB2 | 3.1 |
| K82 | HD1 | 2.9 |
| K82 | HZ2 | 3.1 |
| K82 | HD1 | 2.5 |
| K82 | HD1 | 2.7 |
| K82 | CB | 3.5 |
| K82 | HB2 | 2.4 |
| K82 | HD1 | 3.1 |
| K82 | CE | 3.3 |
| K82 | NZ | 2.9 |
| K82 | HZ2 | 2.0 |
| K82 | HD1 | 2.9 |
| K82 | HE2 | 3.1 |
| K82 | HZ2 | 2.9 |
| K82 | HD1 | 3.2 |
| K82 | HB2 | 3.3 |
| K82 | HD1 | 2.6 |
| V114 | HG13 | 3.3 |
| V114 | HG13 | 2.9 |
| V114 | HG23 | 3.5 |
| V114 | CG1 | 2.8 |
| V114 | CG2 | 3.3 |
| V114 | HG12 | 2.8 |
| V114 | HG13 | 1.9 |
| V114 | HG21 | 3.2 |
| V114 | HG23 | 2.7 |
| V114 | HG13 | 3.5 |
| L130 | HD13 | 3.2 |
| L130 | HD11 | 3.0 |
| L130 | HB1 | 3.4 |
| L130 | HB2 | 3.4 |
| L130 | HD13 | 3.5 |
| L130 | HD11 | 2.8 |
| L130 | CD1 | 3.2 |
| L130 | HD13 | 2.9 |
| L130 | HD11 | 2.6 |
| L130 | HB2 | 3.2 |
| E131 | O | 3.5 |
| E131 | O | 2.9 |
| E131 | O | 3.2 |
| E131 | O | 2.5 |
| C133 | H | 3.2 |
| C133 | H | 2.9 |
| C133 | HB1 | 2.7 |
| C133 | O | 3.1 |
| C133 | H | 2.7 |
| C133 | O | 3.1 |
| C133 | HB1 | 3.2 |
| C133 | N | 2.7 |
| C133 | CA | 3.2 |
| C133 | CB | 2.8 |
| C133 | SG | 3.4 |
| C133 | H | 2.0 |
| C133 | HB1 | 2.0 |
| R135 | HH11 | 3.1 |
| R135 | HH22 | 3.2 |
| R135 | CZ | 3.3 |
| R135 | NH1 | 2.8 |
| R135 | NH2 | 3.0 |
| R135 | HH11 | 1.8 |
| R135 | HH22 | 2.1 |
| R135 | HH11 | 3.0 |
| R135 | HH22 | 2.7 |
| R135 | HH11 | 3.4 |
| R135 | HH11 | 3.3 |
| R135 | NH1 | 3.4 |
| R135 | HH11 | 2.8 |
| R135 | NH2 | 3.4 |
| R135 | HH11 | 2.8 |
| R135 | HH22 | 2.5 |
| R135 | CZ | 3.1 |
| R135 | NH1 | 2.8 |
| R135 | NH2 | 3.1 |
| R135 | HH12 | 3.5 |
| R135 | HH11 | 2.6 |
| R135 | HH22 | 2.9 |
| R135 | NH2 | 3.4 |
| R135 | HH11 | 3.4 |
| R135 | HH22 | 2.5 |
| R135 | NH2 | 2.9 |
| R135 | HH11 | 3.2 |
| R135 | HH21 | 3.5 |
| R135 | HH22 | 2.1 |
| F183 | CE1 | 3.5 |
| F183 | HE1 | 2.9 |
| F183 | HZ | 3.3 |
| F183 | HZ | 3.1 |
| F183 | HZ | 3.5 |
| F183 | HE1 | 3.4 |
| F183 | HZ | 3.2 |
| F183 | CE1 | 3.4 |
| F183 | CE1 | 3.4 |
| F183 | HD1 | 3.4 |
| F183 | HE1 | 3.2 |
| F183 | HE1 | 2.9 |
| F183 | HE1 | 3.2 |
| F183 | HZ | 2.9 |
| D194 | CG | 3.3 |
| D194 | OD2 | 2.5 |
| D194 | OD2 | 3.4 |
| D194 | OD1 | 3.3 |
| D194 | CG | 3.2 |
| D194 | OD1 | 2.6 |
| D194 | OD2 | 3.0 |
| D194 | OD1 | 3.3 |
| D194 | OD1 | 3.3 |
| D194 | CG | 2.7 |
| D194 | OD1 | 2.4 |
| D194 | OD2 | 2.9 |
| D194 | OD2 | 3.4 |
| D194 | H | 3.3 |
| D194 | CG | 2.4 |
| D194 | OD1 | 2.8 |
| D194 | OD2 | 1.5 |
| D194 | CG | 2.3 |
| D194 | OD1 | 1.7 |
| D194 | OD2 | 2.3 |
| D194 | OD1 | 2.4 |

TABLE 8
PLK1 contact model (Maestro) for staurosporine.

| | | | |
|---|---|---|---|
| | | | Contact |
| PLK1 | Staurosporine | Distance | cut-off |

| Residue | Atom | atom | (Å) | ratio |
|---|---|---|---|---|
| C67 | CB | O4 | 3.5 | 1.1 |
| D194 | OD2 | C21 | 4.1 | 1.2 |
| C67 | CB | C23 | 4.2 | 1.2 |
| C67 | SG | C18 | 3.7 | 1.0 |
| C67 | CB | C18 | 3.9 | 1.1 |
| C67 | SG | C19 | 4.3 | 1.2 |
| C67 | CB | C19 | 4.2 | 1.2 |
| D194 | OD1 | C16 | 3.4 | 1.0 |
| D194 | CG | C16 | 4.0 | 1.2 |
| G193 | O | C16 | 3.7 | 1.1 |
| G193 | C | C16 | 4.3 | 1.3 |
| L130 | CD1 | C16 | 4.3 | 1.3 |
| F183 | CE1 | C14 | 3.3 | 1.0 |
| F183 | CZ | C14 | 3.7 | 1.1 |
| F183 | CD1 | C14 | 4.1 | 1.2 |
| L130 | CD2 | C14 | 4.3 | 1.3 |
| L130 | CD1 | C14 | 3.9 | 1.1 |
| L130 | CG | C14 | 4.2 | 1.2 |
| L130 | CB | C14 | 3.8 | 1.1 |
| A80 | CB | C14 | 4.2 | 1.2 |
| D194 | OD1 | C15 | 3.9 | 1.2 |
| G193 | O | C15 | 3.6 | 1.1 |
| G193 | C | C15 | 4.1 | 1.2 |
| F183 | CE1 | C15 | 3.8 | 1.1 |
| L130 | CD2 | C15 | 3.9 | 1.2 |
| L130 | CD1 | C15 | 3.6 | 1.1 |
| L130 | CG | C15 | 4.1 | 1.2 |
| L130 | CB | C15 | 4.3 | 1.2 |
| F183 | CE1 | C13 | 3.8 | 1.1 |
| F183 | CZ | C13 | 4.3 | 1.2 |
| F183 | CD1 | C13 | 4.2 | 1.2 |
| C67 | SG | C13 | 4.6 | 1.3 |
| A80 | CB | C13 | 3.4 | 1.0 |
| C67 | SG | C12 | 3.8 | 1.1 |
| A80 | CB | C12 | 4.0 | 1.2 |
| C67 | SG | C17 | 4.0 | 1.1 |
| C67 | SG | N2 | 3.9 | 1.1 |
| C67 | CB | N2 | 4.0 | 1.2 |
| R135 | CG | C7 | 4.0 | 1.2 |
| L59 | CD1 | C7 | 3.9 | 1.2 |
| L59 | CG | C7 | 4.3 | 1.3 |
| L59 | CB | C7 | 3.8 | 1.1 |
| C67 | SG | C10 | 4.1 | 1.2 |
| A80 | CB | C10 | 4.3 | 1.3 |
| L59 | CD1 | C10 | 3.9 | 1.1 |
| C67 | SG | C11 | 3.6 | 1.0 |
| C67 | CB | C11 | 4.4 | 1.3 |
| A80 | CB | C11 | 4.4 | 1.3 |
| R135 | CG | C6 | 4.0 | 1.2 |
| L59 | C | C6 | 4.4 | 1.3 |
| L59 | CB | C6 | 3.8 | 1.1 |
| G60 | CA | N3 | 4.2 | 1.3 |
| G60 | CA | C20 | 3.8 | 1.1 |
| G60 | N | C20 | 4.2 | 1.3 |
| L59 | C | C20 | 4.3 | 1.2 |
| L59 | O | C20 | 4.1 | 1.3 |
| R135 | CG | C5 | 3.7 | 1.1 |
| G60 | CA | C5 | 4.3 | 1.3 |
| G60 | N | C5 | 4.2 | 1.3 |
| L59 | C | C5 | 3.9 | 1.1 |
| L59 | CB | C5 | 4.1 | 1.2 |
| L59 | O | C5 | 3.7 | 1.1 |
| R135 | CG | C4 | 3.5 | 1.0 |
| R135 | NE | C4 | 3.7 | 1.1 |
| R135 | CD | C4 | 4.0 | 1.2 |
| L59 | C | C4 | 3.8 | 1.1 |
| L59 | CB | C4 | 4.4 | 1.3 |
| L59 | O | C4 | 3.2 | 1.0 |
| R135 | CG | C3 | 4.1 | 1.2 |
| R135 | CD | C3 | 4.4 | 1.3 |
| L59 | C | C3 | 4.1 | 1.2 |
| L59 | O | C3 | 3.2 | 1.0 |
| G60 | CA | C2 | 4.1 | 1.2 |
| L59 | O | C2 | 3.8 | 1.2 |
| G60 | CA | C1 | 3.7 | 1.1 |
| L59 | O | C1 | 4.2 | 1.3 |
| C67 | CB | C25 | 4.4 | 1.3 |
| G60 | CA | C25 | 4.3 | 1.3 |
| D194 | OD2 | C23 | 4.0 | 1.2 |
| D194 | CG | C22 | 4.0 | 1.2 |
| D194 | OD2 | C22 | 3.5 | 1.0 |
| D194 | OD1 | C26 | 3.9 | 1.2 |
| D194 | CG | C26 | 4.0 | 1.2 |
| D194 | OD2 | C26 | 3.4 | 1.0 |
| K82 | CE | C26 | 4.2 | 1.2 |
| K82 | CD | C26 | 3.9 | 1.1 |
| K82 | CG | C26 | 4.4 | 1.3 |
| C67 | CB | C26 | 4.2 | 1.2 |
| G180 | O | O6 | 3.7 | 1.2 |
| N181 | O | C27 | 3.8 | 1.2 |
| N181 | C | C27 | 4.2 | 1.2 |
| N181 | CA | C27 | 3.8 | 1.1 |
| G180 | O | C27 | 3.6 | 1.1 |
| G180 | C | C27 | 4.2 | 1.2 |
| D194 | CG | C27 | 4.4 | 1.3 |
| D194 | N | C27 | 3.9 | 1.2 |
| G193 | O | C27 | 3.1 | 1.0 |
| G193 | C | C27 | 4.0 | 1.2 |
| G180 | O | N4 | 3.6 | 1.2 |
| D194 | OD2 | N4 | 4.2 | 1.3 |
| N181 | OD1 | C28 | 4.1 | 1.3 |
| K178 | NZ | C28 | 3.7 | 1.1 |
| D176 | OD2 | C28 | 4.4 | 1.3 |
| D194 | CG | C28 | 4.1 | 1.2 |
| D194 | CB | C28 | 4.1 | 1.2 |
| D194 | OD2 | C28 | 3.3 | 1.0 |
| C133 | O | C9 | 3.5 | 1.1 |
| C133 | C | C9 | 3.8 | 1.1 |
| C133 | N | C9 | 3.9 | 1.2 |
| A80 | CB | C9 | 3.9 | 1.2 |
| L59 | CD1 | C9 | 3.9 | 1.1 |
| R134 | CA | N1 | 4.0 | 1.2 |
| R134 | N | N1 | 4.0 | 1.3 |
| C133 | O | N1 | 2.8 | 0.9 |
| C133 | C | N1 | 3.4 | 1.0 |
| L59 | CD2 | N1 | 4.2 | 1.3 |
| L59 | CD1 | N1 | 3.8 | 1.2 |
| R135 | CG | O5 | 3.8 | 1.2 |
| R135 | NH2 | O5 | 3.1 | 1.0 |
| R135 | CZ | O5 | 3.4 | 1.1 |
| R135 | CD | O5 | 3.8 | 1.2 |
| L59 | CD2 | O5 | 3.6 | 1.1 |
| L59 | CG | O5 | 4.1 | 1.3 |
| L59 | CB | O5 | 3.9 | 1.2 |
| R135 | CG | C8 | 3.9 | 1.1 |
| C133 | O | C8 | 3.8 | 1.2 |
| R135 | CZ | C8 | 4.4 | 1.3 |
| R135 | NE | C8 | 3.6 | 1.1 |
| R135 | CD | C8 | 4.3 | 1.3 |
| L59 | CD2 | C8 | 3.9 | 1.1 |
| L59 | CD1 | C8 | 3.8 | 1.1 |
| L59 | CG | C8 | 4.0 | 1.2 |
| L59 | CB | C8 | 3.9 | 1.1 |

TABLE 9
PLK1 contact model (Quanta) for staurosporine.

| | | Protein - |
|---|---|---|
| PLK1 | Residue | ligand atom |
| residue | atom | distance (Å) |
| L59 | HB1 | 2.8 |
| L59 | HD13 | 3.1 |
| L59 | HD13 | 3.0 |
| L59 | HB1 | 2.9 |
| L59 | HB1 | 3.2 |
| L59 | O | 3.2 |
| L59 | HB1 | 3.4 |
| L59 | O | 3.2 |
| L59 | HD13 | 2.8 |
| L59 | HD13 | 2.7 |
| L59 | HD22 | 3.3 |
| L59 | HB1 | 3.0 |
| L59 | HD22 | 2.6 |
| L59 | HB1 | 2.9 |
| L59 | HD13 | 2.9 |
| L59 | HD22 | 2.9 |
| L59 | O | 3.4 |
| L59 | HB1 | 3.1 |
| L59 | O | 3.5 |
| L59 | CD1 | 3.3 |
| L59 | HD13 | 2.4 |
| L59 | HD13 | 3.1 |
| L59 | HD22 | 3.2 |
| G60 | HA1 | 3.2 |
| G60 | HA1 | 2.8 |
| G60 | HA1 | 3.5 |
| G60 | HA1 | 3.3 |
| G60 | HA1 | 2.7 |
| G60 | HA1 | 3.3 |
| G60 | HA1 | 2.9 |
| G60 | CA | 3.4 |
| G60 | C | 3.4 |
| G60 | HA1 | 2.5 |
| C67 | HB1 | 2.8 |
| C67 | HB2 | 3.3 |
| C67 | HB1 | 3.3 |
| C67 | HB1 | 3.4 |
| C67 | HB1 | 3.1 |
| C67 | HB1 | 3.5 |
| C67 | CB | 3.4 |
| C67 | HB1 | 2.3 |
| C67 | HG | 3.5 |
| A80 | HB2 | 3.5 |
| A80 | CB | 3.4 |
| A80 | HB2 | 3.0 |
| A80 | HB3 | 2.9 |
| A80 | HB3 | 3.1 |
| A80 | HB3 | 3.4 |
| A80 | HB1 | 3.3 |
| A80 | HB2 | 3.4 |
| A80 | CB | 3.0 |
| A80 | HB1 | 3.0 |
| A80 | HB2 | 2.6 |
| A80 | HB3 | 2.7 |
| A80 | CB | 2.9 |
| A80 | HB1 | 2.2 |
| A80 | HB2 | 3.3 |
| A80 | HB3 | 2.7 |
| K82 | HD2 | 3.0 |
| K82 | HD2 | 3.4 |
| K82 | HD2 | 3.3 |
| K82 | HE1 | 3.1 |
| K82 | CD | 3.5 |
| K82 | HZ3 | 2.9 |
| K82 | HD2 | 2.5 |
| K82 | HE1 | 3.3 |
| K82 | HB2 | 3.1 |
| K82 | HG1 | 3.3 |
| K82 | HD2 | 2.7 |
| L130 | HD12 | 3.3 |
| L130 | HB1 | 3.0 |
| L130 | HD12 | 2.9 |
| L130 | HD22 | 3.5 |
| L130 | HD12 | 2.6 |
| L130 | HD22 | 3.1 |
| L130 | CD1 | 3.4 |
| L130 | CD2 | 3.1 |
| L130 | HD12 | 2.6 |
| L130 | HD22 | 2.2 |
| L130 | HD21 | 3.4 |
| L130 | CB | 3.1 |
| L130 | HB1 | 2.4 |
| L130 | HB2 | 2.8 |
| L130 | HD12 | 3.1 |
| L130 | HD22 | 3.1 |
| E131 | O | 3.4 |
| L132 | HA | 3.5 |
| L132 | C | 3.4 |
| C133 | O | 2.8 |
| C133 | C | 3.4 |
| C133 | O | 3.5 |
| C133 | H | 3.4 |
| C133 | HB1 | 3.1 |
| C133 | N | 3.5 |
| C133 | H | 3.4 |
| C133 | C | 2.7 |
| C133 | O | 1.8 |
| C133 | O | 1.8 |
| R134 | HA | 3.1 |
| R134 | HA | 3.4 |
| R134 | HA | 3.4 |
| R134 | HA | 2.8 |
| R135 | HG2 | 3.0 |
| R135 | HG2 | 3.3 |
| R135 | HG1 | 3.0 |
| R135 | HG2 | 3.3 |
| R135 | CG | 3.5 |
| R135 | HE | 3.2 |
| R135 | HG1 | 2.7 |
| R135 | HG2 | 3.3 |
| R135 | HG1 | 3.1 |
| R135 | HE | 3.4 |
| R135 | NE | 2.8 |
| R135 | CZ | 3.4 |
| R135 | NH2 | 3.1 |
| R135 | HE | 1.7 |
| R135 | HG2 | 3.0 |
| R135 | HH21 | 2.2 |
| R135 | HE | 2.6 |
| R135 | HG2 | 2.9 |
| R135 | HH21 | 3.4 |
| R135 | CG | 3.5 |
| R135 | NE | 3.0 |
| R135 | HE | 2.4 |
| R135 | HG1 | 3.0 |
| R135 | HG2 | 3.2 |
| R135 | HH21 | 3.2 |
| R135 | H | 3.3 |
| R135 | HE | 3.5 |
| R135 | HE | 1.7 |
| K178 | HZ1 | 3.5 |
| K178 | HZ1 | 2.7 |
| K178 | HZ1 | 3.2 |
| K178 | NZ | 3.5 |
| K178 | HZ1 | 2.5 |
| K178 | NZ | 2.9 |
| K178 | HZ1 | 2.0 |
| K178 | HZ2 | 3.2 |
| K178 | HZ3 | 3.5 |
| K178 | HZ1 | 2.0 |
| G180 | O | 3.5 |
| G180 | O | 3.3 |
| G180 | O | 2.6 |
| N181 | HA | 2.8 |
| N181 | CA | 3.4 |
| N181 | OD1 | 3.2 |
| N181 | HA | 2.3 |
| N181 | CA | 3.2 |
| N181 | C | 3.5 |
| N181 | O | 3.2 |
| N181 | HA | 2.3 |
| N181 | OD1 | 3.2 |
| F183 | CE1 | 3.3 |
| F183 | HE1 | 2.8 |
| F183 | HE1 | 2.9 |
| F183 | HE1 | 2.9 |
| F183 | CE1 | 3.0 |
| F183 | CZ | 3.0 |
| F183 | HE1 | 2.6 |
| F183 | HZ | 2.7 |
| G193 | O | 3.1 |
| G193 | HA2 | 3.4 |
| G193 | O | 3.0 |
| G193 | O | 3.3 |
| G193 | O | 2.5 |
| D194 | OD1 | 3.4 |
| D194 | OD2 | 3.4 |
| D194 | H | 3.0 |
| D194 | OD2 | 3.3 |
| D194 | HB2 | 3.3 |
| D194 | CG | 3.0 |
| D194 | OD1 | 2.4 |
| D194 | OD2 | 3.5 |
| D194 | CG | 2.9 |
| D194 | OD1 | 3.4 |
| D194 | OD2 | 2.5 |
| D194 | HB2 | 3.2 |
| D194 | OD2 | 3.3 |
| D194 | CG | 3.0 |
| D194 | OD1 | 2.8 |
| D194 | OD2 | 2.6 |
| D194 | N | 3.2 |
| D194 | H | 2.2 |
| D194 | HB2 | 2.8 |
| D194 | H | 3.5 |
| D194 | H | 3.0 |
| D194 | OD2 | 2.7 |
| D194 | CB | 3.2 |
| D194 | CG | 3.3 |
| D194 | OD2 | 2.9 |
| D194 | HB1 | 3.5 |
| D194 | HB2 | 2.3 |
| D194 | H | 2.2 |

TABLE 10
PLK1 contact model (Maestro) for 4-[4-(4-methyl-2-methylamino-
thiazol-5-yl)-pyrimidin-2-ylamino]-phenol.

| | | | |
|---|---|---|---|
| | | | Contact |
| PLK1 | Ligand | Distance | cut-off |

| Residue | Atom | atom | (Å) | ratio |
|---|---|---|---|---|
| D194 | OD2 | NM1 | 3.8 | 1.2 |
| K82 | CD | NM1 | 4.1 | 1.3 |
| D194 | CG | CM12 | 4.0 | 1.2 |
| D194 | OD2 | CM12 | 3.6 | 1.1 |
| F64 | CG | CM12 | 4.0 | 1.2 |
| D194 | OD1 | CM12 | 3.5 | 1.1 |
| K82 | NZ | CM12 | 4.2 | 1.3 |
| K82 | CD | CM12 | 3.9 | 1.1 |
| F64 | CD1 | CM12 | 4.1 | 1.2 |
| F64 | CB | CM12 | 3.8 | 1.1 |
| D194 | CG | C | 4.0 | 1.2 |
| D194 | OD2 | C | 3.5 | 1.0 |
| D194 | OD1 | C | 3.8 | 1.2 |
| K82 | CD | C | 4.1 | 1.2 |
| C67 | CB | C | 4.0 | 1.2 |
| D194 | CG | N | 3.5 | 1.1 |
| D194 | OD2 | N | 3.4 | 1.0 |
| D194 | OD1 | N | 3.1 | 1.0 |
| K82 | NZ | N | 3.4 | 1.1 |
| K82 | CE | N | 3.4 | 1.0 |
| K82 | CD | N | 3.5 | 1.1 |
| K82 | CB | N | 4.2 | 1.3 |
| D194 | CG | C1 | 3.9 | 1.1 |
| D194 | OD2 | C1 | 3.8 | 1.1 |
| D194 | OD1 | C1 | 3.7 | 1.2 |
| K82 | NZ | C1 | 4.1 | 1.2 |
| K82 | CE | C1 | 4.1 | 1.2 |
| L130 | CD1 | C1 | 4.1 | 1.2 |
| D194 | CG | CM2 | 3.7 | 1.1 |
| D194 | CB | CM2 | 4.3 | 1.3 |
| D194 | CA | CM2 | 4.1 | 1.2 |
| D194 | OD2 | CM2 | 4.0 | 1.2 |
| D194 | NZ | CM2 | 3.6 | 1.1 |
| D194 | OD1 | CM2 | 3.5 | 1.1 |
| K82 | NZ | CM2 | 3.9 | 1.2 |
| K82 | CE | CM2 | 4.1 | 1.2 |
| L130 | CD2 | CM2 | 3.9 | 1.2 |
| L130 | CD1 | CM2 | 3.8 | 1.1 |
| D194 | OD2 | S | 4.2 | 1.2 |
| C67 | SG | S | 3.5 | 1.0 |
| C67 | CB | S | 3.3 | 0.9 |
| D194 | OD2 | C2 | 4.3 | 1.3 |
| F183 | CZ | C2 | 4.1 | 1.2 |
| C67 | SG | C2 | 3.9 | 1.1 |
| C67 | CB | C2 | 4.3 | 1.3 |
| F183 | CZ | N1 | 4.0 | 1.2 |
| F183 | CG | N1 | 4.2 | 1.3 |
| F183 | CE1 | N1 | 3.5 | 1.1 |
| F183 | CD1 | N1 | 3.6 | 1.1 |
| C133 | N | N1 | 3.8 | 1.2 |
| E131 | O | N1 | 3.6 | 1.2 |
| A80 | CB | N1 | 3.3 | 1.0 |
| C133 | O | N1 | 3.4 | 1.1 |
| C133 | C | N1 | 4.2 | 1.3 |
| F183 | CD2 | C3 | 4.3 | 1.3 |
| F183 | CE2 | C3 | 4.2 | 1.2 |
| F183 | CZ | C3 | 4.0 | 1.2 |
| F183 | CG | C3 | 4.3 | 1.3 |
| F183 | CE1 | C3 | 4.0 | 1.2 |
| F183 | CD1 | C3 | 4.1 | 1.2 |
| E131 | O | C3 | 3.5 | 1.1 |
| A80 | CB | C3 | 3.5 | 1.0 |
| F183 | CE2 | C4 | 4.0 | 1.2 |
| F183 | CZ | C4 | 3.8 | 1.1 |
| F183 | CE1 | C4 | 4.2 | 1.2 |
| L130 | CD1 | C4 | 4.1 | 1.2 |
| L130 | CB | C4 | 4.3 | 1.3 |
| A80 | CB | C4 | 3.9 | 1.2 |
| F183 | CE2 | C5 | 4.2 | 1.2 |
| F183 | CZ | C5 | 3.6 | 1.0 |
| F183 | CE1 | C5 | 4.0 | 1.2 |
| C67 | SG | C5 | 3.9 | 1.1 |
| A80 | CB | C5 | 4.1 | 1.2 |
| F183 | CZ | N6 | 3.6 | 1.1 |
| F183 | CE1 | N6 | 3.5 | 1.1 |
| C67 | SG | N6 | 3.5 | 1.0 |
| A80 | CB | N6 | 3.9 | 1.2 |
| F183 | CZ | C7 | 3.8 | 1.1 |
| F183 | CE1 | C7 | 3.3 | 1.0 |
| F183 | CD1 | C7 | 3.8 | 1.1 |
| C67 | SG | C7 | 4.2 | 1.2 |
| A80 | CB | C7 | 3.5 | 1.0 |
| C133 | O | C7 | 3.6 | 1.1 |
| F183 | CE1 | N2 | 3.4 | 1.0 |
| F183 | CD1 | N2 | 3.8 | 1.2 |
| A80 | CB | N2 | 4.2 | 1.3 |
| C133 | O | N2 | 2.8 | 0.9 |
| C133 | C | N2 | 4.0 | 1.2 |
| L59 | CD2 | N2 | 3.9 | 1.2 |
| F183 | CE1 | C8 | 3.9 | 1.1 |
| C133 | O | C8 | 3.6 | 1.1 |
| L59 | CD2 | C8 | 3.8 | 1.1 |
| L59 | CG | C8 | 4.2 | 1.2 |
| L59 | CB | C8 | 4.1 | 1.2 |
| R135 | CB | C9 | 4.4 | 1.3 |
| R135 | N | C9 | 4.1 | 1.3 |
| R134 | CA | C9 | 4.2 | 1.2 |
| C133 | O | C9 | 3.4 | 1.0 |
| C133 | C | C9 | 4.4 | 1.3 |
| L59 | CD2 | C9 | 3.9 | 1.1 |
| L59 | CG | C9 | 4.0 | 1.2 |
| L59 | CB | C9 | 4.3 | 1.2 |
| R135 | NH2 | O11 | 2.9 | 1.0 |
| R135 | NH1 | O11 | 3.4 | 1.1 |
| R135 | CZ | O11 | 3.5 | 1.1 |
| L59 | C | O11 | 3.5 | 1.1 |
| R135 | NH2 | C10 | 4.1 | 1.3 |
| R135 | CZ | C10 | 4.4 | 1.3 |
| L59 | CG | C10 | 4.1 | 1.2 |
| L59 | CB | C10 | 4.2 | 1.2 |
| R135 | NH2 | C11 | 3.4 | 1.1 |
| R135 | NH1 | C11 | 3.5 | 1.1 |
| R135 | CZ | C11 | 3.6 | 1.1 |
| L59 | C | C11 | 3.7 | 1.1 |
| L59 | CG | C11 | 4.4 | 1.3 |
| L59 | CB | C11 | 4.0 | 1.2 |
| L59 | O | C11 | 3.2 | 1.0 |
| L59 | CA | C11 | 4.3 | 1.2 |
| R135 | NH1 | C12 | 4.2 | 1.3 |
| F183 | CE1 | C12 | 4.3 | 1.2 |
| C67 | SG | C12 | 4.3 | 1.2 |
| L59 | C | C12 | 4.4 | 1.3 |
| L59 | CD2 | C12 | 4.4 | 1.3 |
| L59 | CG | C12 | 4.4 | 1.3 |
| L59 | CB | C12 | 3.8 | 1.1 |
| R135 | NH2 | C13 | 4.0 | 1.2 |
| R135 | NH1 | C13 | 3.3 | 1.0 |
| R135 | CZ | C13 | 3.8 | 1.1 |
| G60 | CA | C13 | 4.3 | 1.2 |
| G60 | N | C13 | 3.8 | 1.2 |
| L59 | C | C13 | 3.5 | 1.0 |
| L59 | CB | C13 | 3.8 | 1.1 |
| L59 | O | C13 | 3.4 | 1.1 |
| L59 | CA | C13 | 4.2 | 1.2 |

TABLE 11
PLK1 contact model (Quanta) for 4-[4-(4-methyl-2-methylamino-
thiazol-5-yl)-pyrimidin-2-ylamino]-phenol.

| | | Protein - |
|---|---|---|
| PLK1 | Residue | ligand atom |
| residue | atom | distance (Å) |
| L59 | O | 1.7 |
| L59 | HD23 | 2.9 |
| L59 | HB1 | 3.1 |
| L59 | HD23 | 2.9 |
| L59 | HB1 | 3.5 |
| L59 | HG | 3.3 |
| L59 | HD23 | 3.2 |
| L59 | O | 2.7 |
| L59 | HG | 3.3 |
| L59 | O | 3.2 |
| L59 | HB1 | 3.2 |
| L59 | HB1 | 2.7 |
| L59 | O | 3.4 |
| L59 | HB1 | 2.8 |
| L59 | HD23 | 3.0 |
| L59 | HD23 | 3.3 |
| L59 | HG | 3.5 |
| L59 | C | 2.7 |
| L59 | O | 1.7 |
| L59 | HA | 3.4 |
| L59 | HB1 | 3.1 |
| L59 | C | 3.2 |
| L59 | O | 3.2 |
| L59 | HB1 | 3.1 |
| G60 | N | 3.2 |
| G60 | CA | 3.4 |
| G60 | HA1 | 2.6 |
| F64 | HB2 | 2.9 |
| F64 | HB2 | 3.5 |
| F64 | CB | 3.4 |
| F64 | HB2 | 2.4 |
| F64 | HD1 | 3.2 |
| F64 | CB | 3.2 |
| F64 | HB1 | 3.0 |
| F64 | HB2 | 2.5 |
| C67 | HB1 | 3.3 |
| C67 | HB1 | 3.0 |
| C67 | CB | 3.3 |
| C67 | HB1 | 2.6 |
| C67 | HB2 | 3.4 |
| C67 | SG | 3.5 |
| C67 | HB1 | 3.0 |
| C67 | HB2 | 3.3 |
| A80 | CB | 3.3 |
| A80 | HB1 | 3.0 |
| A80 | HB2 | 3.1 |
| A80 | HB3 | 3.0 |
| A80 | CB | 3.5 |
| A80 | HB1 | 3.3 |
| A80 | HB3 | 2.9 |
| A80 | HB1 | 3.4 |
| A80 | HB3 | 3.4 |
| A80 | HB1 | 3.3 |
| A80 | HB1 | 3.0 |
| A80 | HB1 | 2.9 |
| A80 | HB2 | 3.4 |
| A80 | HB3 | 3.0 |
| K82 | HD1 | 3.0 |
| K82 | HD1 | 2.9 |
| K82 | HD1 | 3.1 |
| K82 | CD | 3.5 |
| K82 | CE | 3.4 |
| K82 | NZ | 3.4 |
| K82 | HZ2 | 2.7 |
| K82 | HB2 | 3.4 |
| K82 | HD1 | 2.7 |
| K82 | HE2 | 3.0 |
| K82 | HE2 | 3.3 |
| K82 | HZ2 | 3.4 |
| K82 | HE2 | 3.3 |
| K82 | HZ2 | 3.0 |
| K82 | HD1 | 3.4 |
| K82 | CD | 3.0 |
| K82 | HZ2 | 3.2 |
| K82 | HD1 | 2.2 |
| K82 | HD2 | 2.9 |
| K82 | HE2 | 3.0 |
| K82 | NZ | 3.1 |
| K82 | HZ2 | 2.6 |
| K82 | HZ3 | 2.9 |
| K82 | HE2 | 3.1 |
| V114 | HG12 | 2.9 |
| V114 | HG12 | 3.5 |
| L130 | HD13 | 3.3 |
| L130 | HD13 | 3.1 |
| L130 | HD22 | 3.2 |
| L130 | HB1 | 3.5 |
| L130 | HB1 | 3.3 |
| L130 | HD13 | 3.0 |
| L130 | HD22 | 3.1 |
| L130 | CG | 3.4 |
| L130 | CD1 | 2.8 |
| L130 | CD2 | 2.9 |
| L130 | HD13 | 2.3 |
| L130 | HD11 | 2.6 |
| L130 | HD22 | 2.3 |
| L130 | HD21 | 2.8 |
| L130 | HB1 | 3.1 |
| L130 | CD1 | 3.3 |
| L130 | HB1 | 2.8 |
| L130 | HD13 | 2.4 |
| L130 | HD22 | 2.8 |
| E131 | O | 3.5 |
| E131 | O | 2.9 |
| C133 | O | 1.8 |
| C133 | O | 3.4 |
| C133 | H | 3.5 |
| C133 | O | 2.8 |
| C133 | O | 3.4 |
| C133 | H | 3.5 |
| C133 | C | 3.0 |
| C133 | O | 1.8 |
| C133 | O | 2.6 |
| R134 | HA | 3.2 |
| R134 | CA | 3.3 |
| R134 | HA | 2.3 |
| R135 | HH22 | 2.0 |
| R135 | H | 3.2 |
| R135 | HB1 | 3.4 |
| R135 | CZ | 3.5 |
| R135 | NH1 | 3.4 |
| R135 | NH2 | 2.9 |
| R135 | HH11 | 2.8 |
| R135 | HH22 | 2.0 |
| R135 | H | 3.5 |
| R135 | NH2 | 3.4 |
| R135 | HH11 | 3.1 |
| R135 | HH22 | 2.8 |
| R135 | NH1 | 3.3 |
| R135 | HH11 | 2.9 |
| R135 | H | 2.9 |
| R135 | H | 3.5 |
| R135 | HH11 | 3.3 |
| R135 | HH22 | 2.8 |
| R135 | NH1 | 2.9 |
| R135 | HH12 | 3.2 |
| R135 | HH11 | 2.4 |
| F183 | HZ | 3.5 |
| F183 | HZ | 3.3 |
| F183 | HE1 | 3.2 |
| F183 | HZ | 3.4 |
| F183 | CE1 | 3.3 |
| F183 | HE1 | 3.0 |
| F183 | CE1 | 3.4 |
| F183 | HE1 | 2.8 |
| F183 | HE1 | 2.9 |
| F183 | HE1 | 3.3 |
| F183 | HE2 | 3.1 |
| F183 | HD1 | 3.3 |
| F183 | HE1 | 3.2 |
| F183 | HE1 | 3.4 |
| G193 | HA2 | 3.3 |
| D194 | OD2 | 3.5 |
| D194 | OD1 | 3.1 |
| D194 | OD2 | 3.4 |
| D194 | H | 3.2 |
| D194 | CG | 2.9 |
| D194 | OD1 | 2.5 |
| D194 | OD2 | 2.6 |
| D194 | N | 3.0 |
| D194 | H | 2.7 |
| D194 | H | 3.4 |
| D194 | N | 3.1 |
| D194 | CA | 3.3 |
| D194 | CG | 3.0 |
| D194 | OD1 | 2.6 |
| D194 | H | 2.7 |
| D194 | HA | 2.7 |

TABLE 12
In vitro activity of flavonoid compounds

| | | | PI3K | CDK2 |
|---|---|---|---|---|
| No | Inhibitor | Plk1 IC₅₀ (μM) | IC50 (μM) | IC50 (μM) |
| 1 | Wortmannin | 0.18 ± 0.1 | 0.0042 | >10 |
| 2 | Staurosporine | 0.8 ± 0.2 | 9 | 0.004 |
| 3 | Purvalanol A | 5 | ND | 0.0009 ± 0.002 |
| 4 | LY2940002 | 9.33 ± 3.7 | 1.4 | ND |
| 5 | Quercetin | 64.25 ± 24 | 3.8 | ND |

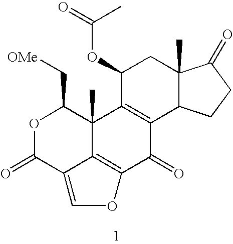

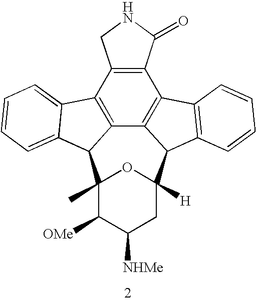

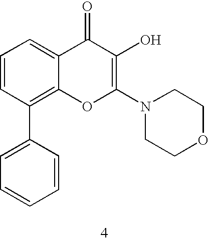

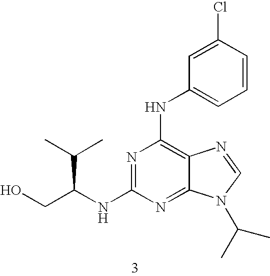

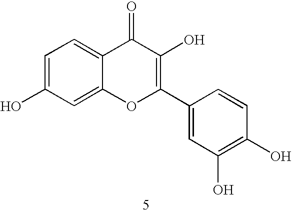

TABLE 13
In vitro potencies for flavonoid compounds.

| Structure | | | | | | | | |
|---|---|---|---|---|---|---|---|---|
| 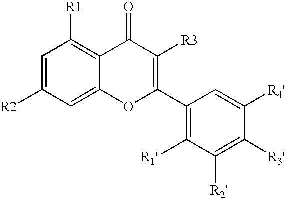 | | | | | | | | Kinase inhibition (μM) |
| | R¹ | R² | R³ | R1′ | R2′ | R3′ | R4′ | Plk1 IC₅₀ (μM) |
| Morin Hydrate | OH | OH | OH | OH | H | OH | H | 12.6 ± 1.4 |
| Datescefin | OH | OH | OH | OH | H | H | H | >100 |
| Quercetin | OH | OH | OH | H | OH | OH | H | 64.25 ± 24 |
| Myricetin | OH | OH | OH | H | OH | OH | OH | >100 |
| Kaempferol | OH | OH | OH | H | H | OH | H | >100 |
| Luteolin | OH | OH | H | H | OH | OH | H | >100 |
| Galangin | OH | OH | OH | H | H | H | H | >100 |
| Robinetin | H | OH | OH | H | OH | OH | OH | 60 |
| Daidzein | H | OH | H | H | H | OH | H | >100 |
| Fisetin | H | OH | OH | H | OH | OH | H | >100 |
| Kaempferide | OH | OH | OH | H | H | Ome | H | >100 |

TABLE 14
In vitro testing of PKA inhibitors

| Compound | PKA (IC50, μM) | Plk1 (IC50, μM) |
|---|---|---|
| Balanol | 0.003, 0.004* | >200 |
| H89 | 0.048** | >500 |
| A3 | 11** | >500 |
| puravalanol A | >100 | 10 |
| 4-Cyano-3-methylisoquinoline | 0.030** | >500 |
| KT5720 | 0.056** | >500 |

1. A method of screening for a modulator of PLK, wherein the method comprises using the structure co-ordinates of Table 2, or a portion thereof.

2. The method according to claim 1 comprising the steps of:
(a) employing the structure co-ordinates of Table 2, or the portion thereof, to design, select or synthesise a putative modulator of PLK;
(b) contacting the putative modulator of PLK with PLK or a mutant, variant, homologue, derivative or fragment thereof, in the presence of a substrate of PLK; and
(c) determining whether said putative modulator of PLK modulates PLK.

3. The method according to claim 1 or claim 2 wherein at least one of the structure co-ordinates of Table 2, the portion thereof, the putative modulator of PLK and the substrate are provided on a machine-readable data storage medium comprising a data storage material encoded with machine readable data.

4. The method according to claim 2 or claim 3 wherein the putative modulator of PLK is selected from a library of compounds.

5. The method according to claim 2 or claim 3 wherein the putative modulator of PLK is selected from a database.

6. The method according to claim 2 or claim 3 wherein the putative modulator of PLK is designed de novo.

7. The method according to claim 2 or claim 3 wherein the putative modulator of PLK is designed from a known PLK modulator.

8. The method according to claim 2 or claim 3 wherein the design or selection of the putative modulator of PLK is performed in conjunction with computer modelling.

9. The method according to any preceding claim wherein the putative modulator of PLK inhibits PLK activity.

10. The method according to any preceding claim wherein the PLK is PLK1.

11. The method according to any preceding claim wherein the putative modulator of PLK is useful in the prevention and/or treatment of a PLK related disorder.

12. The method according to claim 11 wherein the PLK related disorder is a proliferative disorder.

13. The method according to claim 12 wherein the proliferative disorder is selected from the group consisting of cancer, leukemia, glomerulonephritis, rheumatoid arthritis, psoriasis and chronic obstructive pulmonary disorder.

14. An assay for identifying a candidate compound capable of modulating PLK, said assay comprising the steps of:
(a) contacting said candidate compound with PLK; and
(b) detecting whether said candidate compound forms associations with one or more amino acid residues corresponding to PLK amino acid residues L59, G60, A65, C67, A80, K82, L130, E131, C133, R135, F183 and D194.

15. The assay according to claim 14 wherein said candidate compound is selected by performing rational drug design with a 3-dimensional model of PLK in conjunction with computer modelling.

16. The assay according to claim 14 which comprises detecting whether said candidate compound forms an association with the amino acid residue corresponding to PLK amino acid residue C67.

17. A method of identifying a candidate compound capable of modulating PLK, comprising performing an assay using a compound selected from the following:
(i) 5′-thioadenosine, or a derivative thereof;
(ii) staurosporine, wortmannin, purvalanol A, LY294002, quercetin, morin hydrate, or derivatives thereof; and
(iii) 4-[4-(4-methyl-2-methylaminothiazol-5-yl)-pyrimidin-2-ylamino]-phenol; 4-[4-(2,4-dimethyl-thiazol-5-yl)-pyrimidin-2-ylamino]-phenol or 4-[4-(2-amino-4-methyl-thiazol-5-yl)-pyrimidin-2-ylamino]-phenol;
or a pharmaceutically acceptable salt thereof.

18. The method of claim 17 wherein the assay is a competitive binding assay.

19. The method of claim 17 wherein the assay comprises contacting the candidate compound with PLK in the presence of the compound selected from:
(i) 5′-thioadenosine, or a derivative thereof;
(ii) staurosporine, wortmannin, purvalanol A, LY294002, quercetin, morin hydrate, or derivatives thereof; and
(iii) 4-[4-(4-methyl-2-methylaminothiazol-5-yl)-pyrimidin-2-ylamino]-phenol, 4-[4-(2,4-dimethyl-thiazol-5-yl)-pyrimidin-2-ylamino]-phenol or 4-[4-(2-amino-4-methyl-thiazol-5-yl)-pyrimidin-2-ylamino]-phenol;
or a pharmaceutically acceptable salt thereof, and detecting any change in the interaction between (i), (ii) or (iii) and PLK.

20. A PLK modulator identified by the method of any one of claim 1 to 13, or a candidate compound identified by the assay according to any one of claims 14 to 19.

21. The PLK modulator or candidate compound according to claim 20 wherein the PLK modulator or candidate compound inhibits PLK activity.

22. The PLK modulator or candidate compound according to claim 20 or claim 21 wherein the PLK modulator or candidate compound is capable of forming a covalent bond with the amino acid residue corresponding to PLK amino acid residue C67.

23. The PLK modulator or candidate compound according to claim 22 wherein the PLK modulator or candidate compound is capable of forming a disulfide bond with the thiol group of the amino acid residue corresponding to PLK amino acid residue C67.

24. The PLK modulator or candidate compound according to claim 20 wherein the PLK modulator or candidate compound is an irreversible antagonist.

25. A pharmaceutical composition comprising the PLK modulator or candidate compound according to any one of claims 20 to 24 and a pharmaceutically acceptable carrier, diluent, excipient, adjuvant or any combination thereof.

26. A method of preventing and/or treating a PLK related disorder in a subject, comprising administering to said subject a PLK modulator or candidate compound according to any one of claims 20 to 24 and/or a pharmaceutical composition according to claim 25.

27. The method according to claim 26 wherein the PLK modulator or candidate compound is selected from the following:
(i) 5′-thioadenosine, or a derivative thereof;
(ii) staurosporine, wortmannin, purvalanol A, LY294002, quercetin, morin hydrate, or derivatives thereof; and
(iii) 4-[4-(4-methyl-2-methylaminothiazol-5-yl)-pyrimidin-2-ylamino]-phenol, 4-[4-(2,4-dimethyl-thiazol-5-yl)-pyrimidin-2-ylamino]-phenol or 4-[4-(2-amino-4-methyl-thiazol-5-yl)-pyrimidin-2-ylamino]-phenol;
or a pharmaceutically acceptable salt thereof.

28. (canceled)

29. The method according to claim 27, wherein the PLK related disorder is cancer.

30-32. (canceled)

33. A computer for producing a three-dimensional representation of PLK wherein said computer comprises:
(a) a computer-readable data storage medium comprising a data storage material encoded with computer-readable data, wherein said data comprises the structure co-ordinates of Table 2;
(b) a working memory for storing instructions for processing said computer-readable data;
(c) a central-processing unit coupled to said working memory and to said computer-readable data storage medium for processing said computer-machine readable data into said three-dimensional representation; and
(d) a display coupled to said central-processing unit for displaying said three-dimensional representation.

34. A machine-readable data storage medium comprising a data storage material encoded with machine readable data, wherein the data is defined by at least a portion of the structure co-ordinates of Table 2.

35. A method of predicting the structure and/or function of potential modulators of PLK, comprising using the computer of claim 33 or the machine readable data storage medium of claim 34.

36. (canceled)

37. A method of solving the crystalline form structure of a protein with significant amino acid sequence homology to a functional domain of PLK, comprising using at least a portion of the structure co-ordinates of Table 2.

38. The method of claim 37 wherein the method comprises molecular replacement.

39. A method of designing, selecting and synthesizing modulators of PLK, comprising using at least a portion of the structure co-ordinates of Table 2 in molecular design techniques.

40. A method of developing compounds that can isomerise to reaction intermediates in the chemical reaction of a substrate and PLK-binding compound, comprising using at least a portion of the structure co-ordinates of Table 2.

41. A method of screening small molecule databases for chemical entities or compounds that modulate PLK, comprising using at least a portion of the structure co-ordinates of Table 2.

42. The method of claim 27, wherein the disorder is a proliferative disorder.

43. The method of claim 42, wherein the PLK modulator or candidate compound inhibits PLK.

44. (canceled)

45. The method according to claim 27 wherein the PLK related disorder is a disorder associated with increased PLK activity.

46. (canceled)

47. A method of inhibiting PLK in a cell comprising contacting said cell with a compound selected from the following:
(i) 5′-thioadenosine, or a derivative thereof;
(ii) staurosporine, wortmannin, purvalanol A, LY294002, quercetin, morin hydrate, or derivatives thereof; and
(iii) 4-[4-(4-methyl-2-methylaminothiazol-5-yl)-pyrimidin-2-ylamino]-phenol, 4-[4-(2,4-dimethyl-thiazol-5-yl)-pyrimidin-2-ylamino]-phenol or 4-[4-(2-amino-4-methyl thiazol-5-yl)-pyrimidin-2-ylamino]-phenol;
or a pharmaceutically acceptable salt thereof, such that PLK is inhibited in said cell.

48. The method according to claim 47 wherein the cell is a cancer cell.

49. A fragment of PLK, or a homologue, mutant, or derivative thereof, comprising a ligand binding domain, said ligand binding domain being defined by the amino acid residue structural coordinates selected from one or more of the following: L59, G60, A65, C67, A80, K82, L130, E131, C133, R135, F183 and D194.

50. A fragment of PLK, or a homologue, mutant or derivative thereof, according to claim 49 which corresponds to a portion of the structure co-ordinates of Table 2.

51. A method of identifying a candidate compound capable of modulating PLK, comprising performing an assay using the fragment of PLK, or the homologue, mutant, or derivative thereof, according to claim 49.

52-55. (canceled)


SEQUENCE LISTING (8 sequences)

SEQ ID NO: 1 - protein, 19 aa, Artificial Sequence
  Description of Artificial Sequence Synthetic peptide
MSYYHHHHHHGMASDDDDK

SEQ ID NO: 2 - protein, 6 aa, Artificial Sequence
  Description of Artificial Sequence Synthetic 6x His tag
HHHHHH

SEQ ID NO: 3 - protein, 10 aa, Artificial Sequence
  Description of Artificial Sequence Synthetic peptide
RRREEETEEE

SEQ ID NO: 4 - protein, 603 aa, Homo sapiens
MSAAVTAGKLARAPADPGKAGVPGVAAPGAPAAAPPAKEIPEVLVDPRSRRRYVRGRFLGKGGFAKCFEISDADTKEVFAGKIVPKSLLLKPHQREKMSMEISIHRSLAHQHVVGFHGFFEDNDFVFVVLELCRRRSLLELHKRRKALTEPEARYYLRQIVLGCQYLHRNRVIHRDLKLGNLFLNEDLEVKIGDFGLATKVEYDGERKKTLCGTPNYIAPEVLSKKGHSFEVDVWSIGCIMYTLLVGKPPFETSCLKETYLRIKKNEYSIPKHINPVAASLIQKMLQTDPTARPTINELLNDEFFTSGYIPARLPITCLTIPPRFSIAPSSLDPSNRKPLTVLNKGLENPLPERPREKEEPVVRETGEVVDCHLSDMLQQLHSVNASKPSERGLVRQEEAEDPACIPIFWVSKWVDYSDKYGLGYQLCDNSVGVLFNDSTRLILYNDGDSLQYIERDGTESYLTVSSHPNSLMKKITLLKYFRNYMSEHLLKAGANITPREGDELARLPYLRTWFRTRSAIILHLSNGSVQINFFQDHTKLILCPLMAAVTYIDEKRDFRTYRLSLLEEYGCCKELASRLRYARTMVDKLLSSRSASNRLKAS

SEQ ID NO: 5 - protein, 685 aa, Homo sapiens
MELLRTITYQPAASTKMCEQALGKGCGADSKKKRPPQPPEESQPPQSQAQVPPAAPHHHHHHSHSGPEISRIIVDPTTGKRYCRGKVLGKGGFAKCYEMTDLTNNKVYAAKIIPHSRVAKPHQREKIDKEIELHRILHHKHVVQFYHYFEDKENIYILLEYCSRRSMAHILKARKVLTEPEVRYYLRQIVSGLKYLHEQEILHRDLKLGNFFINEAMELKVGDFGLAARLEPLEHRRRTICGTPNYLSPEVLNKQGHGCESDIWALGCVMYTMLLGRPPFETTNLKETYRCIREARYTMPSSLLAPAKHLIASMLSKNPEDRPSLDDIIRHDFFLQGFTPDRLSSSCCHTVPDFHLSSPAKNFFKKAAAALFGGKKDKARYIDTHNRVSKEDEDIYKLRHDLKKTSITQQPSKHRTDEELQPPTTTVARSGTPAVENKQQIGDAIRMIVRGTLGSCSSSSECLEDSTMGSVADTVARVLRGCLENMPEADCIPKEQLSTSFQWVTKWVDYSNKYGFGYQLSDHTVGVLFNNGAHMSLLPDKKTVHYYAELGQCSVFPATDAPEQFISQVTVLKYFSHYMEENLMDGGDLPSVTDIRRPRLYLLQWLKSDKALMMLFNDGTFQVNFYHDHTKIIICSQNEEYLLTYINEDRISTTFRLTTLLMSGCSSELKNRMEYALNMLLQRCN

SEQ ID NO: 6 - protein, 646 aa, Homo sapiens [sequence omitted]

SEQ ID NO: 7 - protein, 326 aa, Homo sapiens
MSAAVTAGKLARAPADPGKAGVPGVAAPGAPAAAPPAKEIPEVLVDPRSRRRYVRGRFLGKGGFAKCFEISDADTKEVFAGKIVPKSLLLKPHQREKMSMEISIHRSLAHQHVVGFHGFFEDNDFVFVVLELCRRRSLLELHKRRKALTEPEARYYLRQIVLGCQYLHRNRVIHRDLKLGNLFLNEDLEVKIGDFGLATKVEYDGERKKTLCGTPNYIAPEVLSKKGHSFEVDVWSIGCIMYTLLVGKPPFETSCLKETYLRIKKNEYSIPKHINPVAASLIQKMLQTDPTARPTINELLNDEFFTSGYIPARLPITCLTIPPRFS

SEQ ID NO: 8 - protein, 320 aa, Homo sapiens
MGNAAAAKKGSEQESVKEFLAKAKEDFLKKWESPAQNTAHLDQFERIKTLGTGSFGRVMLVKHKETGNHYAMKILDKQKVVKLKQIEHTLNEKRILQAVNFPFLVKLEFSFKDNSNLYMVMEYVPGGEMFSHLRRIGRFSEPHARFYAAQIVLTFEYLHSLDLIYRDLKPENLLIDQQGYIQVTDFGFAKRVKGRTWTLCGTPEYLAPEIILSKGYNKAVDWWALGVLIYEMAAGYPPFFADQPIQIYEKIVSGKVRFPSHFSSDLKDLLRNLLQVDLTKRFGNLKNGVNDIKNHKWFATTDWIAIYQRKVEAPFIPKFK